(19) United States
(12) Patent Application Publication
(10) Pub. No.: US 2025/0160971 A1
(43) Pub. Date: May 22, 2025

(54) METHOD FOR MULTI-SYSTEM INTERACTION

(71) Applicant: Cilag GmbH International, Zug (CH)

(72) Inventors: Frederick E. Shelton, IV, Hillsboro, OH (US); Jason L. Harris, Lebanon, OH (US); Jacqueline Corrigan Aronhalt, Loveland, OH (US); Charles J. Scheib, Loveland, OH (US); Kevin M. Fiebig, Cincinnati, OH (US); Taylor W. Aronhalt, Loveland, OH (US); Anthony John Fernandez, Dublin, CA (US); James T. Spivey, Whitehouse Station, NJ (US); Luis Andrade Baez, JR., La Honda, CA (US); Matthew David Cowperthwait, Cincinnati, OH (US); Nicholas James Ross, Franklin, OH (US); Shane R. Adams, Lebanon, OH (US); Chad E. Eckert, Terrace Park, OH (US)

(21) Appl. No.: 18/810,323

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 34/00; G06V 10/74; G06V 20/50; G06V 30/19; G16H 40/63

(52) U.S. Cl. CPC A61B 34/25; G06V 10/74; G06V 20/50; G06V 30/19; G16H 40/63; A61B 2034/256; G06V 2201/034

(57) ABSTRACT

Systems, methods, and instrumentalities are disclosed for displaying a magnitude and location of a resulting tissue tension from an applied force to an instrument and a resulting tissue reaction. A surgical stapling device for monitoring and adapting to tissue tension during a surgical stapling procedure may measure strain on at least one component of the surgical stapling device resultant from one or more of an opposed force applied by a user and a force resisted by tissue associated with the tissue tension. The device may determine a location of the tissue tension based on historical surgical data, the opposed force, and the resisted force. The device may highlight the location of the tissue tension to aid in real-time surgical decision-making.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023.
Provisional U.S. Patent Application No. 63/602,028, filed Nov. 22, 2023
Provisional U.S. Patent Application No. 63/601,998, filed Nov. 22, 2023
Provisional U.S. Patent Application No. 63/602,003, filed Nov. 22, 2023.
Provisional U.S. Patent Application No. 63/602,006, filed Nov. 22, 2023
Provisional U.S. Patent Application No. 63/602,011, filed Nov. 22, 2023
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

This application is related to the following, filed contemporaneously, the contents of each of which are incorporated by reference herein:
U.S. patent application Ser. No. 18/810,036, filed Aug. 20, 2024,
U.S. patent application Ser. No. 18/810,082, filed Aug. 20, 2024,
U.S. patent application Ser. No. 18/810,890, filed Aug. 20, 2024,
U.S. patent application Ser. No. 18/810,133, filed Aug. 20, 2024,
U.S. patent application Ser. No. 18/810,170, filed Aug. 20, 2024,
U.S. patent application Ser. No. 18/810,208, filed Aug. 20, 2024,
U.S. patent application Ser. No. 18/810,230, filed Aug. 20, 2024,
U.S. patent application Ser. No. 18/810,266, filed Aug. 20, 2024,
U.S. patent application Ser. No. 18/810,283, filed Aug. 20, 2024,
U.S. patent application Ser. No. 18/810,960, filed Aug. 20, 2024, and
U.S. patent application Ser. No. 18/810,041, filed Aug. 20, 2024.

### BACKGROUND

Surgical procedures are typically performed in surgical operating theaters or rooms in a healthcare facility such as, for example, a hospital. Various surgical devices and systems are utilized in performance of a surgical procedure. In the digital and information age, medical systems and facilities are often slower to implement systems or procedures utilizing newer and improved technologies due to patient safety and a general desire for maintaining traditional practices.

### SUMMARY

Within healthcare, systems may facilitate an environment conducive to medical practices. A device may enable interaction, coordination, and control among one or more smart and/or legacy system. By implementing algorithms (e.g., dynamic algorithms) and methodologies, the device may adapt system behavior based on variables, conditions, and/or parameters. The operation of the device and interaction between the device, smart system, and/or legacy system may affect (e.g., enhance) the collective performance of the device, smart system, and/or legacy smart system.

The device may include a decision-making mechanism that ascertains whether and how two or more systems may interact (e.g., under varying circumstances). The decisions consider variables, and the variables may include the systems' capacities to cooperate, considerations for data exchange, interrelationships of variables, and prioritization of patient or surgeon parameters (e.g., patient or surgeon needs). When the device recognizes the interdependence of closed-loop variables, the device may transition from a state of cooperation to a state of bidirectional open-loop communication (e.g., in order to safeguard system stability and patient safety).

The device may prevent system instability and predictability failures (e.g., non-correlated predictability failures). Real-time data related to patient conditions and system parameters may be used, and the real-time data of the interaction level between the systems may be adjusted. The device may identify and adapt to an instability cascade failure involving a patient monitoring smart system, a ventilation/sedation system, and/or a heating system. The device may manage non-correlated predictability failures by switching from global control to local control based on a comparison of an energy input rate and a heat bloom expansion rate.

The device may engage with legacy systems. The device may identify features compatible with the legacy system (e.g., employing various sensors and cameras), such as a USB port and/or a wireless connection, and guide a user (e.g., surgeon, operating room (OR) staff to connect the two). The device may control the legacy system or display data from the legacy system on an interface, affecting the user's ability to monitor and/or control a situation arising in the operating room or within a hospital environment.

The device may be integrated with interconnected medical technologies and/or platforms. The user controls of one system may be displayed on the device, imaging and control interfaces between systems may be transferred to the device, synchronized motion of multiple devices may be executed, cooperative interactions among devices may be executed and/or initiated.

For example, discovery of smart surgical systems that support image porting and remote control may be performed (e.g., by smart surgical systems in an operating room). A first surgical system may determine that a second surgical system supports image porting and remote control. A first surgical system may receive a request (e.g., second surgical system may send a request) associated with redirection of imaging and a control interface from the first surgical system to the second surgical system (e.g., remote control surgical system). The second surgical system (e.g., based on the request) may receive imaging and indication of controls (e.g., full control or partial control) associated with the first surgical system. The second surgical system may display imaging from the first surgical system and the control interface of the first surgical system (e.g., based on a received set of operational controls (OCs) that the second surgical system is permitted/enabled to change/modify). The second surgical system may request a control setting change associated with the OC of the first surgical system. The first surgical system may determine whether to validate the control setting change.

In examples, the first surgical system may validate the control setting change. The first surgical system may change the control setting (e.g., operating configuration). The first surgical system may send an acknowledgment to the second surgical system indicating the control setting change. The first surgical system may send an additional (e.g., updated) set of OCs that the second surgical system is enabled (e.g., permitted) to change. The second surgical system may display updated imaging from the first surgical system and an updated control interface of the first surgical system based on the received set of OCs that it is permitted to change. In examples, the first surgical system may determine to reject the requested control setting change. The first surgical system may send a NACK and/or a reason for NACK to the second surgical system. The second surgical system may update (e.g., remove) control settings based on the NACK.

In examples, the first surgical system may determine to terminate remote control of the first surgical system by the second surgical system. The first surgical system may evaluate and/or monitor OCs that are set based on the control settings. The first surgical system may monitor data (e.g., patient biomarker data), to determine control settings. The first surgical system may terminate remote control, for example, based on the patient biomarker data. The first surgical system may send a notification that indicates that the first surgical system is taking control. The second surgical system may update (e.g., remove) the control settings and/or imaging based on the received notification.

In operating rooms, multiple surgical devices may operate in close proximity to one another. In addition, the devices may all be from different manufacturers and may have different control systems. The devices may not be aware of the presence of other devices. Even if the devices are aware of other devices, the devices may not be able to communicate to coordinate their actions. This lack of coordination may cause the surgical devices to become entangled with each other and, in the worst case scenario, injure a patient.

Feature(s) described herein relate to techniques for synchronized motion between surgical devices to manage the interaction between them. For example, multiple devices (e.g., which may have different manufacturers and/independent control systems) may actively synchronize their motions. A user may have simultaneous hybrid control of multiple separate instruments controlled by two independent smart systems. The user may, for example, control the instruments from a single control station.

A device may determine its movements based on movement of another device. For example, a first device may be actively controlled by the user and a second device may be put into a “follow-me” mode in which the second device maintains a certain proximity to the moving first device. The interdependent motions may have limits that are derived from each other. For example, two devices may be configured to maintain a tissue tension. In this case, if a user manually increases the force a first device is applying to the tissue, a second device that is also in contact with the tissue may autonomously reduce the force applied by the second device so that the overall tissue tension remains relatively stable. Similarly, hybrid load-stroke and/or a proportionate, integral, derivative (PID) control loop may be used to maintain the relationship between devices.

In another example, a first device may be put into a “station-keeping” or “position-holding” mode in which the first device maintains its absolute location in a global reference plane. A user may therefore know where the first device is at all times because the location is constant. This may allow the user to move a second device in the vicinity of the first device without causing an unwanted interaction between the devices.

In operating rooms, multiple surgical devices may operate in close proximity to one another. In addition, the devices may all be from different manufacturers and may have different control systems. The devices may not be aware of the presence of other devices. Even if the devices are aware of other devices, the devices may not be able to communicate to coordinate their actions. This lack of coordination may cause the surgical devices to become entangled with each other and, in the worst case scenario, injure a patient.

Feature(s) described herein relate to the synchronization of surgical device operational envelopes. In this case, although the precise movements of different devices are not synchronized, the devices may maintain synchronized operational areas, so as to avoid unwanted interaction between the devices. The shape or location of an operational envelope may be altered. For example, the operational envelope of a first device may change based on a user actively modifying the operational envelope of second device and/or based on the second device's movement. A (pre)defined balance between the two operational envelopes may be maintained by altering one when the other is modified (e.g., by the active control of the user). The operational envelopes may be synchronized by changing the loci of actions or functional limits of a first system based on the movements of a second (e.g., autonomous) system.

In an example, two robotic arms may be operating in the same area of a patient. To avoid the robotic arms becoming tangled, the area in which each arm is able to move may be bounded. In another example, the robotic arms may be configured to maintain at least a minimum distance from one another. In yet another example, the robotic arms may communicate with one another to negotiate for space (e.g., if one arm needs to move into the operational envelope of the other to perform a step in a surgical procedure).

In operating rooms, multiple surgical imaging devices may operate in close proximity to one another. In addition, the imaging devices may all be from different manufacturers and may have different control systems. The imaging devices may not be aware of the presence of other devices. Even if the devices are aware of other devices, the devices may not be able to communicate to coordinate their actions. This lack of coordination may limit the field of view of a user (e.g., surgeon). This limited visibility may cause the user to miss important events during surgery, such as an unintended bleed.

Synchronized imaging of two system may be used to maintain a common field-of-view or perspective for both systems. The synchronized imaging may allow a user to seamlessly transition objects from one field of view to another. Synchronized visualization may involve synchronized motion of the cameras. Synchronized visualization may involve electronic and/or algorithmic field-of-view limiting and/or overlapping imaging to enable each camera to capture a larger field of view than originally possible. The two systems may produce a composite image by adapting the synchronized imaging arrays.

Multiple scopes may use synchronized motion to maintain a relational field-of-view. For example, independent imaging scopes may use couple motion (e.g., the movement of one scope initiates movement of the second scope to maintain the coupled field-of-view of the two scopes). The coupled motion of the two scope may be maintained while the scopes exist in separate anatomic spaces (e.g., on either side of a tissue barrier, such as an organ wall) but are focused on the same tissue in between the scopes. The couple motion may be used when the two scopes are in the same space focused on the same field of view. In this case, the two scopes may cooperatively maintain a field of view that is larger than either scope is capable of capturing independently. A composite image may be created to display the larger field of view to a user. Synchronized motion may be used to maintain the spacing between the scopes to maintain the overall field of view.

In operating rooms, multiple surgical imaging devices may operate in close proximity to one another. An imaging device may have a sensor that tracks the location of the imaging device. Other devices in the operating room may create electromagnetic fields that affect the accuracy of the sensor's ability to track the imaging device. The devices may not be aware of the presence of other devices. Even if the devices are aware of other devices, the devices may not be able to communicate information such as information related to electromagnetic distortion. Without this knowledge, a user (e.g., surgeon) may not know the actual location of an imaging device, which may affect the user's ability to safely perform the operation.

To enable such devices to detect and compensate for distortion, a common reference plane may be created for multiple imaging streams. A reference plane from a first imaging system may be used as a means to compensate for distortion (e.g., electromagnetic distortion) of coordinates by a second imaging system. Multiple oblique reference planes may be aligned and distortion compensation may be performed for at least one of the sensed locations. A first coordinate system may be derived from real-time measurements from a sensor. The distortion compensation may use a second coordinate system originating from an independent system to form the basis for the common coordinate system for both local reference planes.

The first coordinate system may be used to determine the current location of a flexible endoscope distal end. The first coordinate system may accumulate additive errors due to distortion of the measurement. The first and second coordinate systems may be aligned by associating the first system with the second system and compensating for the distortion caused by the second system's measurements (e.g., relative to the first imagine system's detector). The two systems may be aligned using local re-calibration of the flexible endoscope. The distortion correction may involve measuring the first sensor location and the current field distortion measured by at least one other separate sensor (e.g., a redundant sensor). The redundant sensor may be located at a distance from the first sensor. The distance between the sensors may be greater than the size of the patient.

Within healthcare, systems may facilitate an environment conducive to medical practices. A first system may interact and/or coordinate with one or more other system(s). Shared object registration may enable the systems to identify common objects in the systems' respective fields of view. One system may register surgical structures in its field of view and share the registration information with another system.

Systems utilizing shared object registration may have different reference planes (e.g., independent local reference planes). For example, the systems may include respective surgical scopes that are on opposite sides of a tissue barrier (e.g., an organ wall). As a result, the first system may view objects in different locations and/or at different angles than the second system. To enable the systems to accurately use the shared object registrations, the independent local reference planes may need to be aligned with each other.

A first system may have pre-operative imaging and a second system may have intra-operative imaging. The patient may be in a different position in the pre-operative imaging compared to the intra-operative imaging. The change in patient position may cause surgical structures (e.g., organs, tumors, etc.) to shift, thereby exacerbating the differences between the two imaging systems. The shared object registration may enable the systems to use non-moving or less affected objects as baseline landmarks for aligning other surgical structures.

During an operation, a patient may receive therapeutic treatment. The treatment may have unintended primary and collateral effects on the patient's body. Without a method to monitor these effects, a surgeon may not be able to take remedial action in a timely manner.

Feature(s) described herein relate to controlling the boundaries and/or limitations of treatment systems to mitigate or prevent such unintended effects. For example, a first system may be providing a therapeutic treatment, and a second system may be monitoring the effects of the treatment. The controlled interaction between the two systems may allow a surgeon to adjust the treatment (e.g., the location or magnitude of the treatment) if needed.

A user may systematically coordinate the position and/or magnitude of the applied therapeutic treatment to control the shape of the primary and collateral effects of the treatment. For example, the user may control the location and/or operational parameter(s) of the therapy modality to define a three-dimensional therapeutic envelope of primary effect and secondary collateral interaction. Balancing the positional control and therapy effect may allow the user to control the primary and collateral envelope zones. An example intended primary effect may be a control percentage of cell death in a primary treatment zone. An example intended collateral effect may be an intended amount of cellular damage (e.g., but not cellular death) in a collateral treatment zone. The size and/or shape of the relational envelopes of the primary and collateral effects may be adjusted as needed.

During an operation, multiple devices may have an effect on a patient. The devices may impact the functionality of other devices as a result. For example, two separate devices may contribute to a negative feedback loop that lowers the patient's core temperature indefinitely, which will harm the patient if left unchecked. The devices may have no knowledge of each other or the effects each has on the other or the patient. The devices may therefore be incapable of correcting the negative feedback loop.

A first system may apply conditional restrictions on its function or operation based on the function or operation of a second system. Conditional bounding of a first system may be based on the monitoring from the second system. For example, a first system may determine to not use its full operational capabilities based on information from a second system that relates to the first system's behavior.

The first system may include a control system for monitoring and controlling the operation of the first system, and the second system may include an independent control system for monitoring and controlling the operation of the second system. The second system may monitor at least one parameter that is relevant to the operation of the first system. The first system may not be monitoring the parameter(s). The second system may communicate with the first system to provide the first system with access to the data collected by the second system. The first system may use the information to alter operational bounding of the first system operation.

The systems may use directional synchronization to control the effect of the systems' operations on a physiologic parameter of the patient (e.g., when the physiological parameter is out of pre-established bounds). In some examples, predefined upper and/or lower bounds may not be used. Instead, the systems may use the outcome of the system operations as a metric of whether to limit the operations. For example, if the system operations are adjusted and result in better performance/impact on the patient, allow the adjustment. Similarly, if the system operations result in undesired behavior/impact, limit the operation to reduce the undesired consequences.

Systems, methods, and instrumentalities associated with inter-connectivity of data flows between various surgical devices and/or systems are disclosed. The surgical devices and/or surgical systems may be interrelated or independent smart surgical devices and/or surgical systems. Data sourced by a first surgical device/system may be communicated to and/or accessible by a second surgical device/system for interactive use and storage. The data exchange may be bi-directional or unidirectional, which, for example, may enable the surgical devices/systems to interface and use each other's data. The data exchange may affect one or multiple surgical device/system's operation.

For example, a first surgical system may operate using a first operation configuration (e.g., first operation configuration parameters). The first surgical system may determine capability information associated with a surgical environment (e.g., operating room (OR). The surgical environment may include surgical systems (e.g., surgical hub, surgical devices, etc.). The capability information may include information associated with what a surgical system may be capable of generating and/or providing. The first surgical system may receive first data and associated metadata (e.g., first metadata) from a second surgical system. The first metadata may indicate whether the first data is control data or response data (e.g., which portion of the first data is control data or response data). The first surgical system may select an operation configuration (e.g., operation configuration parameter) based on the first data and/or first metadata. For example, the first surgical system may determine the first operation configuration if the first data is response data. The first surgical system may determine the second operation configuration if the first data is control data. The first surgical system may generate second data based on the determined operation configuration. The first surgical system may determine data packages (e.g., including at least a portion of the second data) to send to target systems. The data packages may indicate whether the data in the data package is control data or response data.

For example, the first surgical system may determine capability information associated with the surgical environment based on a discovery procedure. The discovery procedure may include determining the surgical systems present or used in a surgical environment. The discovery procedure may include determining capabilities associated with each surgical system present or used in the surgical environment. The first surgical system may determine capability information based on a pre-configuration (e.g., checklist, boot-up sequence). The pre-configuration may include information indicating surgical systems and their respective capabilities associated with the surgical environment.

The first surgical system may determine that received data is inaccurate and/or incomplete based on the received first data and the determined capability information. For example, the first surgical system may determine that a data or data type is missing. The first surgical system may send an indication indicating that the data is missing or the surgical system that generated and sent the data (e.g., second surgical system) is not operating properly.

### BRIEF DESCRIPTION OF THE DRAWINGS

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 shows an example surgical instrument.

FIG. 6 depicts an example of anatomy and surgical tools during a stomach tumor extraction.

FIG. 7 depicts an example laparoscopic endoscopic cooperative surgery (LECS) to dissect a stomach tumor.

FIG. 8A depicts an example stomach tumor extraction.

FIGS. 8B and 8C depict an example of controlled energy applied on the endoscopic side during the example method of FIG. 8A.

FIG. 8D depicts an example of a tumor pivoted to the laparoscopic side during the example method of FIG. 8A.

FIG. 9 depicts an example oral extraction of a tumor.

FIG. 10A depicts an example of sensed device locations without any interference or distortion.

FIG. 75B depicts an example of misalignment of sensed device locations.

FIG. 11 depicts an example of redundant electromagnetic field monitoring in an operating room.

FIG. 12 depicts an example of reconciling electromagnetic navigation of a flexible endoscope.

FIG. 13 depicts an example anatomy in a supine position.

FIG. 14 depicts an example of endoscopic locations within the example anatomy of FIG. 13 in a Trendelenburg position.

FIG. 15 depicts an example CT scan of a patient in a supine position and an example EBUS scan of the patient in the Trendelenburg position.

FIG. 16 depicts an example EUS guided stone identification method.

FIG. 17 depicts an example of operational envelopes of robots.

FIG. 18 depicts an example thoracic procedure with five robots arrayed around a patient.

FIG. 19 depicts an example of changing battery levels of smart devices over time.

FIG. 20 illustrates a system diagram of the operation of an Operating Room (OR) smart system, with, for example, a surgeon, data center, bed, patient, legacy device, and surgical systems data set.

FIG. 21 illustrates an example architecture diagram of a smart system.

FIG. 22 illustrates an example system diagram of an operating room smart system connected to legacy devices, alongside a surgical systems database.

FIG. 23 illustrates an example database decision-making process within pre-operative, intra-operative, and post-operative modules.

FIG. 24 illustrates an example block diagram of a surgical systems data set.

FIG. 25 illustrates an example of a smart system interfacing with an identified device.

FIG. 26 illustrates and example of a surgical smart system gathering and displaying information from an identified device.

FIG. 27 illustrates an example block diagram of a surgical smart system.

FIG. 28 illustrates an example block diagram of a surgical smart system.

FIG. 29 illustrates a logical representation of surgical systems and/or surgical imaging systems sharing control, information, and porting imaging using techniques described herein.

FIG. 30 illustrates an example surgical operating room with robotic surgical systems.

FIG. 31 illustrates an example of control redirection where a surgical instrument or a tool is part of a primary surgical system.

FIG. 32 illustrates an example of control redirection where a surgical instrument or a tool is part or a secondary surgical system and separated from a primary surgical system.

FIG. 33 is a flow chart illustrating one robotic surgical system (e.g., a laparoscopic robotic surgical system) performing a surgical procedure in tandem with a second robotic surgical system (e.g., an endoscopic robotic surgical system), and a smart surgical system.

FIG. 34 illustrates an example of a surgical procedure being performed by one robotic surgical system (e.g., a laparoscopic robotic surgical system) working in tandem with a second robotic surgical system (e.g., an endoscopic robotic surgical system), and a smart surgical system.

FIGS. 35A-B illustrate an exemplary colorectal tumor removal surgical procedure using techniques described herein.

FIG. 36 illustrates an example tumor removal using techniques described herein.

FIG. 37 is a message sequence diagram illustrating control sharing between two surgical systems.

FIG. 38 illustrates an example of a user controlling a first surgical system (e.g., a robotic endoscopic flexible scope) being able to control a second surgical system or a smart surgical system (e.g., an imaging system).

FIG. 39 block diagram illustrating exchangeability of imaging streams and control between various surgical systems and smart surgical systems (e.g., imaging console systems).

FIG. 40 illustrates an example surgical operating room with robotic surgical instruments.

FIG. 41A illustrates example smart surgical devices

FIG. 41B illustrates example legacy devices.

FIG. 42 is a block diagram illustrating example components of a surgical device.

FIG. 43A is a block diagram illustrating an example control loop in a surgical device.

FIG. 43B illustrates an example of a surgical instrument autonomously moving to maintain a proximity boundary with another surgical instrument.

FIG. 43C illustrates an example of a scope autonomously moving to maintain another surgical instrument in a field of view of the scope.

FIGS. 44A and 44B illustrate an example tumor removal procedure using techniques described herein.

FIGS. 45A-C illustrate another example tumor removal procedure using techniques described herein.

FIGS. 46A-C illustrate yet another example tumor removal procedure using techniques described herein.

FIG. 47 illustrates an example method that may be performed by a surgical instrument.

FIG. 48 illustrates an example layout of a surgical operating room.

FIG. 49 is a block diagram illustrating example processing modes in a surgical device.

FIG. 50 illustrates an example of overlapping zones of movement between devices in a surgical operating room.

FIG. 51 is a block diagram illustrating an example of optimizing device movement based on a constraint.

FIG. 52A illustrates an example API between surgical instruments.

FIGS. 52B and 52C illustrate an example of a scope maintaining a target device in a field of view as the target device moves.

FIG. 53 illustrates an example method that may be performed by a surgical instrument.

FIG. 54 illustrates an example surgical operating room with endoscopic and laparoscopic devices.

FIG. 55 illustrates an example of a smart surgical scope adjusting imaging parameter(s) to maintain a coupled field of view with another imaging scope.

FIG. 56 illustrates an example of a smart surgical scope updating imaging parameter(s) to maintain a coupled field of view with another imaging scope.

FIG. 57 illustrates an example of emitting additional wavelengths through tissue to augment sensors in an endoscope.

FIG. 58 illustrates an example of using color distortion to indicate that an image is a composite image.

FIG. 59 illustrates an example method that may be performed by a surgical instrument.

FIG. 60 illustrates an example surgical operating room with robotic arms.

FIG. 61 illustrates an example of electromagnetic distortion caused by metal staples.

FIG. 62 illustrates example operations and communication by two surgical devices.

FIG. 63 is a block diagram illustrating example components of a surgical device.

FIGS. 64A-D illustrate graphical representations of magnetic distortion and distortion compensation.

FIG. 65 illustrates an example of distortion compensation using sensors in an operating room.

FIGS. 66A and 66B illustrate example distortion caused by a CT machine moving around a patient and example distortion compensation.

FIG. 67 illustrates an overlayed comparison of a sensed location and an adjusted location of an endoscope.

FIG. 68 illustrates examples of a low bandwidth reference plane and a high bandwidth reference plane.

FIG. 69 illustrates an example method that may be performed by a surgical instrument.

FIG. 70 is a data flow diagram illustrating communication between an endoscopic scope and a laparoscopic scope.

FIG. 71 illustrates example components of a landmark identification and matching engine.

FIG. 72 illustrates an example method for object registration.

FIG. 73 illustrates an example of two scopes registering objects in their field of view.

FIG. 74A illustrates example organ positions while a patient is in the supine position.

FIG. 74B illustrates example organ positions while the patient is in the Trendelenburg position.

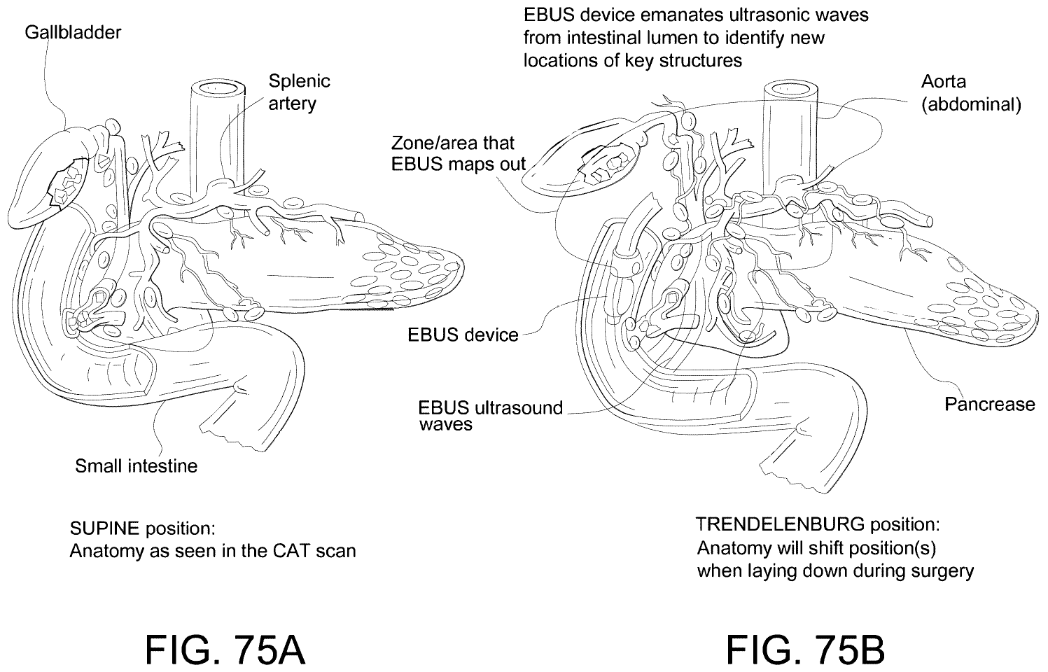

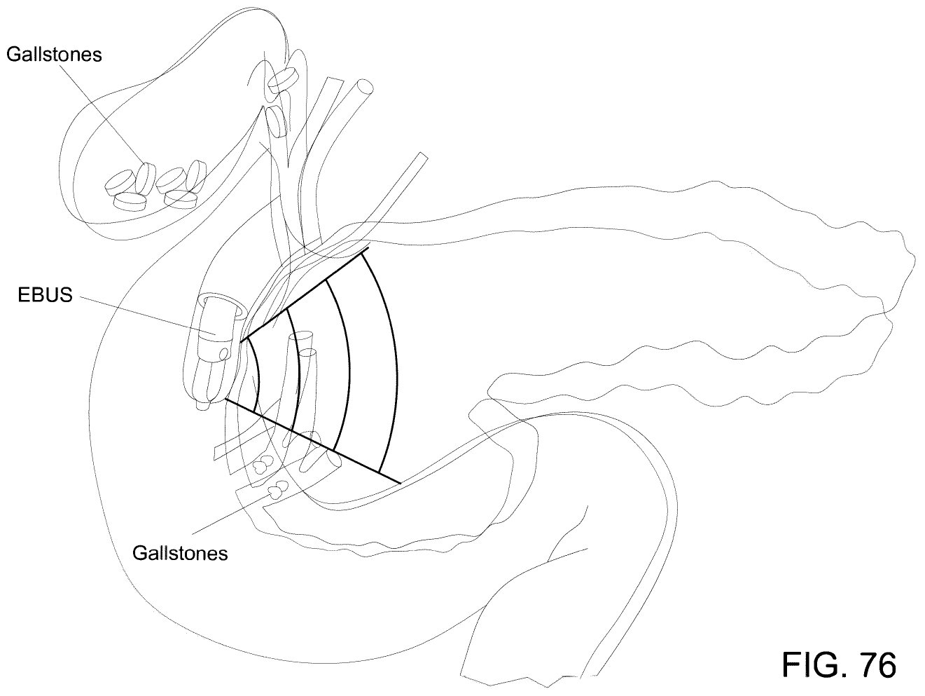

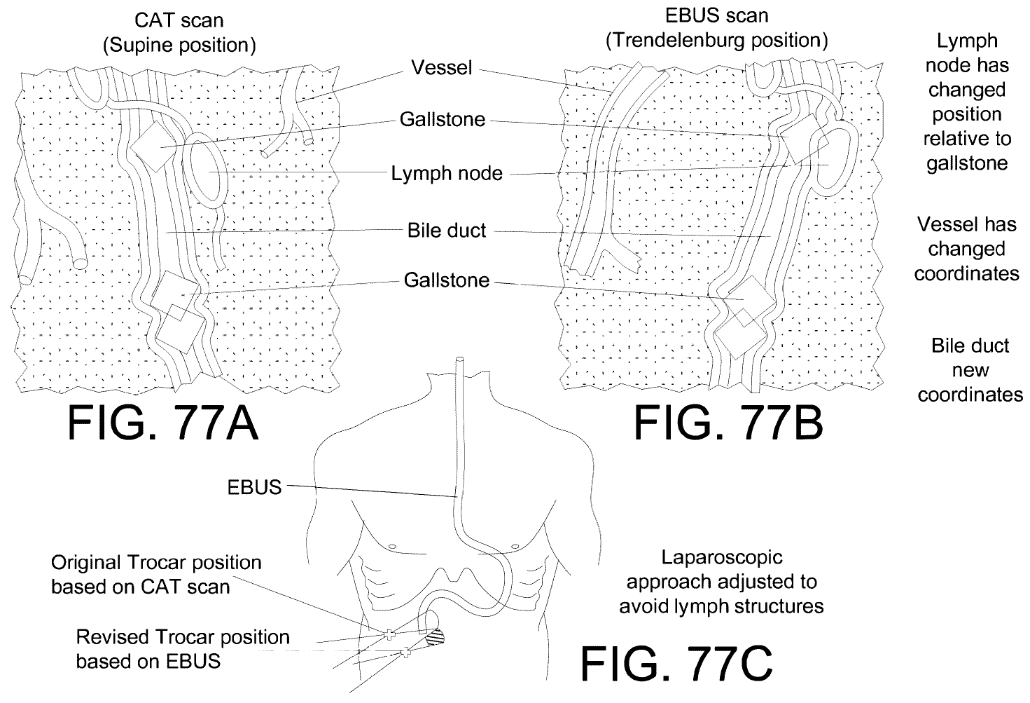

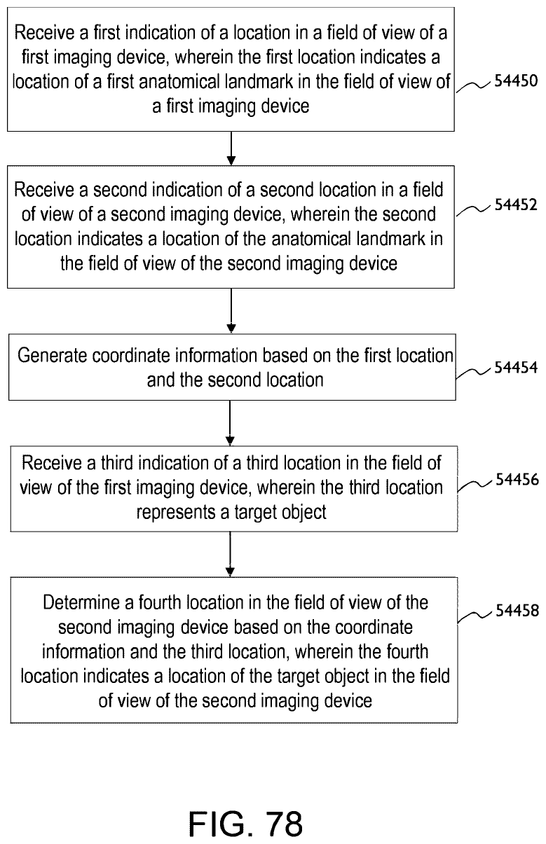

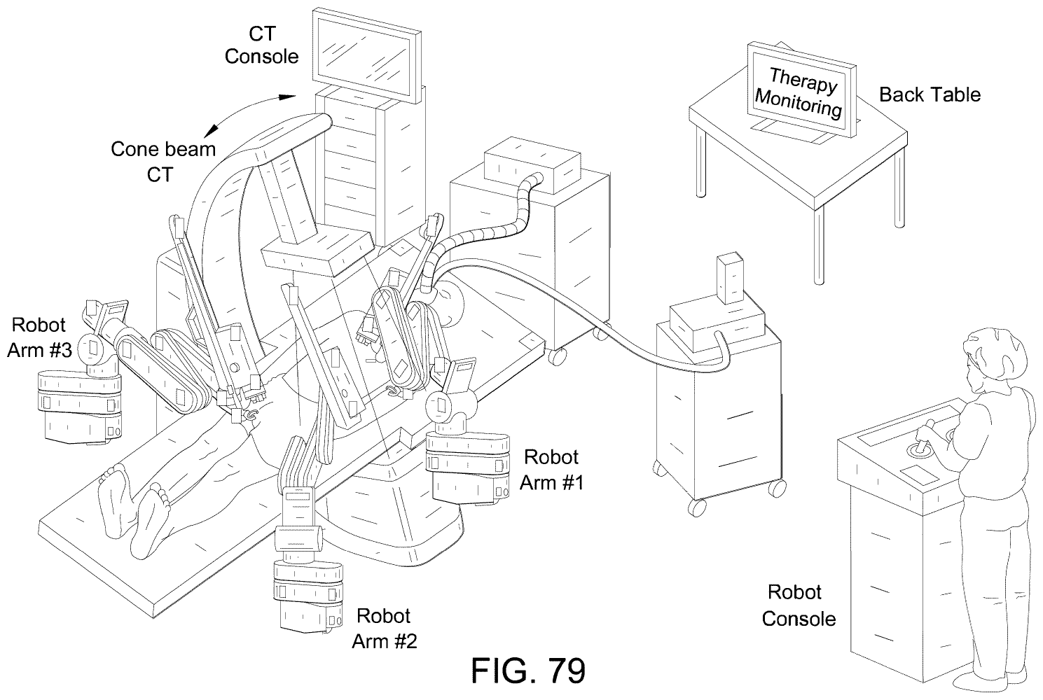

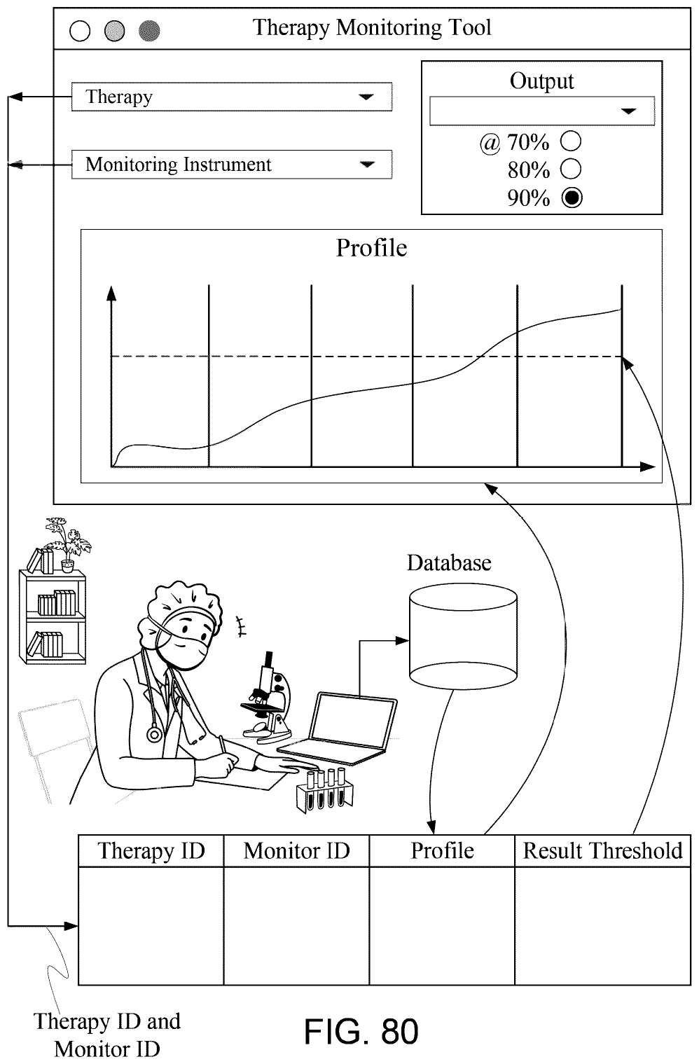

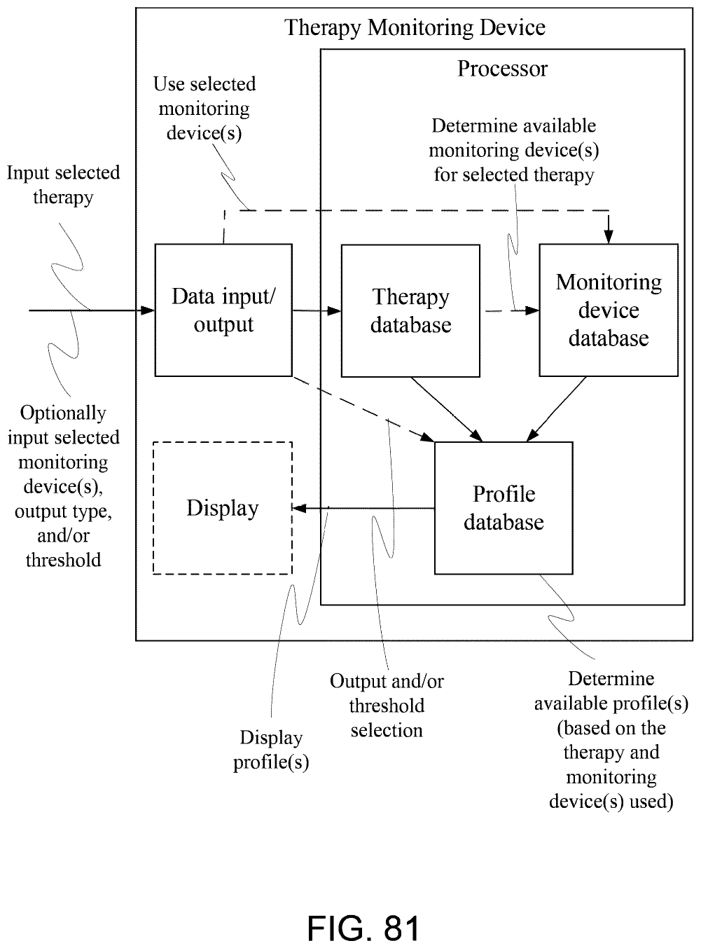

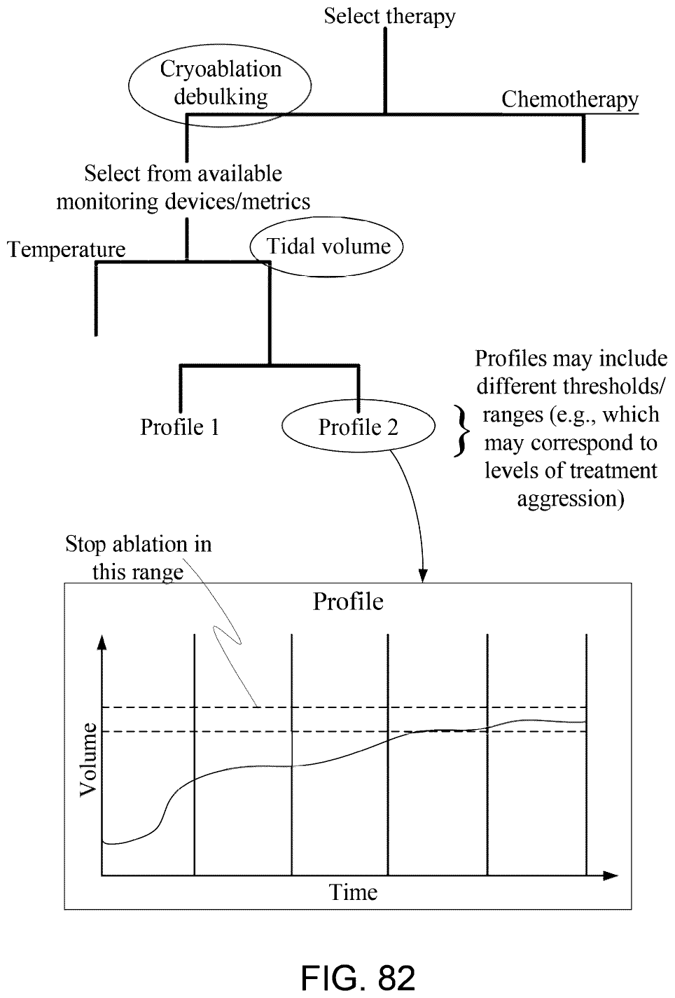

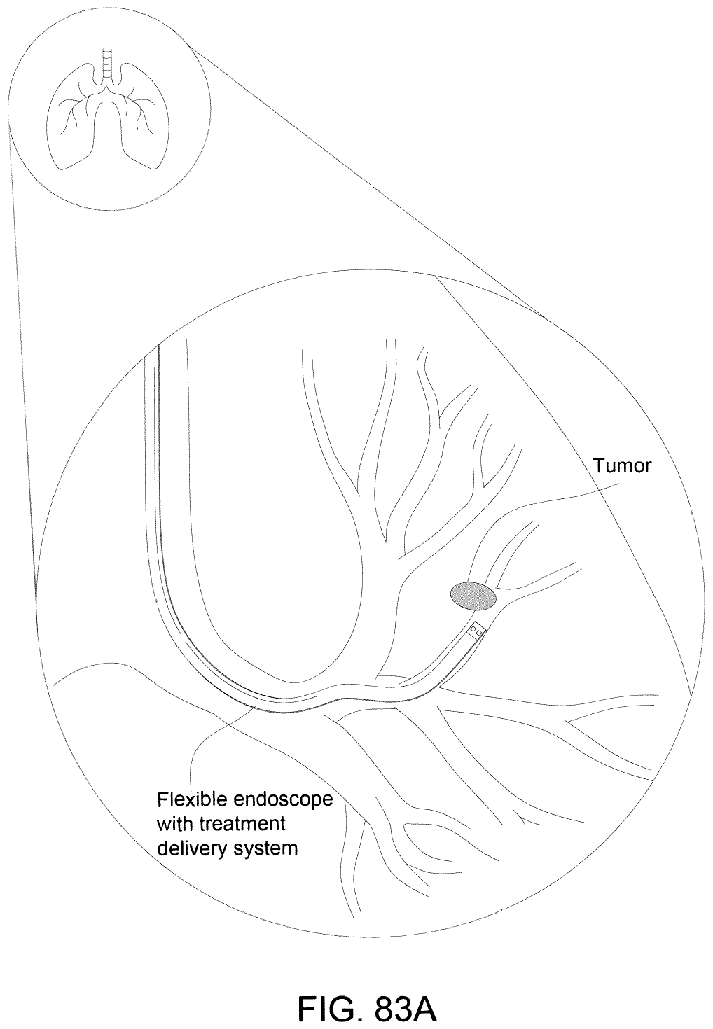

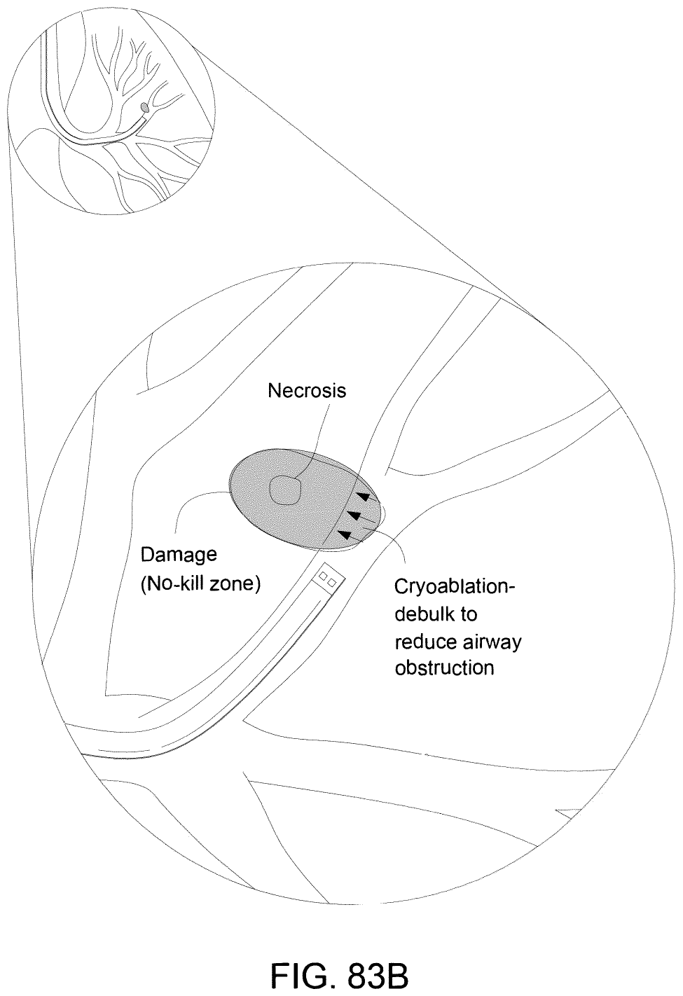

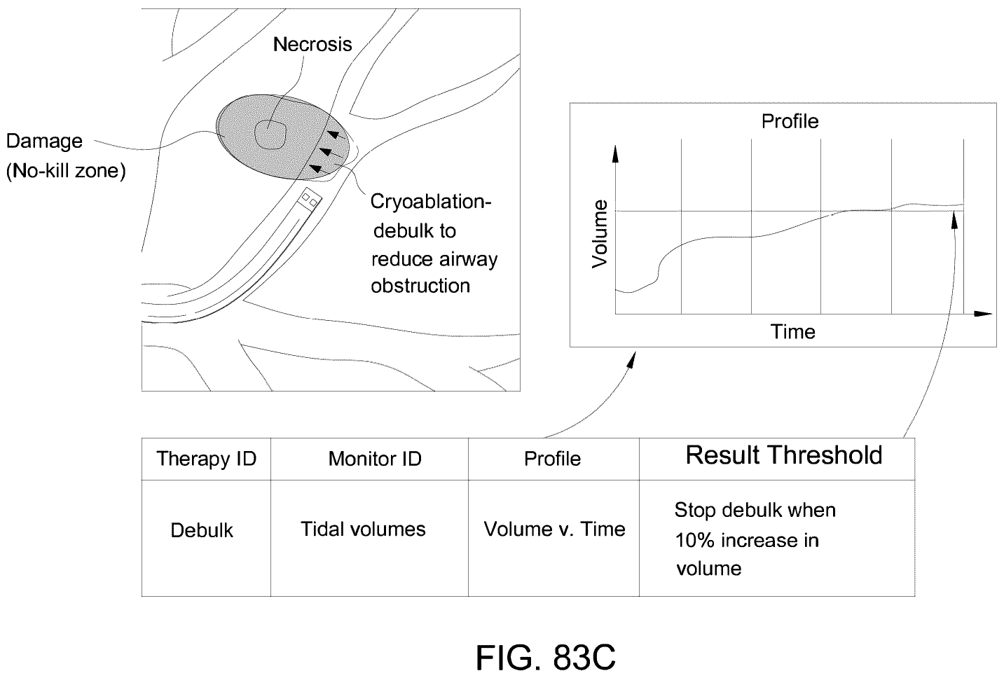

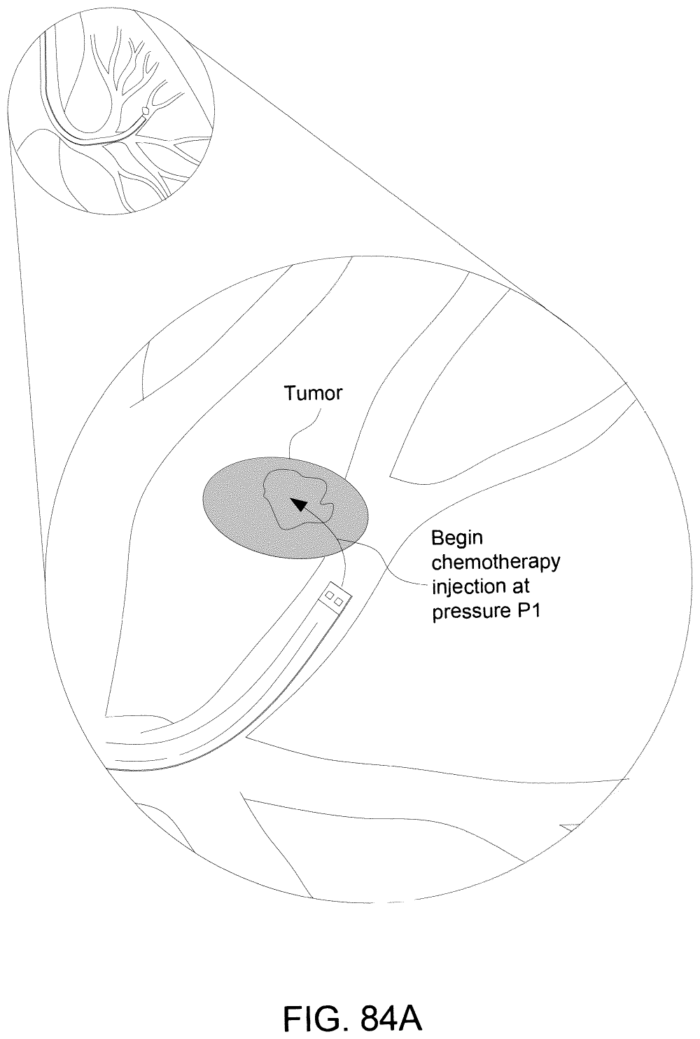

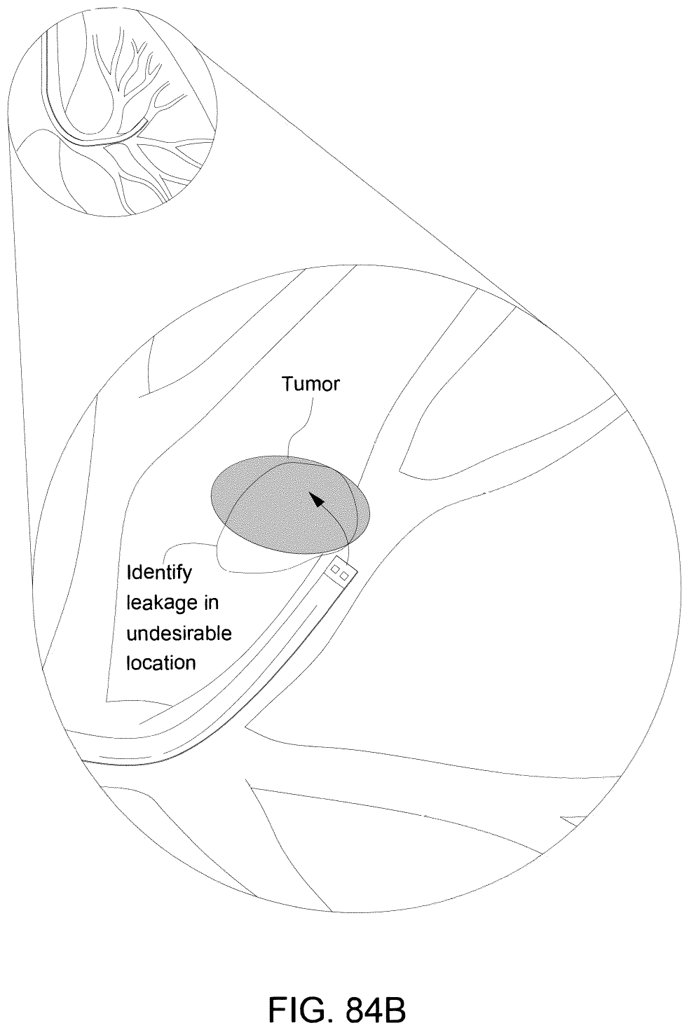

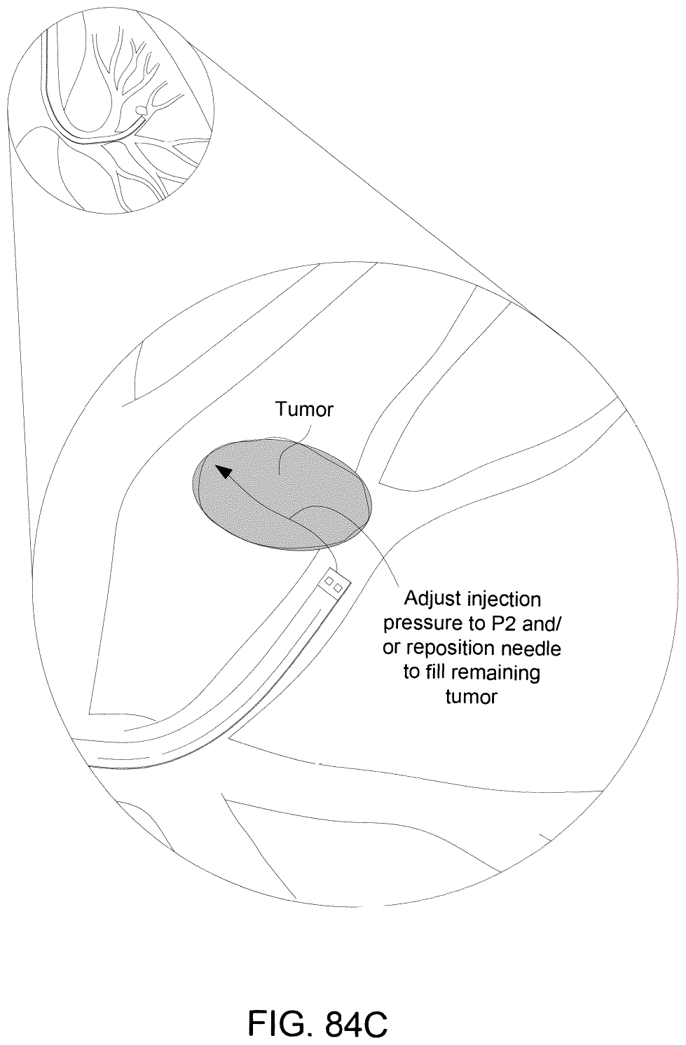

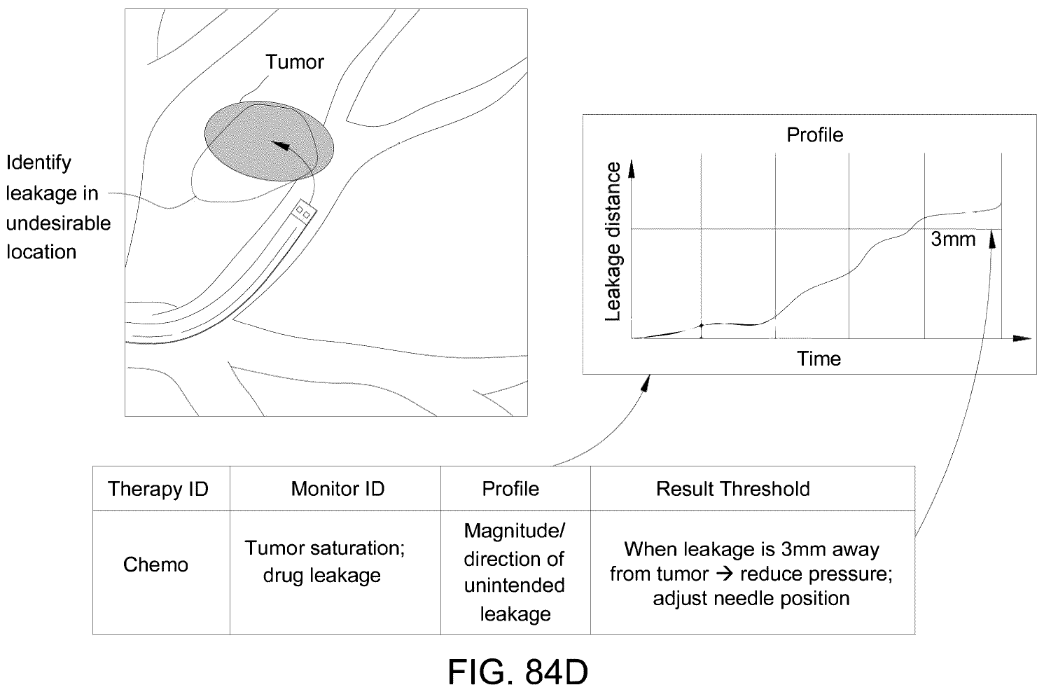

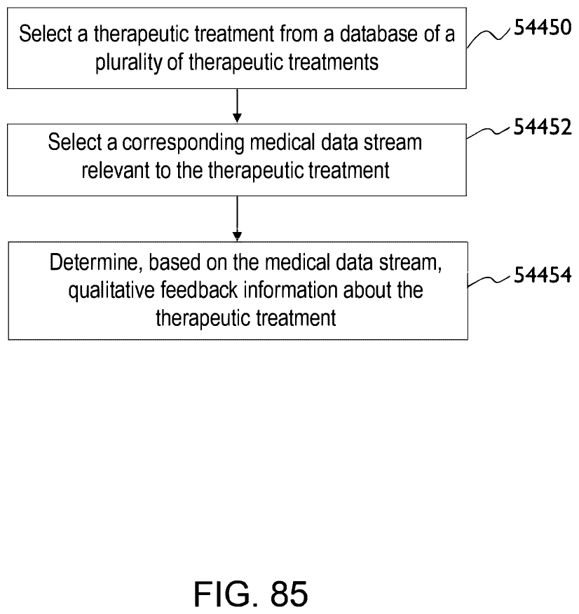

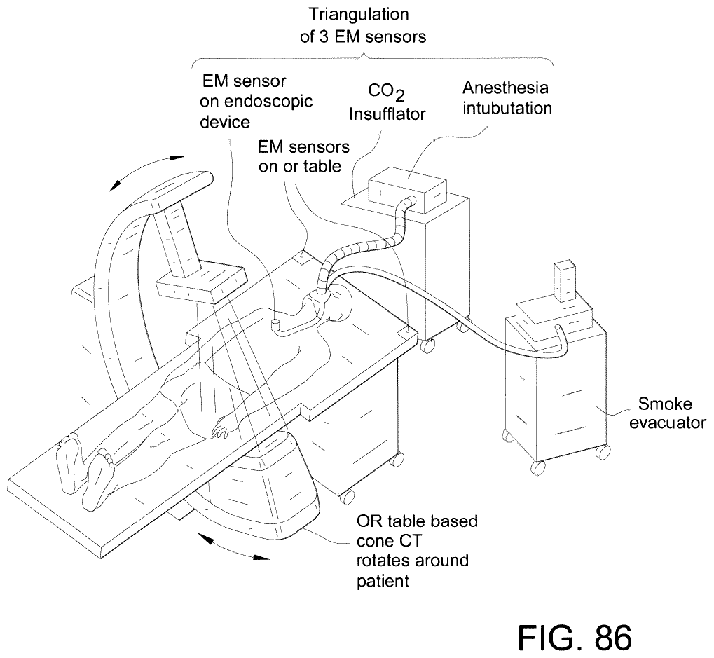

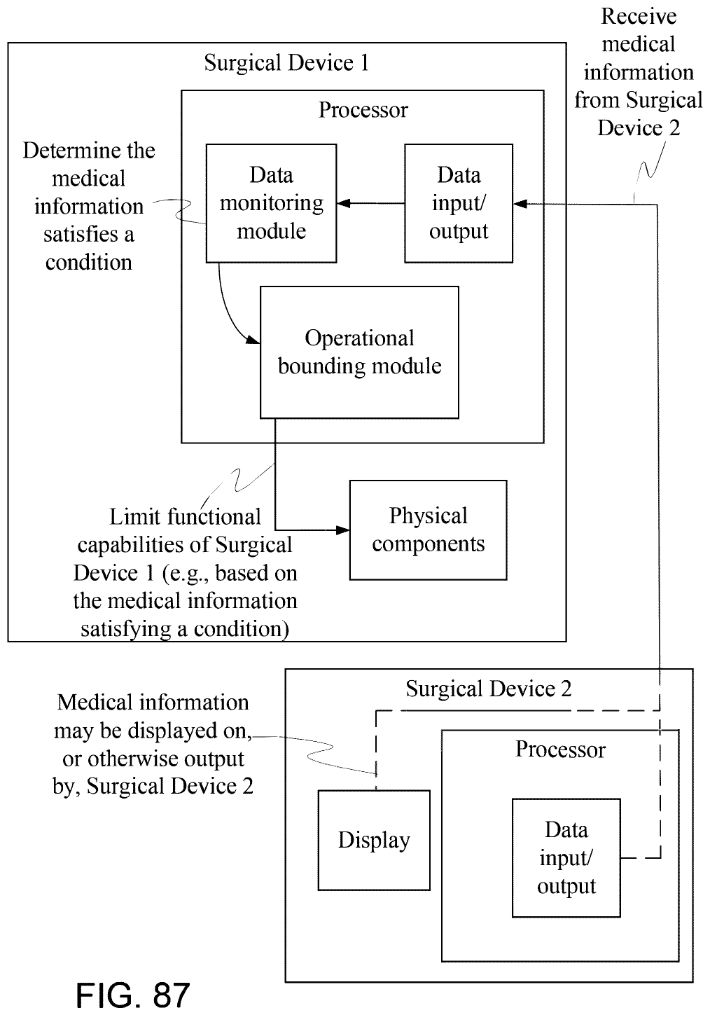

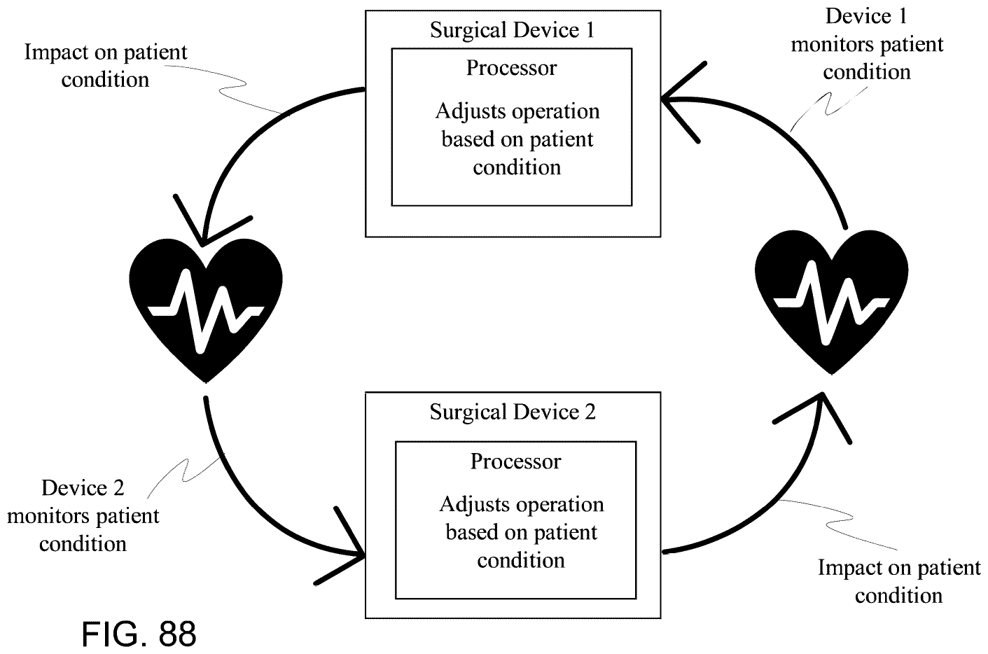

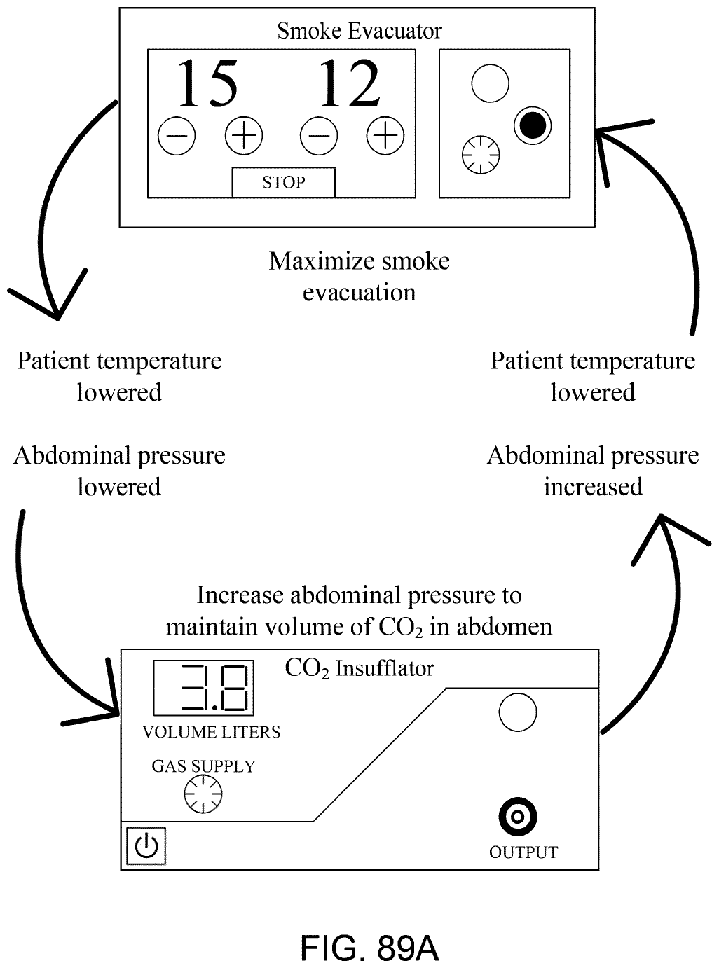

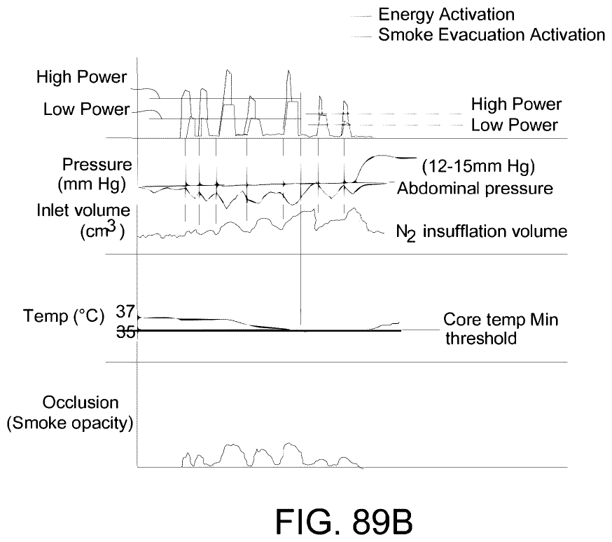

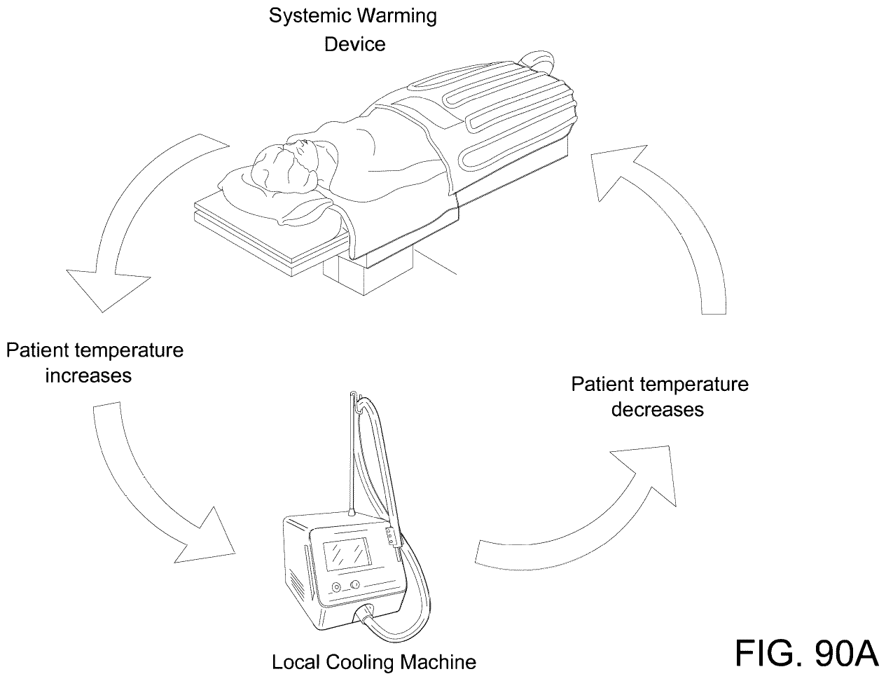

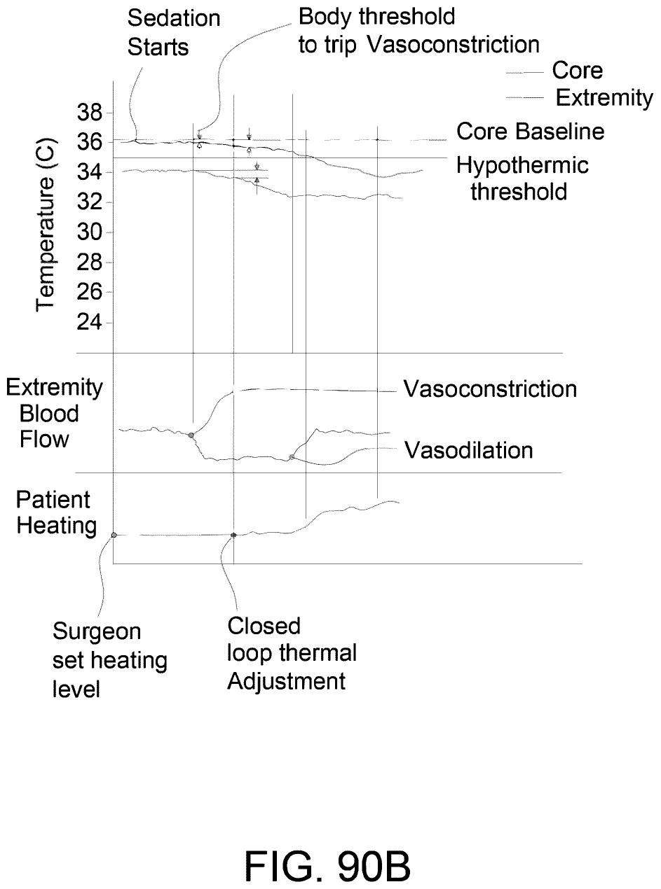

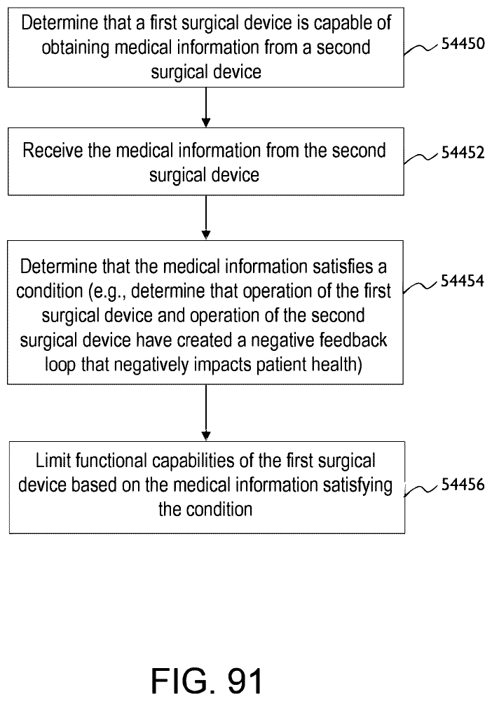

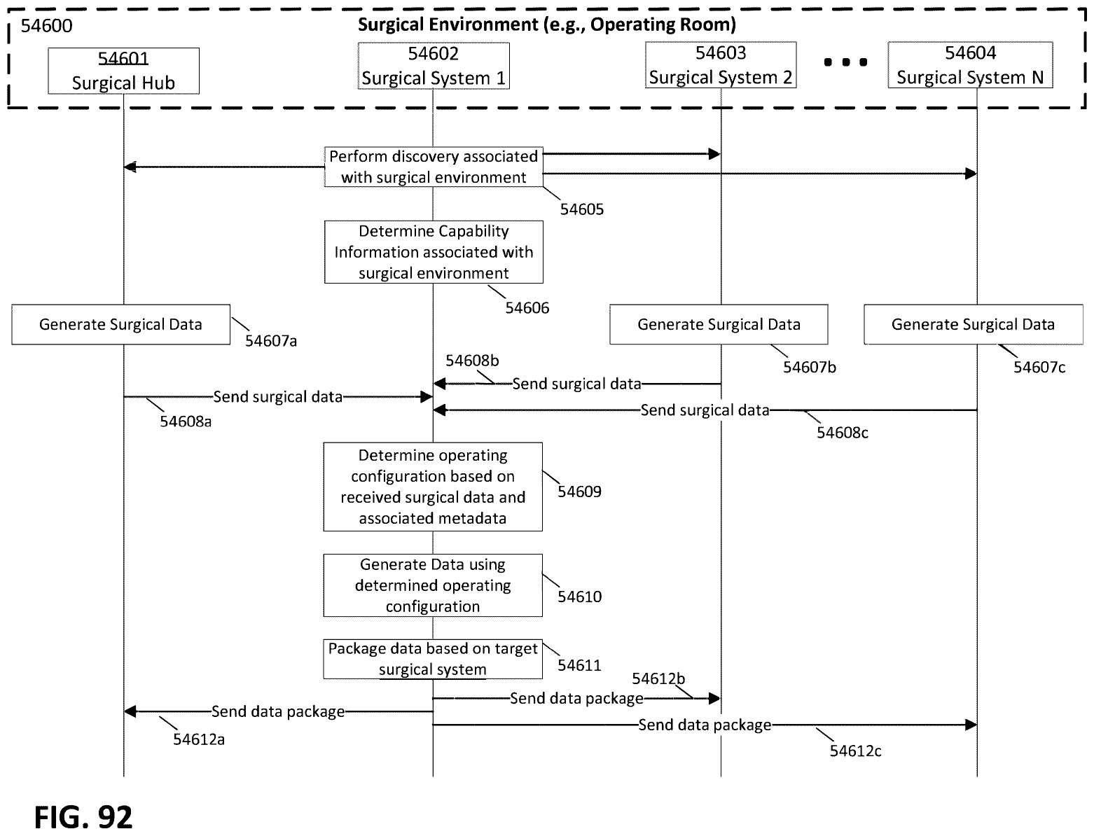

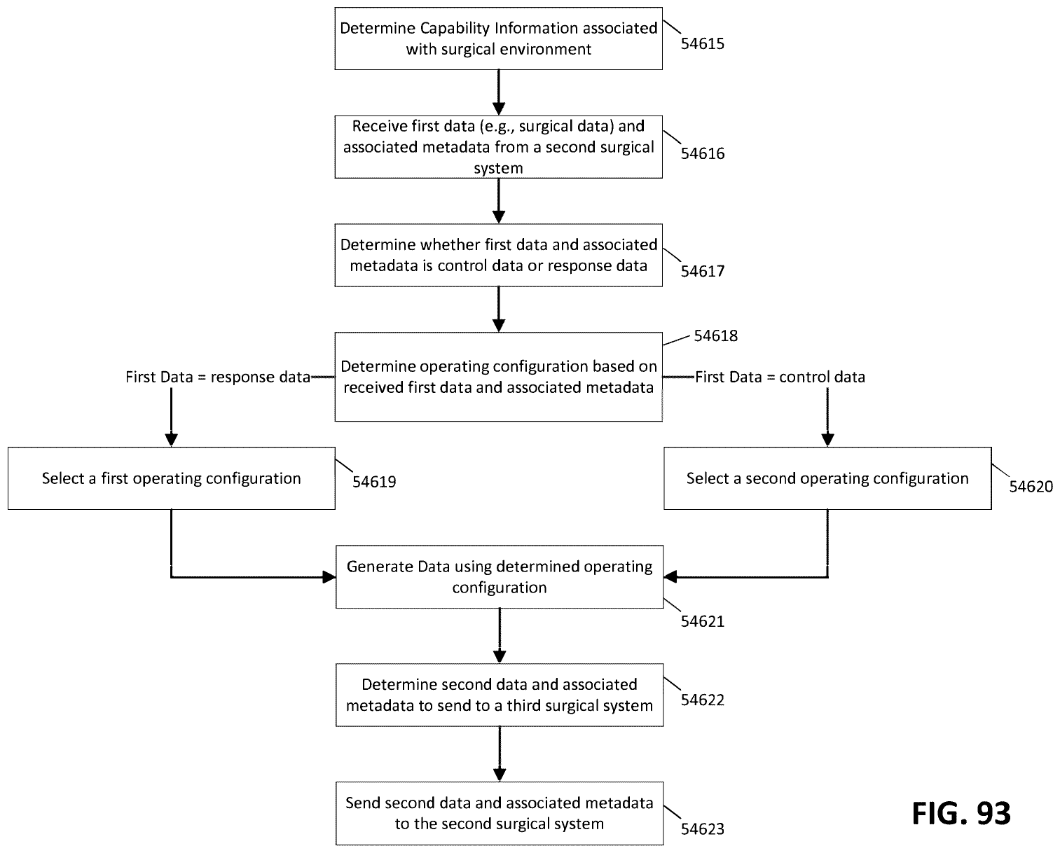

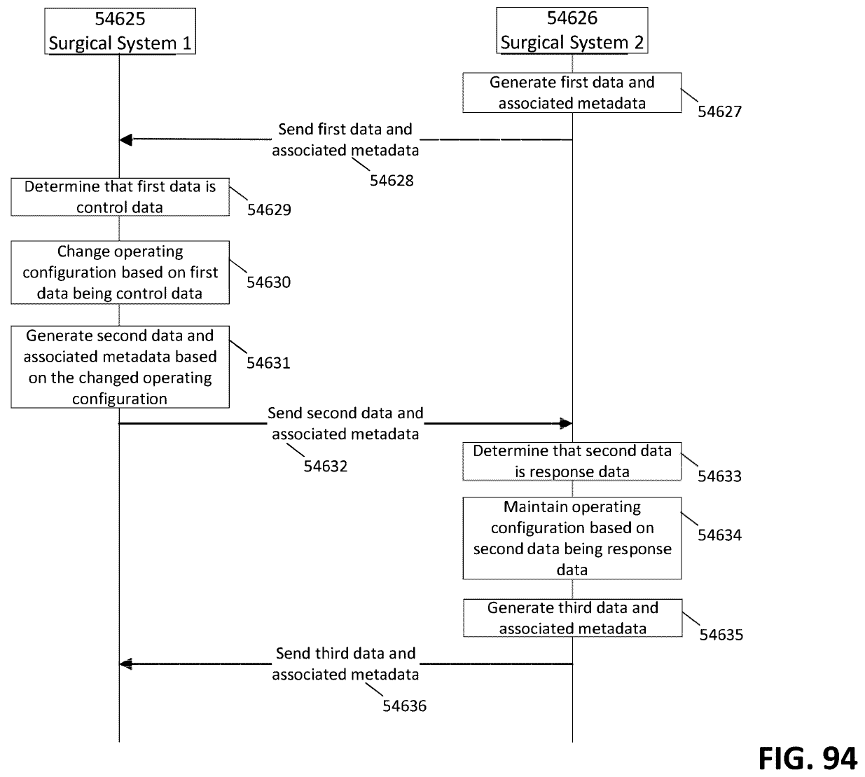

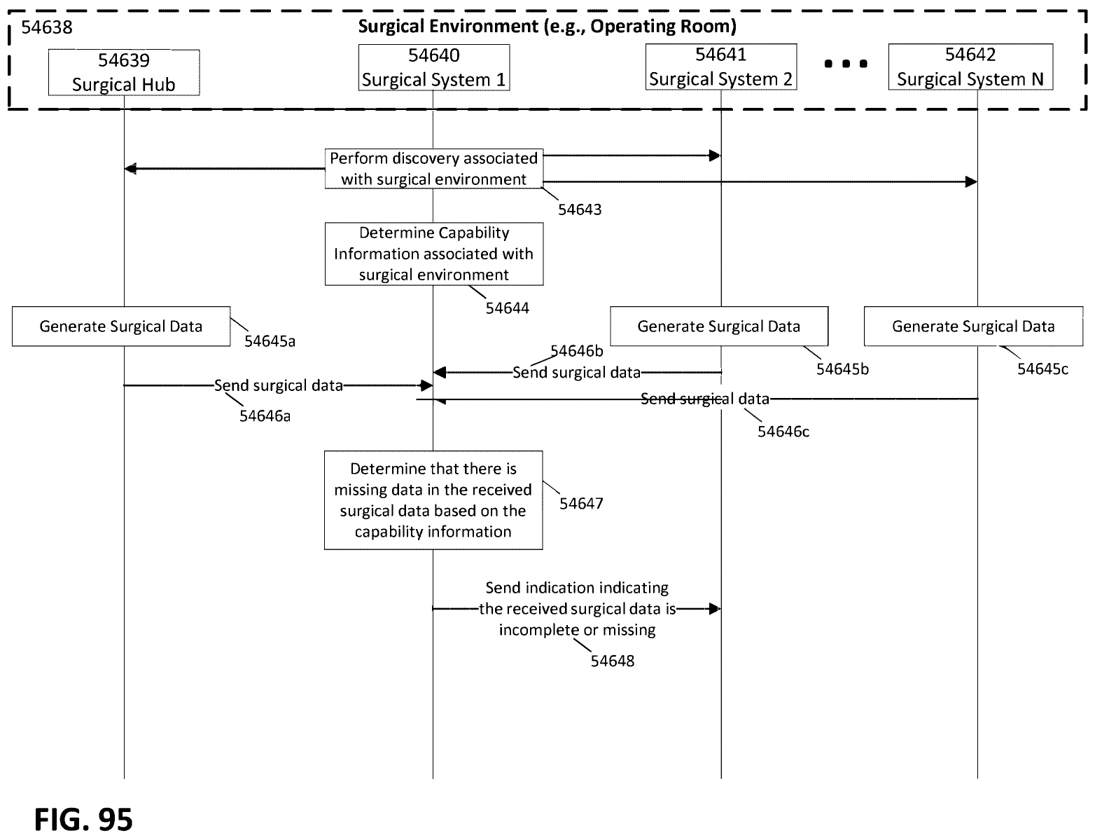

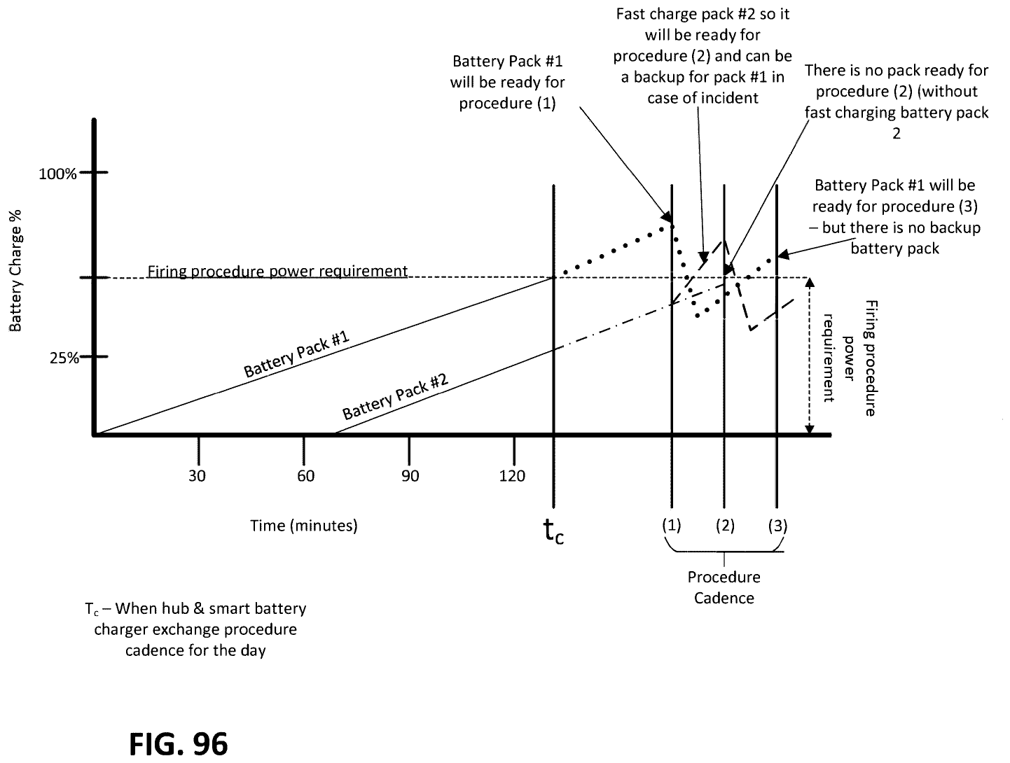

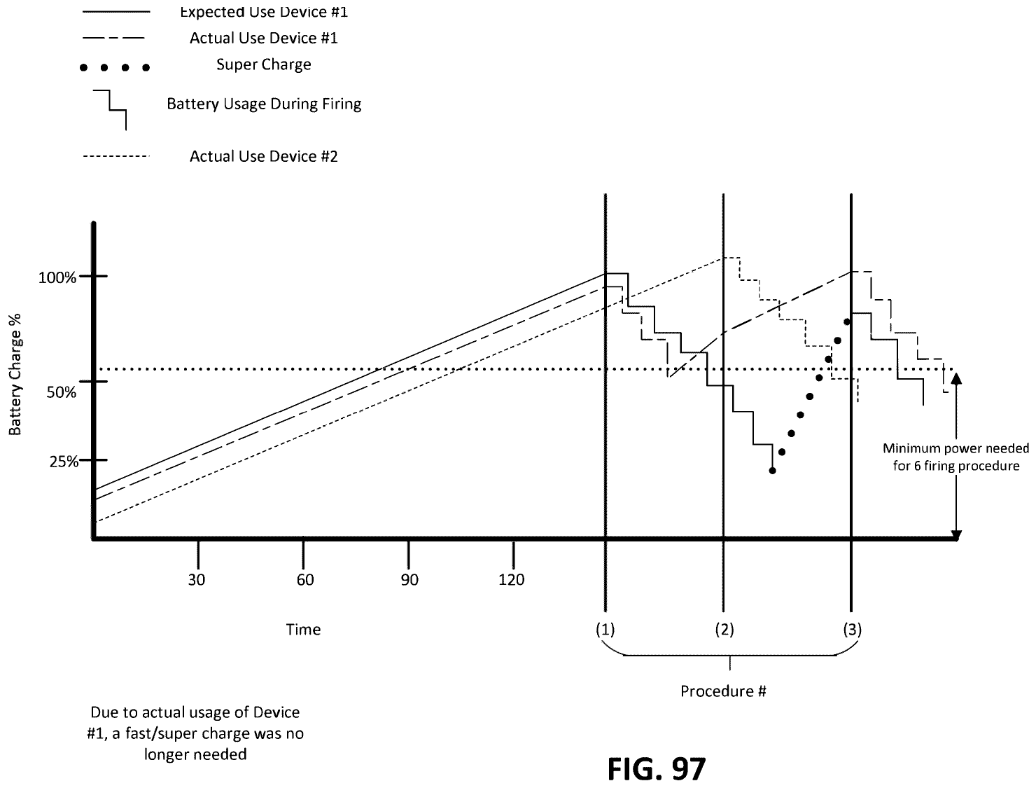

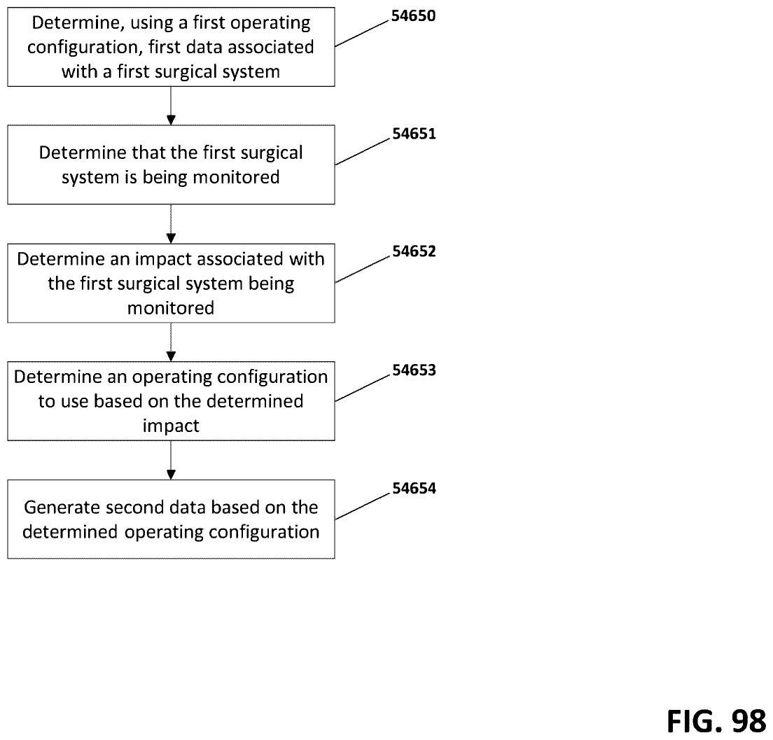

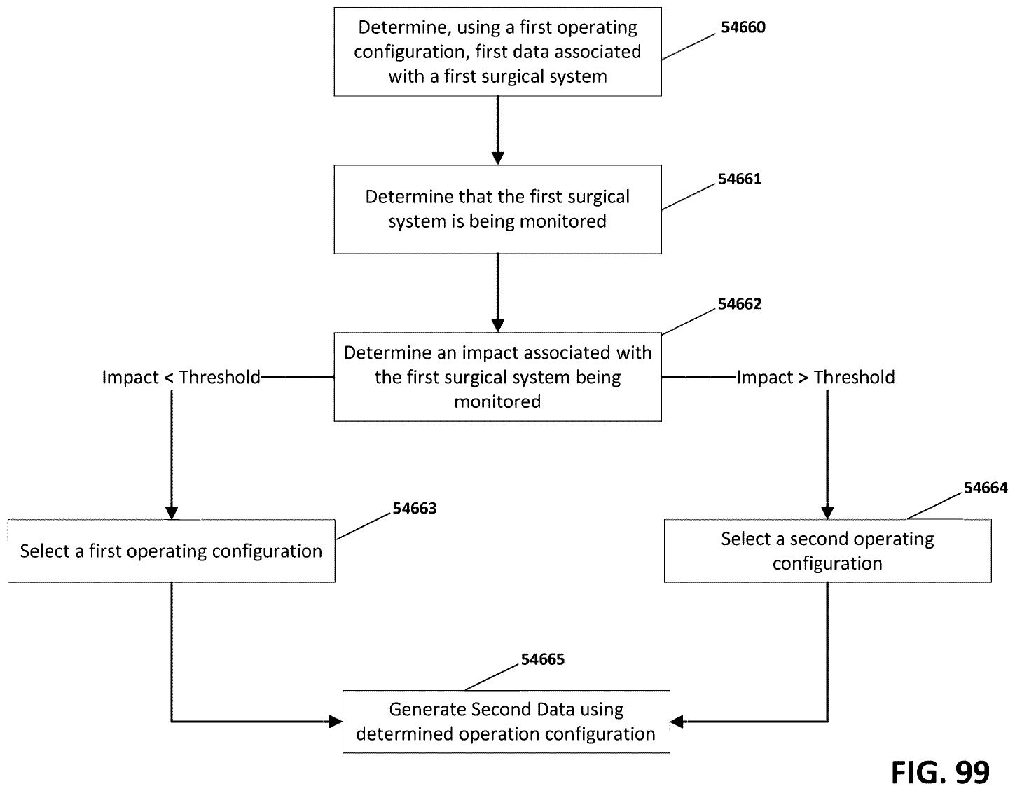

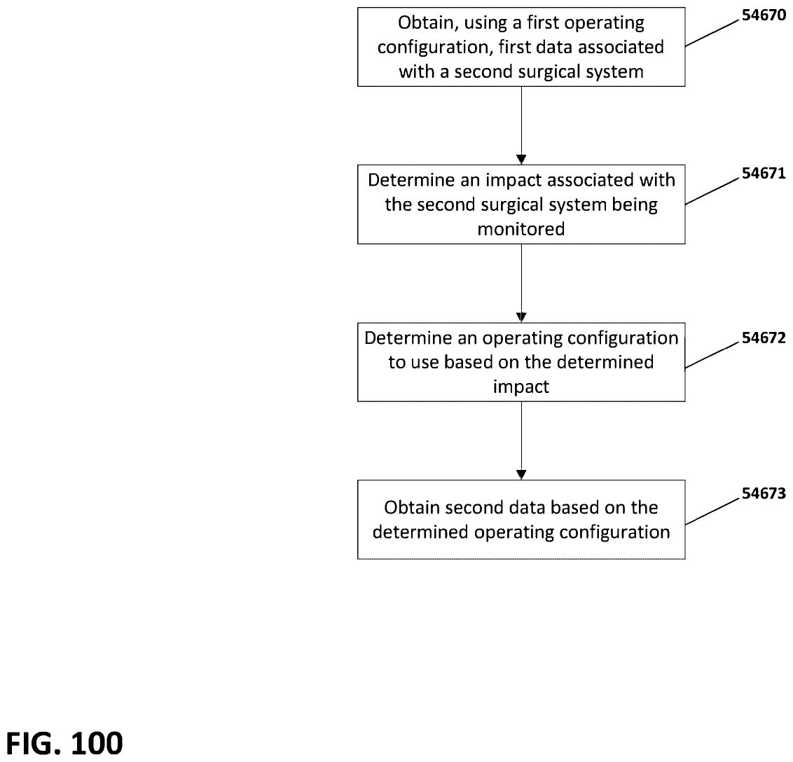

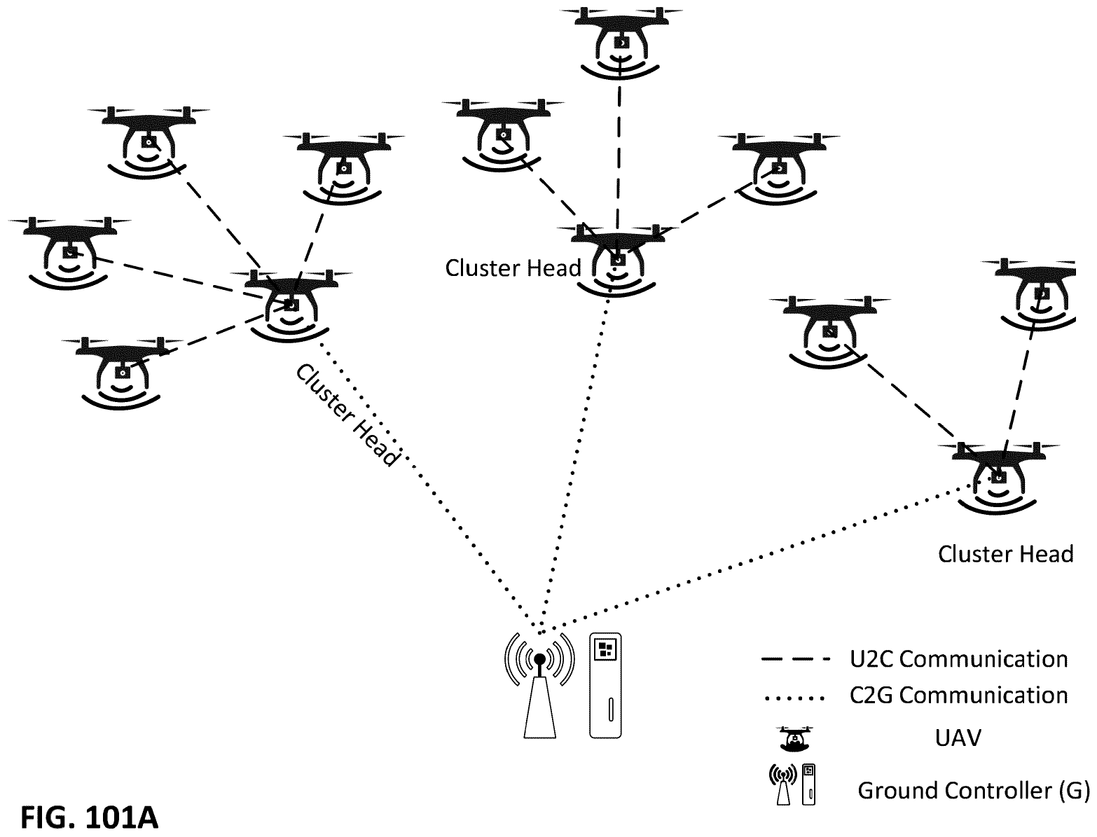

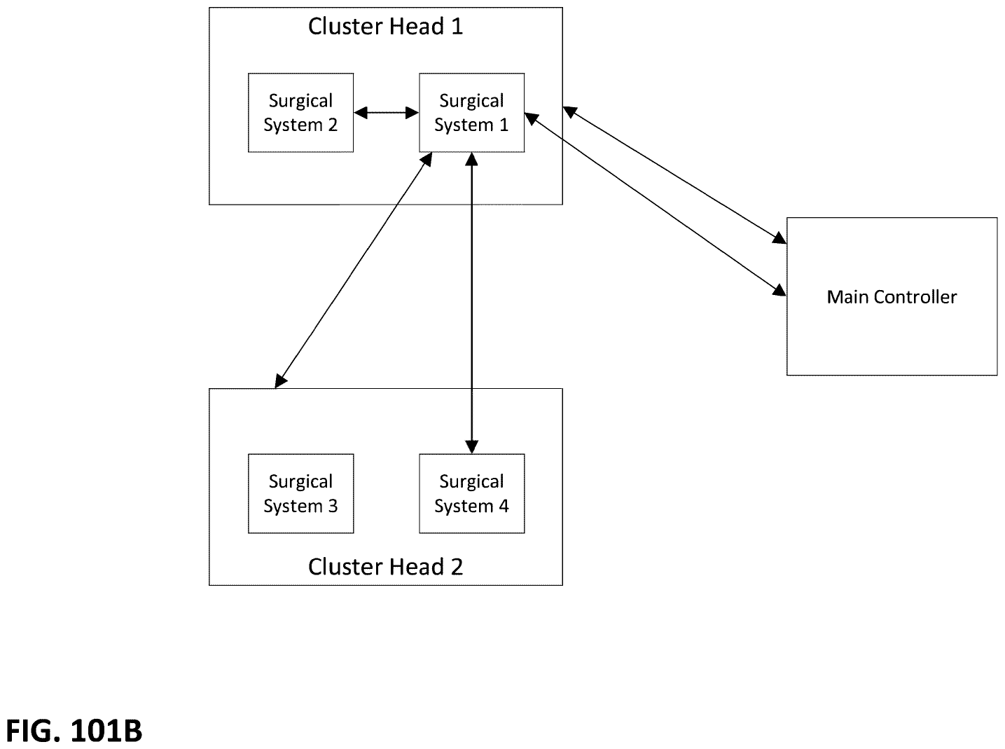

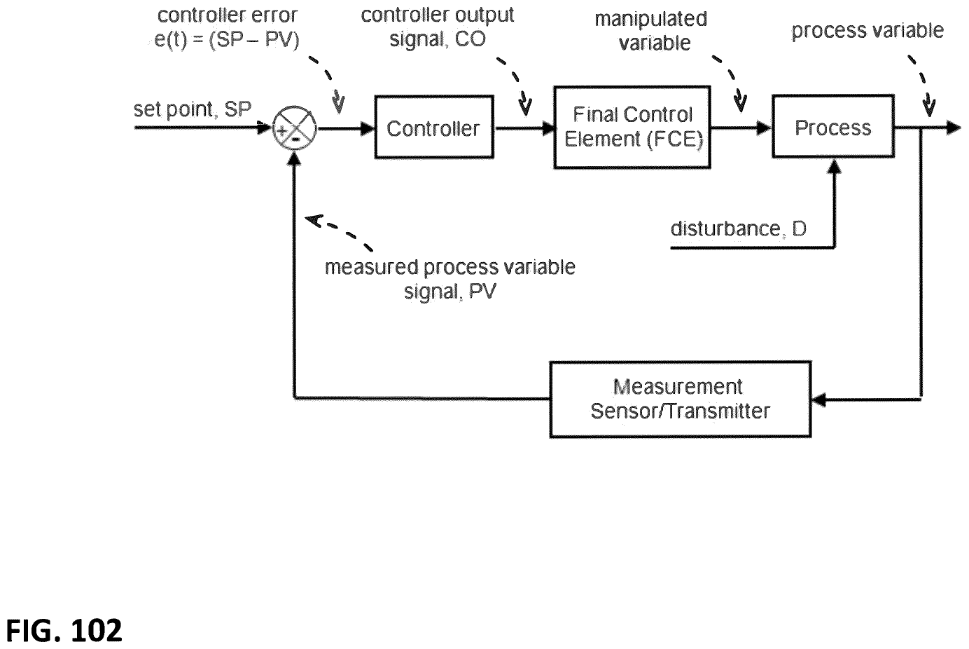

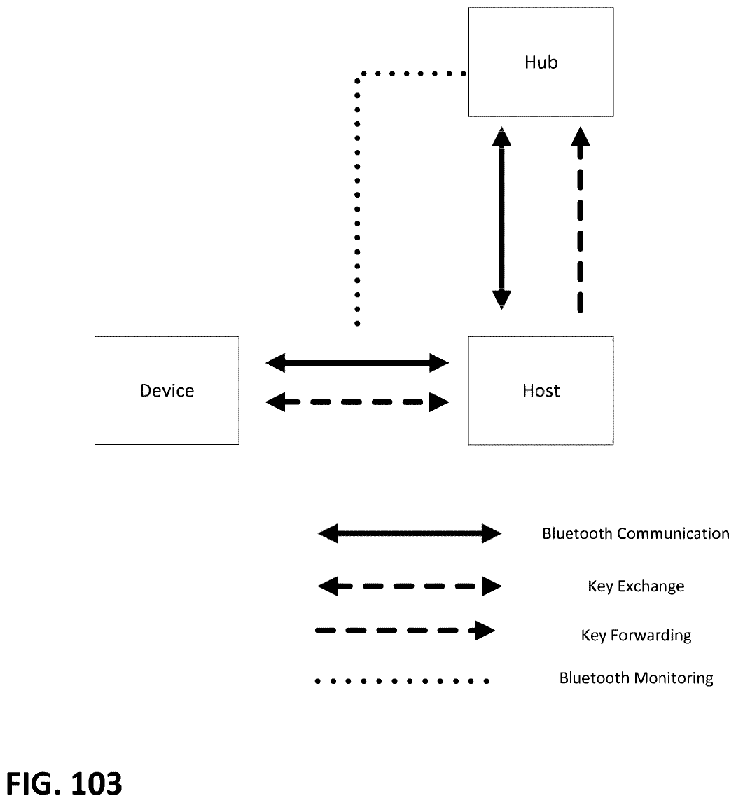

FIG. 75A illustrates example gallbladder anatomy while a patient is in the supine position.

FIG. 75B illustrates example gallbladder anatomy while the patient is in the Trendelenburg position.

FIG. 76 illustrates an example comparison of gallbladder anatomy in pre-operative imaging and gallbladder anatomy in intra-operative imaging.

FIG. 77A illustrates example gallstone positions while a patient is in the supine position.

FIG. 77B illustrates example gallstone while the patient is in the Trendelenburg position.

FIG. 77C illustrates an example of adjusting an imaging location based on a change in patient position.

FIG. 78 illustrates an example method that may be performed by a surgical instrument.

FIG. 79 illustrates an example surgical operating room with therapy monitoring.

FIG. 80 illustrates an example therapy monitoring tool.

FIG. 81 illustrates example components of a therapy monitoring device.

FIG. 82 illustrates example selection options provided by the therapy monitoring device.

FIG. 83A illustrates an example therapy device inserted in bronchial tubes.

FIG. 83B illustrates the example therapy device of 83A providing cryoablation treatment to a tumor.

FIG. 83C illustrates therapy monitoring associated with the example therapy device of FIGS. 83A and 83B.

FIG. 84A illustrates an example therapy device providing chemotherapy treatment to a tumor.

FIG. 84B illustrates the therapy device of FIG. 84A identifying a chemotherapy leakage.

FIG. 84C illustrates the therapy device of FIGS. 84A and 84B adjusting the chemotherapy treatment in response to the identified leakage.

FIG. 84D illustrates therapy monitoring associated with the example therapy device of FIGS. 84A-C.

FIG. 85 illustrates an example method that may be performed by a therapy monitoring tool.

FIG. 86 illustrates an example surgical operating room with surgical instruments that may create a negative feedback loop.

FIG. 87 is a block diagram illustrating example components in surgical devices.

FIG. 88 illustrates an example feedback loop between two surgical devices.

FIG. 89A illustrates an example feedback loop between a smoke evacuator and a CO₂ insufflator.

FIG. 89B is a graph illustrating the feedback loop between the smoke evacuator and the CO₂ insufflator.

FIG. 90A illustrates an example feedback loop between a systemic warming device and a local cooling system.

FIG. 90B is a graph illustrating the feedback loop between the systemic warming device and the local cooling system.

FIG. 91 illustrates an example method that may be performed by a surgical device.

FIG. 92 illustrates an example flow of interrelated surgical systems exchanging data within a surgical environment.

FIG. 93 illustrates an example flow of determining an operating configuration based on received data.

FIG. 94 illustrates an example flow of determining an operating configuration based on received data.

FIG. 95 illustrates an example flow of determining capability information associated with a surgical environment.

FIG. 96 illustrates an example of a smart battery charger sending usage information to a surgical hub.

FIG. 97 example illustrates an example of smart chargers for battery power stapling.

FIG. 98 illustrates an example flow of adapting operating configurations associated with data being monitored or received.

FIG. 99 illustrates an example flow of adapting operating configurations associated with data being monitored or received with respect to an impact on the data.

FIG. 100 illustrates an example flow of a surgical system changing its operating configuration to avoid being detected that it is monitoring a different surgical system.

FIG. 101A illustrates an example system adapting operating parameters based on a detection that the system is being monitored.

FIG. 101B illustrates an example of communication exchanged between cluster heads, the ground controller, and/or devices of a cluster.

FIG. 102 illustrates an example system adapting operating parameters to disturb a detected monitoring.

FIG. 103 illustrates an example encryption key passing to enable discreet monitoring of devices.

### DETAILED DESCRIPTION

A more detailed understanding may be had from the following description, given by way of example in conjunction with the accompanying drawings.

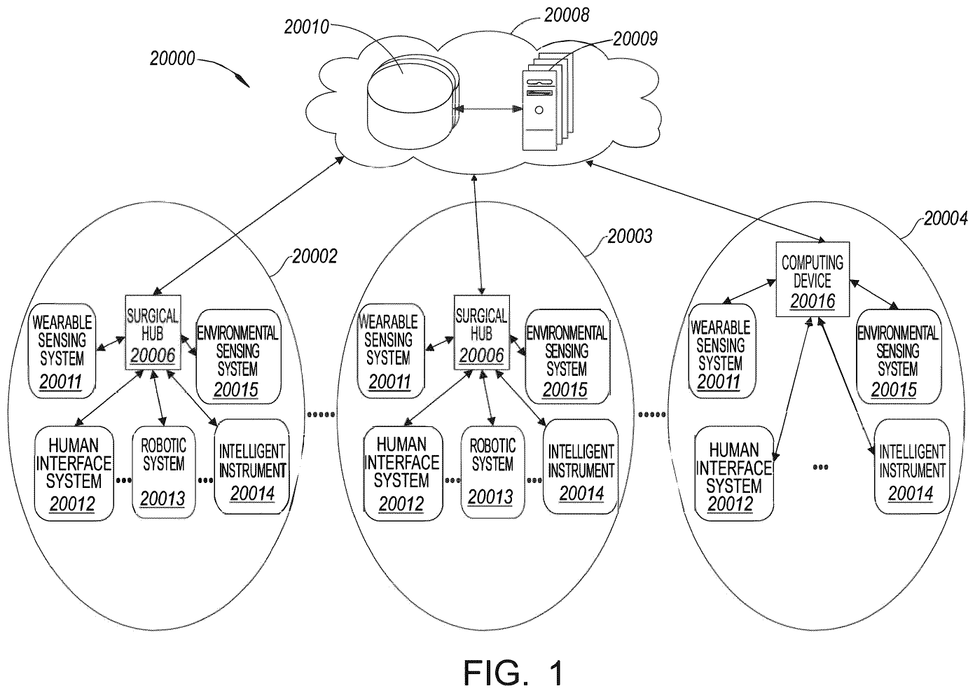

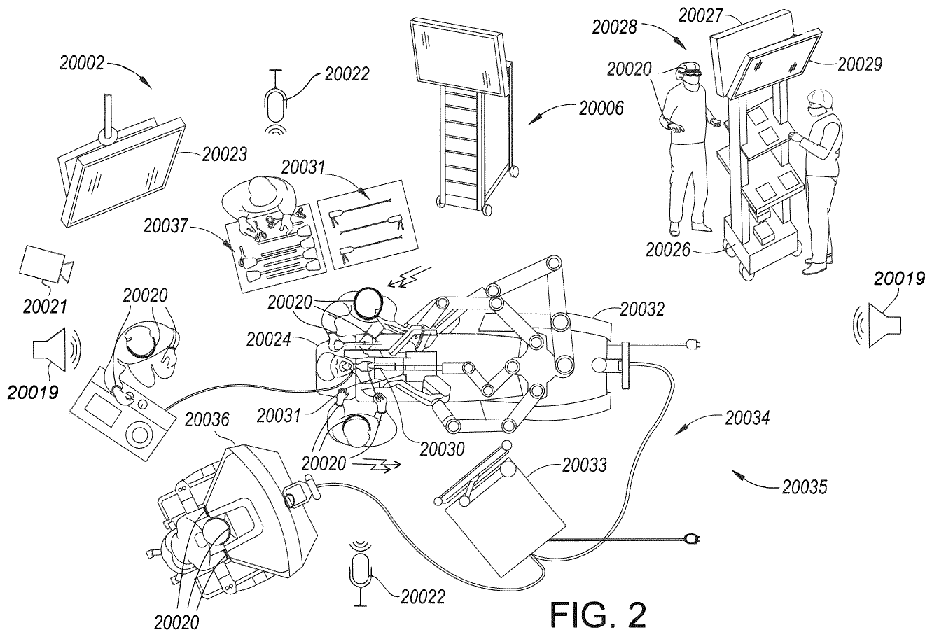

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It may be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is incorporated herein by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is incorporated herein by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 10013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is incorporated herein by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is incorporated herein by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is incorporated herein by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye may respond to wavelengths in air that range from about 380 nm to about 750 nm.

The invisible spectrum (e.g., the non-luminous spectrum) is the portion of the electromagnetic spectrum that lies below and above the visible spectrum (e.g., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is incorporated herein by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It may be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

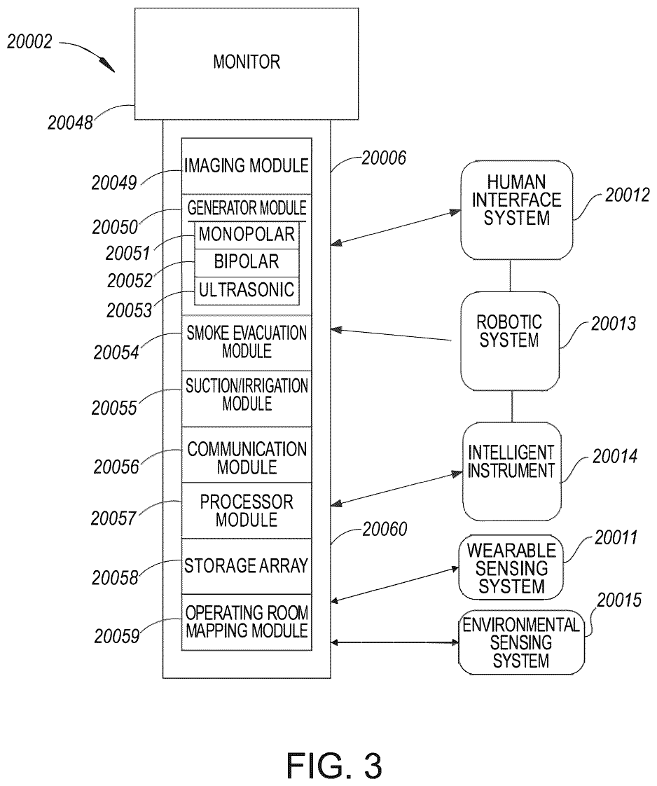

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050, a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is incorporated herein by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module also includes a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

Multiple energy types may be applied to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

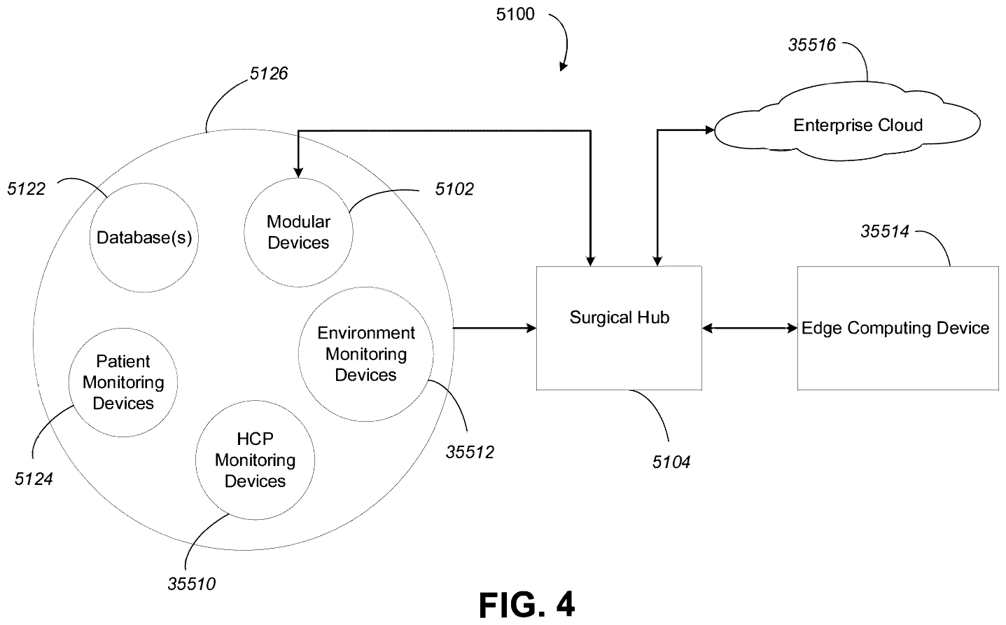

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or may subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

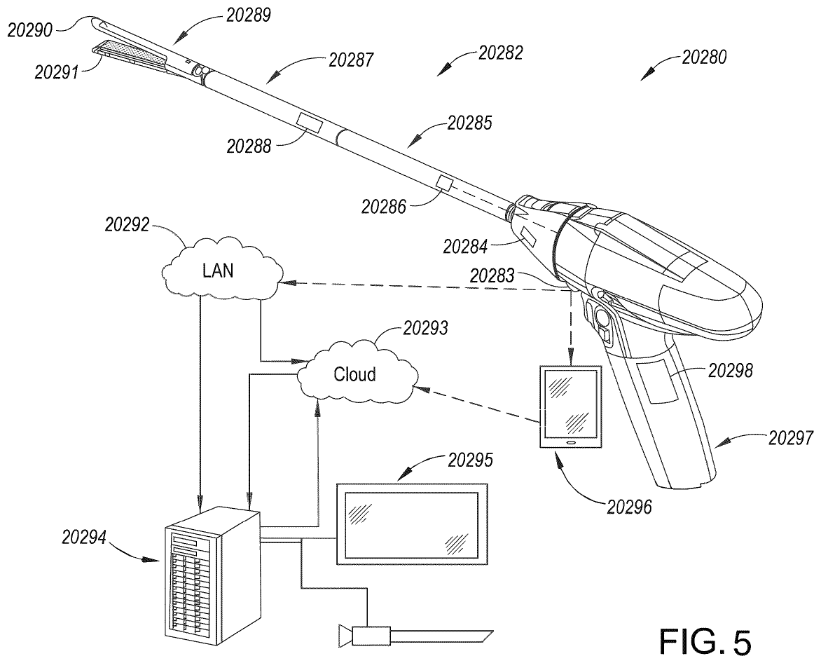

FIG. 5 illustrates an example surgical system 20280 that may include a surgical instrument 20282. The surgical instrument 20282 can be in communication with a console 20294 and/or a portable device 20296 through a local area network 20292 and/or a cloud network 20293 via a wired and/or wireless connection. The console 20294 and the portable device 20296 may be any suitable computing device. Surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It may be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (e.g., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

Aspects of the present disclosure may be integrated into a robot-enabled medical system comprising interconnected smart instruments capable of performing a variety of medical procedures, including both minimally invasive procedures (e.g., laparoscopy) and non-invasive procedures (e.g., endoscopy). In addition to performing a breadth of procedures, the robot-enabled medical system may provide benefits (e.g., enhanced imaging and guidance) to assist a clinician. The robot-enabled medical system may enable a clinician to perform the procedure from an ergonomic position. The robot-enabled medical system may provide the physician with the ability to perform the procedure with improved ease of use. In examples, surgical instrument(s) may be controlled by robot arm(s) (e.g., interconnected robot arms arrayed around a patient).

Feature(s) associated with laparoscopic robotics are provided herein. Traditional laparoscopic surgery may reduce hospital stay, reduce pain, reduce recovery time, lower herniation, and/or lower post operative infection rates. During a laparoscopic procedure the instruments may be introduced and used through sealed ports (e.g., trocars) placed in the abdomen wall. Small trocars may be beneficial and require less closure of the skin and serosal layers post extraction. Trocars may be independent of each other, and the location of trocars may define the triangulation options for the instruments that are used through them. Laparoscopic robotics may utilize trocars (e.g., instrument(s) may be introduced and extracted through these ports). Robotic arms (e.g., each robotic arm) may be configured for use of specific application(s) or instrument(s) (e.g., a trocar, a tool). For example, a tool may be inserted and extracted (e.g., via actuation) by a tool driver which is operable along the axis of a trocar.

Certain robot-enabled medical systems (e.g., laparoscopic robotics) face several limitations. For example, some robot-enabled medical systems often lack awareness of the surrounding operating room space (e.g., clinicians and objects). Robot arm(s) may have limited tool driver insertion/extraction range and/or overall arm length to minimize inter-arm interactions above the patient. Some robot-enabled medical systems (e.g., certain laparoscopic robot systems) may not be capable of removing an instrument from the trocar for external operations, such as sterilization, that must be performed on the tools before their next use.

Robot arm(s) may be large and/or capable of applying substantial loads to a person or object that may come in unintended contact with an arm during operation. Some robot arm(s) may have no kinematic display of where the external portions of the arms are to the user. While a robot may have awareness of the arm locations, in some examples, there may be no opportunity for choosing external arm motion control when the robot may be under control of the surgeon (e.g., no or limited feedback of the external positions and orientations of the robot). Thus, due in part to safety considerations and lack of kinematic display, certain robot-enabled medical systems may have limited access to a patient located near health care personnel.

Certain robot-enabled medical systems may be unable to handle and accurately account for requirements of instruments (e.g., hand-held devices). Hand-held devices may have an interaction and orientation outside the body which must be accounted for to avoid device-to-device collisions and minimize interaction with external environment objects (e.g., the table, the patient body wall, non-sterile portions of the field). A robot-enabled medical system may allow a user to have fine control of the orientation of hand-held device. A user may actuate the controls and displays of a hand-held device with sufficient force, mechanical advantage, and visibility to adequately operate the device as intended.

A limitation of some interconnected robot arm(s) may be the relative proximity of the arm(s) to each other and the patient body. Some approaches often require end-effectors to operate in a mostly fixed (e.g., close) approximation because the robotic arms may intertwine and/or entangle with one another during use. For example, robot arm(s) of certain robot-enabled medical systems for laparoscopic procedures often have a very limited amount of manipulation of trocars with respect to the patient body and fail to minimize inadvertent injury to the abdomen wall.

Feature(s) associated with use of robot arm(s) and robot instrument(s) of robot-enabled medical systems to address certain limitations (e.g., for use multi-port traditional laparoscopic approaches) are provided herein. Robot arm(s) may be arrayed around a patient and/or may have a common interconnection point (e.g., a table, common stand).

Interconnection of robotic arms of a robot-enabled medical system may improve inter-arm awareness and interaction. Each arm of a robot-enabled medical system may have a known (e.g., fixed) relationship with other arm(s) of the medical system. Improved inter-arm awareness and interaction may enable better prediction and control of one or more relative end-effector interactions and locations (e.g., tool placement).

A robot-enabled medical system may include robotic arm(s) are column-mounted, rail-mounted, mounted on a separate unit, and the like. In configurations, the robotic arms may move independently from each other. Robotic arms may each include multiple arm segments. Each arm segment may provide an additional degree of freedom to the robotic arm. The system may position the robotic arms into numerous configurations to access different parts of a patient's body. Robot arm(s) may be radially arrayed around a patient (e.g., a surface the patient is placed on) and may approach the patient from a perimeter. Robot arm(s) may originate from a centralized tower over the patient body and may approach the patient in a spherical envelope.

Robot arm(s) may be radially arrayed around and physically connected (e.g., mounted, attached) to a surface (e.g., table, platform, bed). Example robotic systems that are suitable incorporated into a table are described in U.S. Patent Application Publication No. 2021/0212776 (U.S. patent application Ser. No. 17/127,007), titled FUNCTIONAL INDICATORS FOR ROBOTIC MEDICAL SYSTEMS, filed Mar. 30, 2021, the disclosure of which is incorporated herein by reference in its entirety. Robot arm(s) may be attached (e.g., at a common point) with an improved array of starting connection points. Examples of medical systems incorporating physically interconnected robot arms are described in more detail in U.S. Pat. No. 10,667,875 (U.S. patent application Ser. No. 16/386,098), titled SYSTEMS AND TECHNIQUES FOR PROVIDING MULTIPLE PERSPECTIVES DURING MEDICAL PROCEDURES, filed Apr. 16, 2019, and U.S. Pat. No. 11,464,587 (U.S. patent application Ser. No. 16/708,284), titled SURGICAL ROBOTICS SYSTEM, filed Dec. 9, 2019, the disclosures of which are incorporated herein by reference in their entirety. The tools for radial robot arm systems may be fixed to tool driver(s) and articulating arm(s), or the tool driver may be more concentric to the trocar attachment point (e.g., only the tool occupies the moving space above a patient). Some examples of tools used in conjunction with connected robot arm(s) and/or their capabilities are described in U.S. Pat. No. 11,026,758 (U.S. patent application Ser. No. 16/011,521), titled MEDICAL ROBOTICS SYSTEMS IMPLEMENTING AXIS CONSTRAINTS DURING ACTUATION OF ONE OR MORE MOTORIZED JOINTS, filed Jun. 18, 2018, the disclosure of which is incorporated herein by reference in its entirety.

Robot arm(s) may be connected (e.g., mounted, attached) to a central interconnected pillar (e.g., stalk, tree). The robot pillar may be connected to a control console (e.g., for a user to operate the robot arm(s)) and may be connected to a computer hub for inter-cooperative control of the arms and instruments. For example, in U.S. Patent Application Publication No. 2022/0250242 (U.S. patent application Ser. No. 17/438,377), titled GUIDED TOOL CHANGE, filed Mar. 10, 2020, the disclosure of which is incorporated herein by reference in its entirety. The trocar may be positively affixed to a tool driver frame, for example as shown in U.S. Pat. No. 10,456,208 (U.S. patent application Ser. No. 15/126,725), titled SURGICAL CANNULA MOUNTS AND RELATED SYSTEMS AND METHODS, filed Mar. 17, 2015, the disclosure of which is incorporated herein by reference in its entirety. Trocars may be completely independent or have a common intersection point (e.g., for use in single incision laparoscopy. Instrument(s) that may be attached to a tool driver can be one or more of endocutters, advanced energy devices, or common surgical instruments.

Various examples of endocutters and surgical instruments that may be attached to the tool driver are described in U.S. Pat. No. 8,989,903 (U.S. patent application Ser. No. 13/350,502), titled METHODS AND SYSTEMS FOR INDICATING A CLAMPING PREDICTION, filed Jan. 13, 2012; U.S. Pat. No. 9,072,535 (U.S. patent application Ser. No. 13/118,241), titled SURGICAL STAPLING INSTRUMENTS WITH ROTATABLE STAPLE DEPLOYMENT ARRANGEMENTS, filed May 27, 2011; U.S. Pat. No. 9,072,536 (U.S. patent application Ser. No. 13/536,284), titled DIFFERENTIAL LOCKING ARRANGEMENTS FOR ROTARY POWERED SURGICAL INSTRUMENTS, filed Jun. 28, 2012; U.S. Pat. No. 10,531,929 (U.S. patent application Ser. No. 15/237,740), titled CONTROL OF ROBOTIC ARM MOTION BASED ON SENSED LOAD ON CUTTING TOOL, filed Aug. 16, 2016; U.S. Pat. No. 10,709,516 (U.S. patent application Ser. No. 15/943,226), titled CURVED CANNULA SURGICAL SYSTEM CONTROL, filed Apr. 2, 2018; U.S. Pat. No. 11,076,926 (U.S. patent application Ser. No. 15/927,926), titled MANUAL RELEASE FOR MEDICAL DEVICE DRIVE SYSTEM, filed Mar. 21, 2018; and U.S. Patent Application Publication No. 2022/0273309 (U.S. patent application Ser. No. 17/745,757), titled STAPLER RELOAD DETECTION AND IDENTIFICATION, filed May 16, 2022, the disclosures of which are incorporated herein by reference in their entirety.

A sterile barrier including a plastic sleeve may be placed over a robot arm which remains unsterile. Sterile instruments may be connected through sterile barriers plates (e.g., at each connection point between the sterile instruments and the remaining exposed unsterile portions of the robot arm). Example sterile instruments for robot arm(s) and sterile barrier plates are be described in U.S. Pat. No. 9,839,487 (U.S. patent application Ser. No. 15/121,718), titled METHOD FOR ENGAGING SURGICAL INSTRUMENT WITH TELEOPERATED ACTUATOR, filed Mar. 17, 2015, and in U.S. Pat. No. 10,543,051 (U.S. patent application Ser. No. 15/121,718), titled METHOD FOR ENGAGING SURGICAL INSTRUMENT WITH TELEOPERATED ACTUATOR, the disclosures of which are herein incorporated by reference in their entirety.

Robot arm(s) and robot instrument(s) may be used in close cooperation with one another to accomplish the surgical tasks of mobilization, transection, reconnection, manipulation, and/or retraction. These close interrelated motions, actions, and operations make the precision of interactions between these devices important. Robot arm(s) and robot instrument(s) may be used in concert with some instruments controlled by another surgeon, robot, and/or system. Cooperative (e.g., collaborative) communication of robot arm(s) and robot instrument(s) locations and/or monitoring of their location (e.g., by outside sources, such as operating room cameras) may improve coordination. Robot arm(s) may utilize data streams to mitigate entanglement issues by adjusting kinematics motions and operations sequentially or through a series of coordinated motions over time. Monitoring the physical location and motions of robotic arm(s) and robotic instrument(s) may improve collision avoidance.

Control of patient positioning may be added to a robot-enabled medical system comprising robot arm(s). The medical system may be configured to control (e.g., position) the surface (e.g., table, platform, bed) that one or more robot arms are connected (e.g., mounted) to. For example, the medical system may move the surface relative to the robot arm(s). Control of patent positioning may improve triangulation of the medical system comprising interconnected robot arm(s) as the robot arm(s) may originate around the surface in varied configurations.

Independent robot arms may be arrayed around a surgical field. Independent robot arms may approach the surgical field from angles independent of one another.

Multiple (e.g., separated) robot arm stations (e.g., towers, pillars) may be moveable with respect to one another and may be interconnected to a common (e.g., single) command-and-control station (e.g., hub) which may be connected to console(s) (e.g., hub(s)) for control by a surgeon or healthcare professional. In examples, a robot-enabled medical system may include multiple independent station robot arms with traditional tool drivers.

A robot-enabled medical system including multiple robot arm stations may include one or more independent robot arm stations (e.g., carts), which may have a wired connection to a control hub and surgeon console. A robot arm may be configured to determine alignment and location such that the robot-enabled medical system may determine the relationship of one robot arm to one another. The robot-enabled medical system may operate as a single smart robot. In some examples, the single smart robot may have no physical connections other than the communication wires between robot arm stations. A power system may be independently distributed to each robot arm station, minimizing the need for a high-power connection to a control hub (e.g., a high-power trunk connecting each robot arm station to a power system of the control hub). Tool(s) may be modularly attachable to the robot arm(s). A fully modular approach may enable a robot-enabled medical system to mimic the actions of a surgeon more closely. A modular approach may allow for increased mobility of the trocar relative to the patient, which may improve the flexibility of access. Systems incorporating multiple independent robot arm stations may manage collisions risk of the arms and tools outside of the patient. The management may be difficult (e.g., due to a flexible relationship between robot arms from different robot arm stations).

Smart power systems may be independent and may not have a fixed support point. Smart powered systems may be held and manipulated by a surgeon or health care personnel (e.g., directly). For example, smart powered systems may include one or more of: a handheld powered stapler with powered movement and/or placement (e.g., articulation) aspects and communication to other smart systems (e.g., Bluetooth); a handheld powered stapler with powered movement and/or placement (e.g., articulation and shaft rotation) aspects and communication to other smart systems (e.g., Bluetooth); or a handheld ultrasonic advanced energy device with communication to other smart systems (e.g., Bluetooth).

A handheld system may include a powered endoscopic linear stapler that may be disposable. For example, as described in U.S. Pat. No. 9,804,618 (U.S. patent application Ser. No. 14/226,071), titled SYSTEMS AND METHODS FOR CONTROLLING A SEGMENTED CIRCUIT, filed Mar. 25, 2014, the disclosure of which is incorporated herein by reference in its entirety. A powered endoscopic linear stapler may include a control mechanism for adjusting the speed at which it fires and/or the rate at which the adaptive firing and closing behavior may be applied (e.g., short pauses or stops) based on feedback within the system. The control mechanisms for adjusting the fire rate of a powered endoscopic linear stapler are described in more detail in U.S. Pat. No. 11,607,239 (U.S. patent application Ser. No. 15/130,590), titled SYSTEMS AND METHODS FOR CONTROLLING A SURGICAL STAPLING AND CUTTING INSTRUMENT, filed Apr. 15, 2016, and U.S. Pat. No. 10,052,044 (U.S. patent application Ser. No. 14/640,935), titled TIME DEPENDENT EVALUATION OF SENSOR DATA TO DETERMINE STABILITY, CREEP, AND VISCOELASTIC ELEMENTS OF MEASURES, filed Mar. 6, 2015, the disclosures of which are incorporated herein by reference in their entirety.

Adjustment by the control mechanism may be based on the distance the firing member moved over a predetermined time and/or a load of the firing member (e.g., a force measured within the firing system) or a load motor is experiencing (e.g., an electrical current through the motor). The handheld system (e.g., a powered endoscopic linear stapler) may be configured to detect a tissue within jaws of the handheld system (e.g., by determining thickness, location, and internal properties). Various examples of detecting tissue and determining tissue progression are described in U.S. Pat. No. 11,071,554 (U.S. patent application Ser. No. 15/628,053), titled CLOSED LOOP FEEDBACK CONTROL OF MOTOR VELOCITY OF A SURGICAL STAPLING AND CUTTING INSTRUMENT BASED ON MAGNITUDE OF VELOCITY ERROR MEASUREMENTS, filed Jun. 20, 2017; and U.S. Pat. No. 10,135,242 (U.S. patent application Ser. No. 14/478,895), titled SMART CARTRIDGE WAKE UP OPERATION AND DATA RETENTION, filed Sep. 5, 2014, the disclosures of which are incorporated herein by reference in their entirety.

The handheld system may be configured to provide the user feedback on states of system actuators and monitored aspect(s) of the tissue and/or progression of the staple line. For example, in U.S. Pat. No. 9,439,649 (U.S. patent application Ser. No. 13/712,090), titled SURGICAL INSTRUMENT HAVING FORCE FEEDBACK CAPABILITIES, filed Dec. 12, 2012, the disclosure of which is incorporated herein by reference in its entirety. The handheld system may include an input/output mechanism for communicating collected data stream(s) to external system(s) (e.g., via Bluetooth or direct connection). The handheld robot system may include a closure mechanism independent from a firing mechanism, and one or both of which may be powered and controlled separately.

A handheld system may include an end-user reusable powered stapler. The end-user reusable powered stapler may be configured to detect one or more of a reload configuration, a load from the tissue on the firing system, or control of a firing member's speed and pauses. The handheld system may include a rechargeable battery and may be used within a replaceable sterile barrier shell. A modular end-effector of the handheld system may enable multiple lengths of reloads and/or types of reloads that may have different primary control programs. The handheld system may utilize a single I-beam to close and fire. The handheld system may include an integrated wireless communication array for interacting with external systems.

A handheld system may include an ultrasonic tissue welding and cutting system. The ultrasonic tissue welding and cutting system may be powered by a modular battery. A battery pack and/or control electronics of the ultrasonic tissue welding and cutting system may be part of a first modular portion and an ultrasonic transducer may be part of a second modular portion. The handheld system may accommodate a replaceable wave guide and/or blade that may be disposable (e.g., for each patient). The modularity of a handheld system may be expanded to one or more of radio frequency (RF) monopolar, RF bipolar, and/or combination devices. The energy modalities may be blended, alternated, and/or combined based on one or more of tissue properties, jaw gap, or force. Operational parameters, such as one or more of tissue properties, jaw gap, or force may be communicated via wireless communication to other smart systems or recorded (e.g., stored).

Tethered but independent smart systems may include handheld systems which surgeon or healthcare personnel may handle (e.g., orient) that are connected (e.g., by a wired tether) to a fixed piece of control electronics. A tethered but independent smart system may include an advanced energy generator for adaptive control of one or more of monopolar RF, bipolar RF, or ultrasonic tissue welding.

A tethered but independent system may include one or more of an ultrasonic generator, a RF monopolar generator, or a RF bipolar generator that may be used to control the supply of energy to an attached handpiece (e.g., to control the energy modality). The energy magnitude and energy modality may be controlled by a generator by sensing aspects of the tissue and/or the device, for example as described in U.S. Pat. No. 10,842,523 (U.S. patent application Ser. No. 15/382,515), titled MODULAR BATTERY POWERED HANDHELD SURGICAL INSTRUMENT AND METHODS THEREFOR, filed Dec. 16, 2016; U.S. Pat. No. 11,051,873 (U.S. patent application Ser. No. 15/177,449), titled SURGICAL SYSTEM WITH USER ADAPTABLE TECHNIQUES EMPLOYING MULTIPLE ENERGY MODALITIES BASED ON TISSUE PARAMETERS, filed Jun. 9, 2016; and U.S. Pat. No. 10,765,470 (U.S. patent application Ser. No. 15/177,466), titled SURGICAL SYSTEM WITH USER ADAPTABLE TECHNIQUES EMPLOYING SIMULTANEOUS ENERGY MODALITIES BASED ON TISSUE PARAMETERS, filed Jun. 9, 2016, the disclosures of which are incorporated herein by reference in their entirety.

Aspects of the device and/or tissue (e.g., max applied temperature) may be used to limit the input energy. Some examples of tethered but independent smart systems and/or their capabilities are described in U.S. Pat. No. 11,589,888 (U.S. patent application Ser. No. 16/209,453), titled METHOD FOR CONTROLLING SMART ENERGY DEVICES, filed Dec. 4, 2018; U.S. Patent Application Publication No. 2019/0201136 (U.S. patent application Ser. No. 16/209,395), titled METHOD OF HUB COMMUNICATION, filed Dec. 4, 2018; and U.S. Patent Application Publication No. 2019/0201112 (U.S. patent application Ser. No. 15/940,629), titled COMPUTER IMPLEMENTED INTERACTIVE SURGICAL SYSTEMS, filed Mar. 29, 2018, the disclosures of which are incorporated herein by reference in their entirety.

For example, an example generator may be configured to deliver multiple energy modalities to a surgical instrument. The generator may provide RF and ultrasonic signals for delivering energy to a surgical instrument either independently or simultaneously. The RF and ultrasonic signals may be provided alone or in combination. The RF and ultrasonic signals may be provided simultaneously. The generator output can deliver multiple energy modalities (e.g., ultrasonic, bipolar or monopolar RF, irreversible and/or reversible electroporation, and/or microwave energy, among others) through a single port, and these signals can be delivered separately or simultaneously to the end effector to treat tissue. The generator may include a processor coupled to a waveform generator. The processor and waveform generator may generate a variety of signal waveforms based on information stored in a memory coupled to the processor. The digital information associated with a waveform may be provided to the waveform generator, which may include one or more DAC circuits to convert the digital input into an analog output. The analog output may be fed to an amplifier for signal conditioning and amplification. The conditioned and amplified output of the amplifier may be coupled to a power transformer. The signals may be coupled across the power transformer to the secondary side, which may be in the patient isolation side. A first signal of a first energy modality may be provided to the surgical instrument via a first energy terminal. A second signal of a second energy modality may be coupled across a capacitor and may be provided to the surgical instrument via a second energy terminal. It may be appreciated that more than two energy modalities may be output and thus the subscript “n” may be used to designate that up to n ENERGYn terminals may be provided, where n is a positive integer greater than 1. It also may be appreciated that up to “n” return paths RETURNn may be provided.

Feature(s) associated with smart instrument control relating to single access port robotics (e.g., robots used for smart single-site surgery) are provided herein. Single access port robotics may include multiple smart instruments introduced through a single access port that may be interconnected (e.g., have a common portion, such as a pillar, stalk, or hub). Single access port robotics may include robotics used in laparoscopic-endoscopic single-site surgery (LESS) and/or single site laparoscopy (SSL). SSL may include the introduction of multiple instruments with a single incision. Triangulation of instruments within a limited site space is a challenge of introducing robotics to single access port procedures. Single access port robotics may include multiple instruments (e.g., two or more instruments) and may include a camera. Single access port robotics may have the ability to use articulation point(s) (e.g., of each portion) to spread and triangulate on a local surgical interaction site.

Single access port robotics may include single incision laparoscopic surgery and/or single site laparoscopy robot(s). Single access port robotics may have an interconnection point (e.g., stalk) and may be radially supported from a single hub (e.g., tower). Single access port robotics may be a powered hand-held robot (e.g., with an integrated scope and/or two or more triangulating arms).

Single site laparoscopy (SSL) may be the introduction of multiple instruments with a single incision. SSL may not be a direct replacement of multi trocar lap. A patient may recover from multiple small incisions more efficiently than a single larger incision (e.g., of the same overall diameter, for example five, 5 mm holes may heal faster and with less pain than a single 25 mm hole). With the introduction of robotics, triangulation of instruments with limited site access may be necessary. A multi-instrument or a two instrument plus camera may have the ability for the scope and the multiple instruments to operate in parallel in a portion of their longitudinal space. The ability to use and triangulate at least two articulating instruments on a local surgical interaction site may improve treatment.

Feature(s) associated with flexible endoscopy robotics are provided herein. Flexible endoscopy robotics may be introduced through natural orifice(s). Flexible endoscopy robotics may include one or more of a robotically controlled scope insertion, a manipulation aspect for remote operation of tools (e.g., through the working channel), or an imaging aspect (e.g., at least for part of the time in use).

Robotic flexible endoscopy may include the robotic control of a flexible endoscope. In examples, endoscopes may have a differing size and configuration relative to the surgical and anatomic use location. An example surgical robot used in flexible endoscopy may include removable local imaging component that may be exchanged with insertable tools (e.g., while being monitored by a control of an active controlled catheter and/or outside imaging). The example surgical robot may include a flexible robotic cannula with a working channel and/or integrated imaging.

Thoracoscopes may be used in flexible endoscopy. An example thoracoscope may include a multi-axis controllable distal tip and/or an insertion and extraction control mechanism. The example thoracoscope may have working channel(s) (e.g., one or more). The example thoracoscope may include an integrated camera and a separate working channel that may enable a more compact profile or may include an imaging system (e.g., configured to provide continual or uninterrupted imaging). The example thoracoscope may have a navigation and/or a control system for that may be configured to direct a tip of the thoracoscope from outside. The example thoracoscope may include a stress (e.g., strain) based in the flexible neck and/or an electromagnetic navigation tracking sensor. Other types of endoscopes and/or steerable catheters may include similar controls.

Endoscopes may be used in robotic flexible endoscopy. For example, an endoscope may include one or more of: anoscopes (e.g., used in anoscopy to view the anus and/or rectum); arthroscopes (e.g., used in arthroscopy to view joints); bronchoscopes (e.g., used in bronchoscopy to view the trachea, windpipe, and/or lungs); colonoscopes (e.g., used in colonoscopy to view the length of the colon and/or large intestine); colposcopes (e.g., used in colposcopy to view the vagina and/or cervix); cystoscopes (e.g., used in cystoscopy to view the inside of the bladder); esophagoscopes (e.g., used in esophagoscopy to view the esophagus); gastroscopes (e.g., used in gastroscopy to view the stomach and/or duodenum); laparoscopes (e.g., used in laparoscopy to view the stomach, liver, and/or other abdominal organs, including female reproductive organs); laryngoscopes (e.g., used in laryngoscopy to view the larynx and/or voice box); neuroendoscopes (e.g., used in neuroendocscopy used to view areas of the brain); proctoscopes (e.g., used in proctoscopy to view the rectum and/or sigmoid colon); sigmoidoscopes (e.g., used in sigmoidoscopy to view the sigmoid colon); or thoracoscopes (e.g., used in thoracoscopy to view pleura).

Feature(s) associated with smart controlled irrigation (e.g., for advanced monopolar and bipolar cooling tip devices) are provided herein. Cooling tip devices may include advanced cooperative saline supplemented RF energy instruments, including monopolar RF energy coagulation devices and bipolar RF energy coagulation devices. A monopolar RF energy coagulation device may include an electrode and where the patient may be part of the return path, for example by having a conductive pad or capacitive pad in contact with the patient's skin back to the generator. A monopolar RF energy coagulation device may include an energy application-controlled irrigation (e.g., conductive irrigation like saline) aspect. complementary to (e.g., location, rate, pressure). Bipolar RF energy coagulation devices (e.g., devices that may include an electrode source a return path that may be part of the same device interface) may include an controlled irrigation (e.g., conductive irrigation like saline) aspect. The irrigation may be complementary to the energy application. The operational parameters associated with controlling the irrigation may include location, rate, pressure, and/or the like.

Monopolar electrode configurations may use a patient as the return path such that control of pressure and power density is easier. Conductivity of the return path may control the location and degree to which energy density may be applied. Strictures or insufficient return pad attachment when using monopolar devices may burn the patient.

Bipolar electrode configurations may have a controlled source and return path. Controlling the tissue properties between electrodes in a dual exposed manner (e.g., such as in a bipolar electrode configuration) may be difficult.

PTFE coating may be used on electrode configurations to localize the energy and focus the energy density. These coatings may be used in combination with monopolar electrode configurations and bipolar electrode configurations to further tune energy deployment and focus.

When RF energy is applied, some conditions may occur, such as tissue sticking to electrodes, a large area of collateral thermal damage, and/or conductivity issues (e.g., as tissue dries out or has properties which less salinity). A complementary liquid (e.g., saline) may be released on or near the electrodes cooperatively while the RF energy of the device is in use (e.g., applied). Complementary liquid may differ from suction-irrigation that may occur with monopolar cutting in that an applied complementary liquid (e.g., saline) may be a supplement to the energy application (e.g., rather than a wash). Complementary liquid irrigation may be used with monopolar RF energy devices and bipolar RF energy devices.

Complementary liquid irrigation may be used with point contact devices and/or dual jaw systems. Complementary liquid irrigation may be used with point contact devices to maintain a cool electrode temperature, clean, and/or improve the electrical path between the poles (e.g., when used with bipolar RF energy devices). The control, direction, flow rate, and/or the location of contact of a complementary liquid with the tissue and electrode may be relative to the ejection of the fluid control and may affect the usefulness of the saline drip. Some examples of an electrosurgical instrument with fluid control are described in U.S. Pat. No. 10,751,117 (U.S. patent application Ser. No. 15/274,559), titled ELECTROSURGICAL INSTRUMENT WITH FLUID DIVERTER, filed Sep. 23, 2016, the disclosure of which is incorporated herein by reference in their entirety. Local contact energy may be used for hemostasis touch up, localize RF ablation, or organ surface cauterization.

RF energy devices may be open loop irrigation application systems. In open-loop irrigation application, a user may control (e.g., set) the rate of application of a liquid (e.g., saline) through activation of RF energy (e.g., the liquid starts pumping during application of RF energy). The irrigation control system may monitor the RF generator for the application of energy to determine when liquid should be applied.

RF energy devices may measure tissue impedance and may use measured tissue impedance to adjust a power balance supplied to the system (e.g., to create and maintain a desired energy density).

RF energy devices may measure force to determine an amount of force applied from the electrode to the tissue. The measured force may be used to adjust power levels of an RF generator (e.g., to create and maintain the desired energy density).

A relationship may exist between compression, power, and conductivity between an energy device and a target tissue. Imbalances in this relationship may result in charring, tissue sticking, and inadequate sealing strength. Feature(s) associated with closed loop control on complementary liquid irrigation to address imbalances are provided herein.

Communicating inputs to closed loop control may reduce the frequency of undesirable conditions (e.g., tissue sticking to electrodes, a large area of collateral thermal damage, conductivity issues) during operation of an RF energy device. Communicated inputs may include one or more of: tissue impedance, generator power level, device pressure on tissue, electrode-tissue contact area, or thermal load communicated to the tissue or body as a whole (e.g., based on the temperature and magnitude of the saline). Outputs (e.g., of closed loop control on irrigation) may include one or more of: saline volumetric rate; saline pressure (e.g., pressure may increase the area of effect of the saline and may improve cleaning and/or minimize sticking of tissue to the electrode); saline temperature (e.g., temperature of the saline may cool the tip and may minimize collateral thermal damage to the surrounding tissues); or salinity level (e.g., salinity of the fluid and the volume present in the area may adapt the conductivity of the tissue enabling more energy to be applied in a tighter area). Control of surgical field irrigation is described in U.S. Pat. No. 11,160,602 (U.S. patent application Ser. No. 15/689,853), titled CONTROL OF SURGICAL FIELD IRRIGATION, filed Aug. 29, 2017, the disclosure of which is incorporated herein by reference in its entirety.

Advanced ablation systems may be configured to perform in-situ tissue destruction of tumors and other abnormal tissues. Advanced ablation systems may include one or more of: microwave application (e.g., from a localized probe source radiating outwards) to increase cell temperature above the cellular death threshold; directional overlapping of focused ultrasonic waves to induce heat and cell death (e.g., through the interaction of the ultrasonic waves in a localized portion of the body); cryogenic fluid (e.g., nitrous oxide) to reduce the cellular temperature below the lower cellular death threshold; and/or or electrical potential focused between originating and returning electrodes (e.g., needles) to induce cellular death (e.g., through the breakdown of the external membrane of the cell).

Microwave ablation may include increasing cell temperature above the cellular death threshold (e.g., by radiating microwaves outward from a localized probe source). Microwave ablation may offer benefits over radiofrequency ablation including that the energy may readily propagate regardless of tissue type or desiccation. Microwave ablation energy may be less susceptible to heat sink than radiofrequency ablation, which may enable one or more of more predictable ablations, larger single probe ablations, multi-probe capabilities, or faster ablations (e.g., two to four times faster).

A microwave ablation system may include a microwave generator, a flexible coaxial cable, and/or a microwave antenna. Microwaves may be generated by a magnetron in a microwave generator. A microwave antenna may be connected to a coaxial cable to the microwave generator and may transmit microwaves into the tissue. Antennas may be classified based on physical features and radiation properties. A microwave antenna may be a 14-17-gauge structure that may be placed into the tumor (e.g., during treatment). Total tumor necrosis may be achieved (e.g., when temperature remains at 54° C. for at least three minutes or instantly when temperature reaches 60° C.). A distal tip portion, which may be referred to as the needle, of an antenna may oscillate (e.g., agitate) water molecules causing friction and heat. Examples of microwave ablation systems and their capabilities are described in U.S. Pat. No. 9,877,783 (U.S. patent application Ser. No. 15/395,959), titled ENERGY DELIVERY SYSTEMS AND USES THEREOF, filed Dec. 30, 2016, the disclosure of which is incorporated herein by reference in its entirety.

Therapeutic high intensity ultrasound may include directional overlapping of focused ultrasonic waves that induce heat and cell death through their interaction in a localized portion of the body. A therapeutic high intensity ultrasound system may include a concave, spherical, and/or phased array transducer that may be configured to focus triangulating ultrasonic waves at a distant focal location.

High-intensity focused ultrasound (HIFU) may be a minimally invasive medical procedure that may use ultrasound waves to treat certain conditions (e.g., tumors, uterine fibroids, tremors). High-intensity and highly focused sound waves may interact with targeted tissues in a patient's body to modify or destroy the targeted tissues. HIFU treatment may include delivering sufficient energy to increase a tissue's temperature to a cytotoxic level quickly (e.g., so that the tissue vasculature may not affect the extent of cell killing).

HIFU and magnetic resonance-guided focused ultrasound (MRgFUS) may be effective as non-invasive ablation modalities (e.g., for soft tissues). Tissue damage from HIFU may be based on tissue coagulative thermal necrosis (e.g., due to the absorption of ultrasound energy during tissue transmission, known as thermal effect) and/or ultrasound-induced cavitation damage.

HIFU may impact endobronchial ultrasound (EBUS) sensing means. For example, the EBUS sensing means may lose sight of a therapy target, such as during navigation for, or location of, the therapy target. For example, if the EBUs is the target locating system for a tumor when the HIFU is activated, the target lock may move due to interference and cause ablation of unintended materials (e.g., tissues).

HIFU may be performed by using a transducer outside of the body targeting a tumor inside the body and/or by using a transducer and imaging source on a flexible endoscope (e.g., inside the body). HIFU may suffer from loss of signal when the HIFU is activated (e.g., regardless of transducer location outside or inside the body). Robotic control of a flexible endoscope may be inhibited by loss of a control signal. In some examples, HIFU may be performed using separate treatment and imaging systems that may be unconnected.

A HIFU beam may pass through overlying skin and tissues without harm and may focus on a localized area of a patient (e.g., with an upper size limit of approximately 3-4 cm in diameter for tumors). Lesion coagulative necrosis may occur at an affected area at the focal point of the HIFU beam. When a tumor may be ablated, a sharp boundary between dead and live cells may be created. The boundary width between totally disrupted cells and normal tissue may be no more than 50 μm. Tissue damage from HIFU may result from tissue coagulative thermal necrosis (e.g., due to the absorption of ultrasound energy during tissue transmission, known as thermal effect, and ultrasound-induced cavitation damage).

Cryoablation tissue destruction may include cryogenic fluid (e.g., nitrous oxide) being used to reduce the cellular temperature below the lower cellular death threshold. Percutaneous cryoablation may be performed by inserting cryoprobes into malignant tissue (e.g., under imaging guidance). After targeting the lesions with cryoprobe(s), the cryoprobe(s) are rapidly cooled (e.g., by removing heat from the tissue by conduction via physical contact with the cryoprobe). The Joule-Thompson effect (e.g., rapid expansion of a gas that does not work, known as adiabatic expansion, results in a change in the temperature of the gas) may be used to rapidly cool the cryoprobe(s). The temperature and rate of change of temperature of the cryoprobe(s) may be controlled by manipulating the rate the liquid is introduced and the pressure or rate at which the gas is allowed to expand and escape. Examples of pressure regulation within a cryosurgical system are described in U.S. Pat. No. 11,266,458 (U.S. patent application Ser. No. 16/389,343), titled CRYOSURGICAL SYSTEM WITH PRESSURE REGULATION, filed Apr. 19, 2019, the disclosure of which is incorporated herein by reference in its entirety.

A cryoprobe may be a high-pressure, closed-loop, gas expansion system. When a high-pressure room temperature gas (e.g., argon) reaches a distal aspect of the cryoprobe, the gas is forced through a throttle (e.g., narrow opening) and then allowed to rapidly expand to atmospheric pressure. The rapid expansion of the argon causes a decrease in the temperature (e.g., of surrounding tissue).

Cryoablation may cause cellular damage, death, and necrosis of tissues by direct mechanisms, (e.g., cold-induced injury to cells) and indirect mechanisms (e.g., changes to the cellular microenvironment that may impair tissue viability). As a cryoprobe absorbs heat from the tissue, the tissue may cool, and ice crystals may form in the extracellular space. The ice crystals may sequester free water, which may increase the tonicity of extracellular space. Osmotic tension may draw free intracellular water from cells, dehydrating the cells.

Irreversible electroporation (IRE) may include focusing electrical potential between originating and returning electrodes (e.g., needles) to induce cellular death by breaking down the external membrane of the cell. IRE may kill cells by increasing the electrical potential across the cell membrane for a period of time. IRE may provide an effective method for destroying cells while avoiding some of the negative complications of heat-inducing therapies. IRE may kill cells without raising the temperature of the surrounding tissue to a level at which permanent damage may occur to the support structure or regional vasculature.

Application of IRE pulses to cells may ablate large volumes of undesirable tissue with no or minimal detrimental thermal effects to the surrounding healthy tissue. IRE may be utilized in conjunction with electrodes and/or other electrical ablation devices to perform one or more minimally invasive surgical procedures or treatments. IRE is described in U.S. Pat. No. 10,314,649 (U.S. patent application Ser. No. 13/565,307), titled FLEXIBLE EXPANDABLE ELECTRODE AND METHOD OF INTRALUMINAL DELIVERY OF PULSED POWER, filed Aug. 2, 2012, the disclosure of which is incorporated herein by reference in its entirety.

Ablation may require meeting or surpassing an intensity level threshold for a duration for applied energy to effect cell death. Ablation methods may involve an expanding area of effect that grows the longer the power is applied (e.g., after the minimum power-time requirement is met). Ablation technologies may operate in a closed loop manner (e.g., based on a locally measured aspect of the application device's probes).

Ablation concentration or expansion zones may have an origin of the ablation energy and expand outward from that location. When the outwardly expanding energy modality reaches a magnitude to cause cell death an “effect zone” may be defined. Effect zones may be affected by tissue properties (e.g., density and shape of the abnormal tissue). Unintentionally affected adjacent zones (e.g., unintentional effect zones) may be affected by tissue properties (e.g., density and shape) of tissue surrounding the abnormal tissue. Differentiating between “killing” cells and “damaging but not killing” cells may be difficult for a distributing energy source to predict or monitor.

Energy ablation technologies may use multiple probes to define the effected zone. IRE may use needle proximity to define an inside space (e.g., area of effect).

Laparoscopic endoscopic cooperative surgery (LECS) may be performed using the surgical instrument(s), device(s) and/or system(s) described herein. LECS may be used for procedures such as a gastric wedge resection that is applicable for submucosal tumor resection (e.g., gastric submucosal tumors such as gastrointestinal stromal tumor (GIST)) independent of tumor location and size. For example, LECS may be used to resect an esophageal approached serosal gastric tumor, where the tumor may be too large to be extracted orally and may be extracted laparoscopically.

LECS may be used for stomach tumor dissection, for example, for a tumor that may be located adjacent to the esophageal sphincter on the greater curvature posterior side of the stomach. The tumor may require mobilization and retraction of the stomach into an irregular shape to access, dissect, and/or remove the tumor laparoscopically. In examples, LECS for such a procedure may include endoscopic sub-mucosal dissection with trans organ wall flexible endoscopic access combined with laparoscopic manipulation and specimen removal.

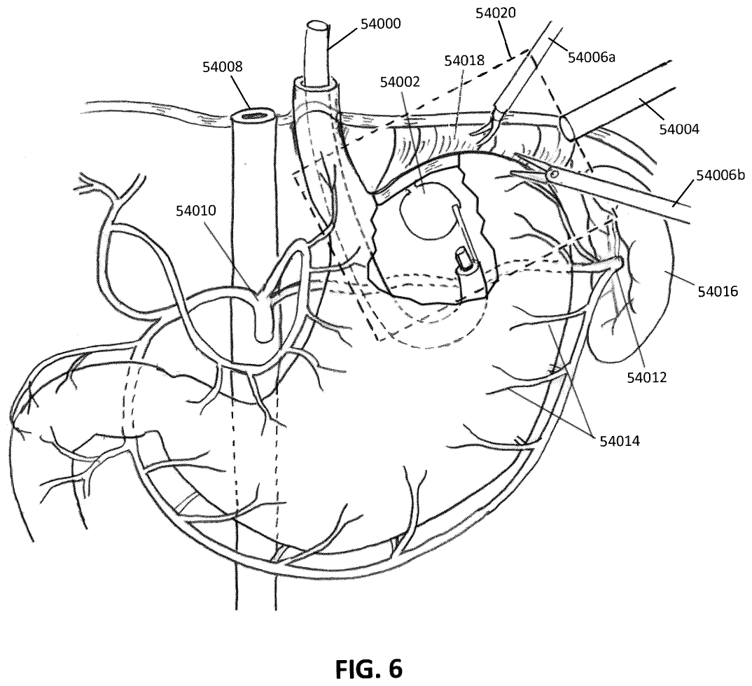

FIG. 6 depicts an example of anatomy and surgical tools during a stomach tumor extraction. Endoscope 54000 may be used to view tumor 54002. Laparoscopic scope 54004 may provide a view of the stomach exterior. Grasping devices 54006a, 54006b may be used to secure the tumor during the procedure. Grasping devices 54006a, 54006b may be controlled by a single robot or may be controlled by separate robots. Depicted anatomy includes aorta 54008, splenic artery 54010, left gastroepiploic artery 54012, short gastric arteries 54014, spleen 54016, and connective tissue 54018. Region 54020 is where the extraction will (e.g., predominantly) occur.

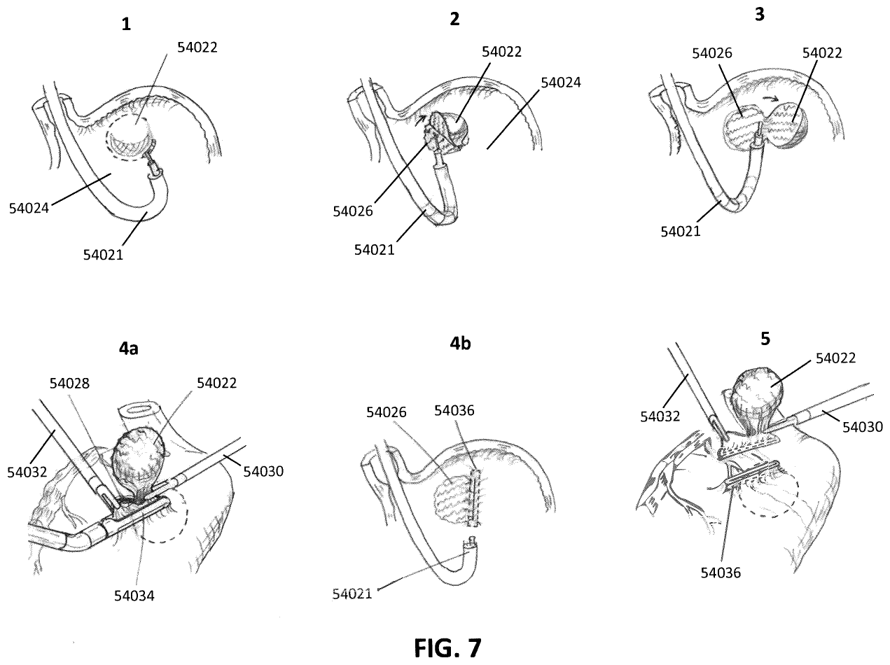

FIG. 7 depicts an example method for performing a stomach tumor dissection (e.g., within region 120 of FIG. 6). Performing a stomach tumor dissection may include one or more of the following operations. At 1, endoscopic monopolar device 54021 may be used to apply monopolar energy to tumor 54022. Tumor 54022 may be completed connected to the stomach wall on mucosal layer 54024 (e.g., tumor 54022 may be a mucosal tumor). At 2, applied monopolar energy may begin to separate mucosal layer 54024 and submucosal layer 54026. Partial disconnection of tumor 54022 from submucosal layer 54026 may increase impedance. At 3, tumor 202 may be connected to submucosal layer 54026 via a small amount of tissue (e.g., which may further increase impedance).

As shown in FIG. 7, at 4a, an exterior view of the stomach is depicted. Incision 54028 may be made through the stomach wall. Tumor 54022 may be pulled through incision 54028. First grasping device 54030 (e.g., an endoscopic grasping device) may grip tumor 54022 (e.g., that may be mostly resected from inside the stomach). Endoscopic articulation may be used to control the tumor position and the orientation of the stomach (e.g., to prevent loss of acid control when the trans-wall incision is performed). Second grasping device 54032 (e.g., a laparoscopic grasping device) may be repositioned from the laparoscopic side into close proximity to the abdominal cavity side of the stomach wall. Second grasping device 54032 may take hold of the outside wall to control the stomach (e.g., during incision). First grasping device 54030 and second grasping device 54032 may be released for autonomous control. First grasping device 54030 may be given a proximity distance to maintain to laparoscopic grasping device 54032. Second grasping device 54032 may be placed in station keeping mode. A cutting element (e.g., laparoscopic monopolar blade, scissors, or the like) (not depicted) may be used to cut along the base of the still intact connection of tumor 54022 and the inside wall of the stomach. A blade force may be resisted by second grasping device 54032 keeping retraction. Orientation may be controlled (e.g., to prevent acid spill) by the endoscope shape and first grasping device 54030's hold on the tumor base. The cutting element may be removed (e.g., after an incision is made) and second grasping device 54032 may be used to grasp the tumor base from the laparoscopic side. The proximity control may be used to keep the endoscopic hold of the tumor relative to second grasping device 54032 (e.g., for fixation). A proximity distance may be adjusted (e.g. shortened) to bring the grasping devices (e.g., an endoscopic grasping device and a laparoscopic grasping device) closer together. First grasping device 54048 may release the tumor hold while maintaining its position (e.g., when second grasping device 54032 has the base of the tumor). The user-controlled second grasping device 54032 may flip the tumor to the abdomen space. The second grasping device 54032 may be placed in a station keeping mode while endocutter 54034 is controlled. Endocutter 54034 may be positioned across the incision and the base of the tumor. Endocutter 54034 may be fired (e.g., to simultaneously cut the tumor loose and seal the incision). Endocutter 54034 may be placed in a station keeping mode (e.g., while clamped on the tissue).

At 4b, the endoscopic tools may be retracted, as shown in an interior view. Second grasping device 212 may be placed in user control, and the jaws of endocutter 54034 may be opened. A portion 54036 of mucosal layer 54024 and submucosal layer 54026 may be stapled (e.g., when inverted). At 5, tumor 54022 may be removed from the surgical site.

A LECS procedure to remove a gastric tumor may include one or more of the following operations.

A gastroscope (e.g., 5-12 mm over tube with working channel sizes of 2-4 mm and having a local visualization scope) may be introduced. Laparoscopic trocar(s), a laparoscope, and/or tissue manipulation and dissection instruments may be introduced. The stomach may be manipulated and held (e.g., in a position where stomach acids are not over the portion of the stomach where the tumor resection may be performed, and the intra-cavity cut may be made). Stomach acids may be managed (e.g., with respect to gravity) to prevent inadvertent escape of the acids into the abdomen cavity. Blood vessels in the excision area may be prepared. The gastroepiploic artery (e.g., which surrounds the perimeter of the stomach and may be fixed to surrounding structures) may be freed (e.g., to enable mobilization and/or separation of connective tissues). During this operation, bleeding may occur. Bleeding may require intervention from the laparoscopic side. Endoscopic submucosal resection around the tumor may be performed. The tumor location from the endoscope side may be located and communicated to the laparoscope side. The stomach may be mobilized on the laparoscope side to facilitate stomach retraction and manipulation. The perimeter of the tumor may be marked (e.g., using an energy modality, for example RF, Argon plasm, laser, or the like). Glycerin (e.g., 10% glycerin) or saline may be injected into the submucosal layer to separate the tumor from the serosal layer (e.g., for dissection). An energy supplemented device may be used to separate the mucosal layer and tumor from the serosa (e.g., by separating the sub-mucosal layer and dissecting the tumor from the underlying tissues).

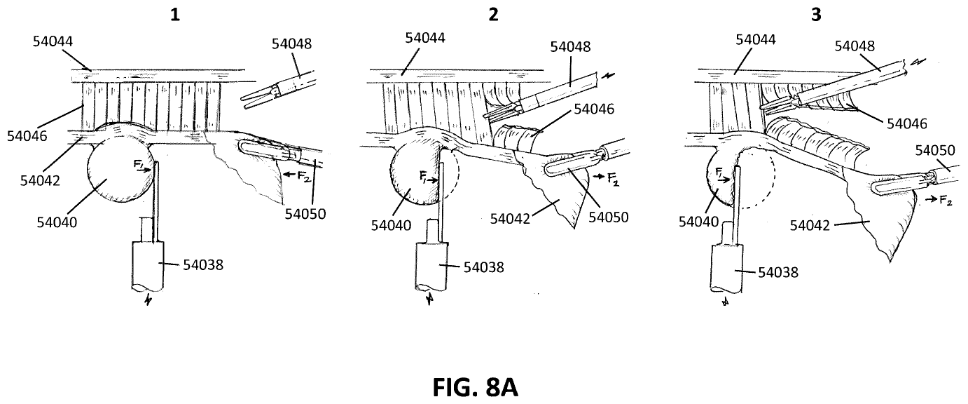

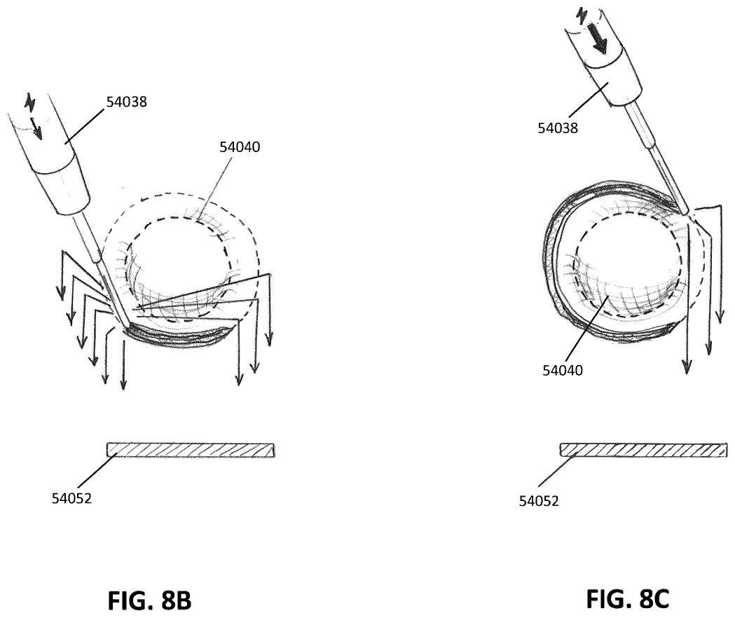

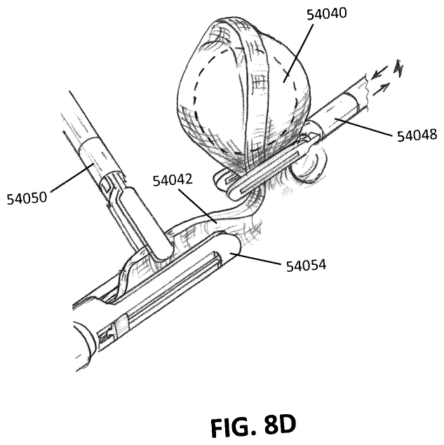

FIG. 8a depicts an example method for stomach tumor extraction. As shown, at 1, endoscopic monopolar device 54038 may be introduced. Tumor 54040 may be connected to stomach wall 54042. Stomach wall 54042 may be connected to diaphragm 54044 by connective tissue 54046. Laparoscopic tools, including laparoscopic bipolar dissecting device 54048 and laparoscopic grasper 54050 may be introduced on the laparoscopic side of the tumor. Laparoscopic grasper 54050 may be used to secure stomach wall 54042.

As shown in FIG. 8a, at 2, mucosal and sub-mucosal dissection of tumor 54040 may be performed (e.g., based on the depth of the tumor in the stomach wall). Inadvertent perforation of the serosal tissue layer may create leaks from the stomach to the abdomen. Controlled energy usage (e.g., by endoscopic monopolar device 54038) may precisely remove the tumor without burning through entire stomach wall 54042. FIGS. 8b-c depict an example of controlled energy applied on the endoscopic side, where non-dissected tissue is represented by dotted lines. Monopolar device 54038 may apply energy (e.g., RF energy) to gradually dissect tissue. Applied energy may be captured by return pad 54052 located under the patient. As tissue is dissected (as depicted in FIG. 8c), there may be less of a return path for the applied energy and more power may be needed.

Referring back to FIG. 8a, at 3, energy assisted dissection may be stopped while a portion of tumor 54040 remains in connection with stomach wall 54042 (e.g., stomach lining). An opening in the serosa may be cut and tumor 54040 may be passed (e.g., flipped) through the opening to the laparoscopic side (e.g., for final specimen transection and removal). Tumor 54040 may be pivoted through the incision (e.g., to keep hold of the tumor during extraction). FIG. 8d depicts an example of the portion of tumor 54040 pivoted to the laparoscopic side and held by bipolar device 54048. Laparoscopic grasping device(s) 54050 may be used to hold and orient stomach wall 54042.

As shown in FIG. 8d, laparoscopic seromuscular dissection of tumor 54040 may be performed. Artificial hole(s) may be created from the laparoscopic side to enable introduction of bipolar device 54048 from the laparoscopic side. Bipolar device 54048 may be used to dissect portions of the interface that are not fully accessible from the endoscopic side. A laparoscopic incision may be made at the base of the remaining connection between the tumor and the serosa layer. An incision may be made outside the tumor margins (e.g., to allow for removal of the tumor from the laparoscopic side) while ensuring the tumor (e.g., all of the tumor) is removed. The location of the incision may be initiated from the laparoscopic side and coordinated with the endoscopic side and/or the tumor location. Tumor 54040 may be controlled and manipulated during the incision by laparoscopic grasper(s) 54048, 54050 (e.g., to prevent inadvertent cutting of the tumor).

Tumor 302 may be passed (e.g., flipped about the point of remaining attachment to the serosa) to the laparoscopic instruments for extraction. The stomach orientation may be controlled (e.g., by laparoscopic grasper(s) 54050) to prevent stomach acid from escaping into the abdomen. Localized bleeding may be controlled (e.g., with advanced energy from the laparoscopic and/or the endoscopic spaces) when pivoting tumor 54040 from the control and interaction of the endoscopic instruments to the control and interaction of the laparoscopic instruments. During the hand-off there may be point(s) in time where both sets of instruments are interacting with the same tumor tissue.

As shown in FIG. 8d, a dissecting device 54054, such as an endocutter, may be introduced from the laparoscopic side. Bipolar device 54048 may be positioned to transect tumor 54040 from the remaining stomach wall and to seal the opening through which the tumor was passed. Poor positioning of the endocutter may result in a hole remaining in the organ that may be closed before completion of the procedure.

A laparoscopic stapler 54054 may be introduced over the incision and the remaining tumor attachment. The laparoscopic stapler 54054 may be fired to release tumor 54040 from the tissue on the laparoscopic side and/or seal the incision with staples. If the stapler jaws are overloaded by tissue thickness, the overload may result in inadequately formed staples (e.g., staples that may not seal the organ and may result in localized bleeding).

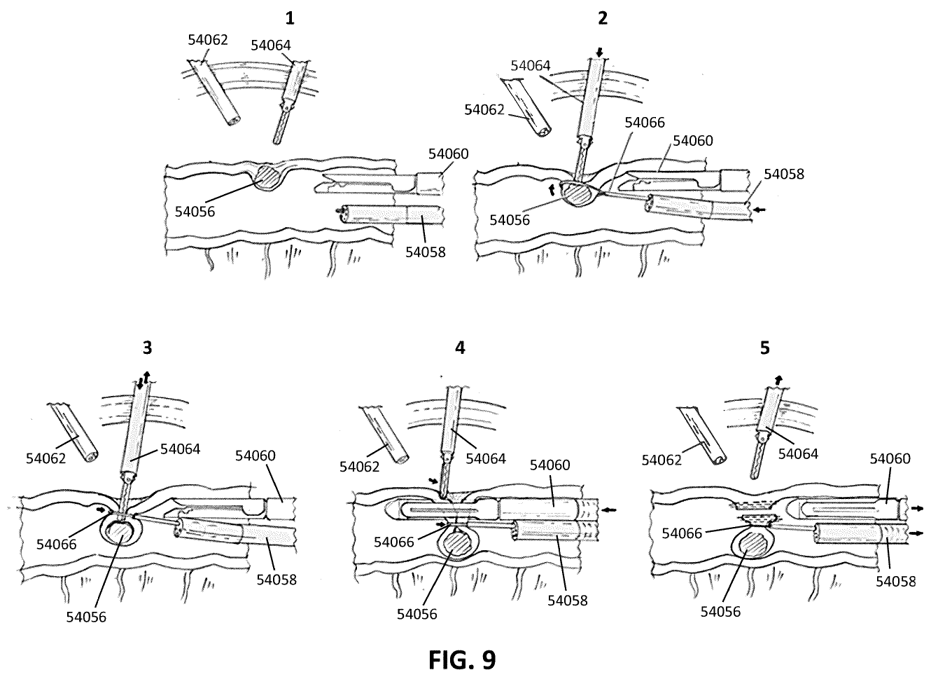

In examples, the tumor may be removed by oral extraction (e.g., as opposed to laparoscopically). FIG. 9 depicts an example method of oral extraction of a tumor (e.g., that has penetrated the serosal layer).

At 1, instruments may be introduced proximal to tumor 54056, including endoscope 54302, endoscopic stapler 54060, laparoscope 54062, and laparoscopic grasper 54064. The instrument(s) may be robot controlled. At 2, tumor 54056 may be inverted by laparoscopic grasper 54064. Snare 54066 may be applied by endoscope 54302. At 3, snare 54066 may sinch tumor 54056. Tumor 54056 may be positioned (e.g., using endoscope 54302) to be accessed by the endoscopic stapler 54060 and laparoscopic grasper 54064 may release tumor 54056. At 4, the jaws of endoscopic stapler 54060 may be placed between tumor 54056 and the serosal layer. The endoscopic stapler 54060 may be fired (e.g., to separate tumor 54056 from the serosal layer). At 5, the instruments may be retracted and tumor 54056 may be removed.

If the tumor is too large for oral extraction, hybrid natural orifice trans-luminal endoscopic surgery (NOTES) may be performed. The NOTES portion of the procedure may involve an inversion of the tumor through the incision and a hand-off to the laparoscopic side instruments for final separation from the wall and removal. NOTES may include entering the peritoneal cavity or the abdominal cavity through the gastrointestinal tract (e.g., using a natural orifice).

Instrument operation and inter-connectivity may be implemented when performing flexible endoscopic bronchoscope tumor biopsy or treatment requiring mid advancement CT recalibrating of a guidance system.

An ultrasound (e.g., 3D US) system may aim to achieve augmented reality (AR) visualization during laparoscopic surgery (e.g., for the liver). To acquire visual data (e.g., 3D US data) of the liver, the tip of a laparoscopic ultrasound probe may be tracked inside the abdominal cavity, such as by using a magnetic tracker. The accuracy of magnetic trackers may be greatly affected by magnetic field distortion that results from the proximity of metal objects and electronic equipment. Magnetic field distortion in an operating room may be determined and dealt with.

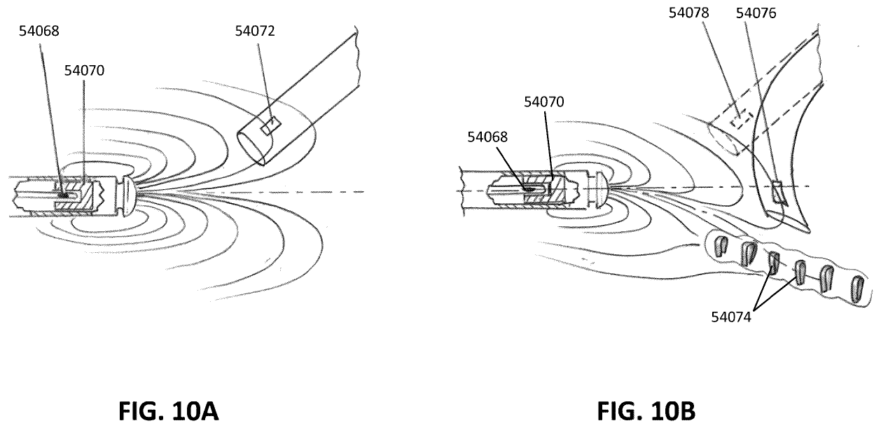

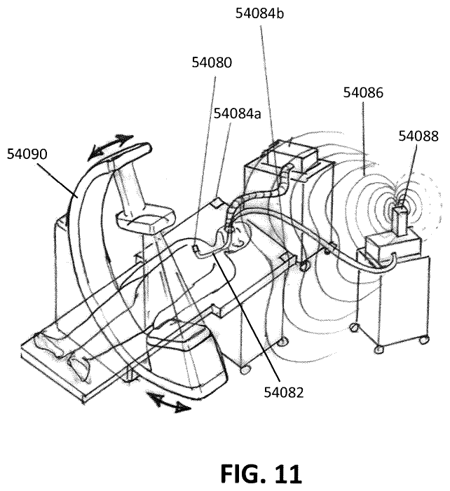

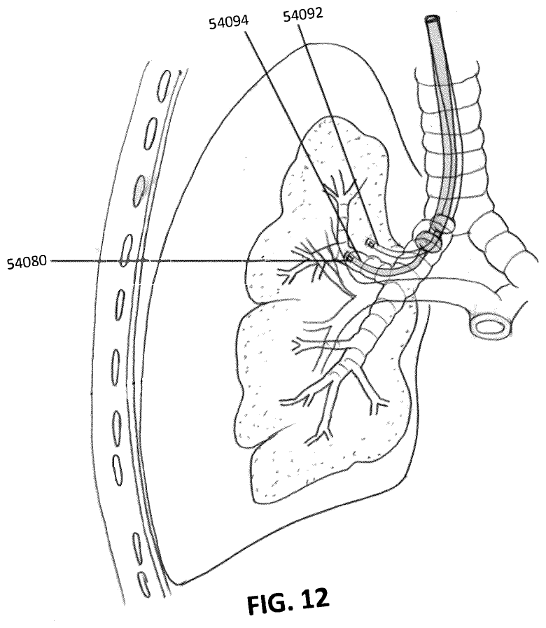

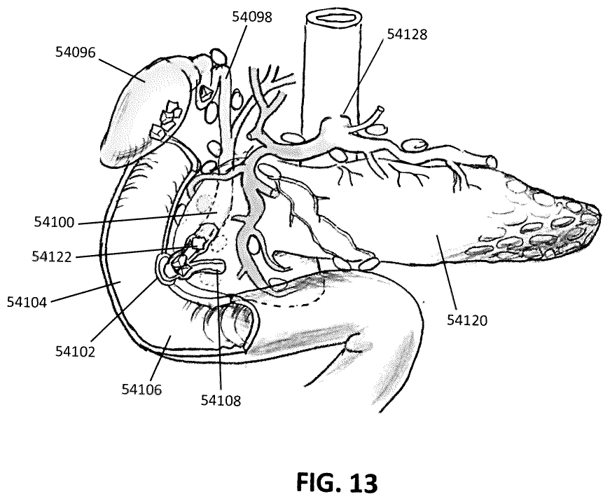

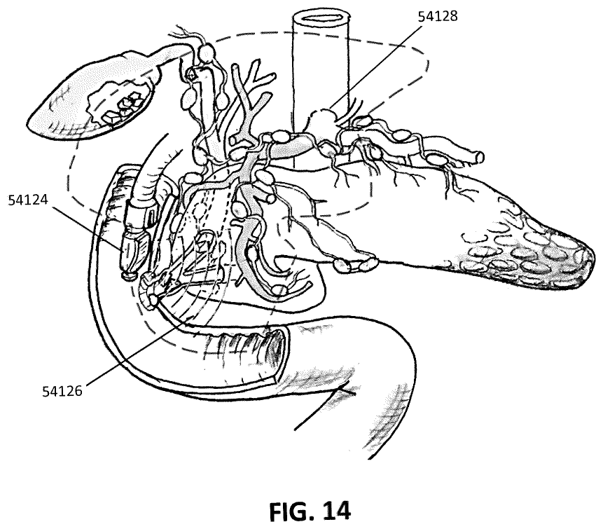

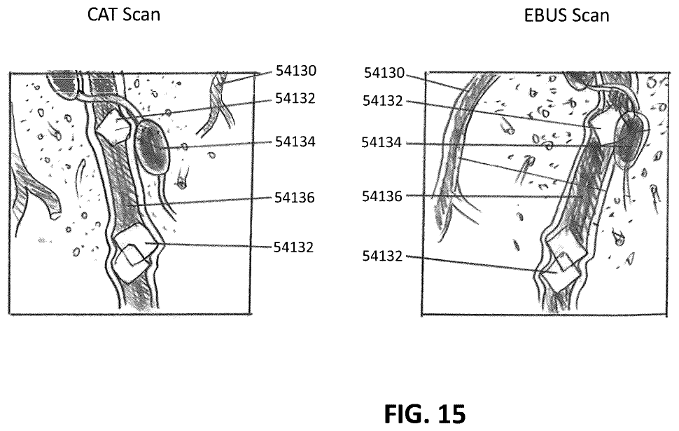

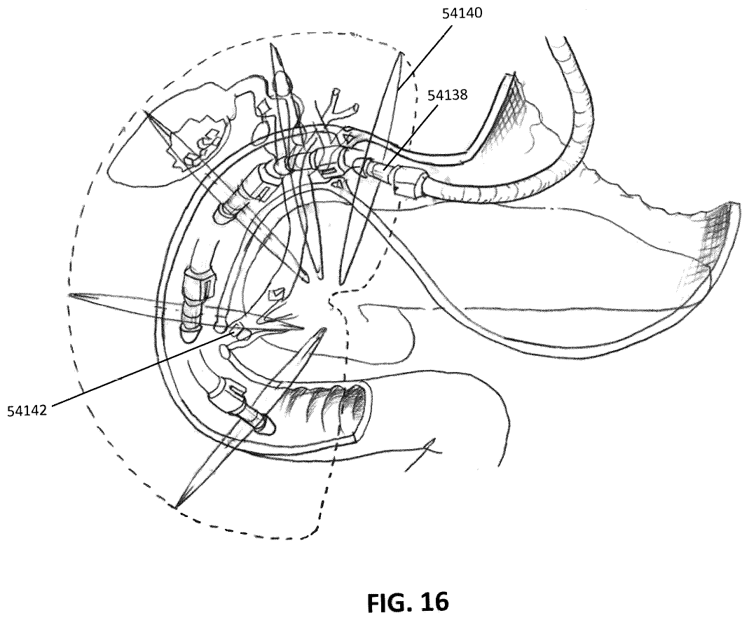

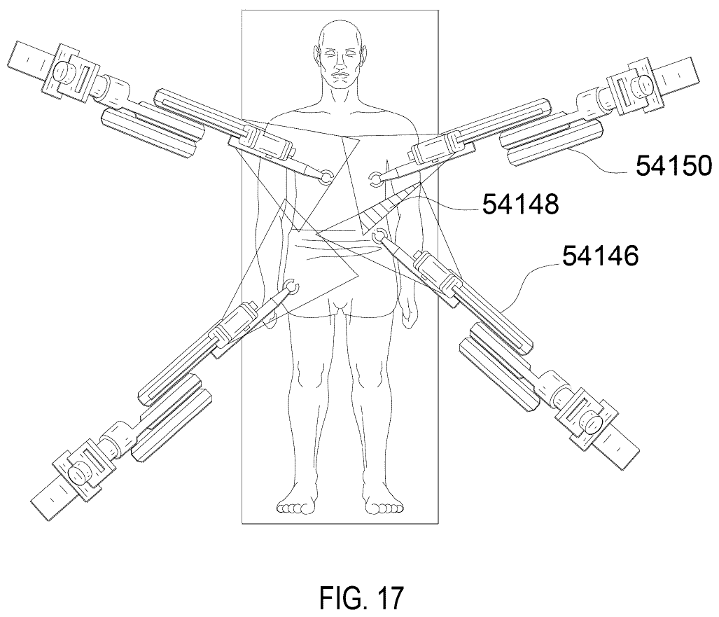

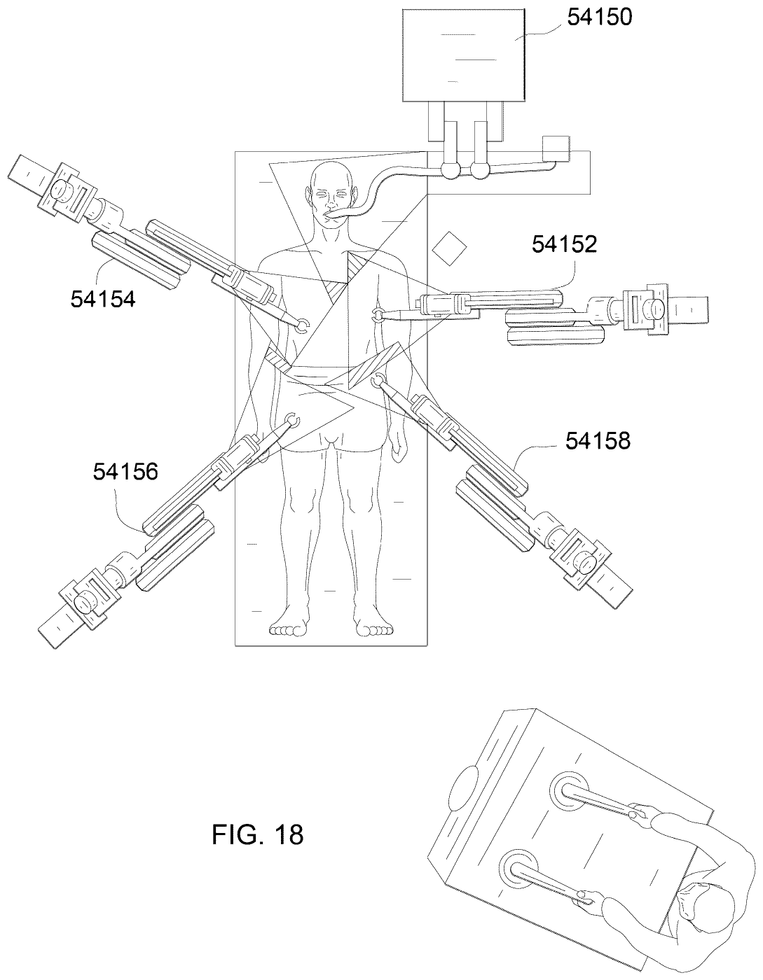

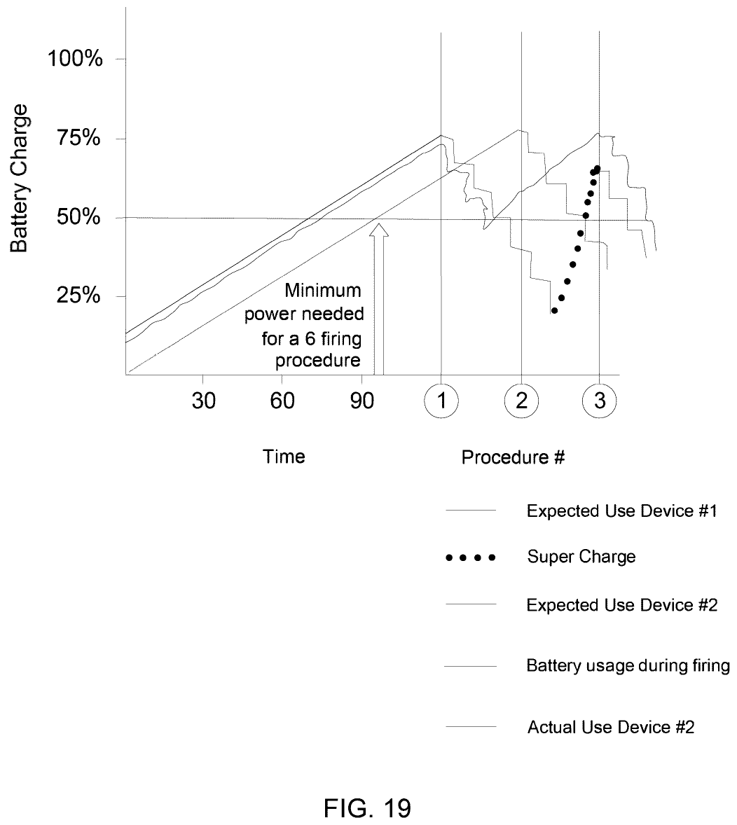

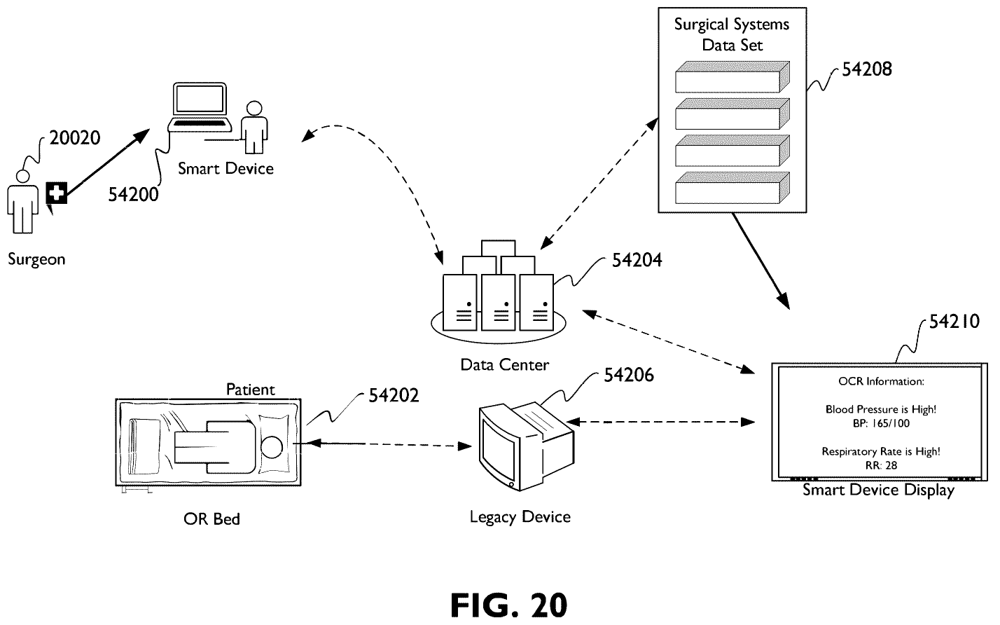

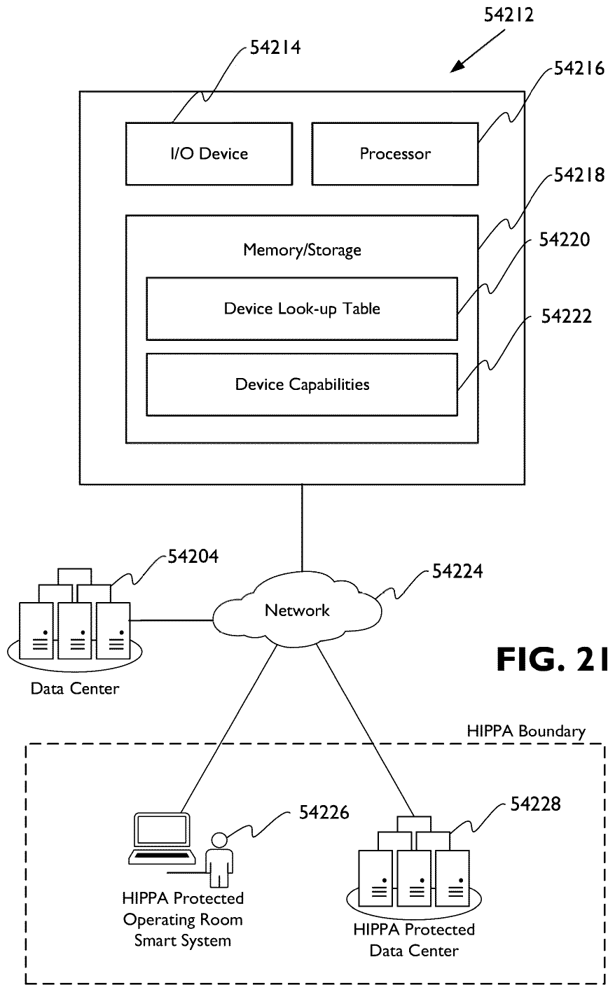

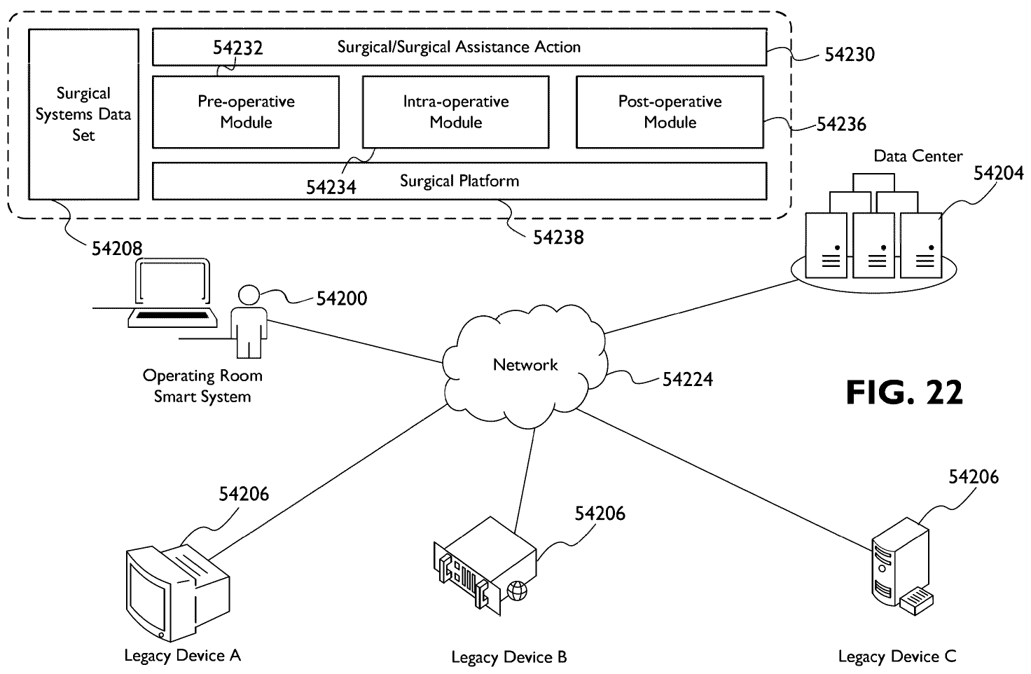

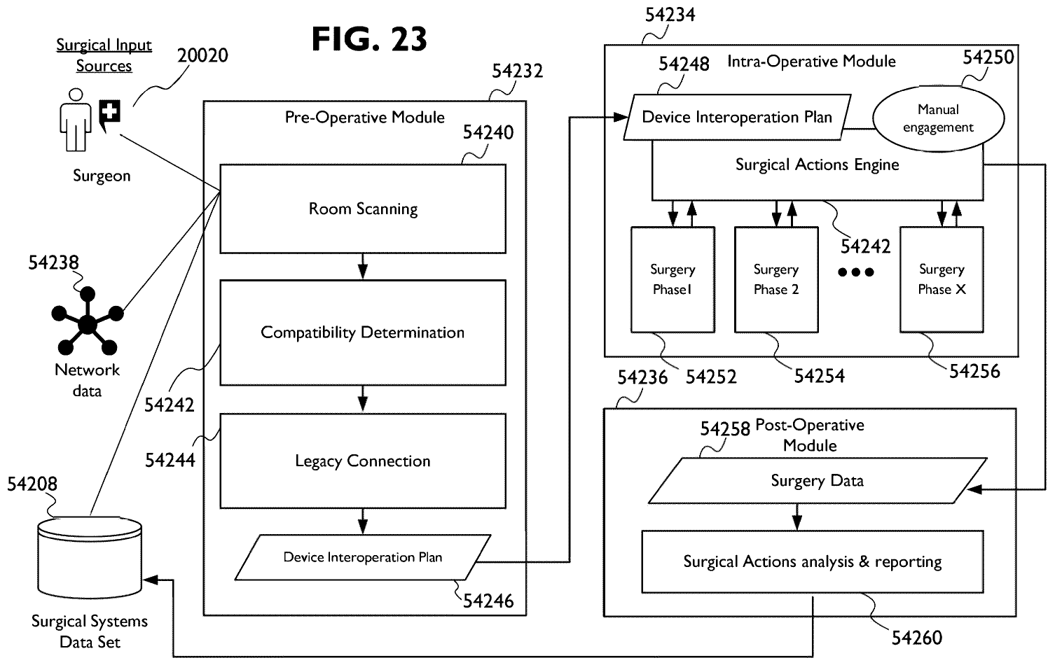

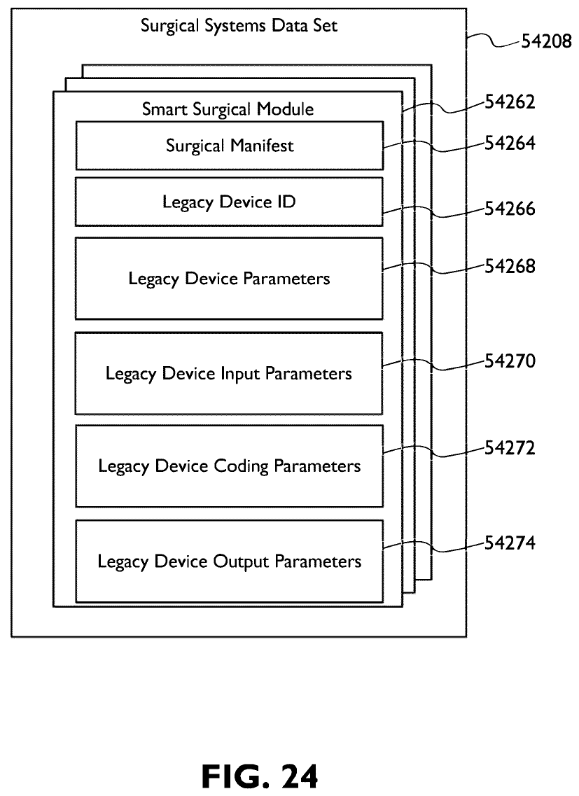

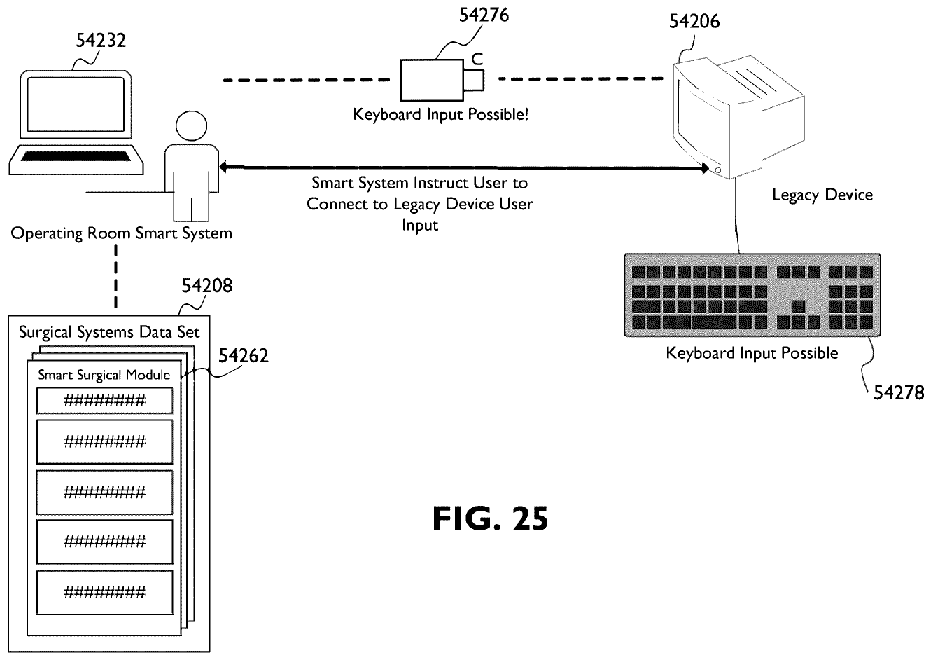

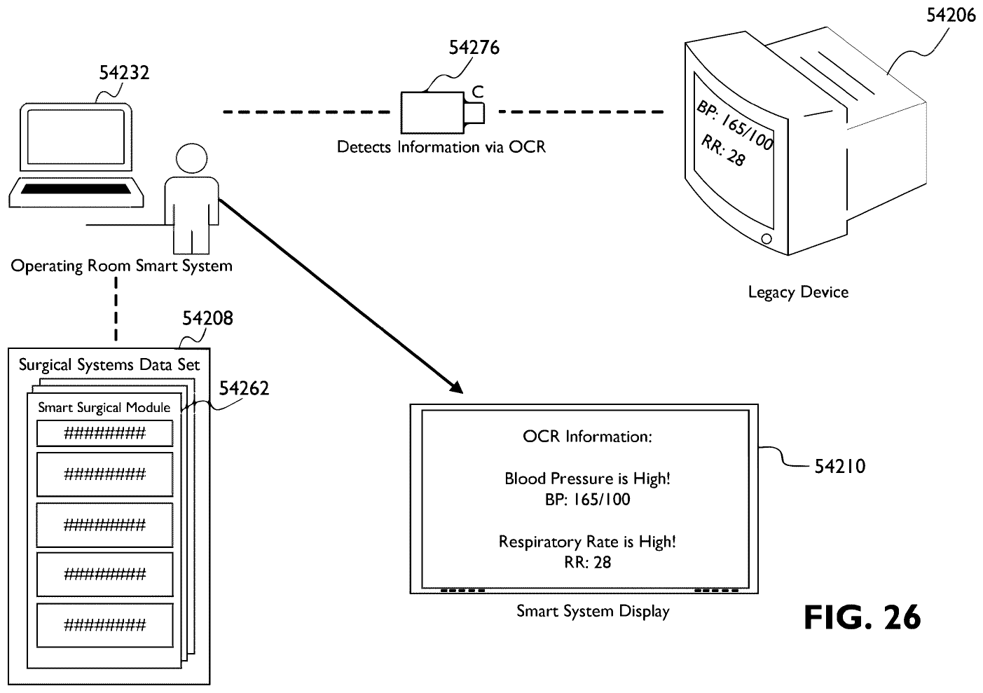

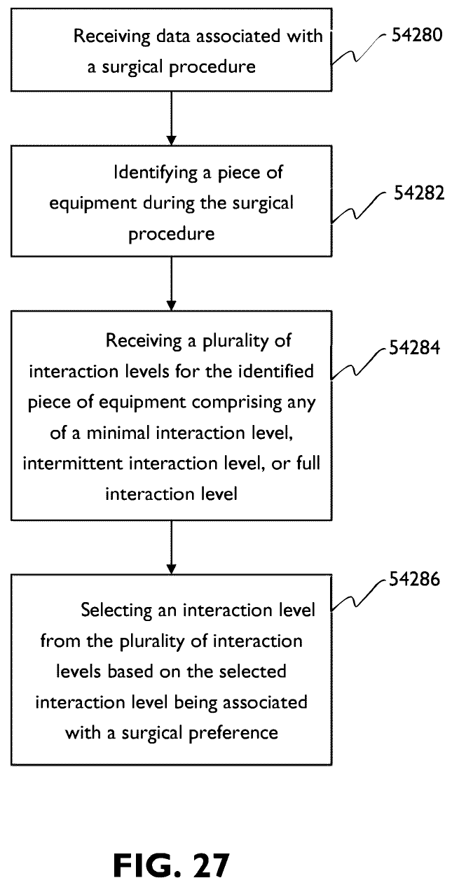

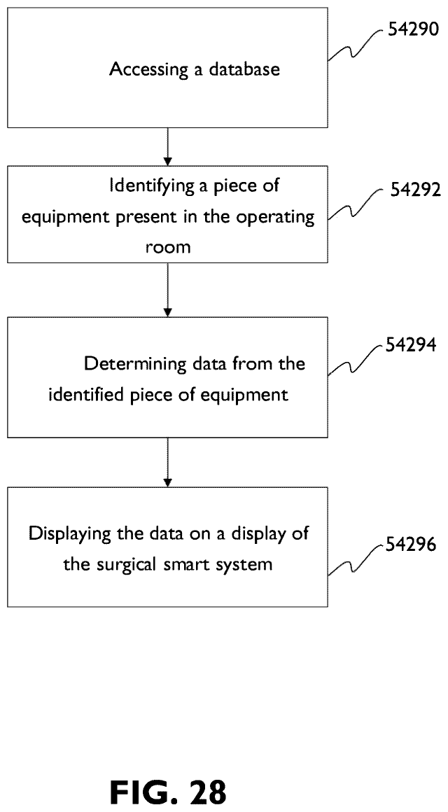

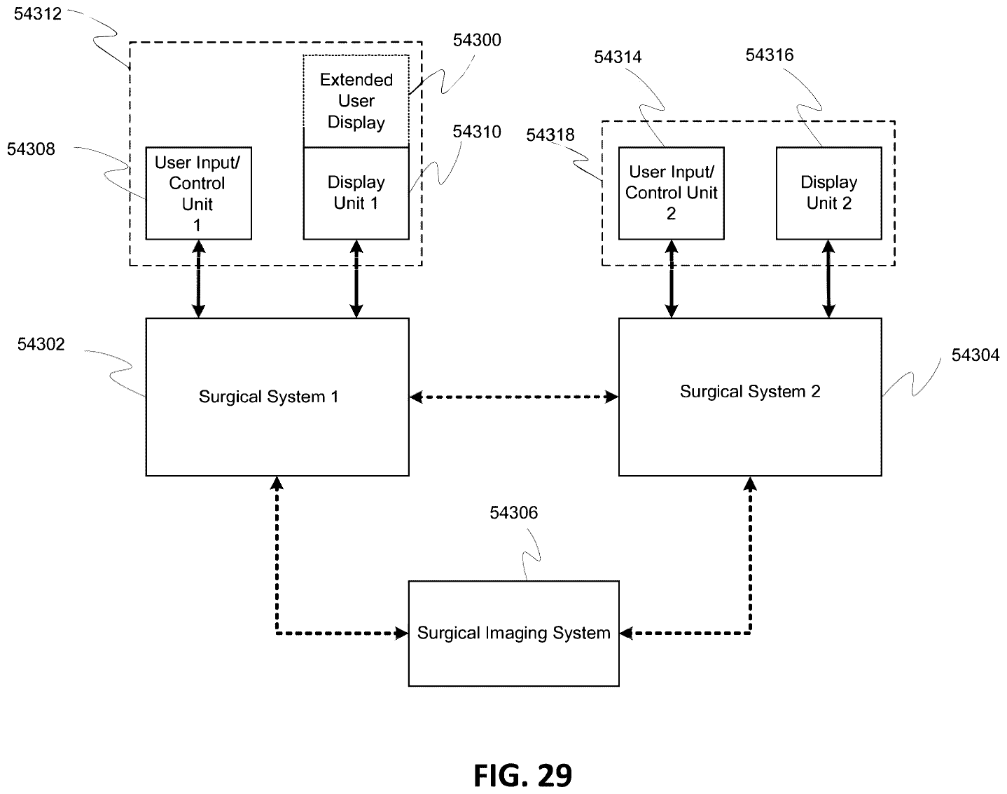

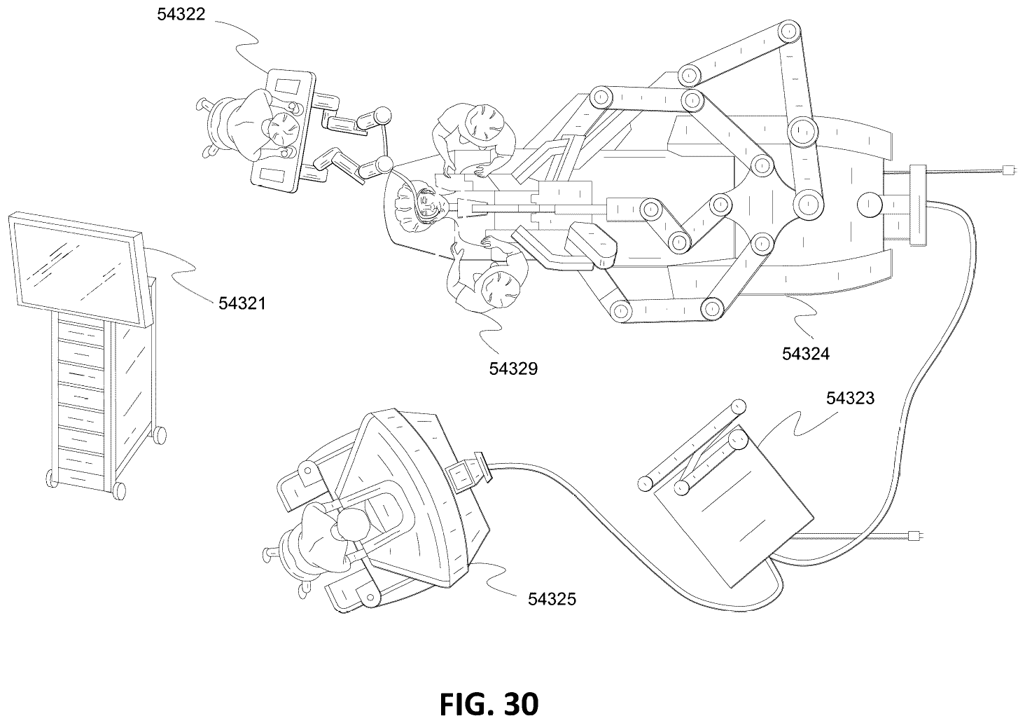

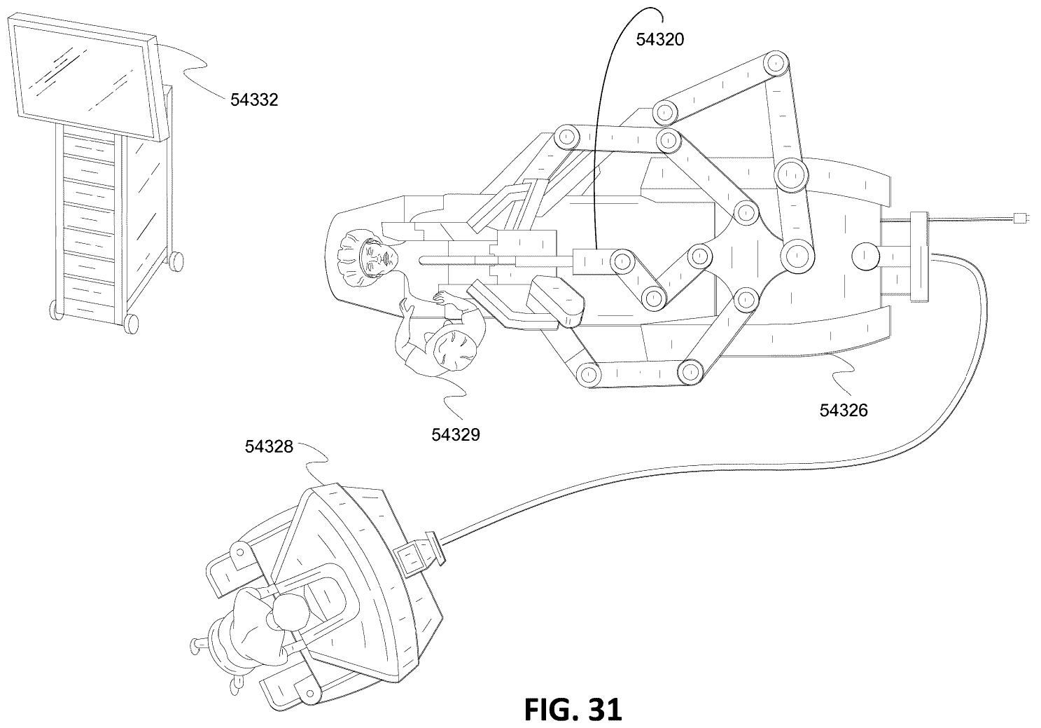

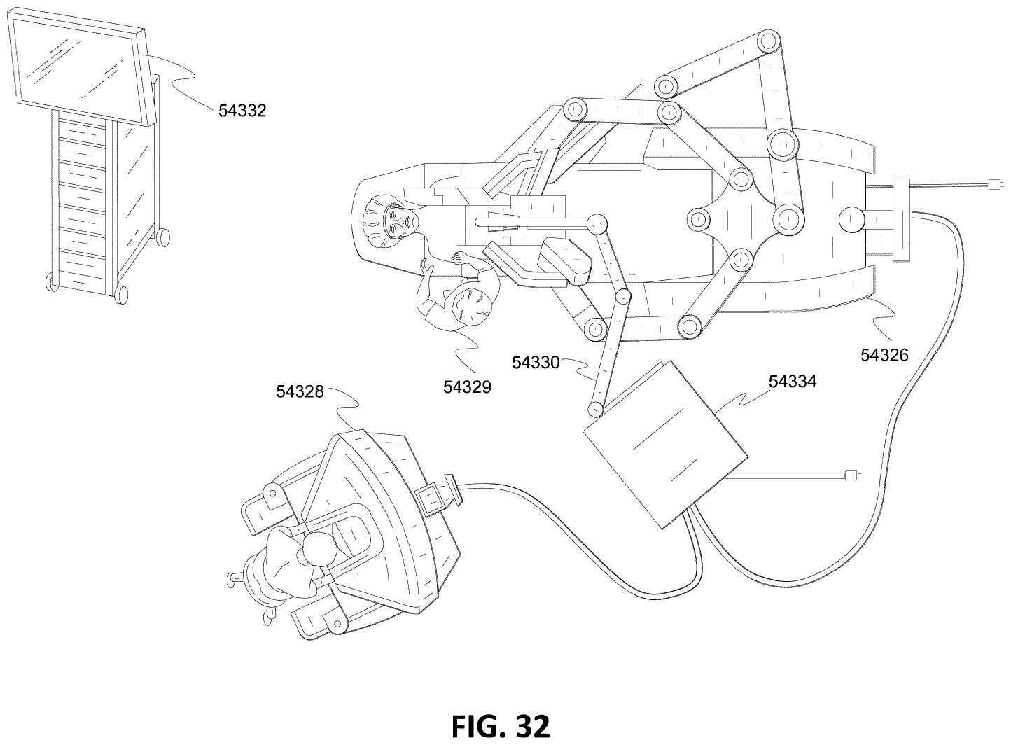

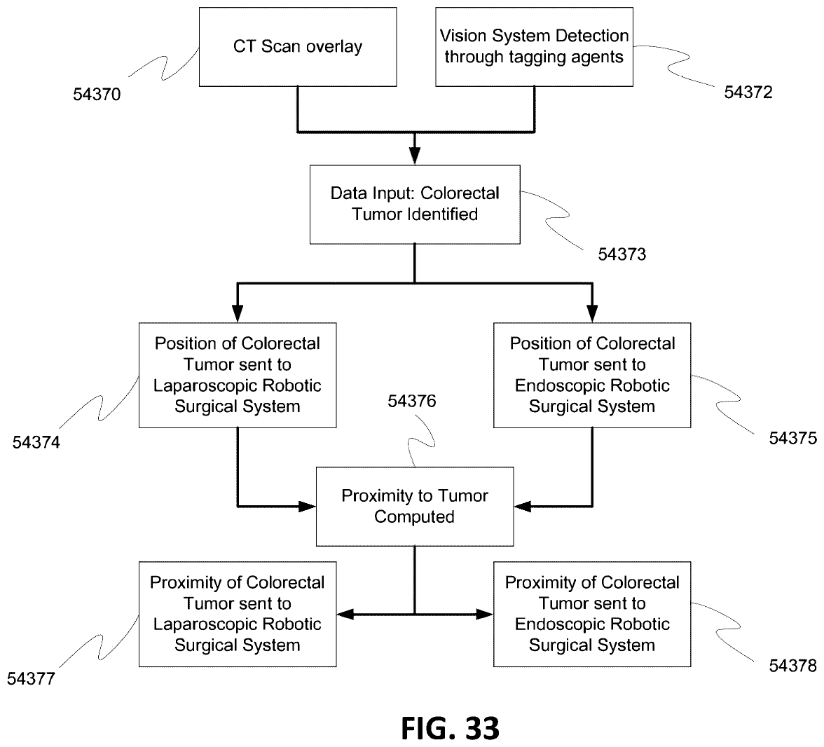

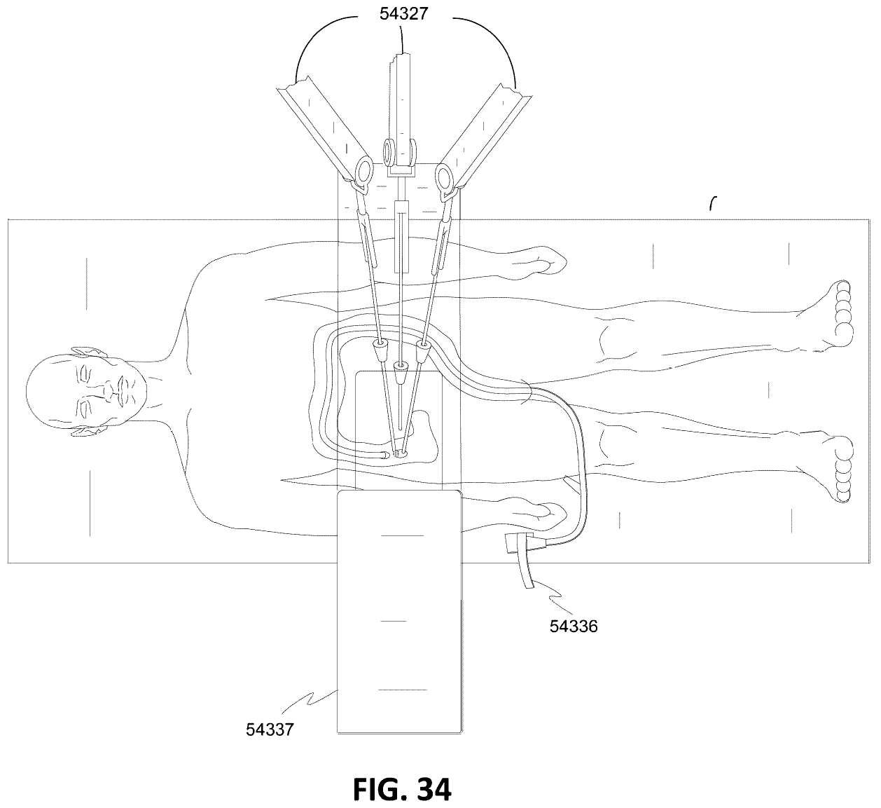

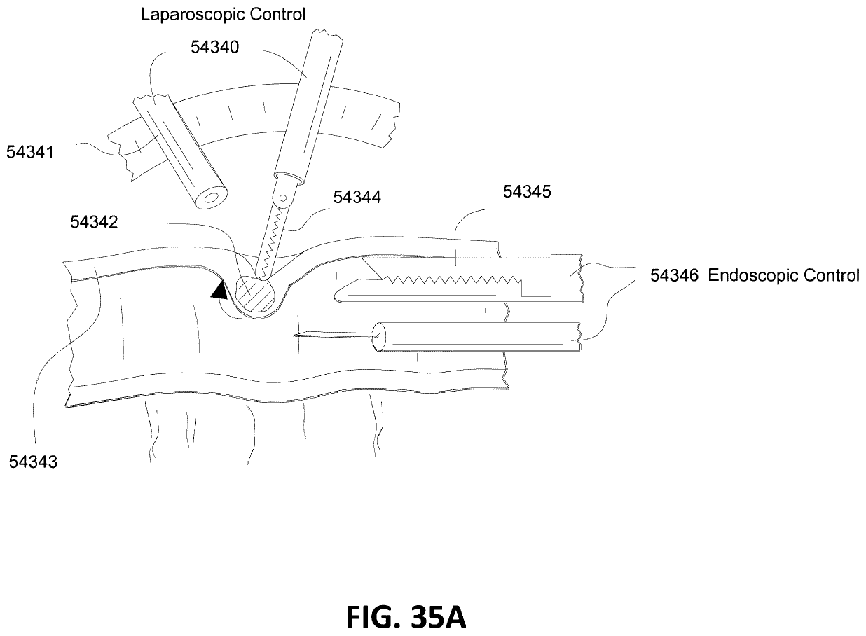

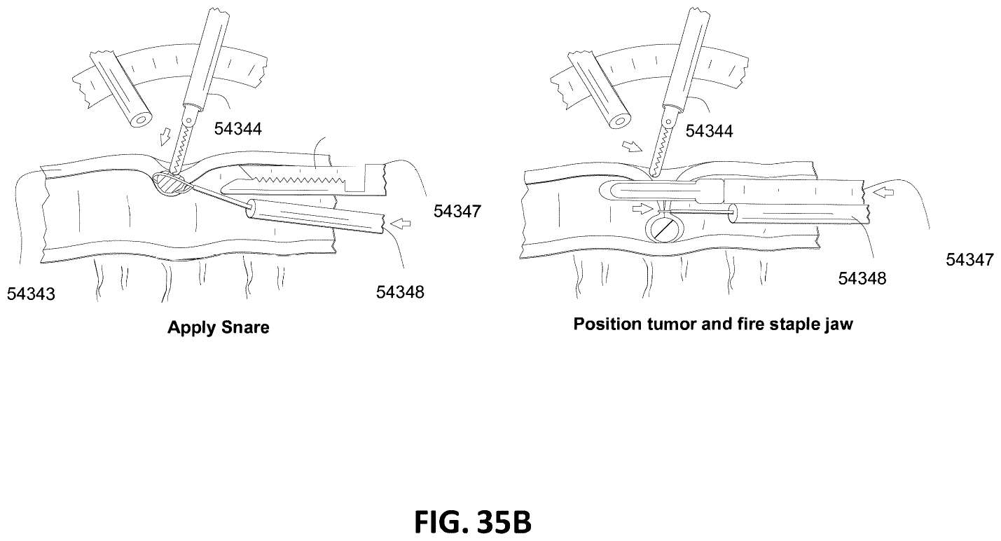

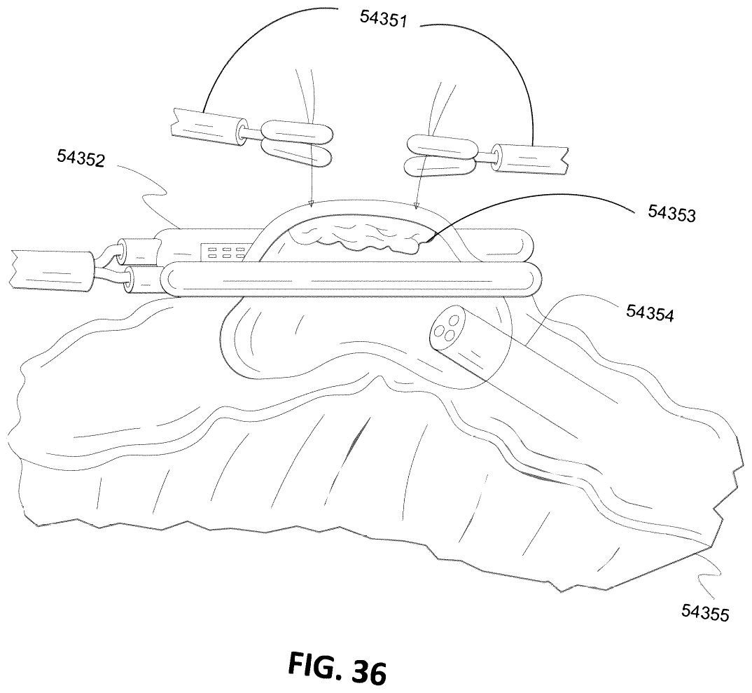

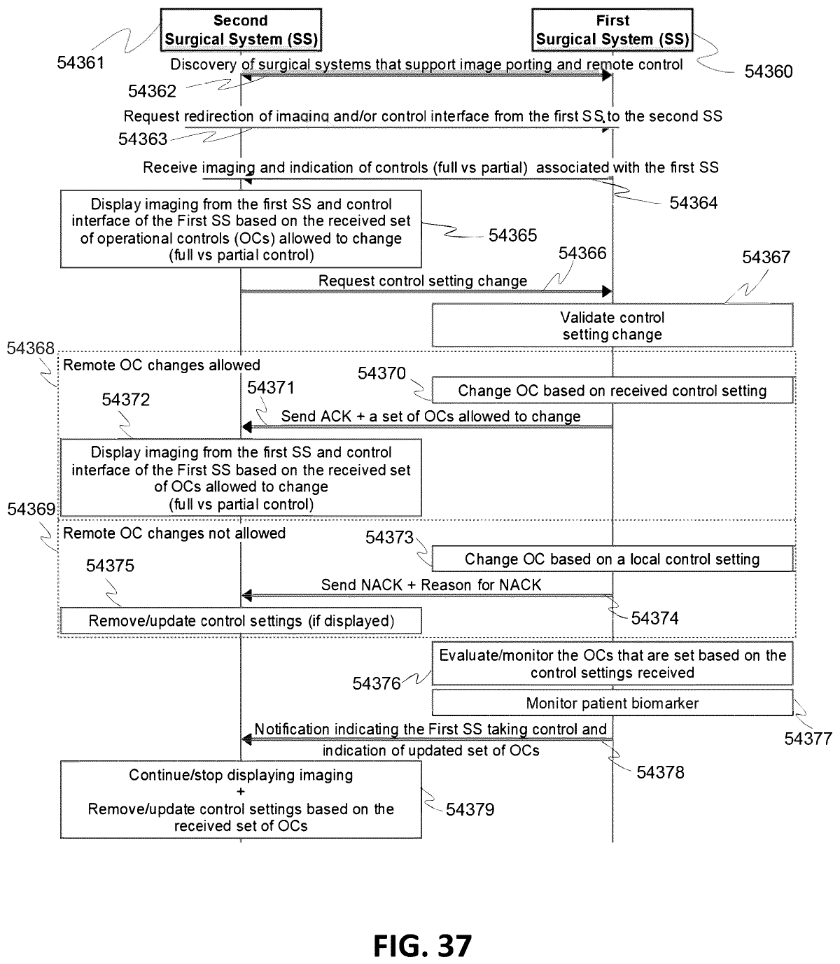

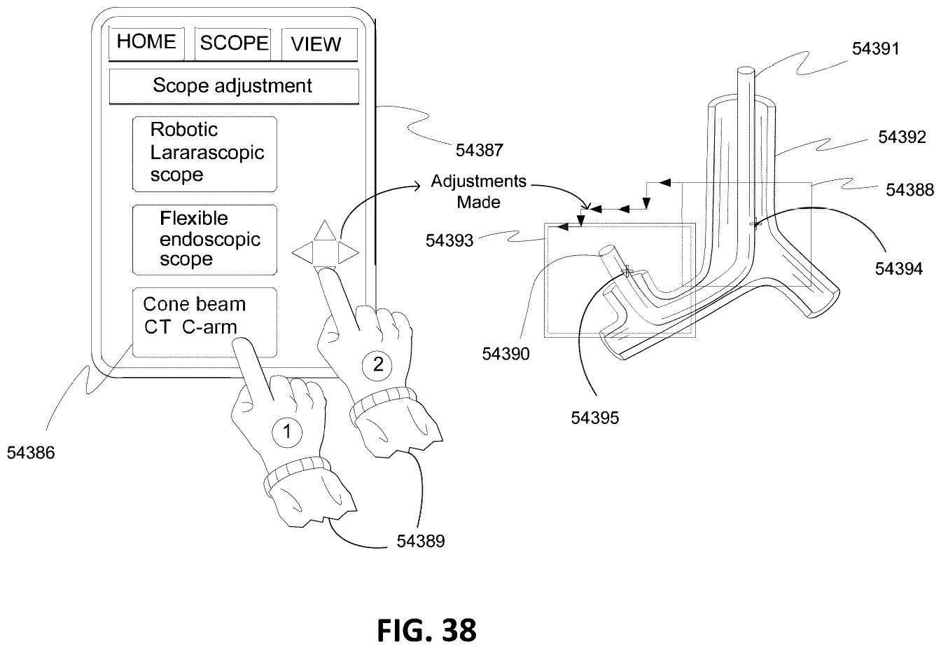

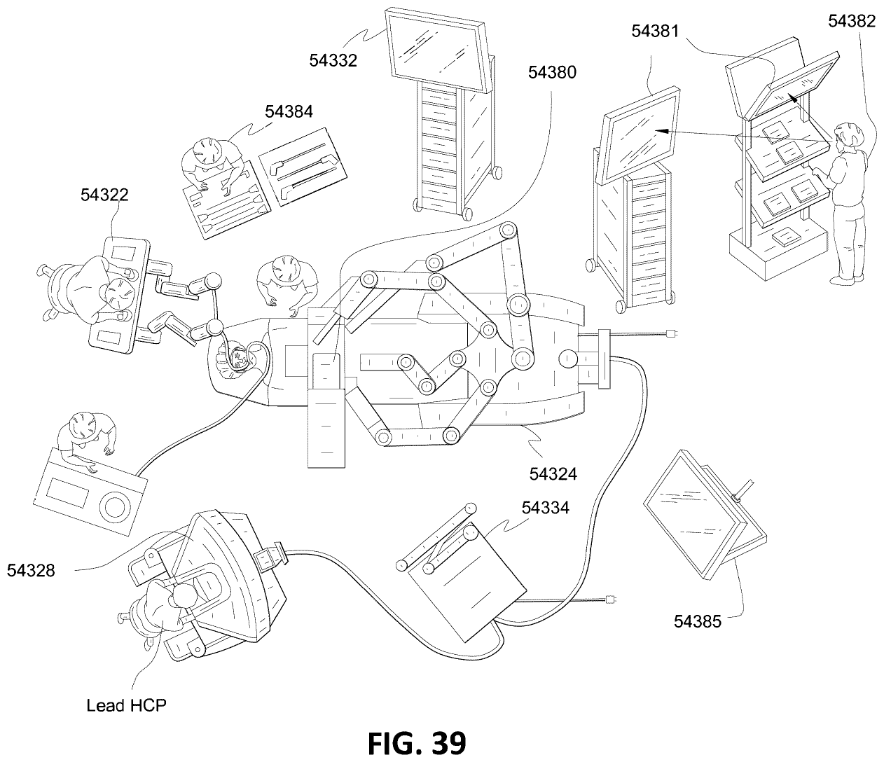

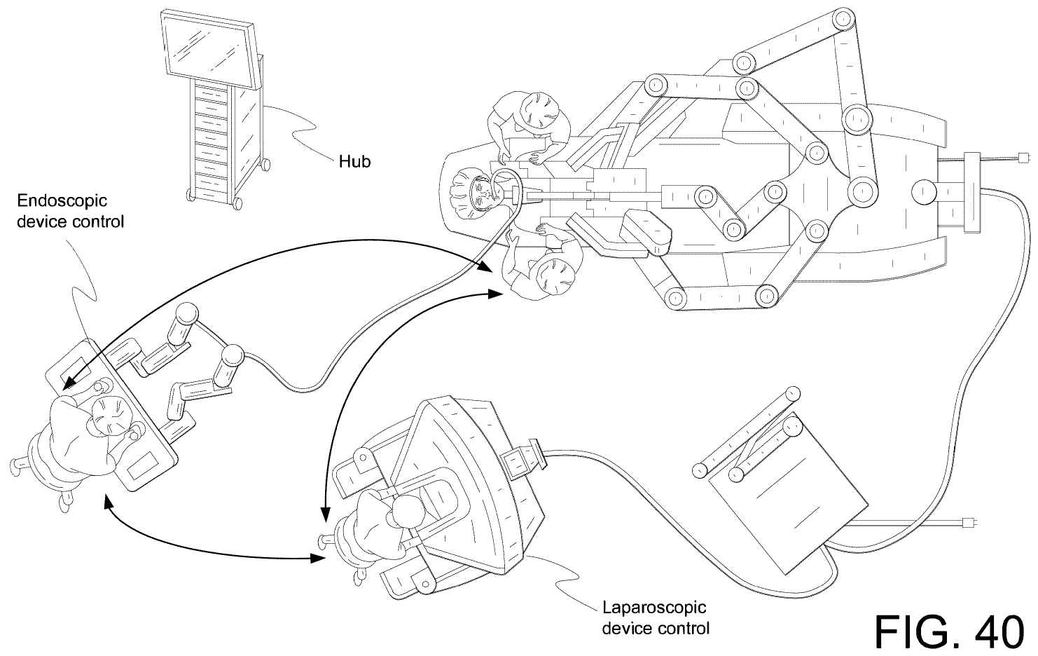

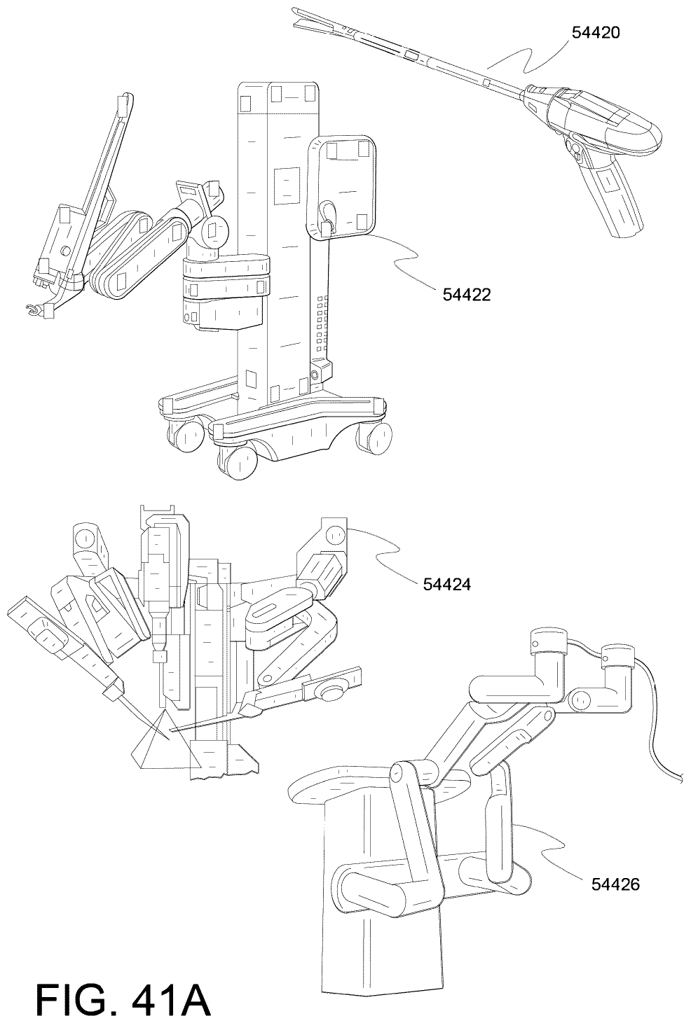

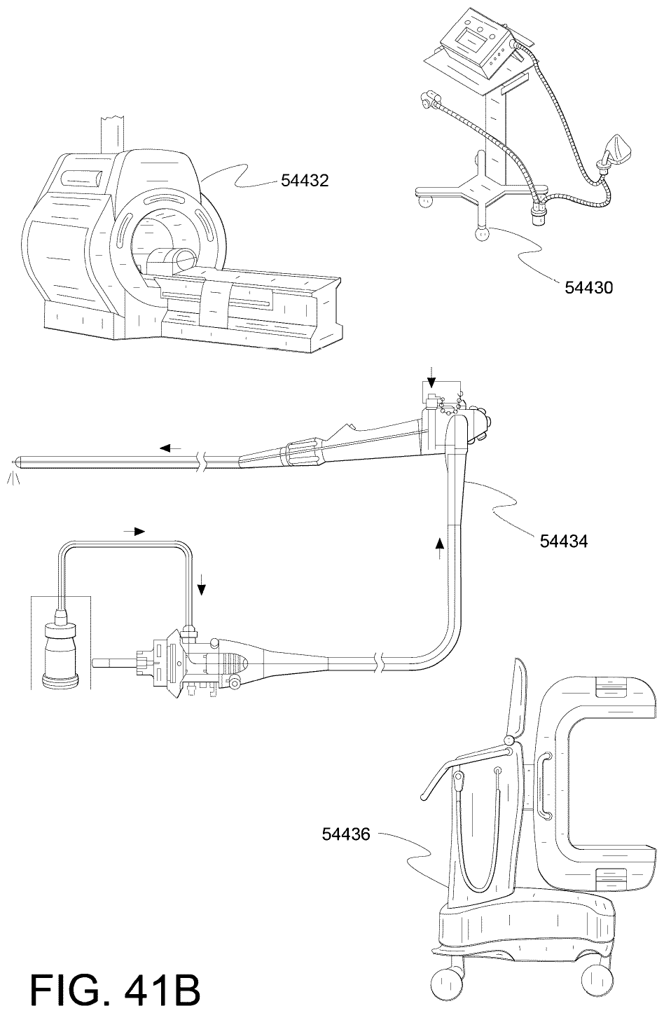

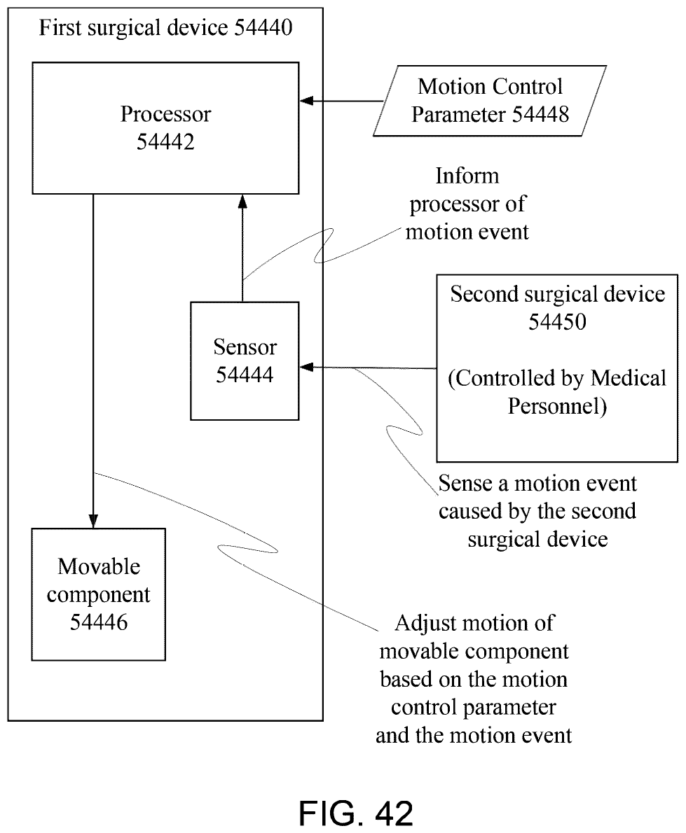

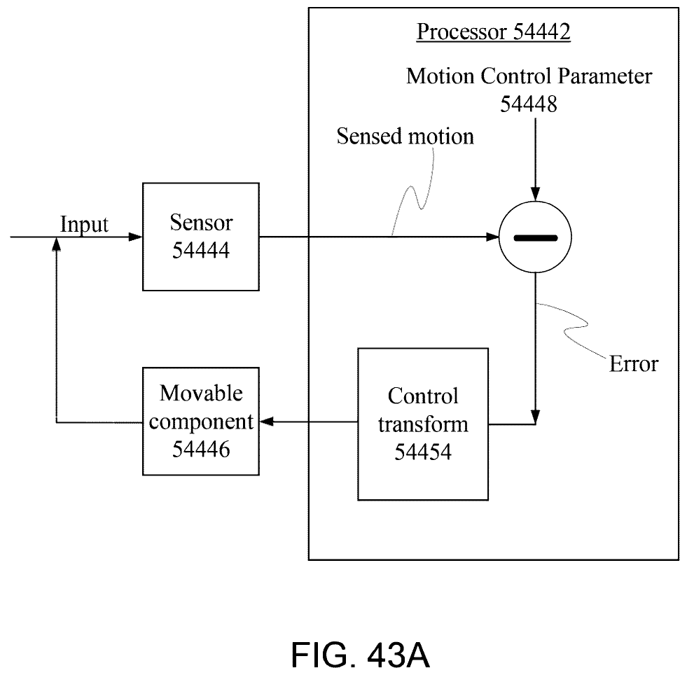

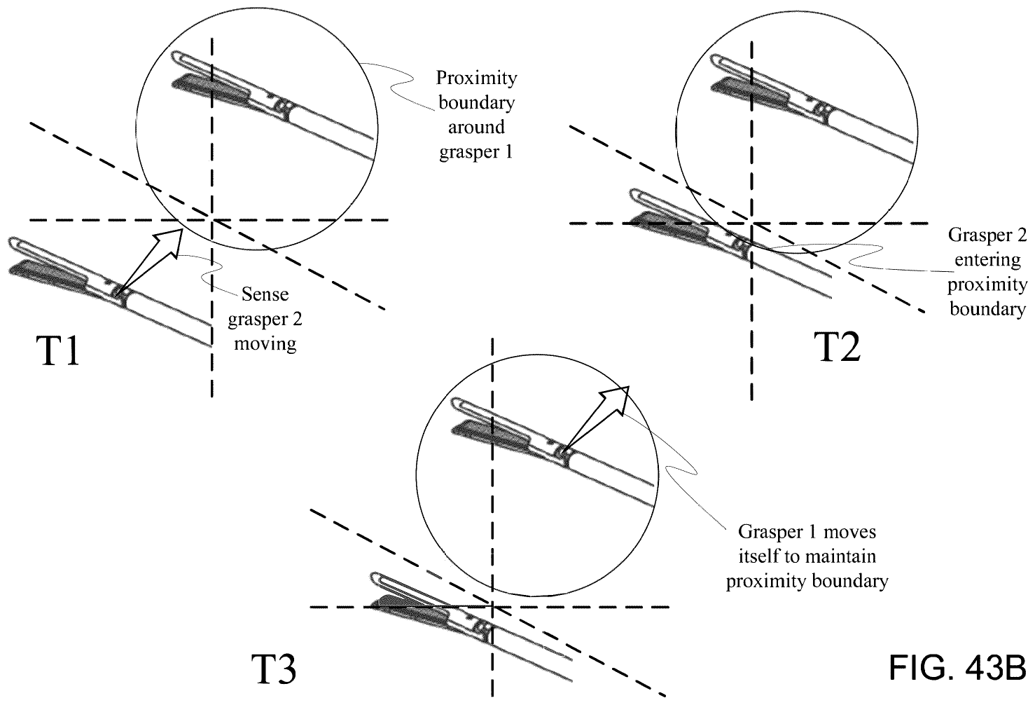

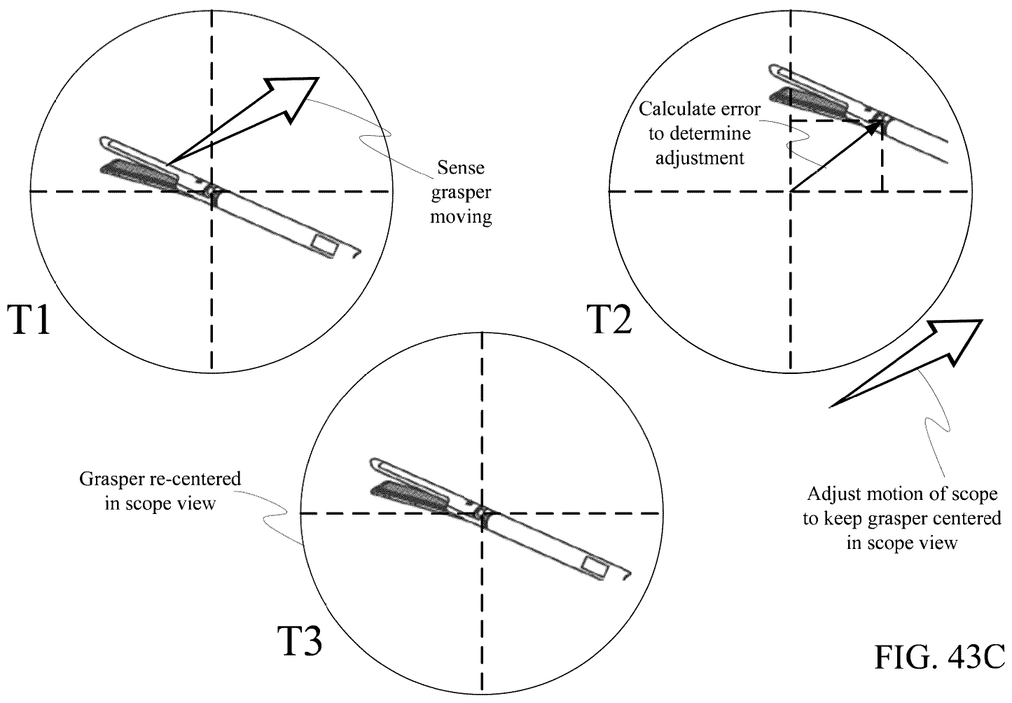

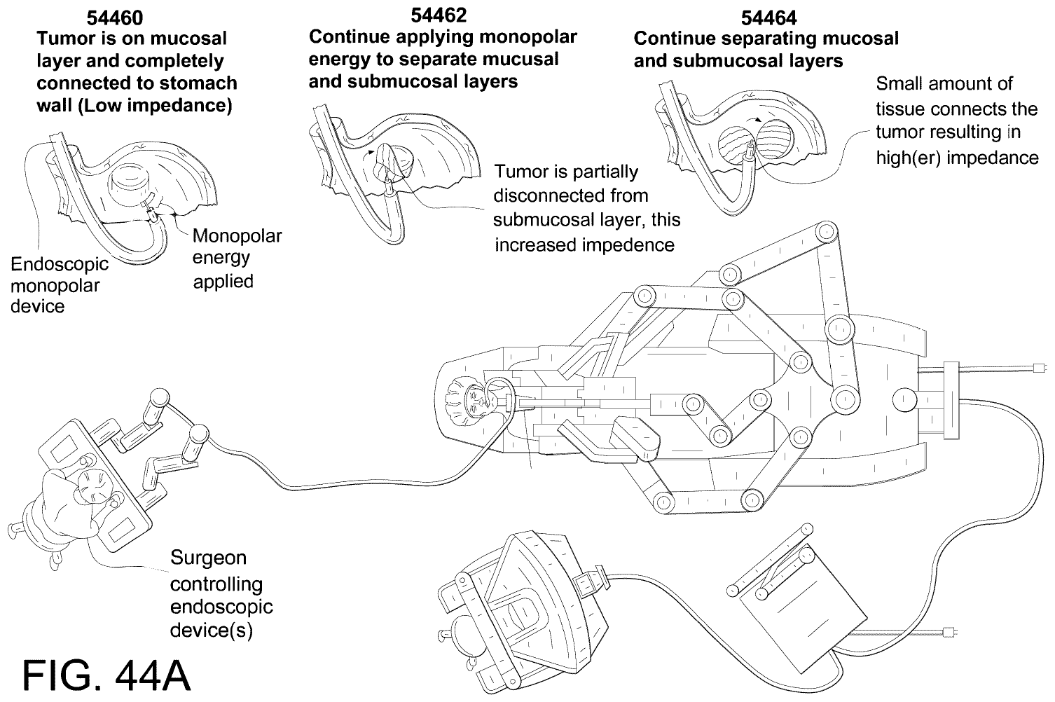

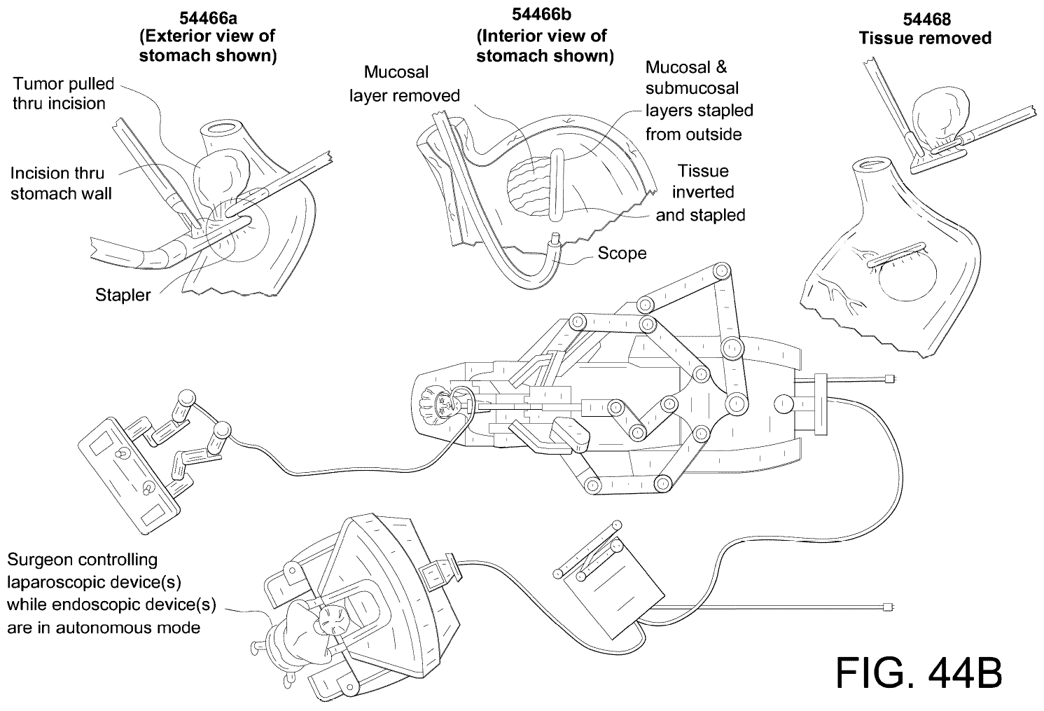

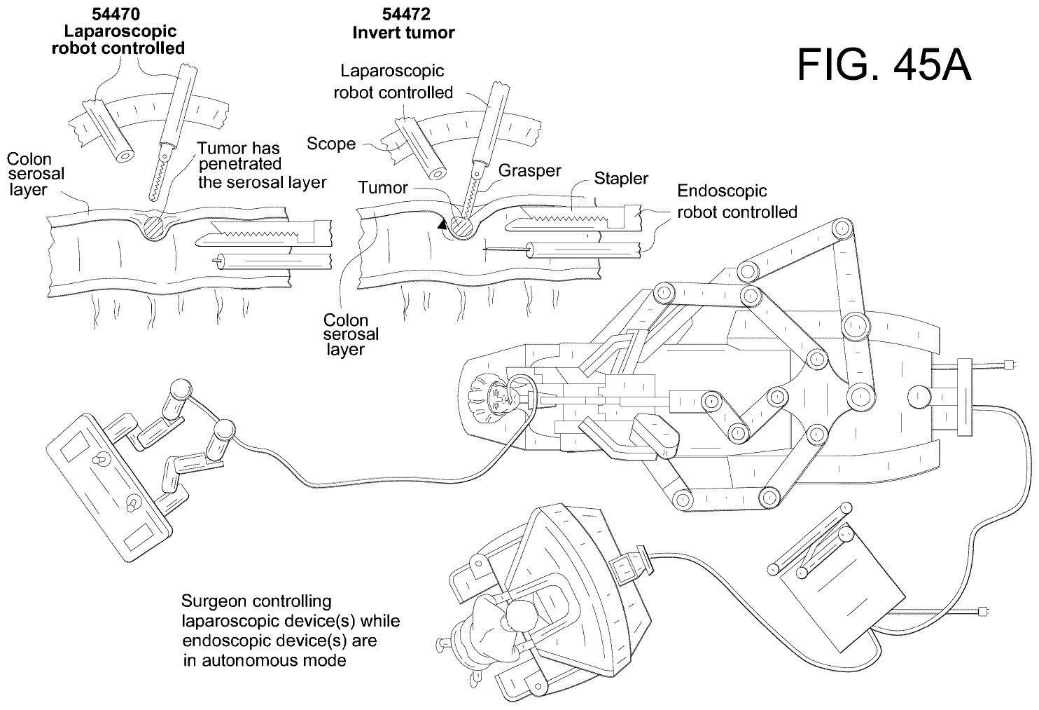

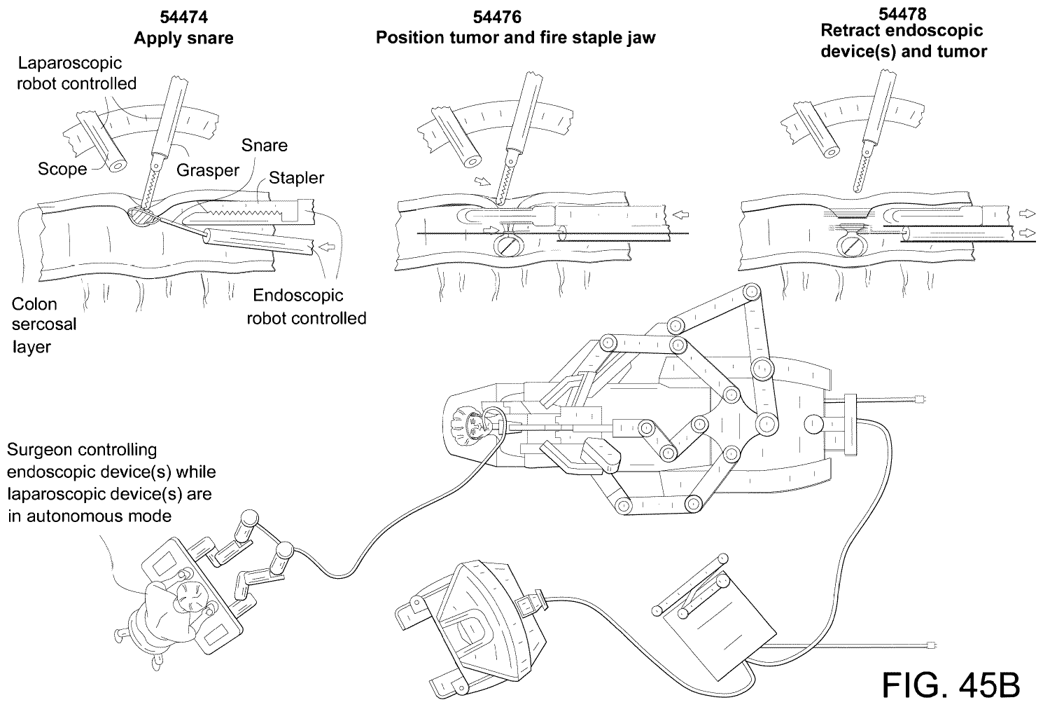

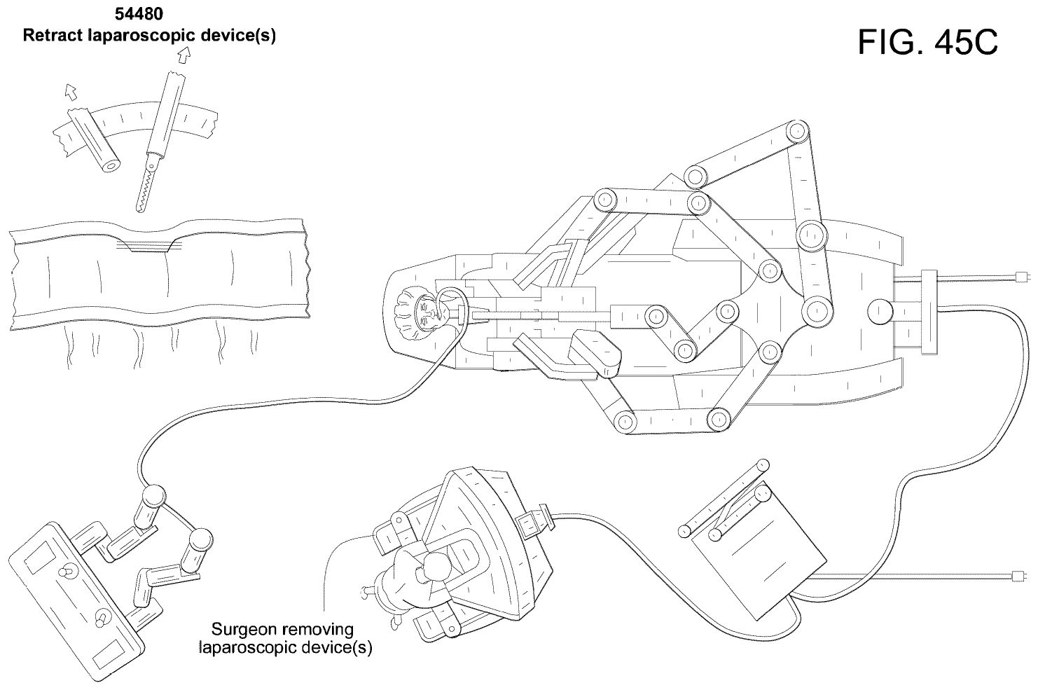

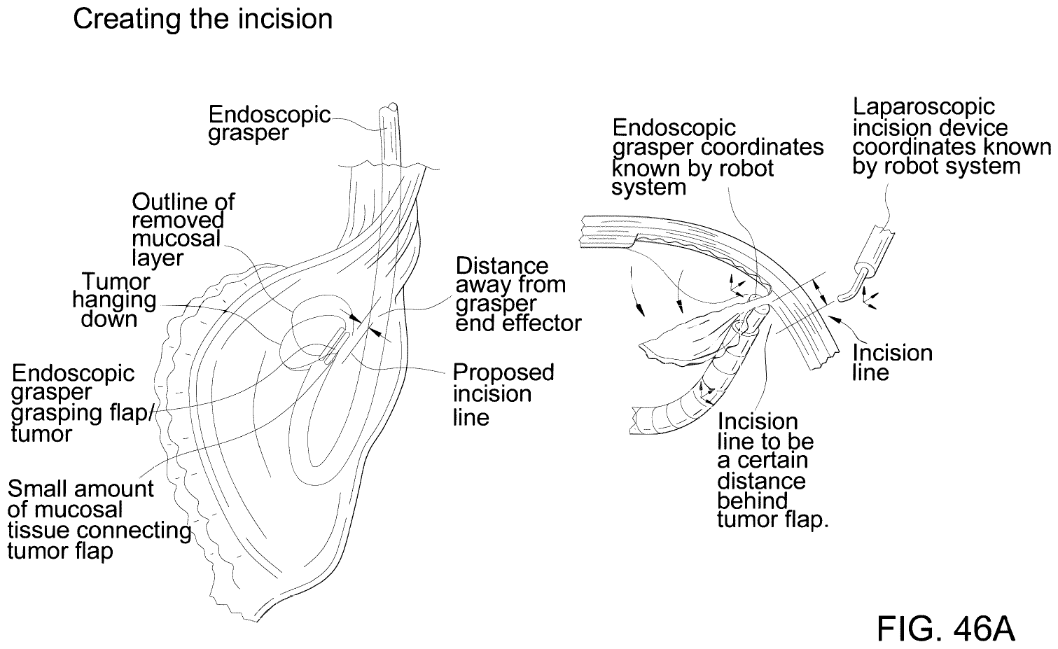

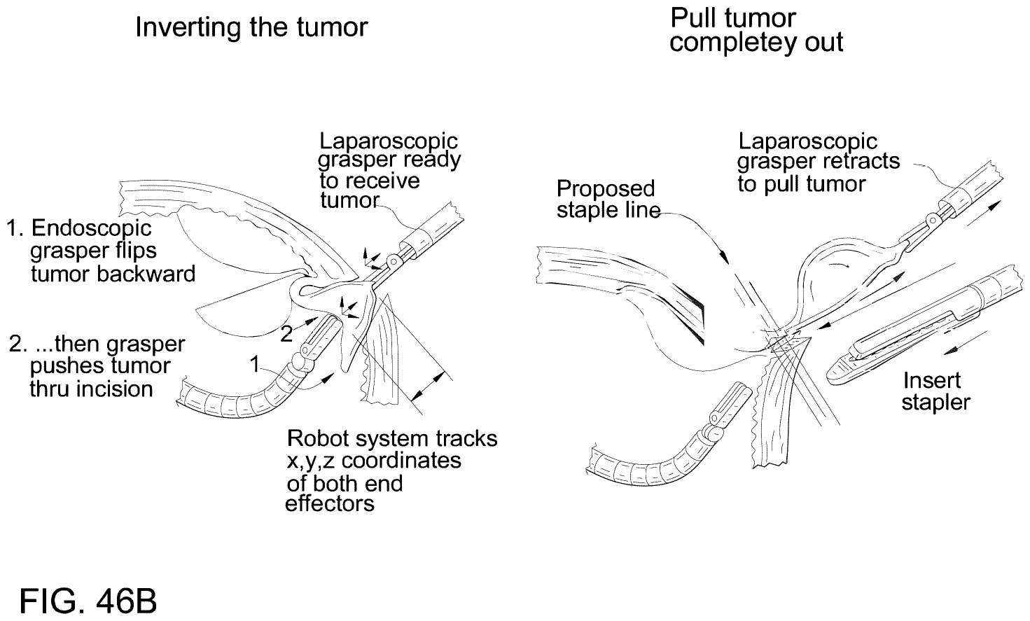

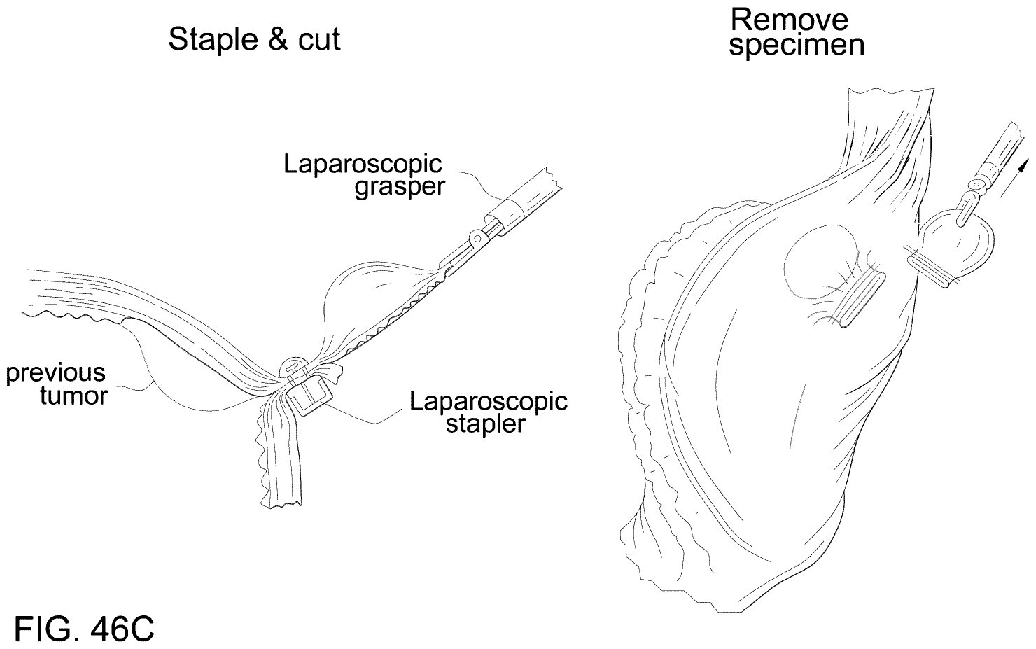

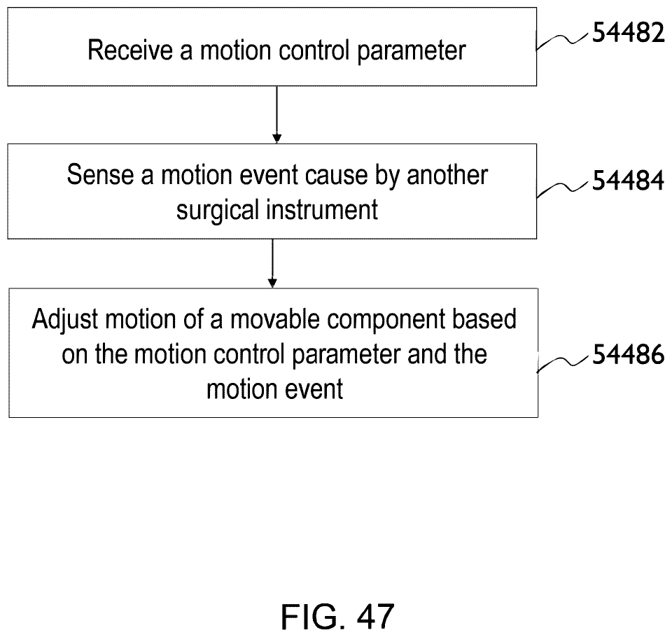

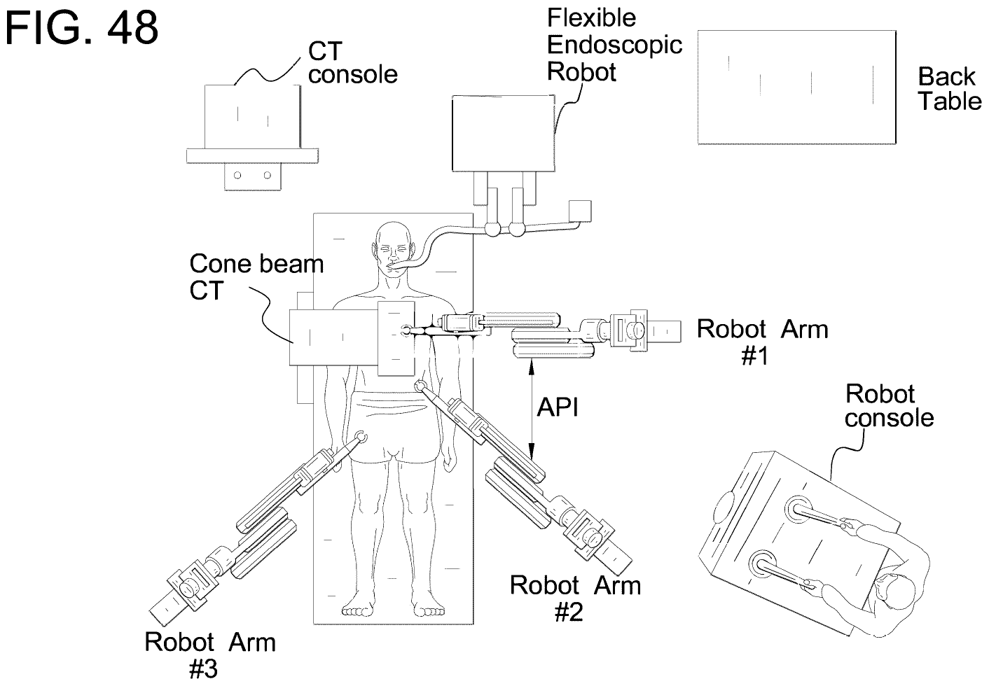

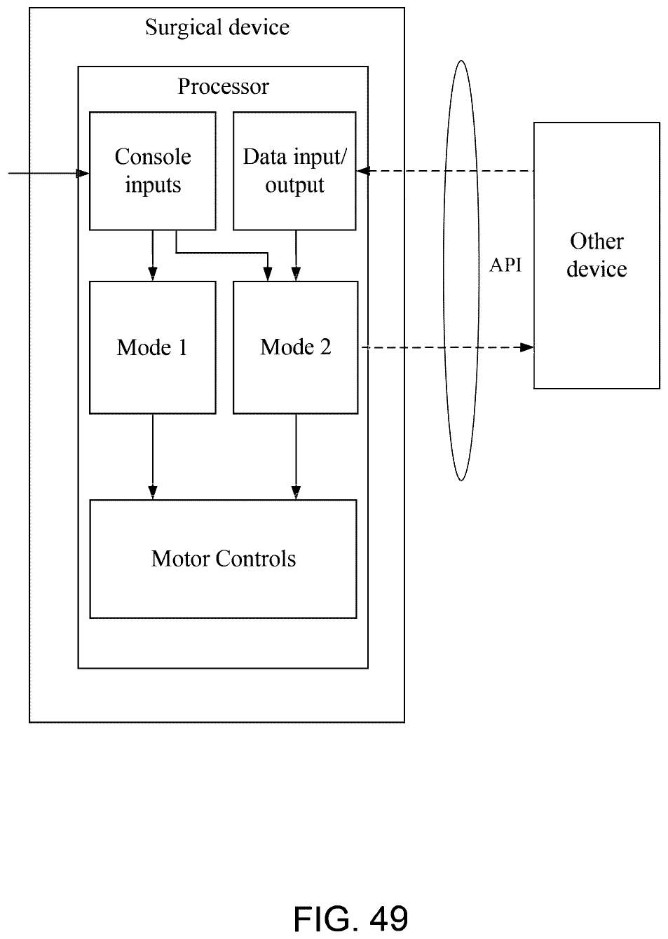

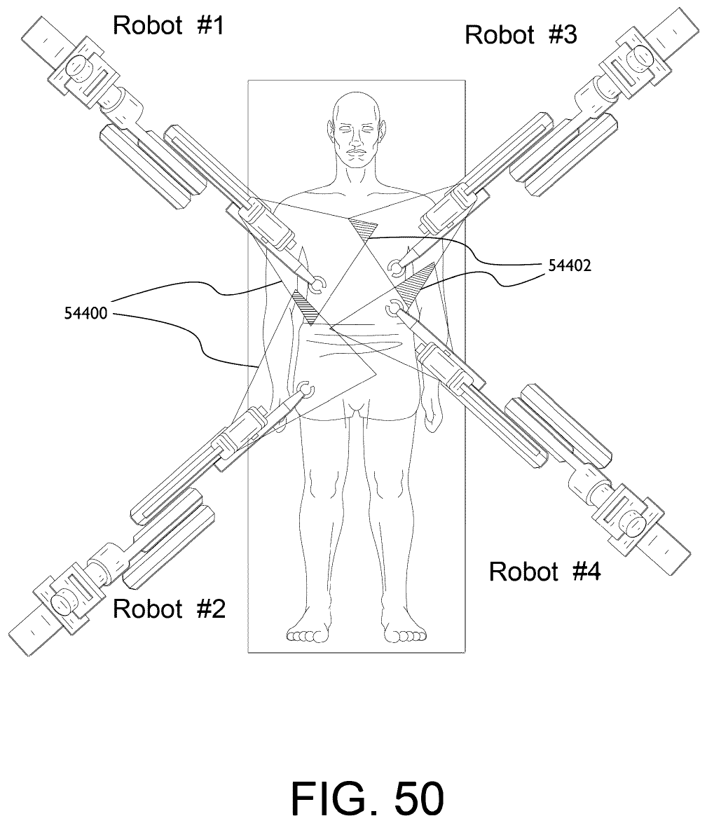

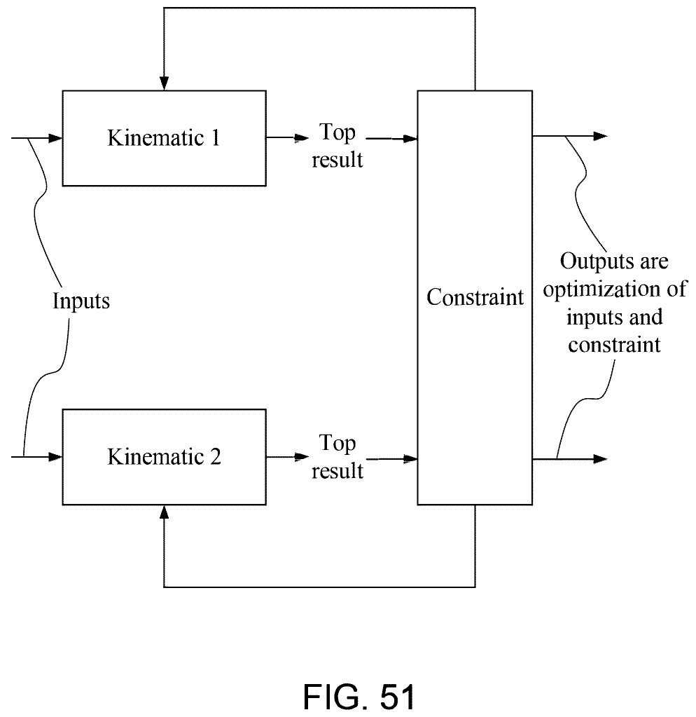

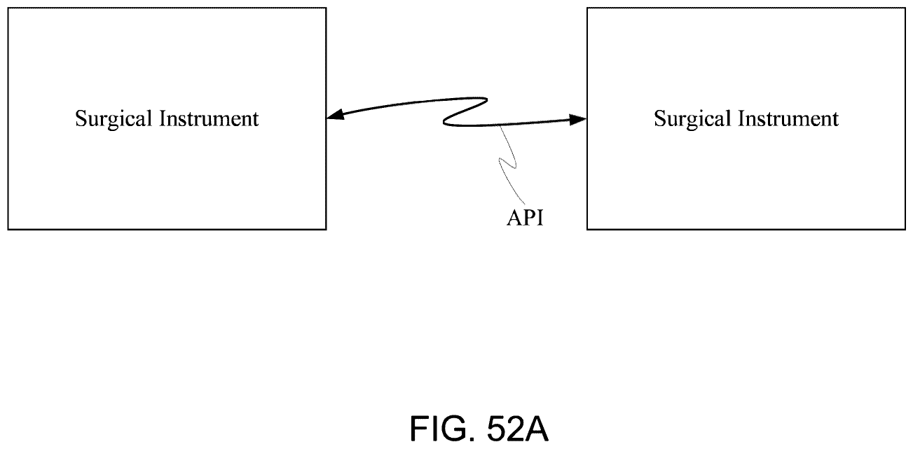

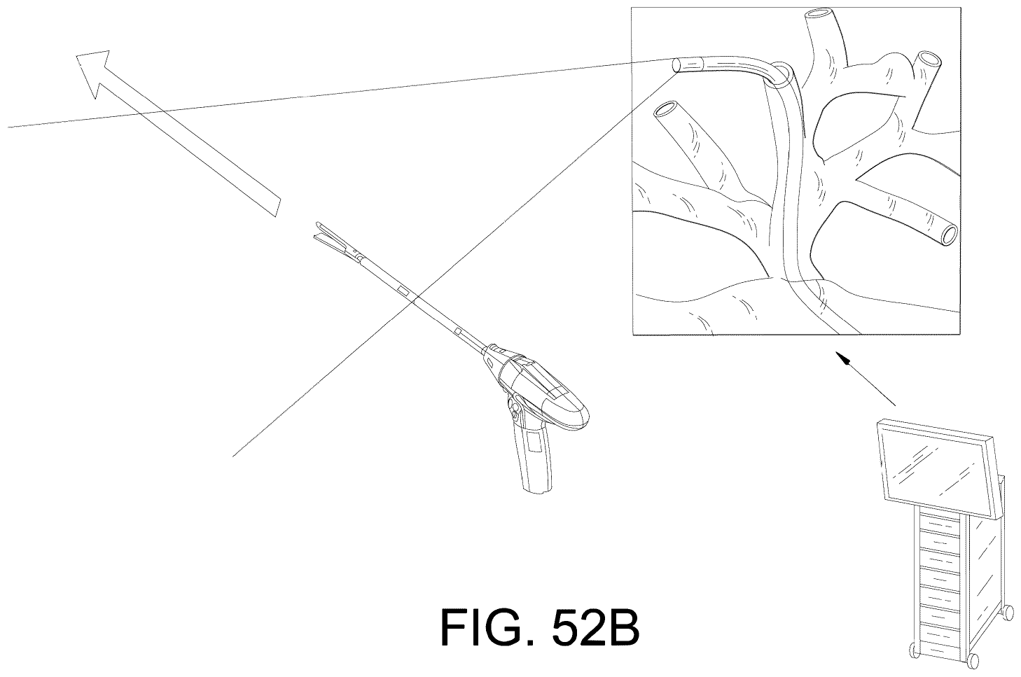

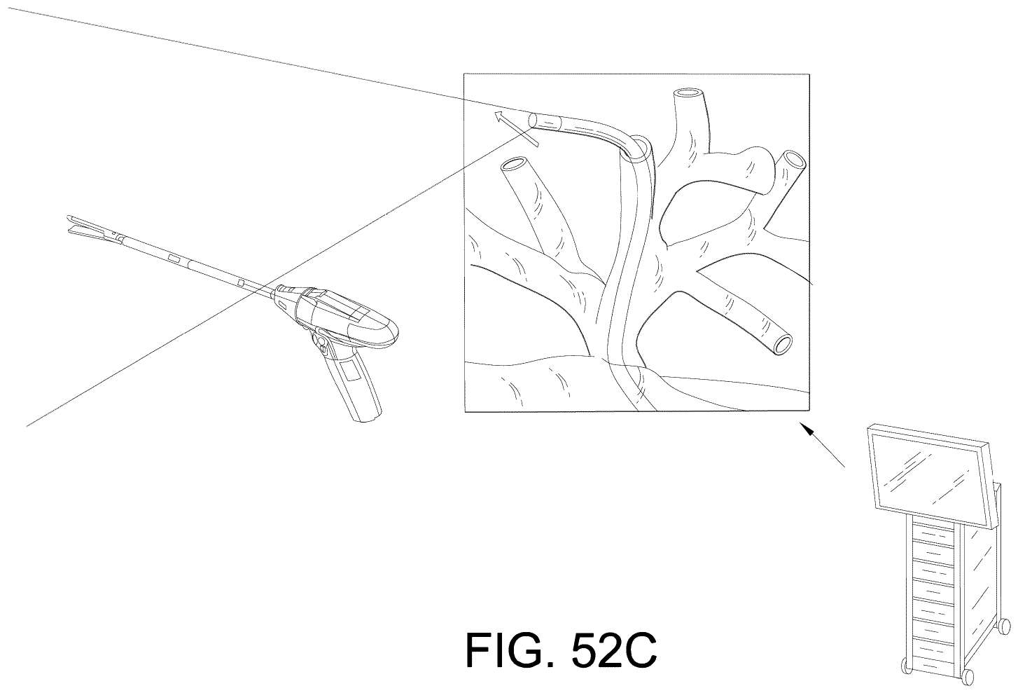

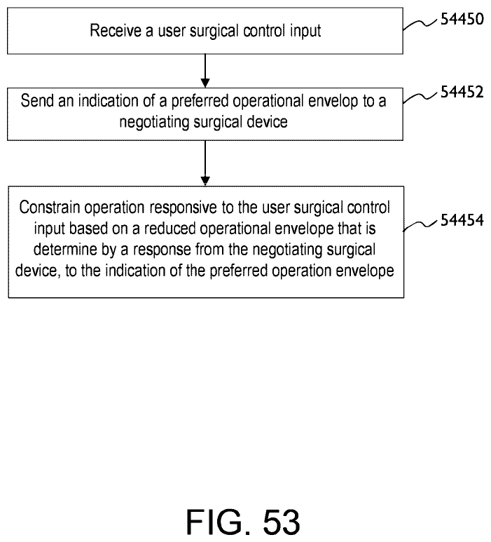

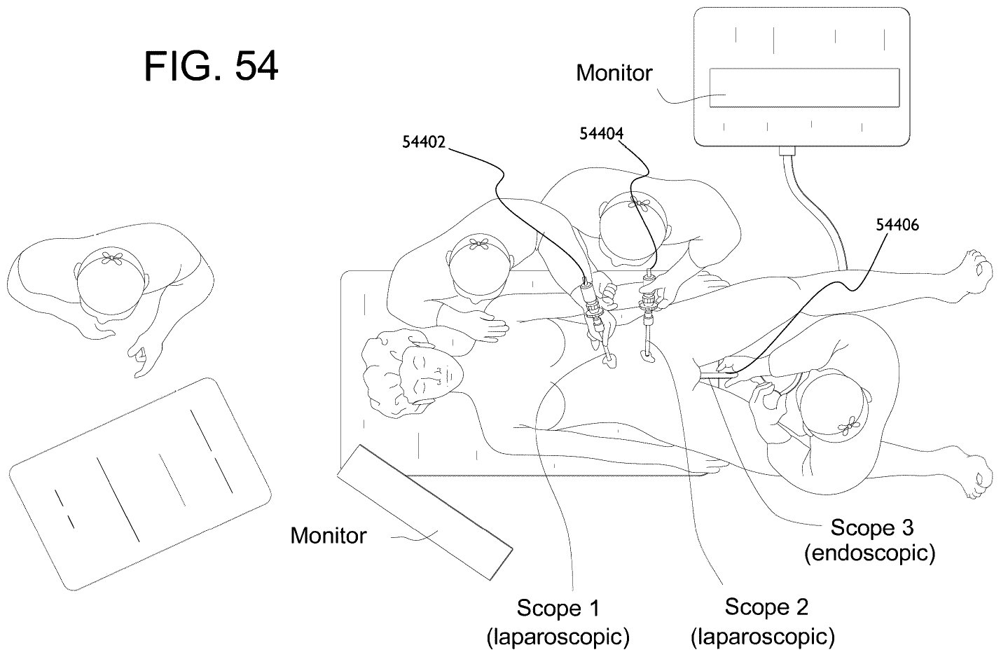

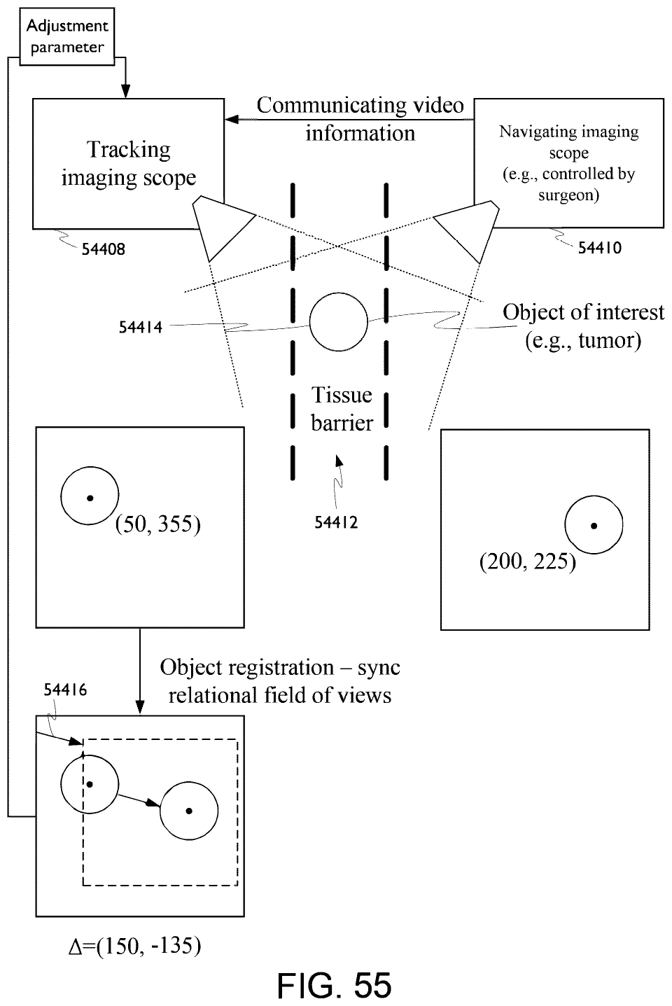

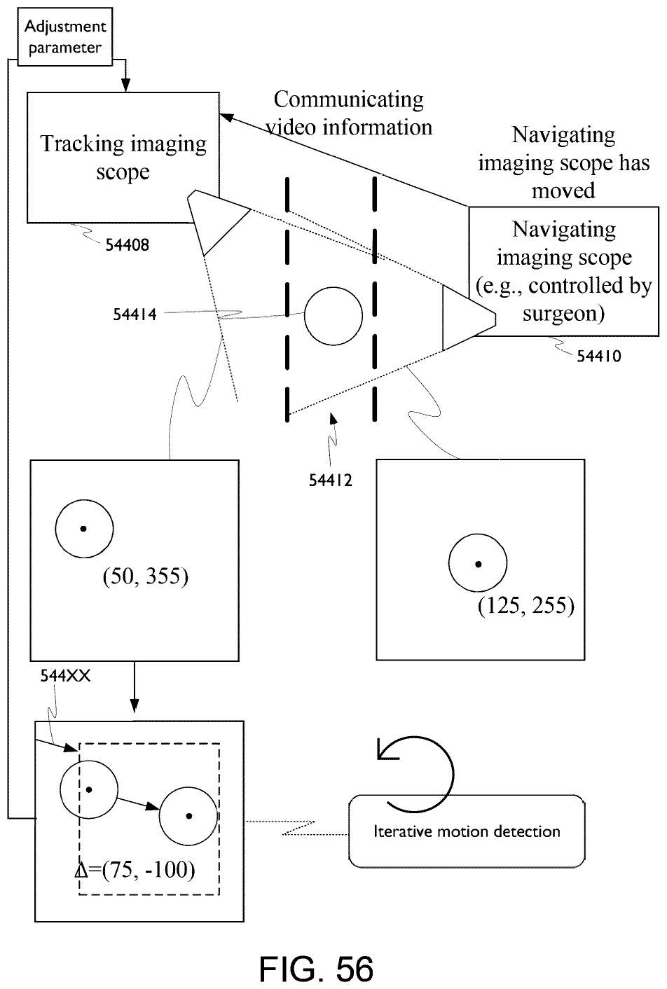

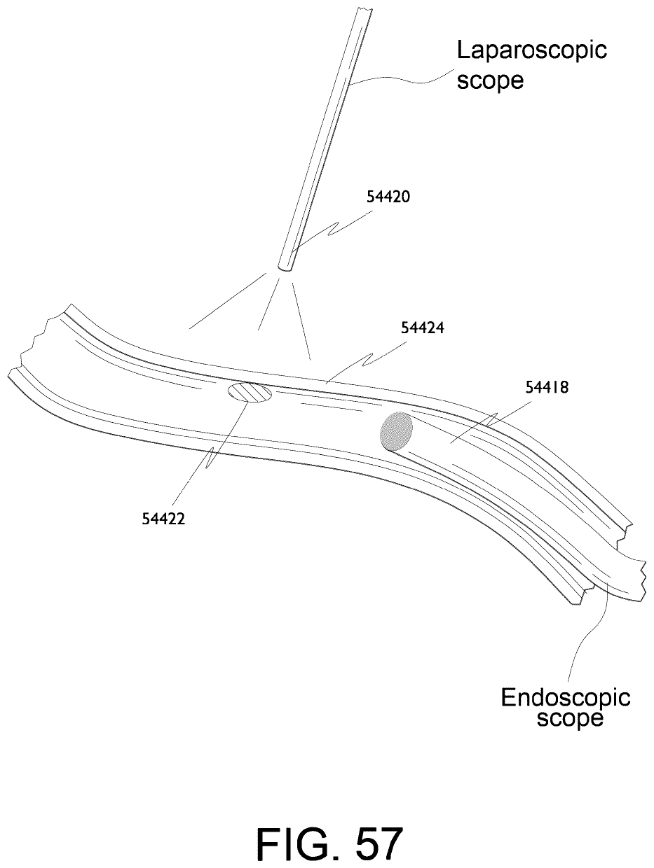

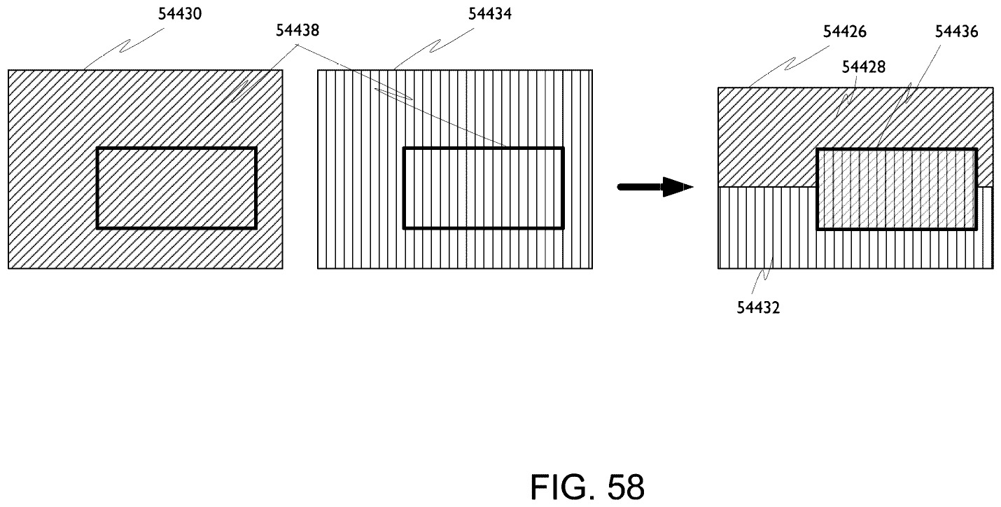

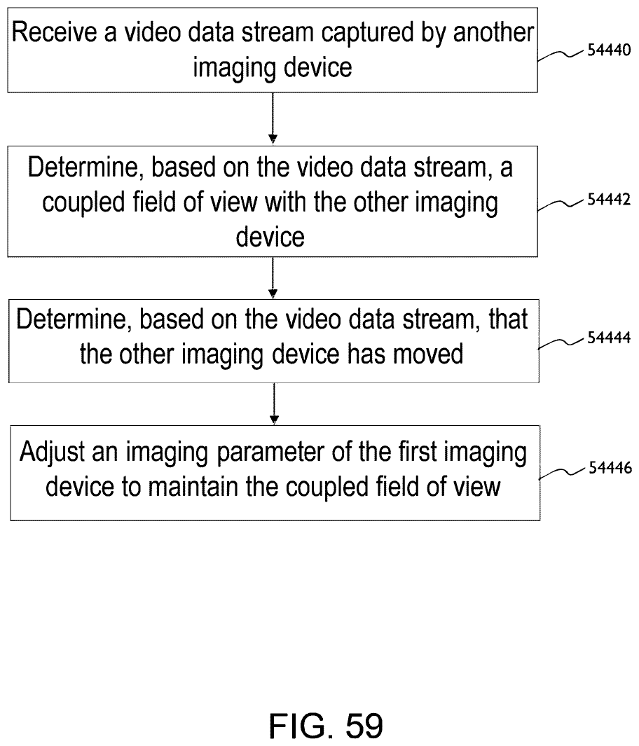

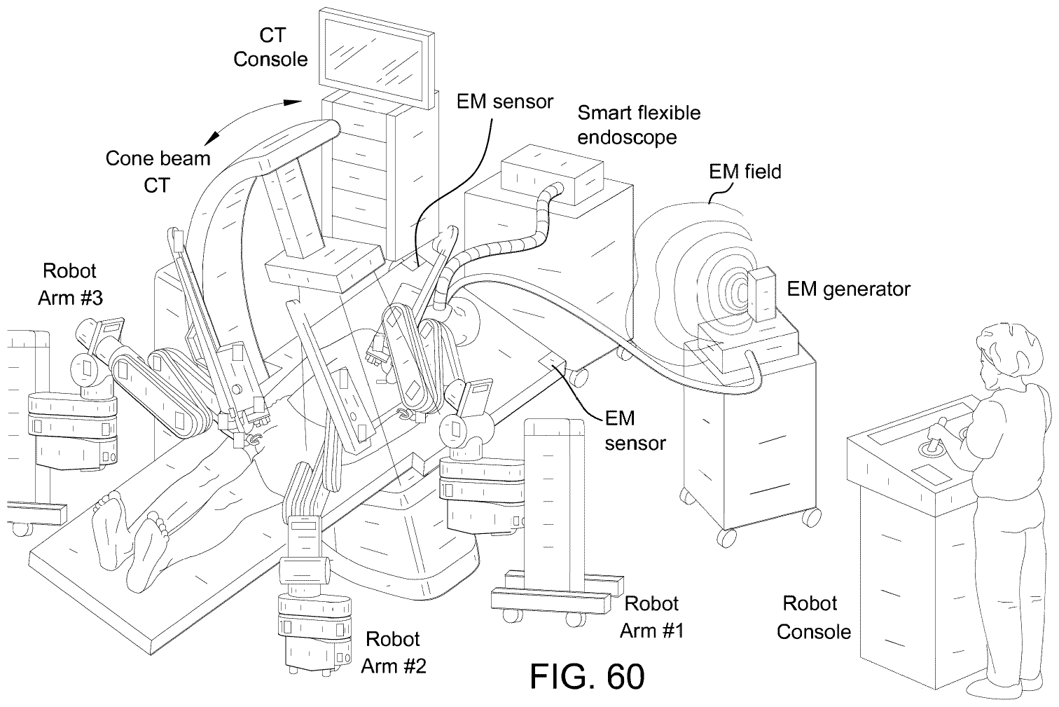

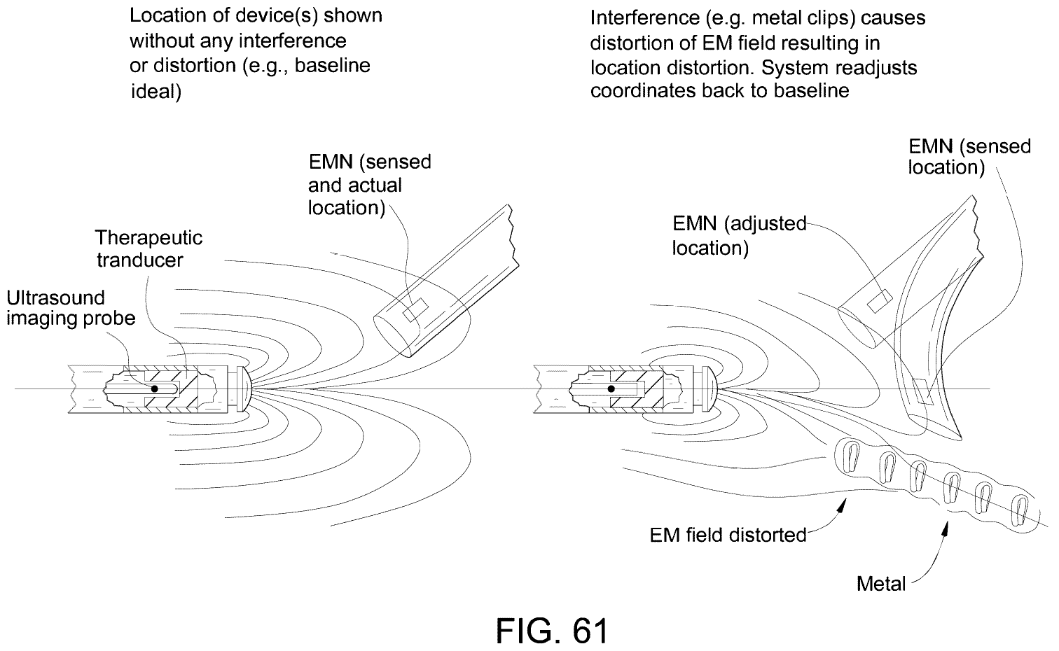

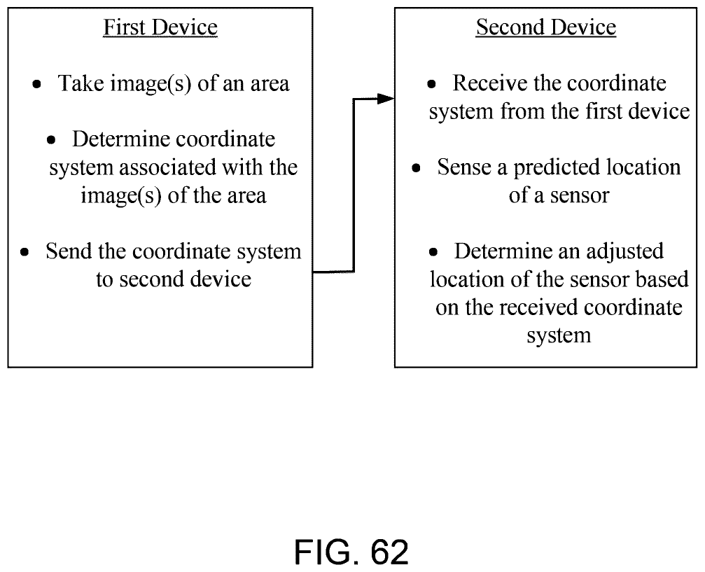

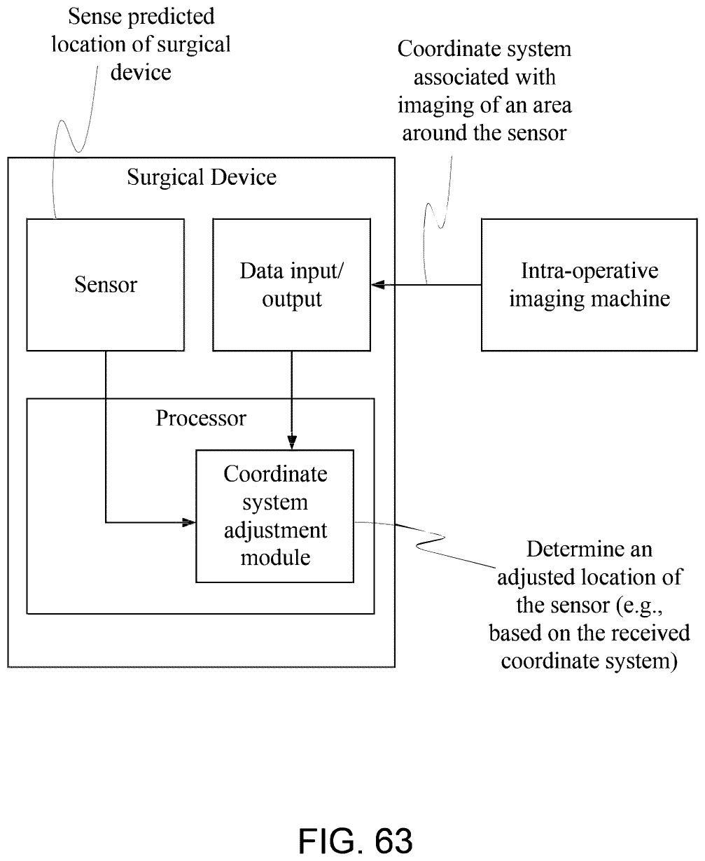

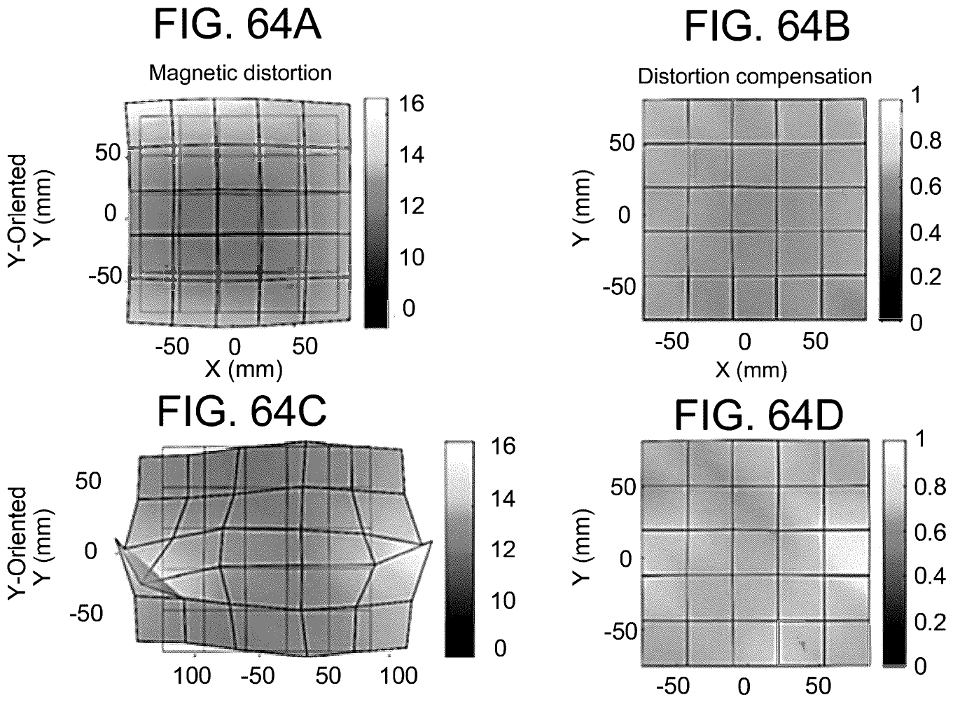

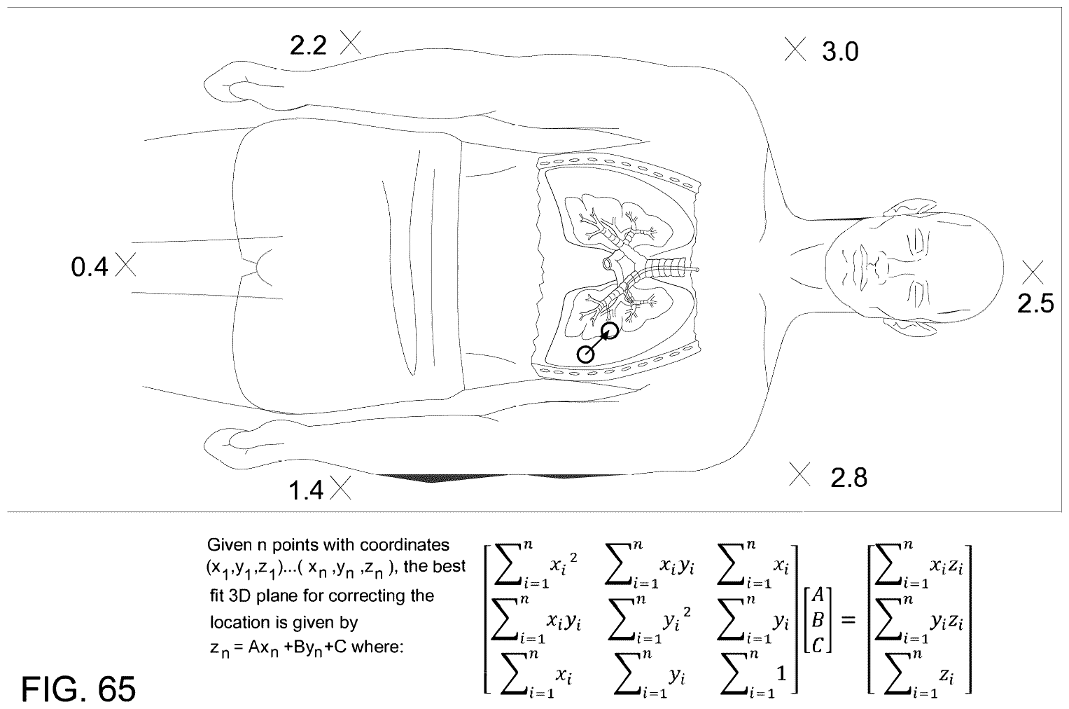

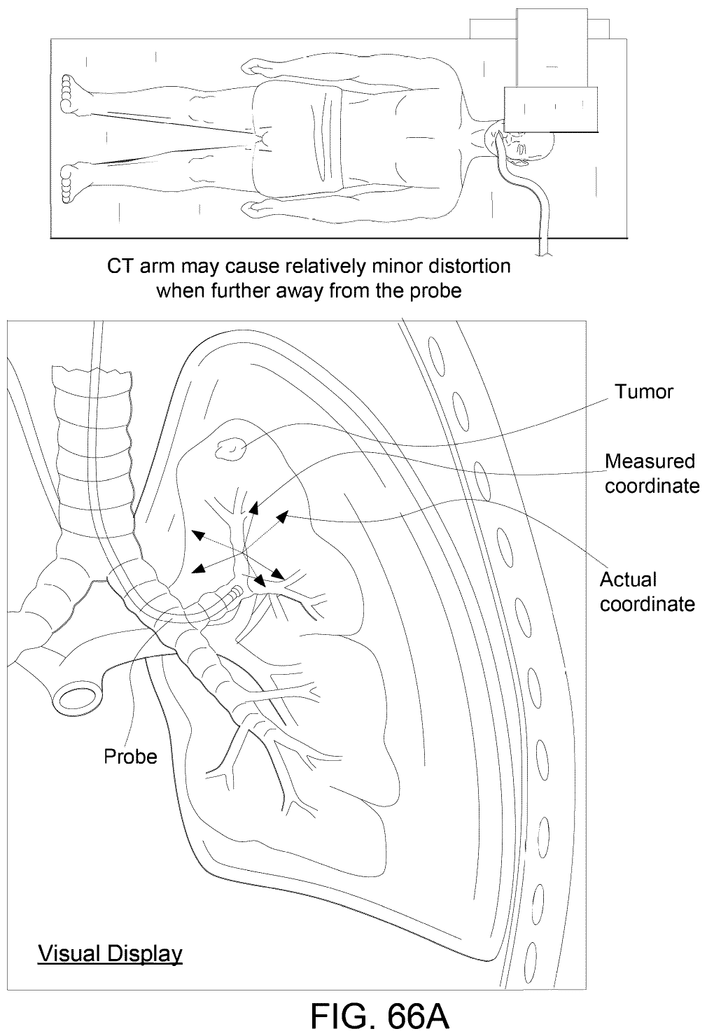

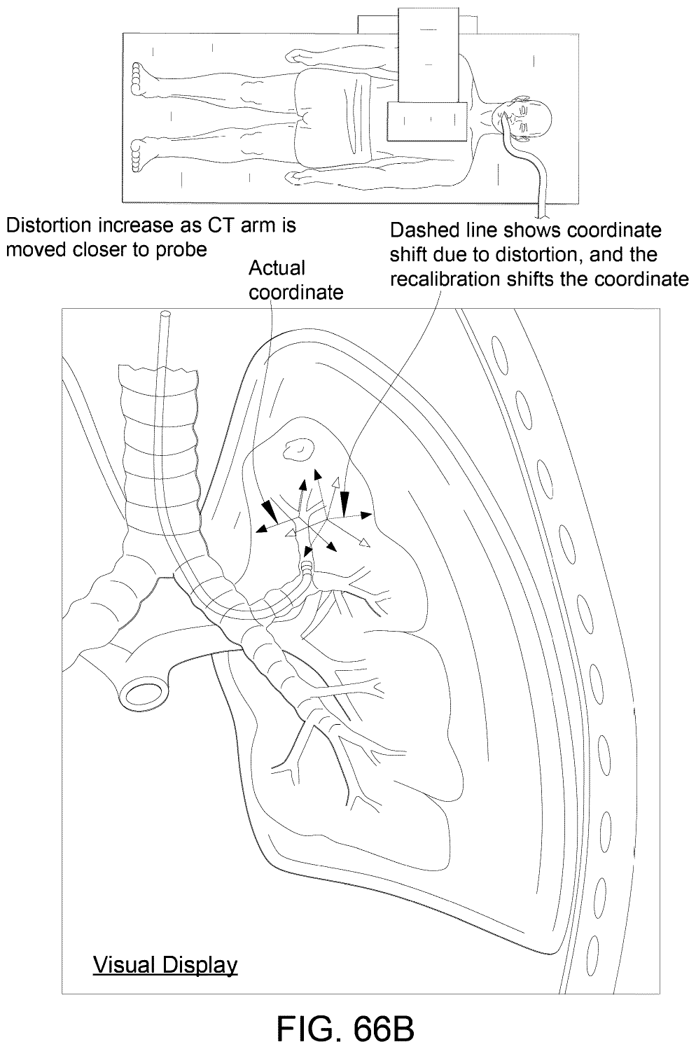

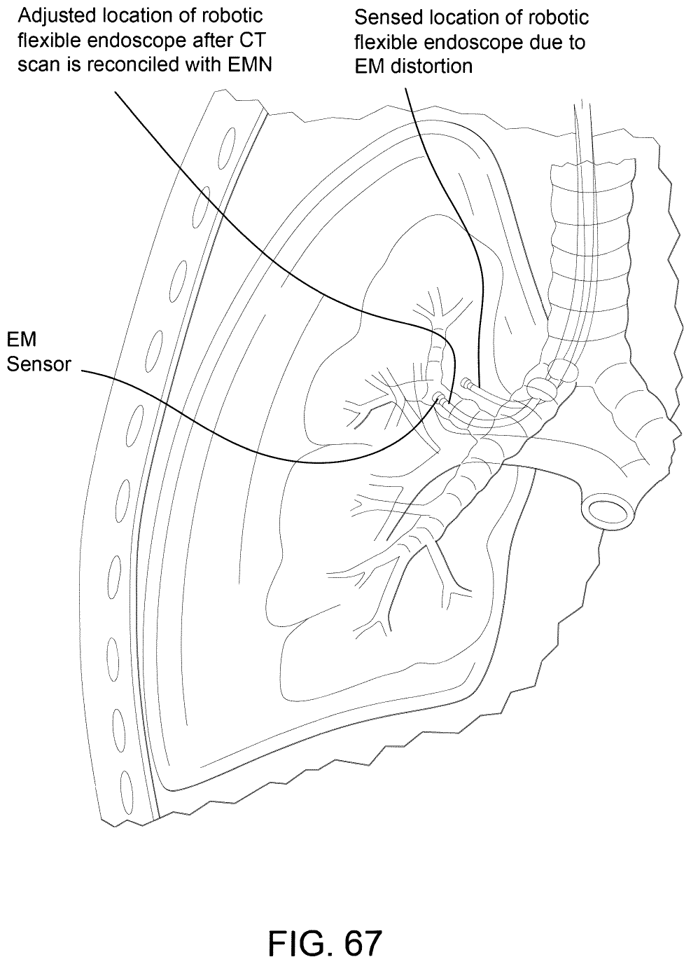

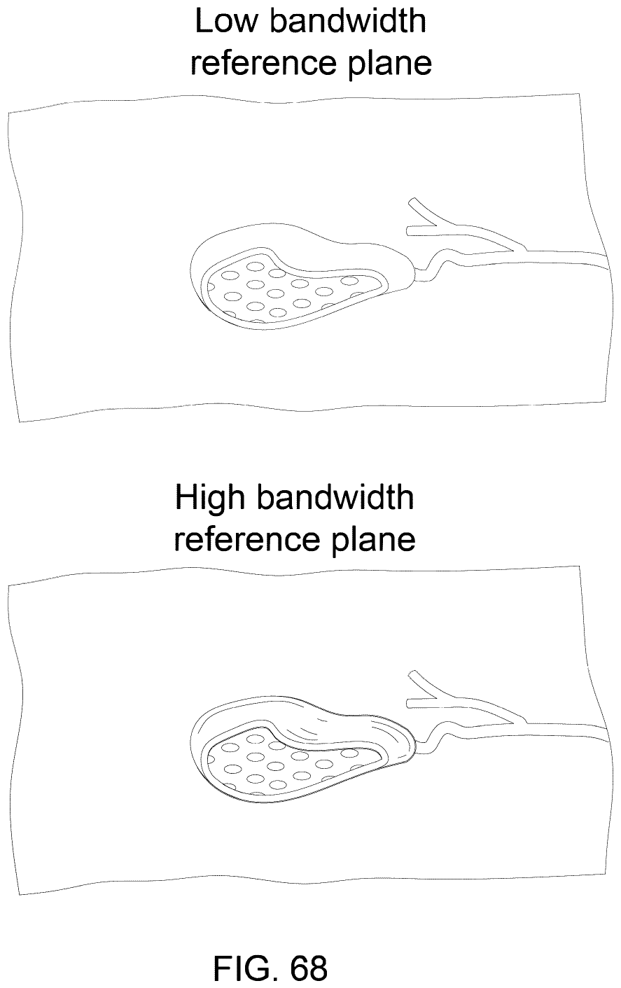

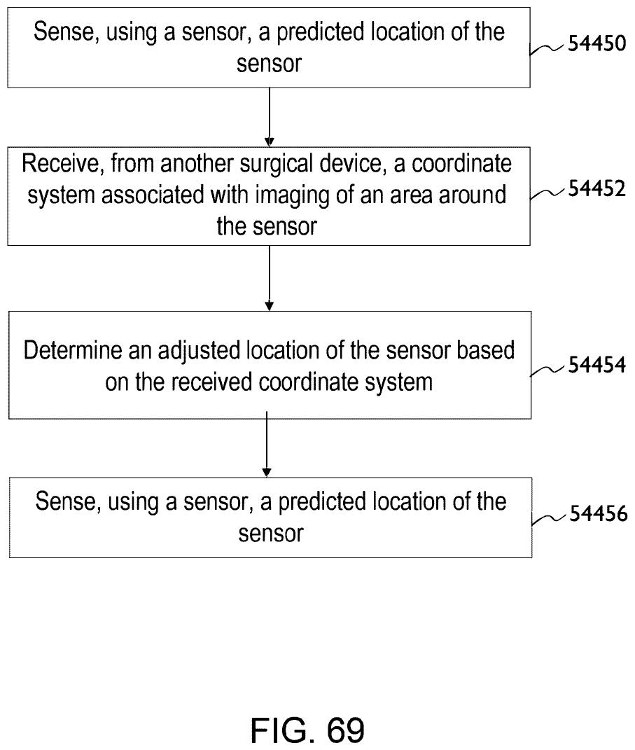

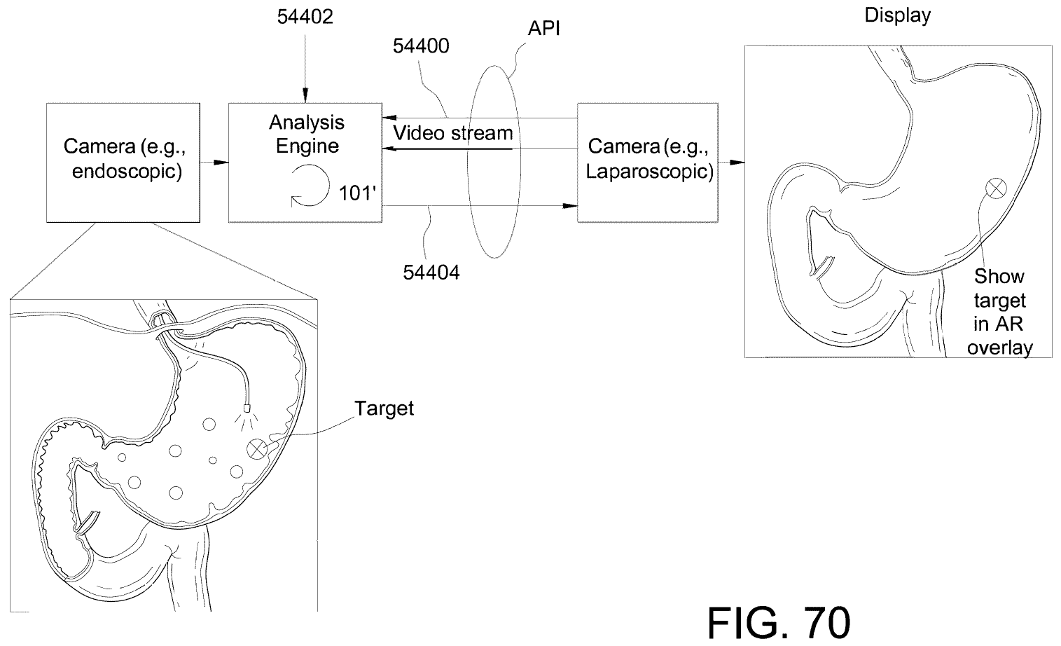

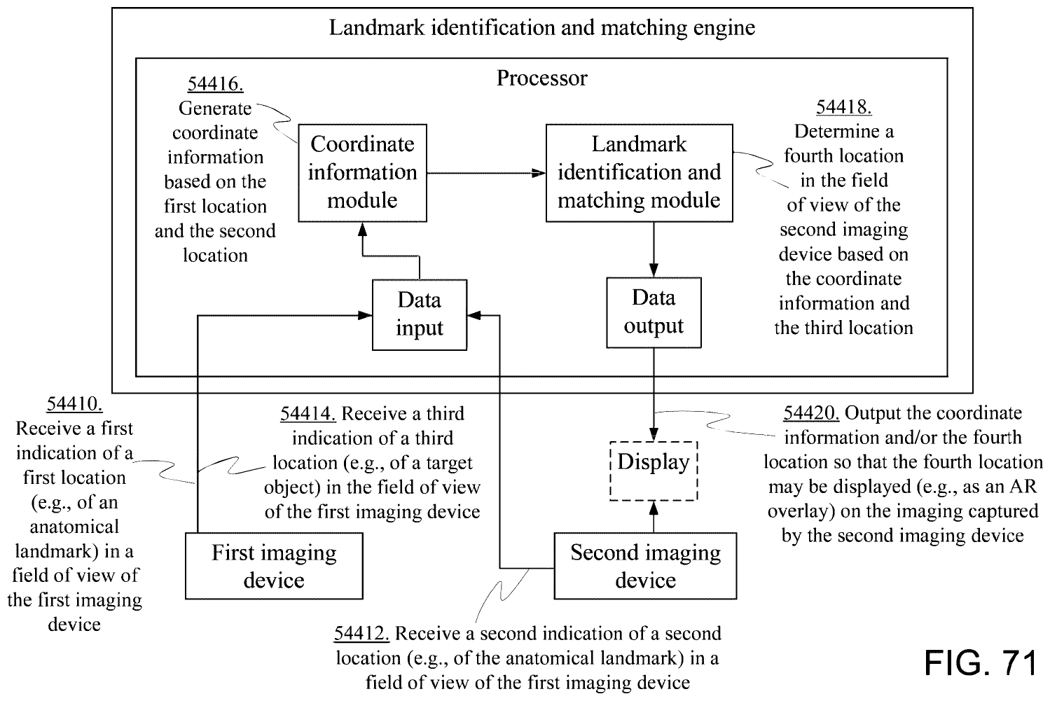

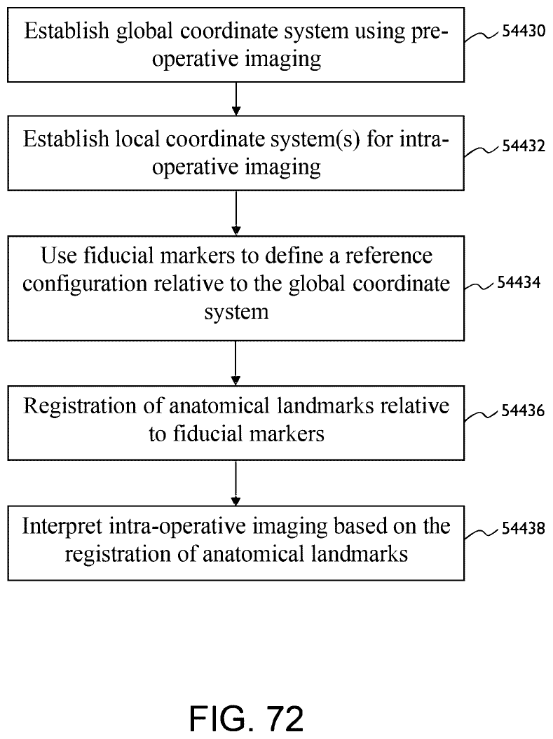

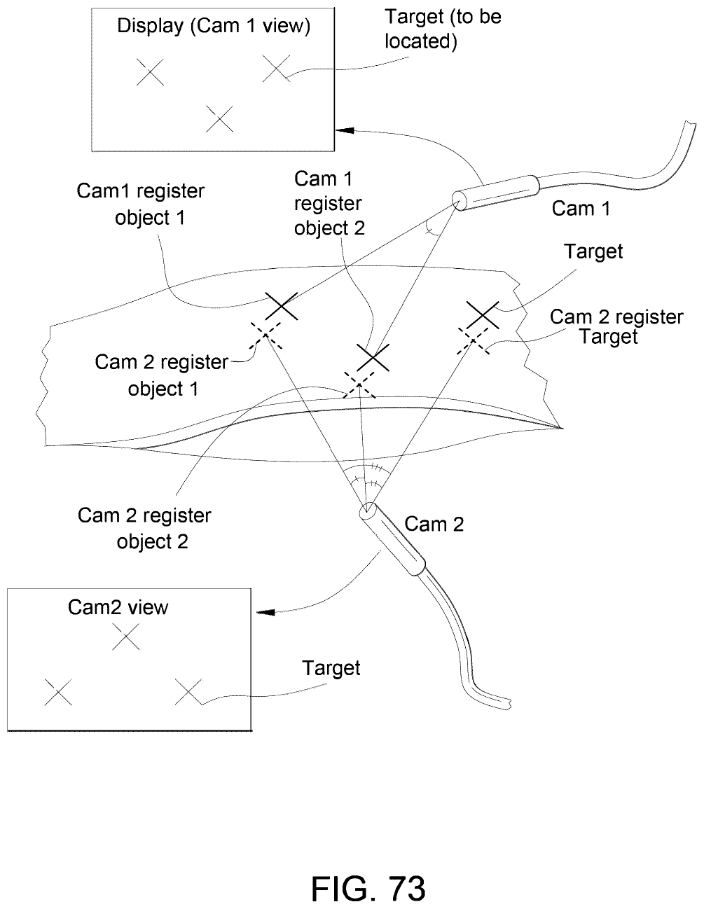

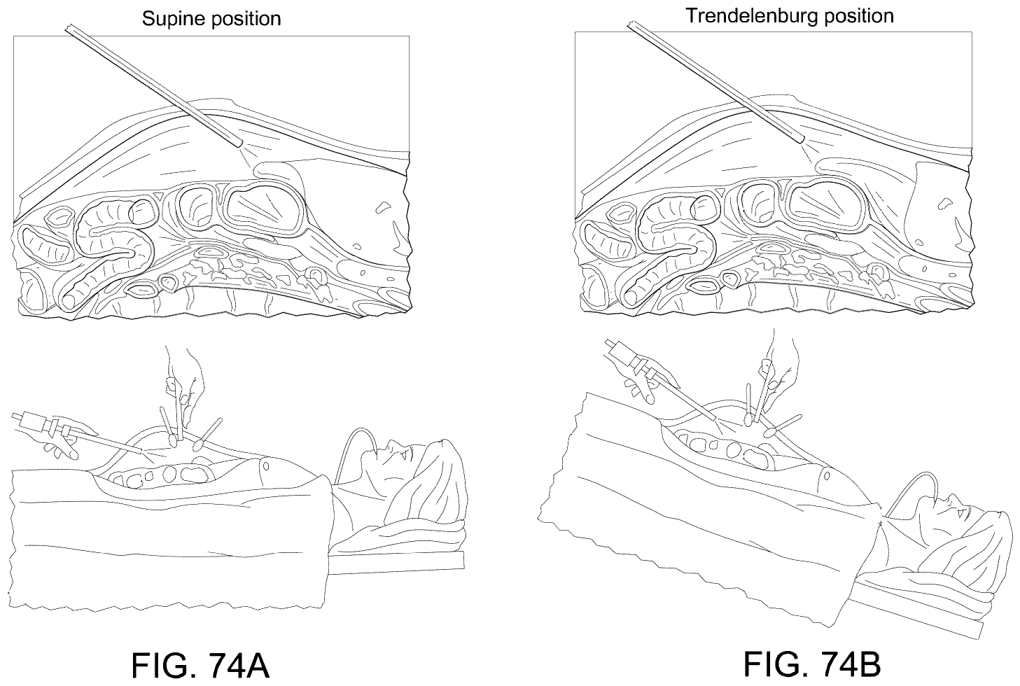

FIG. 10a depicts an example of sensed device locations without any interference or distortion. FIG. 75B depicts an example of distortion of an electromagnetic filed resulting in misalignment of sensed device locations. As depicted in FIG. 10a, ultrasound imaging probe 54068 including therapeutic transducer 54070 may accurately sense the location of device 54072 using electromagnetic navigation. As depicted in FIG. 75B, the introduction of metal, such as metal clips 54074, may cause electromagnetic field distortion. Distortion may result in inaccurate electromagnetic navigation. For example, therapeutic transducer may determine a sensed device location 54076 different than actual device location 54078 (e.g., due to the presence of metal clips 54074).

Temporal calibration may be used to estimate a time delay and may be integrated into a motion control program of a motorized scope control, for example, to enable artifact magnitude identification. Artifact magnitude identification may be used to limit the magnitude's effect on the physical measurement of position.

Redundant electromagnetic field monitoring may be used, for example, from a second magnetic sensor that is positioned at a distance to the primary source. Redundant measures may be affected differently than the primary source by the metallic in the vicinity. Field distortions may be identified by the comparison of the two measures (e.g., the primary source and the second magnetic sensor). Field distortions may be minimized from the primary measure, for example based on the comparison of the two measures.

FIG. 11 depicts an example of redundant electromagnetic field monitoring in an operating room (e.g., during a CT). Global imaging for re-calibrating electromagnetic local navigation may be performed. Electromagnetic sensor 54080 on flexible endoscope 54082 may be triangulated with electromagnetic sensor(s) 54084a, 54084b on an operating table. The electromagnetic sensors may be used to triangulate electromagnetic field 54086 produced by electromagnetic generator 54088. This triangulation may be used to re-calibrate electromagnetic local navigation.

Flexible endoscope 54082 (e.g., a robotic flexible endoscope) may anticipate its tip location based on the insertion of the scope. Flexible endoscope 54082 may account for additive errors based on time and surrounding metal objects. Arm movement from CT 54088 (e.g., a cone-beam CT) may amplify the errors as it moves into place to re-calibrate.

FIG. 12 depicts an example of reconciling electromagnetic navigation of flexible endoscope 54082. As shown in FIG. 12, flexible endoscopy 54082's local coordinate system may deviate from the global anatomy of the body. CT 54090 may be used to determine the location of the local coordinate system. CT 54090 may communicate the local coordinate system to flexible endoscope 54082 (e.g., for flexible endoscope 54082 to correct its coordinate system). Re-alignment of the coordinates may include re-alignment from an (e.g., inaccurate) sensed location 54092 to an (e.g., accurate) adjusted sensed location 54094. Re-alignment of the coordinates may include re-alignment of the expected anatomy to that of the real anatomy.

Referring back to FIG. 11, CT 54090 moving away may impact the signal. For example, misalignment may occur due to the impact of CT 54090 on the signal when CT 54090 is being moved. The monitored misalignment as CT 54090 approached may be used as a template to computationally adjust for CT 610 motion. Flexible endoscope 54082 coordinates may be monitored as the CT arm is brought into position. Flexible endoscope 54082 coordinates may include an estimate of the CT arm interference with electromagnetic field 54086. Flexible endoscope 54082 may (e.g., automatically) compensate for the same adjustment as the CT arm is removed from the field. The compensation may result in the flexible scope having a true position of its coordinate system so it may continue advancement.

Hybrid endoscopic-laparoscopic treatment with external image guidance may be performed using the surgical instrument(s), device(s), and/or systems(s) described herein. Hybrid endoscopic-laparoscopic treatment with external image guidance may be used for procedures such as a gall stone occlusion of the common bile duct.

FIG. 13 depicts an example anatomy in a supine position. Anatomy relevant to treatment of gallstones (e.g., hybrid endoscopic-laparoscopic treatment), generally includes gall bladder 54096, cystic duct 54098, common bile duct 54100, sphincter of Oddi 54102, ampulla of Vater 54104, duodenum 54106, pancreatic duct 54108, pancreas 54120, and gallstones 54122.

FIG. 14 depicts an example of endoscopic locations within the example anatomy of FIG. 13 in a Trendelenburg position. Endobronchial ultrasound (EBUS) device 54124 may emanate ultrasonic waves 54126 to identify key structures and landmarks 54128.

Hybrid endoscopic-laparoscopic treatment with external image guidance may be used to remove gallbladder stones occluding common bile duct. Removing gallbladder stones occluding the common bile duct may include one or more of the following operations. The common bile duct may be opened and the gall bladder (e.g., the source of the stones) may be removed. Cooperative smart system(s) (e.g., a robotic flexible endoscope, a robotic laparoscope, a high intensity ultrasound therapeutic system) may be incorporated in the procedure. At least two cooperative smart systems may interchange data, registrations, and the like, may work from both sides (e.g., of the patient). Cooperative smart system(s) may receive targeting information from other cooperative smart system(s). In some examples, a cooperative smart system may not provide data back to other cooperative smart system(s) (e.g., if the cooperative smart system receives target information from the other cooperative smart system(s)).

Gallstones may cause pain (e.g., biliary colic) and gallbladder infections (e.g., acute cholecystitis). Gallstones may migrate out of the gallbladder and become trapped in the tube between the gallbladder and the small bowel (e.g., common bile duct). In the common bile duct, gallstones may obstruct the flow of bile from the liver and gallbladder into the small bowel and cause pain, jaundice (e.g., yellowish discoloration of the eyes, dark urine, and pale stools), and/or severe infections of the bile (e.g., cholangitis). People undergoing cholecystectomy for gallstones may have common bile duct stones.

Treatment may involve removal of the gallbladder as well as the gallstones from this tube. There may be several methods of treatment. Surgery may be performed to remove the gallbladder. This may be performed through a single large incision through the abdomen (e.g., open cholecystectomy). Keyhole techniques (e.g., laparoscopic surgery) may be used to remove the gallbladder. Removal of the trapped gallstones in the common bile duct may be performed at the same time as the open or keyhole surgery.

Removal of the trapped gallstones may be performed independently of the open or keyhole surgery. An endoscope (e.g., a narrow flexible tube equipped with a camera) may be inserted through the mouth and into the small bowel to allow removal of the trapped gallstones from the common bile duct. This procedure may be performed before, during, or after a surgery to remove the gallbladder. Feature(s) associated with removal of the common bile duct stones during surgery to remove the gallbladder as a single-stage treatment or as a separate treatment before or after surgery (e.g., two-stage treatment) are provided herein.

Pancreatitis is inflammation of the pancreas. The pancreas is a long, flat gland that sits tucked behind the stomach in the upper abdomen. The pancreas produces enzymes that help digestion and hormones that help regulate the way a person processes sugar (glucose). Pancreatitis may occur as acute pancreatitis (e.g., it may appear suddenly and may last for days). Chronic pancreatitis may be developed (e.g., pancreatitis that occurs over many years). Mild cases of pancreatitis may improve with treatment. Severe cases of pancreatitis may cause life-threatening complications. Pancreatitis may occur if the bile duct is clogged, and the pancreas enzymes cannot be transferred into the small intestines. The enzymes may begin to break down the pancreas itself from the inside out.

Performing gallstone removal may include one or more of the following operations (e.g., identification, tagging, and management of common bile duct stones).

A computed tomography (CT) scan of the abdomen may be performed. A computed tomography (CT) scan is an imaging test that may use X-rays and a computer to produce detailed images of the body. A CT scan may show details of the bones, muscles, fat, soft tissues, organs, and/or blood vessels. CT scans may be more detailed than X-rays. During a CT scan an X-ray beam may move circumferentially around a patient's body. CT scans may allow for different views of the same part of the body. The X-ray information may be sent to a computer. The computer may interpret the X-ray data. The computer may display X-ray data (e.g., interpreted X-ray data) on a monitor. In examples, a patient may receive a contrast dye (e.g., prior to a CT scan). The contrast dye may be given orally and/or intravenously. The contrast dye may make part(s) of the patient's body show up better in the produced image(s). CT scans of the abdomen may give more detailed information than an X-ray. CT scans may give healthcare providers more information about injuries or diseases of the abdominal organs.

Performing a CT scan of the abdomen may include one or more of the following operations. The patient may be asked to remove any clothing, jewelry, or other objects that may interfere with the scan. The patient may be given a gown to wear (e.g., if the patient was asked to remove clothing. If the patient is to have a scan done with contrast, the contrast dye is provided to the patient. For intravenous contrast, an IV line may be started (e.g., in the hand or the arm for injection of the contrast dye). For oral contrast, the patient may be given a liquid contrast to drink. In examples, the contrast dye may be given rectally. The patient may lie on a scan table that may slide into a circular opening of a scanning machine. Pillows and/or straps may be used to help prevent movement during the scan. The technician may be in another room where the scanner controls are located. The patient may be able to see the technician through a window (e.g., during the entirety of the CT scan). Speakers may be incorporated with the scanner to allow the technician to talk to and/or hear the patient. The patient may be given a call button (e.g., so that the patient can let the technician know if the patient has any problems during the CT scan). The technician may be watching the patient (e.g., throughout the CT scan) and may be in constant communication. X-rays may pass through the patient's body for short amounts of time (e.g., as the scanner begins to rotate around the patient). The X-rays absorbed by the body's tissues may be found by the scanner and sent to the computer. The computer may produce an image using the information. The image may be interpreted by a radiologist. The patient may be asked to remain still during the scan. The patient may be asked to hold their breath for a short time at various times during the scan. It may be important that the patient stay still during the scan (e.g., such that a more accurate image may be produced). The patient may be removed from the scanner after the first set of scans has been completed. A second set of scans may be taken after the contrast dye has been given (e.g., if contrast dye is used). The patient may feel effects from contrast dye if administered. These effects may include a warm, flushing sensation; a salty or metallic taste in the mouth; a brief headache; and/or nausea. These effects may be temporary. If contrast dye is administered intravenously, the patient may feel some effects when the dye is injected into the IV line. The patient may alert the technician if the patient has any trouble breathing, sweating, numbness, and/or heart palpitations. When the scan has been completed, the patient may be removed from the scanner. If an IV line was inserted (e.g., for administering contrast dye), the IV line may be removed. The patient may be asked to wait for a short period of time while the radiologist examines the scans (e.g., to make sure the scans are clear).

FIG. 15 depicts an example CT scan of a patient in a supine position and an example EBUS scan of the patient in the Trendelenburg position. CT scans and EBUS scans may include vessels 54130, gallstones 54132, lymph node 54134, and bile duct 54136. Change in a patient's position (e.g., from supine position to Trendelenburg position) may change the relative location of certain anatomical features (e.g., to the viewer and to other anatomical features). For example, lymph node 54134 may occlude part of a gallstone 54132 when the patient is in a first position (e.g., Trendelenburg position) but not when the patient is in a second position (e.g., supine position).

Adjustment of pre-operation imagines (e.g., an image produced during a CT scan) to fit real time imaging may be performed. Local coordinate systems may be defined for the available images (e.g., updated in real time). An analysis of the available images (e.g., from multiple sources) may be performed to identify key structures of interest. A CT scan may be used to show key structural elements and landmarks, such as the common bile duct, biliopancreatic duct, ampulla of vater, gall bladder, liver, pancreas, duodenum, and some gall stones (e.g., the CT scan can be inconclusive). With a known local coordinate system (e.g., relative to an adjusted global coordinate system), information from EBUS may identify one or more of gall stones, ampulla of vater, common bile duct, biliopancreatic duct, or the like. This identification information can then be used to confirm the identity of structures from other views. For example, with coordination, a lap view may be used to identify structures for the surgeon, for example, based on the information gathered and interpreted from the CT image and the ultrasound image. A similar approach may be used to identify lymph nodes and/or other structures of interest.

Pre-operation imaging of organs and a surgical site in a supine position may be distorted in the reverse Trendelenburg position (e.g., when used for surgery). This distortion may not be uniform with the retroperitoneal (e.g., structures behind the peritoneum) and peritoneal (e.g., structures in front of the peritoneum) organs, for example, due to their levels of fixation to the more rigid portions of the body. Retroperitoneal structures may move less with changes in anatomic position (e.g., because retroperitoneal structures may be more rigidly fixated to the back wall of the cavity).

To utilize the pre-operative imaging for guidance, registration, and/or identification of differing real-time surgery imaging, the anatomy may be adjusted to that of the surgical position. Pre-operative imaging may be adjusted by identification of common stationary landmarks (e.g., less moving, or non-moving landmarks), 3D shape comparison, and/or synchronization of local fiducial markers.

Multiple imaging platforms and technologies may be used. Communication between the systems may improve coordination between platforms and technologies. The endoscopic view (e.g., flexible endoscopic view) may have visualization and/or ultrasound imaging onboard. The laparoscopic camera may have visual image(s) (e.g., stored onboard or accessible). Connecting and communicating the information from the endoscopic view, the laparoscopic camera, and imaging from the pre-operation CT, which may provide the most detailed information, may enable the real-time identification of structures within either visualization platform (e.g., endoscopic, or laparoscopic).

Fiducial alignment may be performed. The global coordinate system may be established for the pre-operation CT. The laparoscopic system and/or the endoscopic system may have their own coordinate systems (e.g., locally). Multiple fiducial markers seen by each system may be used to establish a reference configuration (e.g., that may be defined relative to the global coordinate system). The reference configuration may account for deformations based on the current image. Coordinating the fiducials in real time may provide greater clarity and accuracy on the local coordinate systems for each system. Registration may allow for interpretation of the images (e.g., a real-time image) for structural elements and landmarks that a technology (e.g., visual laparoscopic camera) may not otherwise be configured to identify. The system may re-establish registration and compensate for patient positioning changes.

Discrimination of gallstones from other structures may be capable under local ultrasound (e.g., more capable than on a full body CT). Improved identification (e.g., for tagging) of gallstones for communication to other smart system(s) may be performed. Utilizing the new identification capabilities in surgery may improve registration for location and positioning.

Detection of the presence of kidney and gallstones via CT may be performed a majority (e.g., 95-98%) of the time. Low dose CT accuracy may be lower. Low dose CT may be able to discriminate each stone from adjacent normal anatomic structures at a lower percentage of the time (e.g., compared to CT). External ultrasonic imaging may be about as accurate as CT. External ultrasonic imaging may be considered less accurate in identification of gallstones.

Endoscopic ultrasound (EUS) guided stone extraction may be performed. EUS may be applied in therapeutic interventions of hepatopancreatobiliary problems. Removal of CBD stones under EUS guidance may be performed to minimize the use of fluoroscopy and contrast medium injection. EUS-guided techniques may be preferable in conditions of previous failed biliary cannulation attempts or difficulty in accessing the papilla (e.g., malignant duodenal obstruction, altered surgical anatomy, large duodenal diverticulum).

FIG. 16 depicts an example EUS guided stone identification method. EUS 54138 may use ultrasound waves 54140 to detect gallstones 54142.

EUS-guided stone extraction may include one or more of the following operations. A biliary system may be punctured under EUS guidance from the stomach or from any location where dilated left intrahepatic duct may be accessed easier from the duodenal bulb. A wire may be passed through the FNA needle into the duodenum (e.g., can be performed under fluoroscopy guidance). This procedure may be performed with a balloon-pushed antegrade (EUS-AG) (e.g., when the papilla cannot be accessed) or with rendezvous technique (EUS-RV) (e.g., when the papilla is accessible). The gallstone may be pushed with a retrieval balloon.

Local ultrasound (e.g., on a flexible endoscope) may be significantly more accurate in the final identification and location of gallstones. The local system may be used to determine the in-surgery registrations, make a final determination of stone vs anatomy, and/or help the other imaging systems be adjusted to match the imaging seen in real time.

An EUS-guided approach may be propitious (e.g., in cases with surgically altered anatomy). In surgically altered anatomy patients, EUS-guided approach may yield better results when the procedure is performed with various therapeutic options (e.g., EUS-AG, EUS-RV, peroral cholangioscopy with intraductal lithotripsy, and EUS-guided enterobiliary fistula) rather than performed as a single procedure.

Extracorporeal shockwave lithotripsy (ESWL) may be performed. ESWL may generate high-pressure electrohydraulic shockwaves outside the body (e.g., to fragment gallstones). The waves may be produced by piezoelectric crystals of electromagnetic membrane technology. The waves may be directed by elliptical transducers through a liquid medium. This procedure may be conducted under the guidance of ultrasound machine and/or fluoroscopy. A nasobiliary tube (NBT) may be inserted for better visualization. The success of a single session of ESWL procedure may depend on the size and structure of the gallstones and/or the presence of bile duct stenosis. ESWL may allow for fragmentation of multiple gallstones simultaneously.

A high success rate of ESWL procedures may be demonstrated. ESWL may show minimal and mild adverse events. More serious adverse events (e.g., transient biliary colic, subcutaneous ecchymosis, cardiac arrhythmia, haemobilia (often self-limiting), cholangitis, ileus, pancreatitis, perirenal hematoma, bowel perforation, splenic rupture, lung trauma, and necrotizing pancreatitis) may be anticipated. Considerably low recurrence rate of CBD stones after CBD clearance may demonstrated.

ESWL may be beneficial for patients with anatomically abnormal structures. For example, in patients with inaccessible papilla (e.g., due to history of Billroth-II or Roux-en-Y surgeries). The size of CBD may often be large in cases with surgically altered anatomy (e.g., in addition to the size of bile duct stones). In these cases, endoscopic nasobiliary drainage tube placement may often be required to guide ESWL. Percutaneous transhepatic biliary drainage (PTBD) or endoscopic ultrasound (EUS)-guided intraductal lithotripsy may be performed (e.g., if an optimal result is not achieved with ESWL).

Endoscopic biliary stenting may be performed. Endoscopic biliary stenting may be an alternative approach (e.g., for stone removal) for patients with difficult bile duct stones and/or high risk of complications (e.g., the elderly, patients with serious comorbidities, patients on anti-thrombotic, or patients who are frail). Endoscopic biliary stenting may be a definitive therapy for those who cannot undergo a surgical approach. Biliary stents may contribute towards stone removal. Stone fragmentation may be caused by mechanical friction against the stones.

Cholecystectomy (e.g., gallbladder removal surgery) including one or more of the following steps: dissecting the hepatocystic triangle, establishing acritical view of safety, clipping and dividing the cystic artery, performing operative cholangiography and dividing cystic duct, separating gallbladder from the liver bed, and removing specimen and port.

For example, dissection of the hepatocystic triangle may be performed. The gallbladder may be retracted over the liver with cephalic traction, while inferior-lateral traction on the neck of the gallbladder may be applied through the midclavicular port site. An assistant may maintain constant tension on the retractor (e.g., unless adjustments are required for changes in visualization). The surgeon may (e.g., using T3) manipulate the neck of the gallbladder to expose anterior (e.g., medial) and posterior (e.g., lateral) aspects as needed. If the gallbladder is distended, the gallbladder may be decompressed with a needle aspiration device (e.g., to avoid perforation with spillage of bile and gallstones). If adhesion(s) are present, the adhesion(s) may be taken down bluntly and/or with monopolar energy (e.g., while taking care to avoid energy use near the duodenum which can be adherent to the gallbladder). The dissection may begin by incising peritoneum along the edge of the gallbladder on both sides to open up the hepatocystic triangle. This may be carried up posteriorly along the wall of the gallbladder at its interface with the liver. A combination of blunt dissection and judicious use of cautery may be needed (e.g., to clear the triangle of fat and fibrous tissue).

A critical view of safety may be established. The critical view of safety may be associated with one or more of the following criteria to be met. The hepatocystic triangle (e.g., defined as the triangle formed by the cystic duct, the common hepatic duct, and inferior edge of the liver) may be cleared of fat and fibrous tissue (e.g., all fat and fibrous tissue). The common bile duct and common hepatic duct may be looked for but not exposed by dissection. The lower portion (e.g., one third) of the gallbladder may be separated from the liver to expose the cystic plate. The cystic plate may be defined as the liver bed of the gallbladder and may represent the gallbladder fossa. The cystic duct and the cystic artery may be seen entering the gallbladder. Once this view is established, aberrant anatomy may be identified. Variations in cystic duct position and entry into the common bile duct and/or variants in arterial anatomy may be common. One common consideration may be to ensure the right hepatic artery is not mistaken for the cystic artery or accessory branch posteriorly in the area of the cystic plate.

Next, the cystic artery may be clipped and divided. A reusable clip applier (e.g., with 8 mm clips) may be utilized to clip the cystic artery. Two clips may be applied on the proximal side and one clip on the distal (e.g., specimen) side (e.g., with an adequate gap between to allow for division). Hook scissors may be used to divide the artery. A small cuff of tissue may be left beyond the edge of the clips to prevent accidental dislodgement. A clip may be placed on the neck of the gallbladder at the upper end of the cystic duct-GB junction. Division right at the clip may predispose the distal specimen clip to become dislodged.

Operative cholangiography and division of the cystic duct may be performed. It may be routine to perform cholangiography. Cholangiography may be performed selectively. Indications to perform cholangiography may include one or more of suspicion of CBD stones (e.g., history of abnormal liver function tests or gallstone pancreatitis), a dilated common bile duct, uncertainty of anatomy or concern for biliary injury, or a history of Roux Y gastric bypass (e.g., which precludes subsequent ERCP). If no cholangiogram is performed, the cystic duct may be clipped with three clips, two on the stay side and one on the gallbladder specimen side (e.g., similar to the cystic artery). If the cystic duct is dilated or thickened or there are bile duct stones, a pre-tied endoloop suture may be used to secure the duct on the proximal side.

If an intraoperative cholangiogram is to be performed, a single clip may be placed at the junction of the cystic duct and infundibulum of the gallbladder. The cystic duct may be partially incised with hook scissors. The cystic ductotomy may be dilated with microscissors (e.g., to disrupt any valves in the cystic duct). A ureteral catheter may then be inserted into the cystic duct (e.g., using an Olson cholangioclamp to secure it). Single spot films may be used to adjust the position of the C-arm so the entire biliary tree and duodenum may be visualized in the center of the frame. One syringe containing saline and another syringe containing a 50-50 mixture of saline and iodinated contrast media may be attached (e.g., via a three-way stopcock) for injection. The duct may be flushed with saline and then contrast under fluoroscopy. If there is no entry of contrast into the duodenum, glucagon may be given intravenously to relax the sphincter of Oddi and the injection may be repeated after a short period (e.g., after two to three minutes). If difficulty is encountered with retrograde filling of the duct, one or more of placing the patient in Trendelenburg, gently compressing the distal duct with an atraumatic grasper, or injecting morphine intravenously may to facilitate contracture of the sphincter. If a stone is visualized in the bile duct, a trans-cystic common bile duct exploration, laparoscopic choledochotomy, or referral for postoperative ERCP may be performed.

Once a satisfactory cholangiogram is obtained, the cholangiogram catheter may be removed. The cystic duct may be doubly clipped and divided. A pre-tied loop suture may be used to secure the duct.

Gallbladder separation from the liver bed may be performed. Retrograde dissection of the gallbladder from the liver bed may be performed. An L-hook monopolar energy device may be used to dissect gallbladder off the liver. Entry into the liver bed may result in bleeding and/or bile leakage from a superficial subparenchymal duct. Entry into the gallbladder with spillage of bile and stones may make subsequent dissection more difficult. An advanced energy device, such as an ultrasonic coagulator, may maintain hemostasis basis better and produce less smoke plume (e.g., in the setting of acute cholecystitis). The last attachment may be left in place to allow for retraction of the liver cephalad and clear visualization of the cystic plate to allow for any needed hemostasis (e.g., prior to complete disassociation of the gallbladder from its bed). The liver bed may be irrigated, and any blood or bile aspirated from the field. The gallbladder may be placed in an entrapment bag and removed at the port site.

Specimen and port removal may be performed. The specimen (e.g., in an entrapment bag) may be removed at the port site (e.g., whether at the umbilicus or epigastric region). Enlargement of the skin and fascial opening may be needed (e.g., if there are multiple or larger stones and/or a thickened gallbladder). Once the specimen is removed, the ports (e.g., all ports) may be vented to eliminate any residual CO₂ gas. The fascia at the extraction port site and the skin may be closed with sutures.

Laparoscopic and endoscopic co-imaging control may be performed using the surgical instrument(s), device(s), and/or system(s) described herein. Laparoscopic and endoscopic co-imaging control may be used for procedures such as a malignant airway obstruction (MAO) or central airway obstruction (CAO) diagnosis, debulking, and post treatment continued therapeutic destruction. A MAO or CAO may require tumor debulking.

Radio opaque saturation (e.g., on a tumor to a desired density) may be performed using one or more of the following operations. Endoscopic robotic bronchoscopy may be performed, for example, with a biopsy needle and the ability to deliver local drugs to the tumor for treatment at the time and site of biopsy. A flexible endoscope may be guided to the tumor through the bronchi. The needle may be extended through the working channel to biopsy the tumor. Local drug delivery may be decided. The system may aggregate the needle position, needle angle, and needle depth with the externally derived drug fluid injection pressure and the radio opaque monitoring of tumor saturation and drug leakage to control the overall full saturation of the desired area of the tumor with the desired dosage of the local drug delivery.

Temperature based debulking of a portion of the tumor obstructing the airway and physiologic cleanup (e.g., of the remaining portion of the tumor) may be performed using one or more of the following operations. Endoscopic robotic bronchoscopy with cryoablation, RF monopolar ablation, LASER, Argon plasma coagulation (APC) or microwave ablation may be used to reduce the size of the tumor. The size of the tumor may be reduced to re-establish air exchange and/or to reduce the tumor size for other tumor treatments (e.g., radiation therapy, chemotherapy, immuno-therapy). A portion of the tumor may be fully killed (e.g., while debulking the tumor). The remaining portion of the tissue may be damaged, but not killed, for the body's immune response to engage the remaining cancerous material. The killed portion of tissue may be exposed to sufficient levels of heat, cold, or electrical field to cause the destruction of the cells. Collateral interactions with adjacent cancerous cells may be limited in temperature, cold, or potential to leave the cells alive and damaged (e.g., to prevent undesired destruction of protein(s) that may help the immune system target the remaining cells). Damaging adjacent cancerous cells may help initiate and direct the immune system to the area to destroy the remaining cells.

For example, a lung cancer tumor may partially restrict the bronchus airway of a lung segment. A robotic flexible endoscope may use cryoablation to reduce the obstruction (e.g., by 75%). A robotic flexible endoscope may kill a portion of a tumor (e.g., 45%). A robotic flexible endoscope may merely damage the (e.g., remaining) cells, such as by not allowing the local temperature to go below a predetermined thresholds that may kill the remaining tumor cells. Damaging the remaining tumor cells may expose them to the immune system (e.g., and the immune supporting proteins may not be destroyed). A closed loop may use multi spectral imaging from either the laparoscopic or flexible side to monitor local temperatures within the kill zone, the damage zone, and the surrounding tissue zone. The closed loop may balance the magnitude of the liquid (e.g., argon, nitrogen, or carbon dioxide), the pressure of the liquid, and the direction of the liquid (e.g., to keep the zones within desired ranges and minimize bleed over from one zone to the next).

Heat ablation (e.g., greater than 60 degrees Celsius for kill zones and 35 to 45 degrees Celsius for damage zone) or cold ablation (e.g., less than negative 30 degrees Celsius for kill zones and 0 to negative 10 degrees Celsius for damage zones) may be used. Duration and temperature may be example control parameters on heat ablation and cold ablation treatments. Full therapeutic range for cryoablation may be negative 30 degrees Celsius to negative 75 degrees Celsius. Thrermoablation may revolve around denaturation of tissue temperature (e.g., 60 degrees Celsius to 95 degrees Celsius).

Ablation systems may use a combination of power and time to create temperatures that over time kill the cells. Cell death may be indicated by a time at temperature transform. If more effect (e.g., a larger kill zone) is desired, time and/or temperature may be increased. If less effect (e.g., a smaller kill zone) is desired, time and/or temperature may be decreased.

Thermal effects on tissue may vary at different temperatures. For example, hyperthermia may occur at 40 degrees Celsius and may cause one or more of reversible cell injury, conformational changes of cells, shrinking of collagen, or deactivation of enzymes. Devitalization may occur at 42 degrees Celsius and may cause one or more of reversible cell injury, conformational changes of cells, shrinking of collagen, or deactivation of enzymes. Coagulation may occur at 60 degrees Celsius and may cause one or more of denaturation of proteins, hyalinization of collagen, or membrane permeability changes. Desiccation may occur at 100 degrees Celsius and may cause one or more of tissue drying, extracellular vacuoles, or rupture of vacuoles. Carbonization may occur at 200 degrees Celsius and may cause one or more of tissue ablation or carbonization. Vaporization may occur between 300 to 1,000 degrees Celsius and may cause vaporization of carbon.

A proportion of lung cancer patients may develop obstruction(s) of the central airways at some point in the course of the disease. Malignant central airway obstruction (CAO) may result from primary lung cancer or any primary or metastatic intrathoracic malignancy. Malignancies adjacent to the airways, such as esophageal carcinoma, thyroid cancer, and primary mediastinal tumors, may cause airway obstruction by external compression or direct tumor growth into the airways. Extra thoracic cancers (e.g., breast, colorectal and renal malignancies) may metastasize to the airways. Malignant CAO may also occur from primary airway malignancies.

Technical success in therapeutic bronchoscopy may be defined as a post-intervention endoluminal diameter of at least half of the original airway.

Laparoscopic and endoscopic co-imaging control may be used for procedures such as a CAO tumor debulking, which may include one or more of: imaging of the obstruction, assessment of the airway obstruction, diagnostic flexible bronchoscopy, therapeutic bronchoscopy and/or introduction of an airway stent.

Imaging of the obstruction may be performed (e.g., via a chest radiograph and/or computer tomography (CT)). A basic chest radiograph may not provide significant information in the evaluation of CAO and may be far less sensitive that CT. CT may provide valuable information for procedural planning through one or more of estimations of lesion length, degree of airway narrowing, and anatomic relationships to structures surrounding the airways.

Assessment of the airway obstruction may be performed (e.g., via spirometry). Spirometry may be a useful tool to assess airflow limitation(s) from CAO and to document post treatment effect. CAO may not result in significant reductions in the forced expiratory volume at 1 second (FEV1) or vital capacity (VC) until obstruction is relatively severe (e.g., unlike in peripheral airway disease). The peak inspiratory (PIF) and peak expiratory (PEF) flow rates may be significantly reduced from CAO.

Diagnostic flexible bronchoscopy may be performed. White light flexible bronchoscopy is a diagnostic tool that may provide a real-time assessment of CAO with the ability to distinguish tumor from associated blood, secretions, and/or necrotic tissue. White light flexible bronchoscopy may be used to assess morphology and degree of CAO. Flexible bronchoscopy may biopsy lesions when obstructing pathology is unknown (e.g., histological subtyping may be a factor when contemplating therapeutic intervention).

Systemic chemotherapy, radiotherapy, and surgery may be long-term options in the management of malignant CAO. Bronchoscopic modalities may be used in the acute phase and may result in dramatic and rapid symptomatic improvement.

Therapeutic bronchoscopy may be performed. The bronchoscope may be introduced and navigated to the imaged area. Probes, needles, or ablation system(s) may be extended into contact with the tumor or adjacent to the tumor (e.g., depending on the energy modality chosen).

The treatment probe location may be determined and controlled relative to the tumor. Depth of insertion and/or distance from the tumor may be used to control the magnitude of the energy's (e.g., hot or cold) effect on the tumor and surrounding tissues. Time, energy, and/or activated control of the heat/cold propagation from the point of impact may control the magnitude of the effect. For example, a target effect may include a damaged but not killed area and/or adjacent tissues that may be unaffected.

For therapeutic bronchoscopy systems, magnitude of energy applied, treatment location, angle of attack, and/or orientation of the treatment probe may be determined. Location plus angle may define the focal location or nexus of the treatment, which then radiates outward from the focal location. Location plus angle may define the location most effected by the energy applied.

The treatment probe may be energized or may apply heat or cold to drive the tissue to the desired destructive level. The progress of the ablation and reduction may be monitored through observation with the scope, imaging through cone-beam CT, and/or monitoring of the temperature level via internal probe measurement and/or external assessment like multi-spectral imaging. Treatment may be ceased if the tumor is reduced enough to restore a portion (e.g., at least 50%) of the original airway and/or if treatment has damaged but not destroyed the adjacent remaining tumor.

An airway stent may be placed. Airway stents are prosthetic devices that may be used to maintain patency of the airway lumen. A stent may buttress the airway wall against tumor ingrowth or extrinsic compression (e.g., when patency of the airway has been partially or completed established).

Stent placement may be based on one or more conditions. The objective of an airway stent may be to palliate or to treat and prevent symptoms of CAO to allow an individual to receive systemic therapy (e.g., as stenting may not treat the tumor). Stents are foreign bodies, which may worsen certain symptoms, such as cough, and place the patient at risk for late complication. Stents may be used when symptoms are attributable (e.g., primarily attributable) to airway obstruction. There may be a risk of tumor growth or recurrence.

Thermal control of patient interaction with ventilation, ischemia, and metabolism using impact controls may be performed.

Sedation may cause hypothermia. A bear-hugger heating vest for a patient may be used to prevent hypothermia caused by sedation. A surgeon may set the thermal load input (e.g., at the beginning of the surgery) to compensate for the sedation loss. Local cooling may be used to prevent ischemia of the colon due to interruption of blood flow (e.g., for colorectal surgery). The combination of sedation cooling and local cooling may result in vasoconstriction to maintain core temperature with the aid of systemic patient warming. As the procedure progresses, the local cooling may have a more global effect on the body. When the core temperature of the body drops more than 2 degrees Celsius, the body may reverse the vasoconstriction to a vasodilation state. Open flow to the cooler extremities may rapidly increase the core temperature loss. If the patient's heating system lags too much, the rapid re-heating may cause additional cold blood flow to the heart (e.g., which may cause arrythmias and/or heart failure).

Metabolic uptake of medicates may be based on thermal levels of a patient's core temperature. As the core temperature is reduced, the medication dosage may have to be automatically adjusted (e.g., based on the lower metabolic uptake). However, when the body then reheats itself, the dosing and the existing levels of medicate may have to be reduced before the metabolic uptake re-invigorates.

Physiologic compensation causing reverse closed loop control adjustments may be performed. Both the patient core temperature and extremity temperatures may be monitored for closed loop control of the local cooling and the patient heating. When the system detects the vasodilation trigger, the system may decrease (e.g., reverse the previous increases it had indicated) local cooling and increase the patient's systemic heating to prevent the excessive rapid loss of heat (e.g., that may trigger a secondary rapid heating response).

Sedation of a patient may cause the patient to lose core body temperature (e.g., 1 to 2 degrees Celsius). To compensate for loss of core body temperature, a surgeon may use a patient heating device that is pre-set to compensate for the cold room temperature and the impacts of sedation. The body may vasoconstrict the blood flow to the extremities as it loses temperature. If the body loses more temperature (e.g., 33 to 34 degrees Celsius), the body may vasodilate and restore full blood flow to the extremities, which may increase the core temp loss rate. The hypothermic thresholds may be around 35 degrees Celsius. When the body falls below the hypothermic limit, other physiologic processes may begin to trip sequentially. The patient heating system may operate in an open loop manner (e.g., set by the surgeon) or a closed loop manner which may change by request or change automatically (e.g., after the body temperature falls below a threshold or the difference exceeds a limit). The patient heating system may use extremity temperature or blood flow as a basis to preemptively adjust heating so that the heating system does not fall too far behind the body's decrease in temperature. If the patient heating system heats the body too rapidly after falling behind the body's decrease in temperature it may cause a rapid heating physiologic issue. In examples, ΔT_{c} may represent the temperature drop of the core. ΔTₑ may represent the temperature drop of the extremities. ΔT_{c} or ΔTₑ may be used as an open-closed loop control of the heating.

Feature(s) associated with instrument usage for multiple independent controlled robots and arms with no-fly zones or cooperative use zones (e.g., within the abdomen working space and/or space above the body) are provided herein.

In examples, multiple laparoscopic multi-cart robots may be used for a procedure. A first laparoscopic multi-cart robot may be used for dissection and resection of a mid-parenchyma tumor that is on the junction of two segments. The surgeon may attempt to separate out the tumor from the artery and vein (e.g., to refrain from removal of two full segments). The tumor may invade the bronchus, which may not be appreciated until the surgery is underway. The surgeon may incorporate a second robot in the procedure to determine penetration depth and the extent of invasion of a flexible endoscopy scope. The introduction of the second robot may not require repositioning of the existing first robot cart, but one of the carts (e.g., positioned towards the head of the patient) may have a working envelope outside of the body that encompasses some of the space now occupied by the second robot and/or the second robot's required operating envelope. The second robot (e.g., when operational) may establish communication with the first robot and may identify its location and size dimensions. The second robot may define the minimum space it requires to operate and inform the first robot of the reduced operational envelope within which the first robot may operate the conflicting arm to avoid entanglement.

Regulation of the first robot by the second robot may include determining the space reduction and/or active monitoring of the first robot arm. The restriction may be as limited as defining a portion of the full operating envelope the first robot may no longer operate in (e.g., while the second robot is present). The restriction may include actively regulating (e.g., adjusting) the space of operation. The space of operation may change as the two robot arms coordinate their operation. The second robot may reduce the operating space when/as it is required (e.g., to move through its configurations) while allowing the first robot to occupy shared space as long as the second robot does not require use of that space. When both robots need to occupy the same shared space, the two robots may negotiate based on priority, user input, and/or computational ordering that would allow the motions to be choreographed without adverse interactions (e.g., a set of motions that may allow the robots to move around each other, such as by a series of pre-calculated alternating motions).

The location of smart systems relative to each other may be identified. For example, a user may input the location and/or operational window for operation of a robot (e.g., a flexible endoscopy robot). For example, the smart system may define its location and operational envelope or the location and operational envelope of two robots (e.g., relative to each other). The location and operational envelopes of robot(s) may be defined when a robot (e.g., each robot) is brought into an operating room and/or a setup for a procedure.

A surgical hub and/or a room-based camera may be used to identify the exact location, shape, and operational window based on a setup (e.g., arrangement and purpose) of devices (e.g., robot towers, robot carts, robot arms, and smart systems). This identification may be accomplished using multiple perspective cameras (e.g., with overlapping image coverage) or may be accomplished (e.g., with less cameras) with the use of Lidar as a means to detect distances. Structured light may be used to define shapes and volumes. Examples of operational window detection and perspective imaging are described in U.S. Patent Application Publication No. 2023/0116781 (U.S. patent application Ser. No. 17/493,909), titled SURGICAL DEVICES, SYSTEMS, AND METHODS USING MULTI-SOURCE IMAGING, filed Oct. 5, 2021; U.S. Patent Application Publication No. 2023/0102358 (U.S. patent application Ser. No. 17/493,919), titled SURGICAL DEVICES, SYSTEMS, AND METHODS USING FIDUCIAL IDENTIFICATION AND TRACKING, filed Oct. 5, 2021; and U.S. Pat. No. 10,413,373 (U.S. patent application Ser. No. 15/237,902), titled ROBOTIC VISUALIZATION AND COLLISION AVOIDANCE, filed Aug. 16, 2016, the disclosures of which are incorporated herein by reference in their entirety.

Robot systems (e.g., robot towers) may integrate laser alignment and Lidar positioning to define the location of arms, carts, and control systems. For example, a robot system may use laser alignment and positioning systems to know where its arms are arrayed around the patient. A flexible endoscopy robot may be brought into and positioned in the operating space. The robot system may use its alignment system in combination with any data the flexible endoscopy system might provide to identify the flexible endoscopy system's location within the room and around the patient (e.g., in addition to its own location).

Physical docking locations or mechanical linkages may be used to place the movable robot carts and towers in known predefined locations (e.g., relative to stationary larger robot systems). For example, a table-based robot may have a docking location with physical aligning and locking systems that enables a mobile flexible endoscopic robot to be placed in a location where the location of the tower (e.g., relative to the mobile flexible endoscopic robot) is known.

Various examples of robotic systems and surgical tools that are suitable for use with spatial awareness techniques are described in U.S. Pat. No. 11,678,881 (U.S. patent application Ser. No. 15/940,666), titled SPATIAL AWARENESS OF SURGICAL HUBS IN OPERATING ROOMS, filed Mar. 29, 2018, the disclosure of which is incorporated herein by reference in its entirety.

The working envelope of the arms, instruments, and end-effectors of each of the smart systems may be identified. The overlapping space(s) of the operational envelope as shared space may be identified. The manner of organizing utilization of the shared space by each of the systems may be determined. Individualized reactive isolated step operation may be used to coordinate robot movement. In examples, individualized reactive isolated step operation may be used to assess the robot(s) arrangement after each step. For example, the system may analyze one step at a time rather than plan and/or choreograph multiple moves at once. Subtractive operational envelope reduction may be used to reduce the operational space of a smart system. Subtractive operational envelope reduction may include disallowing a smart system from occupying space it may otherwise be capable of using. Subtractive operational envelope reduction may be based on the need and/or priority of another smart system (e.g., a robot tower) to use the shared envelope.

FIG. 17 depicts an example of operational envelopes of robots. The operational envelopes of a first robot 54144 and a second robot 54146 may overlap at zone 54148. Robot 54144's operational envelope has been reduced by the overlap with robot 54146. In examples, operational envelopes of robots may be reduced, based on the adjacent robot having a higher priority of the space. For example, in FIG. 17 robot 54144 may have lower priority than robot 54146, and the operational envelope of robot 1 has been reduced.

A reduction of a robot's operational envelope may be temporary and may be reversed or reallocated as “shared” space in real-time. Each overlap of operational envelopes may be partially owned by a system. Operation in the shared space may be choreographed over time (e.g., when one robot moves through and leaves a shared space, the other robot may move through the shared space). Shared motions (e.g., choreographed movement between robots) may be performed stepwise (e.g., with each robot making a move in order to better utilize the shared spaces).

FIG. 18 depicts an example thoracic procedure with five robots arrayed around a patient. A thoracic procedure may involve flexible endoscopy robot 54150 and four robots 54152, 54154, 54156, 54158. At least one robot may access the space over the patient's head. FIG. 18 depicts the operational envelopes for each robot and the zones of overlap that the robot may manage in-system. An overlap zone may be present in a shared space where multiple (e.g., three or more) robots' operational envelopes intersect. A robot may designate a first portion of its operational envelop as space in which it must operate (e.g., extension and retraction space) and a second portion of its operational envelope as a shared space that it or another robot may use (e.g., one at a time).

Robots around a patient may be repositioned to allow for a robot (e.g., flexible endoscopy robot 1000) to be positioned by a portion of the patient's body. For example, a flexible endoscopy scope may be introduced in the mouth.

FIG. 18 depicts several “shared” spaces, where the robot arms may both operate (e.g., sequentially, not simultaneously) shown as stripped. FIG. 18 depicts a “reserved” zone is shown as dotted (e.g., a zone representing a portion of the flexible endoscopy robot's operational envelope that it may not share). The reserved zone may be reserved for the flexible endoscopy robot (e.g., as it must access the patient's mouth), and the flexible endoscopy robot may not move out of the way for the adjacent robot station to share the space during operation. Adjacent robots may avoid operating in the reserved zone. The shared spaces (e.g., where operational envelopes of two arms of a robot may overlap and collide) may be accessed by adjacent robots (e.g., if the robot controller can limit one robot arm at a time in the spaces).

Instrument inter-connectivity for operation room (OR) usage and battery utilization, needs, charging timing, and choreographing impacts on workflow may be performed. Smart chargers may be used for battery powered stapling. Smart chargers may communicate status of the battery. Smart chargers may receive anticipated demand (e.g., energy demand) between one or more of the smart charger, the smart battery, the smart stapler, and the smart surgical hub. Smarts systems may operate out of sequence with the procedure itself (e.g., ensuring that the devices are fully capable of performing the in-surgery operation).

Charger detection of battery recharge capacity, status of current charge level, and/or completion timing may be communicated to a smart hub (e.g., from a smart charger) and may be used to define procedure start times and/or operating room throughput.

The smart hub may communicate data (e.g., regarding utilization rate and timing data) to the smart charger. Utilization rate and/or timing data may be used by the smart charger to perform one or more of: toggling trickle charging (e.g., that may be better for battery capacity) or determining when to finish charging (e.g., that may improve battery health longevity). For example, the smart charger may maintain a battery at a partial charge (e.g., 80% capacity) in maintenance mode and may complete the charge (e.g., 100% capacity) close to an anticipated start time for a procedure.

The smart charger may determine or receive scheduling for a series of procedures that may require powered devices (e.g., powered stapler(s) or ultrasonic hand-held device(s)). The smart charger may accelerate the charging or may sequence the charging of different batteries connected to it based on received or determined usage requirements (e.g., such that that a fully charged battery may be ready at the time each procedure is needing to be prepared for). The smart charger may review the usage cycle of the batteries and charge the battery most appropriate for use. Appropriateness for use may be determined based on balancing the usage between each of the batteries or intentionally depleting one battery to allow time for a replacement battery to be acquired (e.g., without other batteries requiring replacement simultaneously).

The smart battery charger for the powered staple may have two batteries on the charge (e.g., at 25% & 55% of charge respectively). The hospital hub may inform the charge of the number of upcoming procedures (e.g., based on the expected number of firings and the timing of the procedures). The smart charger may inform a smart hub when it can have a battery ready. The smart charger may inform the hub whether the smart charger may have to move into fast charge mode for a battery (e.g., each battery) to be ready for the second procedure. The smart charger may inform a smart hub that the first battery has sufficient charge for two procedures (e.g., a first procedure and a third procedure) which do not overlap in timing.

The smart charger may communicate with the smart hub regarding procedure plans, timing, and/or cadence. The smart charger may communicate with smart hand-held device(s) in use, for example, to receive real-time updates on battery levels and power consumption. The smart charger may adjust charging rates and timing of charging batteries to balance the need for sufficiently charged batteries with the desire to charge slowly (e.g., to increase the overall life of the battery). For example, if a procedure uses more or less power than predicted, charging rates and charging timing may be adjusted by the smart charger for other batteries.

FIG. 19 depicts an example of changing battery levels of smart devices over time. Device 1 (e.g., a powered stapler) may require about 50% of its charge for six uses (e.g., 60 mm thick tissue firings, as in a gastric sleeve). Device 2 may be ready for procedure 1 with around 75% charge. Device 2 may not require fast charging to be ready for a second use (e.g., in procedure 3) because device 2 was used for three firings in procedure 1. Device 2 may not require any charging for the three procedures.

Real-time updates from a smart device may be provided by any non-wired battery powered smart device (e.g., a smart stapling device). Real-time updates may include indications of higher or lower than anticipated battery use. A smart charger may adjust the plan for cleaning and charging of devices based on real-time updates (e.g., to support all usage needs). Real-time updates may be used by the smart charger to monitor the rate and/or magnitude at which a battery is discharged, which may allow the smart charger to adjust its conditioning during recharging (e.g., to minimize the effects on the battery chemistry for heavy use procedures). Adjustments to conditioning may be a slow charge, a partial charge that may be held for a period of time, and/or a small incremental pining on/off charging to induce the chemistry within the battery to re-balance (e.g., after being heavy used or fully discharged). Adjustments to conditioning may reduce the effects on the electrolyte mix and/or may prevent reverse corrosion or damage to the electrodes.

Localized organ cooling for protective temperature control may be performed (e.g., selectively for heart ablative resurfacing). For example, catheter ablation may be performed to treat heart atrial fibrillation (Afib). Afib may be an irregular heart rhythm that begins in the heart's upper chambers (atria). Afib may be caused by extremely fast and irregular beats from the upper chambers of the heart (e.g., more than 400 beats per minute). A normal, healthy heartbeat may involve a regular contraction of the heart muscle.

Persistent Afib may last more than a week and may require treatment. Long-standing persistent Afib may last more than a year and may be difficult to treat. Symptoms of Afib may include one or more of: extreme fatigue, an irregular heartbeat, heart palpitations, an unsettled feeling in the chest, dizziness, lightheadedness, fainting (e.g., syncope), shortness of breath (e.g., dyspnea), or chest pain (e.g., angina).

Synchronization and positioning of a RF ablation application while the heart is beating may be critical to the proper localization of treatment and prevention of secondary complications.

An HCP may send or measure the small electrical impulse through an electrode catheter to locate the abnormal tissue causing arrhythmia. A full heart map or a localized activation of the abnormal tissue that is causing the arrhythmia may be used to locate abnormal sites. Other catheters may record the heart's electrical signals to locate the abnormal sites.

Maps (e.g., heart maps) may be acquired during stable sinus rhythm (SR). The left atrial-pulmonary vein (PV) junction may be angiographically defined by hand injection (e.g., of 5 to 10 mL) of contrast medium in the anteroposterior view. Three or more of the following anatomic and electrophysiological characteristics may be used to define the ostium: the fluoroscopic position of the catheter tip corresponding to the angiographically defined ostium; the point where the catheter tip dropped into the chamber during pullback; the appearance of an atrial potential in the case of a silent PV segment; or an impedance decrease.

Localized ablation (e.g., focal ablation) of the tissue of the heart map be performed in an area of high movement of the heart. The movement of the heart may change the target ablation location. Proper treatment of the atrial fibrillation (Afib) (e.g., via catheter ablation) may require the direct destruction of the heart conduction pathway causing the irregular heart contraction without overly damaging the collateral heart muscle and structures.

The efficacy of catheter ablation may depend on accurate identification of the site of origin of the arrhythmia. An ablation catheter (e.g., 7 French in size with a tip electrode size of 4 mm) may be positioned in direct contact with the site or origin and radiofrequency energy may be delivered to ablate the site. After a period of time (e.g., 30-60 second) a lesion (e.g., of 5-mm depth) may be formed, which may be enough to destroy the full thickness of the atrial myocardium in that location.

The size of the lesion created may be determined (e.g., may be based on) a balance between conduction of heat from the radiofrequency electrode on the end of the ablation catheter through to the tissue, and convective heat loss to the blood pool. The lesion size may be proportional to the delivered power, the diameter of the distal ablation catheter electrode, and/or the contact pressure of the distal ablation catheter electrode with the cardiac tissue.

The catheter may be placed at the exact site of abnormal cells inside the heart. Mild radiofrequency (RF) energy may be delivered to the tissue. The RF energy may destroy heart muscle cells in an area (e.g., about ⅕ of an inch) that are responsible for the extra impulses causing rapid heartbeats.

Sweeping ablation (e.g., ablation remodeling) of the heart over a wider area where the heart conduction systems may be causing non-synchronized contraction may be performed. Sweeping ablation over a wider area may include moving the ablation tip over a predefined area to create a diffused reduction of the irregular or uncoordinated contractions. This active ablation activity while moving may require precise control force and location prediction while balancing time-on-target with power levels (e.g., to achieve the penetrative effects desired).

Size of distal electrode and saline cooling of the electrode may minimize impedance increases. Controlled pressure application and power level may allow for creation of larger and/or deeper lesions and may provide better control of the lesion.

The electrode-tissue interface may be greater than or equal to 50 degrees Celsius to cause tissue necrosis. At temperatures around 100 degrees Celsius and above a coagulum of denatured proteins and plasma may form on the catheter tip and may impede the delivery of current. The formation of coagulum may increase the risk of thromboembolic complications.

RF ablation in stable sinus rhythm (SR) may be performed. RF pulses may be delivered with an ablation catheter (e.g., with a temperature setting that may be up to 55 degrees Celsius and RF energy that may be up to 50 W for an 8-mm-tip ablation catheter). RF pulses may be delivered with an irrigated-tip catheter (e.g., with a temperature setting that may be up to 43 degrees Celsius and RF energy that may be up to 35 W). RF pulses may be applied until local electrogram amplitude is reduced (e.g., greater than or equal to 80%) or decreased below a threshold for a period (e.g., 0.1 mV for up to 120 seconds).

Control of the rate of saline deposition, the temperature of the saline, and/or the volume of the saline (e.g., thermal mass) may be used to control the local cooling effects adjacent to the intended ablation location.

Cauterization magnitude, depth, and collateral damage may be balanced by interconnected and/or interactive control of one or more of: power level, saline heat mitigation, electrode contact zone, or pressure (e.g., energy density).

FIG. 20 illustrates an integrated surgical environment and depicts interconnectivity of legacy and smart systems within a medical procedure.

The surgeon 20020 may interface with various items during the surgical procedure. The surgeon 20020 may be referred to herein as the medical practitioner. The surgeon 20020 may interface directly with the smart device 54200 and may suggest an interactive system where the surgeon may input surgical parameters or procedural protocols. The smart device 54200 may provide real-time feedback or surgical guidance based on the data it (e.g., the smart device) processes.

The smart device 54200 may have a bidirectional communication channel with data center 54204. A (e.g., synchronous) data exchange may exist in the smart device 54200, wherein the smart device 54200 may transmit procedural metrics or receive updates based on system algorithms or surgical guidelines stored within the data center 54204.

Regarding the smart device's adaptability, the smart device 54200 may interface with a legacy device 54206 through a bidirectional link. The connection between the smart device 54200 and the legacy device 54206 may indicate that the smart device 54200 may integrate with and/or adapt to surgical equipment within the operating room. The smart device 54200 and the legacy device 54206 may be compatibility and expand utility across a broad range of medical settings.

The data center 54204 may not be a passive data repository. With a bidirectional link to the surgical systems data set 54208, the data center 54204 may query the dataset for relevant surgical protocols and updating the surgical systems data set 54208 with data (e.g., to refine procedural algorithms or record surgical outcomes).

The smart device display 54210 may be (e.g., directly) connected to the data center 54204 through a bidirectional channel and act as a visual interface for the smart device 54200. The smart device display 54210 may (e.g., dynamically) present data-driven insights, procedural guidance, or real-time metrics derived from the data center 54204. Direct connectivity to the Legacy device 54206 may enable the display to visually represent metrics or statuses from surgical systems. Legacy equipment (e.g., non-smart equipment) may be integrated into a feedback loop as described herein.

The surgeon's bidirectional communication with the OR bed may indicate a dynamic interface where the surgeon may adjust patient positioning with the potential to receive direct feedback on patient vitals or procedural progress, thereby modifying (or adapting to) the surgical process.

FIG. 21 illustrates an architecture diagram outlining the configuration of a surgical smart system operating within a HIPAA-protected operating room. The architecture may facilitate surgical procedures while upholding patient data privacy regulations.

The Input/Output (I/O) device 54214 may function as an intermediary that establishes communication between the surgical team and the smart system. Equipped with controls, dynamic displays, and interactive interfaces, the I/O device 54214 may enable real-time communication, allowing surgical personnel to issue commands, receive feedback, and access data during surgical procedures.

Embedded within the smart system device, a processor 54216 may operate as a computational hub, enabling task management, data processing, and system coordination. The processor 54216 may adeptly manage incoming data streams, execute algorithms, and synchronize component interactions, such that task execution and responsiveness may not be compromised.

The memory and storage 54218 within the device may serve as repositories for essential data, software applications, and surgical records. The memory and storage 54218 may house a device look-up table 54220, which may include information about surgical equipment and interaction configurations that are compatible with existing surgical equipment. The memory and storage 54218 may retain historical data, usage patterns, and operational logs for informed decision-making, predictive analytics, and continuous process modification.

Within the memory/storage device 54218, the device look-up table 54220 may compile an exhaustive repository of available surgical equipment, encompassing information such as specifications, compatibility criteria, usage guidelines, and interaction preferences of devices (e.g., legacy devices). The repository may be used for equipment selection and interaction customization, enabling precision and modification of (and for) surgical procedures.

The device capabilities 54222 (e.g., complementary to the device look-up table) may offer insights into the functionalities of surgical instruments. The device capabilities 54222 may be used for tailoring interaction levels in alignment with surgical requirements and modifying procedural efficiency by enabling equipment to be employed in the most suitable manner (e.g., as suggested by the smart surgical device 54200.

The data center 54204 may serve as a hub for data processing, storage, and analytics. The data center may host an expansive database including aggregated operational data, patient records, and procedural insights. Analytics, machine learning algorithms, and predictive modeling may be used to generate actionable insights for process refinement.

The network 54224 may be interconnected with the architecture components to facilitate data exchange and communication. Th network may enable the transmission of operational data, alerts, and performance updates across the smart system device, the data center, and interconnected systems, supporting remote monitoring, collaboration, and informed decision-making during the surgical procedure.

In order to address data security and patient privacy concerns, the architecture may incorporate a HIPAA-protected operating room smart system 54226, adhering to regulatory healthcare standards. This system 54226 may enable the confidentiality of patient data while enabling surgical procedures.

Mirroring the operating room system's security, the data center may be HIPAA-protected 54228, assuring confidentiality and compliance of patient data stored within repositories of the data center. The dual layer of protection may uphold secure management of patient records and operational data throughout use of the data center 54228.

In smart systems, including in medical applications, the capability for autonomous operation may be described herein. The systems may achieve performance through strategic interaction. The interaction may be governed by a multifaceted decision-making process, which may be contingent upon questions: Can the systems interact? Should the systems interact? Is it beneficial for both or just one of the systems to engage in the interaction? Would the integration compromise the safe operation of the systems?

The circumstances that define how two smart systems interact are based on factors intrinsic to the systems themselves—can they or should they cooperate at a specific level? The factors may be delineated by the information a system uses to operate in a closed-loop format and whether both systems include closed-loop operation for optimal functionality. The interrelation between the systems may be governed by constraints based on the nature of the data exchanged, the interrelationship of the variables involved, or the relative importance of one system over the other as determined by an operator, such as a surgeon.

Examples may include an algorithm designed to manage an instability cascade failure. Examples may include a patient monitoring smart system, which may keep track of oxygen levels, carbon dioxide levels, and temperature. The vital(s) parameters may be shared with both the patient heating system and the ventilation/sedation systems. The ventilation/sedation system may use the patient's temperature and gas levels to adjust the sedation medication rate, tidal volume, and oxygen supplementation. The heating system may utilize the temperature data to modulate thermal load input. The interplay of the systems may include the patient's metabolism—affected by factors like sedation uptake and oxygen consumption—which may be temperature-dependent. A loop of interaction may be included, where the patient heating system, responding to temperature data, may indirectly influence the patient's metabolism. The modification in metabolism may impact the ventilator's function, which may attempt to maintain a stable sedation level. The modification may lead to an oscillating and unstable dynamic in both systems, which may be associated with a shift from cooperative interaction to a bi-directional open-loop communication mode.

Examples of system interaction may include a non-correlated predictability failure. A flexible endoscopic robot (e.g., a Type D smart device), may be used for the (e.g., precise) placement and control of RF needles during lung tumor ablation. The process may be monitored by an (e.g., advanced) visualization system, which may track the local external lung parenchyma temperature. Examples may include inhomogeneity in the density and conductivity of both the tumor and the parenchyma. The tumor's inhomogeneity may stem from its internal growth patterns, and the parenchyma's variation may be attributed to factors such as adhesion and chronic tissue remodeling. The disparity may lead to a misinterpretation by the laparoscopic camera, which may underestimate the heat penetration during ablation due to dense adhesions, to (e.g., to then) detect a sudden increase in temperature. The increase may not be due to a change in the ablative needles' activity and (e.g., and rather) a shift in the parenchyma's density. Examples may include a comparison between the energy input rate and the rate of heat bloom expansion. If the energy input rate and the rate of heat bloom expansion variables are found to be uncorrelated to a significant degree, the system may transition from relying on global temperature control data to a local bi-directional data exchange focused on the specific measurements.

The decision to maintain or alter the operational state of these systems may be based on circumstances surrounding the patient or a procedural occurrence. The determination may be triggered when a parameter related to either the patient or the operational device, based on a tissue parameter, deviates outside a predetermined acceptable range (e.g., a threshold). Parameters that may trigger such a shift in operational state may include heart rate, heart rate regularity, variability in heart rate, levels of oxygen or carbon dioxide in the blood, blood pressure, changes in correlations between two measurements of essentially the same patient variable (e.g., core temperature compared to extremity temperature, local temperature versus visualization temperature, etc.), tidal volume, pressures during inhalation or exhalation, blood sugar levels, and various inflammation indicators like Erythrocyte Sedimentation Rate (ESR), C-reactive protein (CRP), Plasma Viscosity (PV), and/or heart damage indicators including Cardiac Troponin, Creatinine Kinase (CK), CK-MB, and/or Myoglobin.

FIG. 22 illustrates a system diagram of an example configuration of an operating room smart system 54200 designed to interface, for example, with legacy devices. The capabilities of legacy surgical instruments may be bridged with smart systems.

The surgical systems data set 54208 may be a repository for data related to surgical protocols, equipment configurations, patient information, and more. Through the repository, a smart system may access data to inform its operations across multiple surgical phases.

In examples, alongside the surgical systems data set may be the surgical/surgical assistance action 54230. The surgical/surgical assistance action 54230 may guide or direct actions within the surgical environment. The surgical/surgical assistance action 54230 may operate by interfacing with the data set, extracting information, and providing instructions or support based on that data to either surgical instruments or medical personnel during procedures.

In examples, running parallel to the surgical/surgical assistance action may be the surgical platform 54238. The platform may oversee the integrated system, enabling coordination and operation of individual components and modules.

Three distinct modules are depicted nestled between the Surgical/Surgical Assistance Action 54230 and the Surgical Platform 54238 as follows.

The pre-operative module 54232 may facilitate preparatory activities leading up to the surgical procedure. Functions may include planning, device calibration, and patient preparation.

The intra-operative module 54234 may conduct actions facilitating the surgical procedure. The actions may include the guidance of surgical maneuvers, equipment monitoring, real-time decision-making support, etc.

The post-operative module 54236 may facilitate actions after the surgical procedure. The post-operative module 54236 may handle activities related to post-surgery care. The post-operative module 54236 may oversee patient monitoring, data storage, equipment cleanup, and analysis.

The Network 54224 may facilitate communication and data transfer among components. The network 54224 may connect the operating room smart system 54200 with the data center 54204, enabling an exchange of information. Furthermore, the connections to legacy devices, denoted as legacy device A (54206), B (54206), and C (54206), may allow the smart system to integrate the functionalities of the older devices into the current surgical workflow. The integration may confirm that no existing capability is unused or overlooked in the updated system (e.g., the system after recognizing legacy devices).

FIG. 23 illustrates an example database decision-making process within pre-operative, intra-operative, and post-operative modules.

The pre-operative module 54232 may have cooperative interactions with various integral components for preparing the surgical environment and enabling surgical planning. The pre-operative module 54232 may communicate with components like room scanning at 54240, aiding in capturing spatial details of the surgical environment, for example, in real-time. The interactions may involve extracting high-resolution images, determining spatial positions of surgical equipment, and identifying legacy systems present within the environment.

At 54242, a compatibility determination may be performed, for example, within the pre-operative module 54232. The compatibility determination may be programmed to interface with surgical devices having compatibility specifications, functional capabilities, and operating parameters. The compatibility determination may deduce effective communication strategies (e.g., for devices categorized as legacy), minimizing potential operational conflicts during surgical procedures.

At 54244, to enable legacy devices to be integrated with the smart system, a legacy connection may be initiated with communication protocols tailored for the devices (e.g., between the smart device and the legacy device). The legacy connection may (e.g., dynamically) determine the data transmission protocol, initiate handshake processes with devices, and communicate using software drivers designed for the specific legacy devices.

The pre-operative preparations may be orchestrated by the device interoperation plan at 54246, which may be tailored based on prior compatibility checks and environmental scanning. The interoperation plan while nested within the pre-operative module, may produce outputs, as indicated by 54248, may be relevant in surgical modules.

In the intra-operative module at 54234, the device interoperation plan, at 54248, may serve as a guide, for example, offering real-time command sequences. The relationship with the surgical actions engine 54242 may indicate a continuous feedback mechanism. The surgical actions engine 54242 may integrate decision-making algorithms, real-time data processing, and sensor-driven insights to interface between surgical phases ranging from surgery phase 1 to surgery phase x.

At 54250, manual engagement within the intra-operative module 54234 may allow surgical professionals to exercise discretion. Surgeons, through this component, may have the ability to modify or override the pre-determined operation plan, addressing real-time surgical nuances and unforeseen challenges that may arise during the surgical operation.

The system may consolidate surgical data at 54258. The data may capture details, from device interactions, surgical maneuvers, patient responses, to ambient conditions during the procedure, offering a data-driven perspective of the entire surgical event.

The post-operative module 54258 may use data analysis and insight extraction. Surgical actions analysis and reporting, at 54260, may employ computational models, comparative analytical techniques, and machine learning algorithms to interpret and process the data from the surgery data 54258. By interfacing with the surgical systems data set 54208, the post-operative module may derive actionable insights, retrospective evaluations, and predictive markers, adjusting/modifying surgical procedures in future surgical procedures.

FIG. 24 illustrates an example of the surgical systems data set 54208. Within this data set, there may be the smart surgical module 54262. The smart surgical module 54262 may serve as an integrated component that houses and manages subsets of surgical data and related parameters.

The surgical manifest 54264 may function as a catalog or repository, for example, containing (e.g., detailed) records associated with surgical tools, procedures, and methodologies. The manifest may facilitate data retrieval and analysis for surgical professionals, allowing the surgical professionals to access relevant instrument-related information. The manifest may also be used to help detect legacy devices.

The legacy device ID 54266 may offer an identification mechanism for a legacy device, allowing for tracking and management of the legacy device. By equipping a legacy device with a unique identifier, interactions and data retrieval related to the device within a surgical or clinical environment may be streamlined.

The legacy device parameters 54268 may be a part of the smart surgical module 54262. The parameters may encompass a range of technical attributes associated with the legacy devices. The parameters may detail operational aspects such as device dimensions, functional thresholds, and other technical characteristics. By centralizing the parameters, the smart surgical module 54262 may provide an overview of a device's technical capabilities.

The legacy device input parameters 54270 may specify the nature and format of data inputs that a legacy device is configured to receive. Understanding the input parameters may enable data to be provided to the legacy device such that the data provided aligns with operational parameters of the legacy device.

The smart surgical module 54262 may include the legacy device coding parameters 54272. The parameters may relate to the coding or programming instructions tailored for a legacy device. By delineating the coding requirements and associated algorithms, the parameters may offer insight into the device's computational operations and its integration potential with other systems.

The smart surgical module 54262 may include the legacy device output parameters 54274. The legacy device output parameters may detail the nature, format, and structure of data outputs generated by the legacy device post-operation. Interpreting the output parameters may done by the interfacing system, enabling the output data or feedback from the device to be processed (e.g., accurately) in surgical stages.

FIG. 25 illustrates an example configuration wherein the operating room smart system 54232 interfaces with interdependent components. FIG. 25 illustrates the system's capability to interact and interface with diverse devices, specifically legacy devices, and harness their functionalities.

The operating room smart system 54232, as depicted, may be in direct or indirect communication with the surgical systems data set 54208. Within this dataset may reside the smart surgical module 54262, potentially acting as an information repository. The smart surgical module 54262 may store an array of surgical metadata, encompassing specifics related to surgical instruments, protocols, and for example, legacy devices.

The operating room smart system 54232 may interface with a camera 54276. The camera 54276 may be embedded with optical and analytical capabilities and may monitor the surgical room's spatial area. The camera 54276 may detect, analyze, and categorize the features and functionalities of legacy devices 54206 within its view.

Through integration of (e.g., advanced) algorithms, the camera 54276 may discern the presence of legacy devices 54206 and their functionalities. In examples, upon inspection of a legacy device 54206, the camera 54276 may detect the possibility of keyboard input at 54278. The detection may be facilitated through pattern recognition algorithms or visual markers that are identifiable on the legacy devices by the camera 54276.

The operating room smart system 54232 may autonomously, or upon command, generate prompts or directives for the surgical staff. The directives may include actions that bridge the communication gap between the detected legacy device and the operating room smart system 54232 (e.g., instructing the staff to connect, via a cable/wireless communication medium, the operating room smart system 54232 to the legacy device 54206). The communication channel may enable data transfer, command execution, or remote manipulation of the legacy device.

Recognizing the keyboard input capabilities of the detected legacy devices 54206 may permit the operating room smart system 54232 to introduce or modify interactive mechanisms. In examples wherein manual input is possible, the system may use the legacy device's keyboard input, thus facilitating command sequences or data input operations.

The operating room smart system 54232 may interface with systems or sub-systems not illustrated in FIG. 25. In examples, the smart system may communicate with remote servers or data centers to harness computational power or storage capabilities, such as cloud computing. The interactions may be used when processing data streams in real-time or when synchronizing data across multiple surgical platforms.

The operating room smart system 54232 may support diverse communication protocols. Whether via wired connections or wireless transmission, including GSM, LTE, Bluetooth, WiFi, the system may maintain communication with peripheral devices or remote systems.

FIG. 26 illustrates a configuration emphasizing the data acquisition and display capabilities of the operating room smart system 54232 in relation to legacy devices. The architectural framework may demonstrate visualization and information synthesis, contributing to informed surgical processes.

FIG. 26 illustrates operating room smart system 54232, which may have an (e.g., dynamic) association with the surgical systems data set 54208. Included with the data set may be the smart surgical module 54262, a repository that may store, process, and manage surgical details and parameters. The module may enable integration with surgical tools, protocols, and specificities pertinent to legacy devices.

The camera 54276, may be linked to the operating room smart system 54232. The camera 54276 may be equipped with optical sensors and computational algorithms enabling the camera to scan the surgical room environment. The camera 54276 may interpret and discern characteristics of legacy devices 54206 present in its field of view.

The camera 54276 may recognize and analyze data visualizations on the legacy devices 54206. Through a combination of pattern recognition, optical character recognition (OCR), and/or machine learning models, etc., the camera may ascertain if a legacy device is actively presenting data relevant to the ongoing surgical procedure. The data may include vital metrics, graphical representations, or real-time feedback, etc.

When the relevant data is identified on the legacy device, the operating room smart system 54232 may conduct actions to access the relevant data. The smart system may capture, process, and/or project the data onto the smart system display 54210. The display, which may include high-resolution graphics (e.g., higher resolution than the legacy device) and customizable interface options, may present the data in a more digestible, interactive, and/or relevant format (e.g., than the legacy device), aiding surgical professionals in real-time decision-making.

The operating room smart system 54232 may employ multiple channels or protocols to enable data fidelity and communication. From data acquisition to visualization, error-checking, validation, and security measures may be in place to enable data integrity and relevance to the surgical context.

FIG. 27 illustrates an example block diagram of a surgical smart system. The surgical smart system, when implemented in an operating room, may modify the interaction between surgical staff and equipment. At 54280, the system may receive data associated with a surgical procedure.

At 54282, during the course of the procedure, the system may identify a piece of equipment in use. The identification may be based on direct input from the surgical team, sensor data, etc. When the equipment is identified, at 54284, the system may receive interaction levels for the piece of equipment. The interaction levels may be categorized as minimal, intermittent, or full interaction levels.

The surgical smart system's algorithms may process the interaction levels in the context of the provided surgical data. In examples, by cross-referencing the identified equipment with the surgical manifest, the system may determine the best use case scenario for the equipment in the ongoing procedure.

At 54286, the interaction levels associated with a piece of equipment may not be static. The system may provide flexibility, allowing for the selection of an interaction level from the interaction levels based on the selected interaction level being associated with a surgical preference. The dynamic adjustment may enable positive equipment functionality and surgical outcomes.

The system's interface may display the interaction levels for the surgical staff's reference. The distinct visualization of the interaction level may correlate with the best use case of the identified equipment. The visualization may aid in quick decision-making, allowing surgical staff to select the most appropriate interaction level from the display directly.

As the surgical procedure progresses, the surgical smart system may (e.g., continuously) monitor the operation of the identified equipment. The surgical smart system may gather operational data, which may include metrics like operational time, power consumption, performance metrics, etc.

The continuous monitoring may extend beyond data collection. The system may actively analyze the operational data to detect deviations or performance changes in the equipment. The continuous monitoring may enable (e.g., immediate) intervention, should an equipment malfunction or not operate as intended (or in a manner not beneficial for the patient).

Modules within the system may process the operational data to deduce the impact of the equipment's operation on patient physiological parameters. If a performance change is detected, the system may modify the interaction level (e.g., dynamically), enabling patient safety and positive procedure outcomes.

Predictive analytics may be used by the system. By scrutinizing the usage patterns of the identified equipment, the system may forecast the future performance of the equipment. The predictions may enable preemptive measures, minimizing surgical procedure interruptions.

For record-keeping and future reference, the system may display the analyzed operational data, which may include parameters like estimated time to failure for equipment. The data may not be confined to the system alone. The system may transmit the analyzed operational data to an external database (e.g., forming a comprehensive digital record of the equipment's operation during the surgical procedure).

In examples where a direct correlation between received data and predefined parameters is sought, the surgical smart system may employ a lookup table. The table may store pre-characterized information and, when queried with specific inputs, return the corresponding data. The mechanism may enable the retrieval of data sets or parameters (e.g., facilitating decision-making during procedures).

FIG. 28 illustrates a method for operating a surgical smart system in an operating room. At 54291, a database may be accessed. The database may include a surgical manifest and a lookup table of surgical equipment which may serve as a repository for storing and retrieving information regarding surgical assets and their respective specifications.

At 54292, a piece of equipment present in the operating room may be identified. Utilizing a camera of the surgical smart system, the identification of the piece of equipment may include comparing the piece of equipment with the information included in the database. The comparison may facilitate the recognition and verification of the equipment's presence and characteristics within the operating environment.

At 54294, data may be determined from the identified piece of equipment. The determination may be executed using Optical Character Recognition (OCR) (e.g., which may enable the extraction of text or numeral data from the images captured by the camera of the surgical smart system). The OCR may, for example, aid in recognizing identification numbers, labels, or data inscribed on the equipment.

At 54296, the data may be displayed on a display of the surgical smart system. The displayed data may include information extracted from the piece of equipment, which may provide the surgical personnel with details regarding the equipment's specifications, operational status, or other relevant data that may be associated with the surgical procedure.

A degree of interactivity between the surgical smart system and the piece of equipment may be quantified. The operation of the surgical smart system may be modified based on the quantified degree of interactivity. The quantification and modification may affect the utilization and coordination of the equipment associated with the surgical smart system.

The surgical manifest within the lookup table may be updated using the data from the identified piece of equipment, and the updated surgical manifest may be saved in the database. The operations may contribute to keeping the surgical manifest updated and accurate, reflecting the (e.g., most recent) data regarding the equipment present in the operating room.

The identified piece of equipment may be cross-referenced with the surgical manifest, and an alert may be generated on a condition that the piece of equipment is not found in the surgical manifest (e.g., serving as a safeguard, verifying that authorized or suitable equipment is utilized within the surgical procedure).

An operational status of the piece of equipment may be determined using the OCR-determined data, displaying the operational status on the display of the surgical smart system, and generating an alert on a condition that the operational status indicates that the identified piece of equipment is not suitable for use. The operations may affect the safety of the surgical procedures by indicating the readiness and appropriateness of the equipment in use.

The feasibility of wired communication with the piece of equipment may be determined, and instructions may be provided via a user interface of the surgical smart system for connecting the surgical smart system to the piece of equipment based on the determination that wired communication with the piece of equipment is possible. An operation of any of the surgical smart system or the piece of equipment may be adjusted based on the user connecting the surgical smart system to the piece of equipment.

FIG. 29 illustrates a logical representation of surgical systems and/or surgical imaging systems sharing control, information, and porting imaging, as described herein. Surgical systems may include laparoscopic robotic surgical systems, endoscopic robotic surgical systems, etc. that may be used for carrying out minimally invasive surgical procedures. The laparoscopic robotic surgical systems may include a smart robotic arm coupled surgical device, a multi-arm robot, The endoscopic robotic surgical systems may include a smart flexible endoscope, etc. The surgical imaging systems may include a magnetic resonance imaging (MRI) system, a computed tomography (CT) imaging system (e.g., a cone-beam CT scanner), a robotic bronchoscope.

As illustrated in FIG. 29, each of the surgical systems (surgical system 1 54302 and surgical system 2 54304) may be connected with respective user input/control units (54308 and 54314) and display units (54310 and 54316). In an example, the user input/control unit 54308 and display unit 54310 may be separate physical units. In an example, the user input/control unit 54308 and display unit 54310 may be a combined physical unit 54312, and the user input/control unit 54314 and display unit 54316 may be a combined physical unit 54318.

In an example, the functional user controls of surgical system 2 54304 may be displayed and interacted with on surgical system 1 54302. This function may enable an HCP controlling surgical system 1 54302 to request movements, activations, or operation of surgical system 2 without surgical system 2 surrendering internal operational control of the equipment to surgical system 1.

In FIG. 29, the sub-system or internal control of each of the surgical system 1 54302 and the surgical system 2 54304 may be operating in a default mode (e.g., in normal state). The surgical system 1 54302 may operate and control its system fully independent of any other surgical systems or other smart systems that may be present in its vicinity (e.g., in an operating room). The surgical system 1 may be controlled by a healthcare professional (HCP) by interacting with the user input/control unit 1 54308. Similarly, the surgical system 2 may also operate and control its system fully independent of all other surgical systems or other smart systems that may be present in its vicinity.

In an example, the surgical system 1 54302 may be allowed to request control of the surgical system 2 54304. For example, an HCP controlling the surgical system 1 54302 may be allowed as a user proxy to operate the surgical system 2. In such cases, the HCP operating the surgical system 1 54302 may establish a user proxy with the surgical system 2 54304. Once the HCP is established as a user proxy, the surgical system 1 54302 may generate command requests associated with the surgical system 2 54304. The command requests generated by the surgical system 1 54302 may be sent to the surgical system 2 54304. The command requests may include control information that may be used for controlling one or more aspects or functions associated with surgical system 2 54304. A command request generated and sent from surgical system 1 to surgical system 2 may include control information for controlling movements, activations, and/or operations associated with surgical system 2 54304. For example, a command request generated by surgical system 1 54302 may be sent to the surgical system 2 54304 for controlling user interface controller of surgical system 2 54304 (e.g., a device for entering data, a device for moving pointer on the user interface of surgical system 2 54304, a controller interface for controlling cameras, light sources, and other sensors associated with surgical system 2 54304, etc.)

After establishing the user proxy, the display unit 1 54310 associated with the surgical system 1 54302 may be modified in order to accommodate user display or a part of the user display from the surgical system 2 54304. In an example, an extended user display 54300 may be added to the display unit 1 54310 for displaying the information or a part of the information being displayed at display unit 2 54316 of the surgical system 2 54304. The information or a part of the information being displayed by display unit 2 may be exported and/or streamed from surgical system 2 54304 and displayed on the display unit 1 54310. In an example, imaging data may be ported from surgical system 2 and displayed on the display unit 1 54310 or the extended user display 54300 of surgical system 1 54302.

In another example, imaging data may be ported (e.g., streamed) from the surgical imaging system 54306 and processed and displayed on the display unit 1 54310 or the extended user display 54300 of surgical system 1 54302 and/or display unit 2 54316 of surgical system 2 54304.

FIG. 30 illustrates an example surgical operating room with robotic surgical systems or instruments and other surgical instruments that may be part of the surgical hub 54321. As illustrated in FIG. 30, a surgical procedure (e.g., a thoracic lung segmentectomy) may be performed on a patient using an endoscopic robotic surgical system 54322 controlled by one HCP and a laparoscopic robotic surgical systems (e.g., a smart robotic arm 54323 and/or a multi-arm robot 54324) that maybe controlled by another HCP using a controller 54325. In an exemplary setup, one or more of the systems shown in FIG. 30 may be configured for performing a surgical procedure. In such a setup, an HCP (e.g., the lead HCP) may control the laparoscopic robotic system 54324 and/or the single arm robotic surgical system 54323 using the console 54325. The endoscopic robotic system 54322 may be controlled by a second HCP (e.g., an assisting HCP), as illustrated in FIG. 30.

In examples, the lead HCP controlling the one or two laparoscopic robotic surgical systems may also send commands to the endoscopic robotic surgical system to controls some aspects of the endoscopic robotic surgical system. The lead HCP may control the endoscopic robotic surgical system either locally or remotely with or without the help of the second HCP 54329. In examples, the second HCP may be in vicinity of the patient.

FIG. 31 and FIG. 32 illustrate a stapler device (e.g., a linear or circular stapler) that may be controlled by an HCP together with the main laparoscopic robotic surgical system using a robotic surgical system console.

FIG. 31 illustrates an example where a robotic system with a linear stapler may be configured together with a robotic system with a circular stapler and a smart hub 54332 for performing a robotic colorectal surgical procedure. As illustrated in FIG. 31, the circular stapler may be attached to one of the robotic arms 54320 of a multi-arm robotic surgical system 54326.

In another example, as illustrated in FIG. 32, the circular stapler may be independent and not attached to one of the arms of the multi-arm robotic surgical system. As illustrated in FIG. 32, the circular stapler may be attached to an arm 54330 of a different robotic surgical system 54334 (e.g., a single arm robotic surgical system). The main robotic surgical system 54326 may communicate with the secondary robotic surgical system 54334 via a wired or wireless interface (e.g., via a Wi-Fi interface or a Bluetooth Low Energy (BLE) interface).

As illustrated in FIG. 31, where the circular stapler is attached to one of the arms 54320 of a multi-arm robotic system 54326, which is connected to the main console 54328. A lead HCP (e.g., the main surgeon heading the surgical procedure) may sit behind the main console 54328 to direct and control the robotic arms and operate the surgical instruments attached to the robotic arms of the multi-arm robotic system 54326. The lead HCP may operate the circular stapler with help of the second HCP 54329 who may be in proximity to the patient and the circular stapler itself.

In either of the setups illustrated in FIG. 31 and FIG. 32, the HCP operating the main multi-arm robot may begin the surgical procedure by laparoscopically dissecting the area surrounding the colon of the patient. The HCP using various surgical instruments attached to the laparoscopic robotic surgical system may transect the colon with a linear stapler or a circular stapler and remove the damaged areas, as described herein.

In case of FIG. 32, where the circular stapler is attached to an independent robotic surgical system 54334, the HCP operating the primary robotic surgical system 54326 may request control of the circular stapler from the secondary robotic surgical system 54334. Once the lead HCP operating the primary robotic system establishes control with the secondary robotic surgical system and the circular stapler, the lead HCP at the console 54328 of the primary robotic surgical system may be presented with controls that are specific to the secondary robotic surgical system 54334 and the surgical instruments attached to the secondary robotic surgical system, for example, anvil closure, firing, opening, etc. A second HCP 54329 (e.g., a resident HCP) may be in proximity of the secondary robotic surgical system 54334 and may be in physical control of the circular stapler attached to the secondary robotic surgical system 54334.

In either of the setups illustrated in FIG. 31 and FIG. 32, the lead HCP may instruct the second HCP 54329 about physical placement of the stapler. The lead HCP may instruct the second HCP 54329 about initial placement of the stapler with respect to the surgical site. The lead HCP may provide further instructions to the second HCP 54329 about the next steps that are to be performed in the surgical procedure. Under the lead HCP's direction, the second HCP 54329 may perform one or more of the following steps: position a circular stapler or device at a desired position; insert the anvil; or attach the anvil to the trocar of the circular stapling surgical instrument. The circular stapling surgical instrument may now be prepared for a circular firing. The lead HCP may be presented on the main console controls associated with the primary laparoscopic multi-arm robotic surgical system, which may be overlayed with the controls associated with the circular stapler attached to the secondary robotic surgical system. The lead HCP may send commands to the secondary robotic surgical system or the smart surgical system attached to one of the arms of the secondary robotic surgical system using the wired or wireless communication path established between the console and the secondary robotic surgical system or circular stapler.

In either of the setups illustrated in FIG. 31 and FIG. 32, the lead HCP may send a command to the circular stapler (either directly or via the secondary robotic surgical system) for beginning of the closure. In addition to the lead HCP sending the command to the secondary robotic surgical system or the circular stapling device, the second HCP may monitor the mechanical aspects of the closure (e.g., tissue compression scale, etc.) The circular stapling device may communicate an indication to the main console indicating that appropriate compression value has been reached and the stapling device closure stroke is stopped and held at the compression reading. In addition, the second HCP may visually verify the compression is within an acceptable zone (e.g., green zone) of the tissue compression scale. The lead HCP may use its console 54328 to visually verify that the two tissue ends are captured correctly, and circular stapler is ready for the firing step. In FIG. 32, where the circular stapler is part of a different robotic system than the one the lead HCP is directly controlling, the lead HCP on its console 54328 may have access to an imaging stream being shared by the single arm robotic surgical system 54334 that is housing the surgical stapler. Using the shared imaging stream, the lead HCP may also visually verify that the two tissue ends are captured correctly and circular stapling device is ready for the firing step.

Once the readiness of the circular stapling device is verified, the lead HCP (either directly/locally or remotely) may send commands to the circular stapler to fire. Once firing of the circular stapler has been completed, the lead HCP may command the circular stapler to open the anvil. The lead HCP may then relinquish control of the circular allowing the second HCP 54329 to remove the circular stapler.

The position of the circular stapler may be determined by attaching it to a robotic arm, as illustrated in FIG. 31 and FIG. 32. As illustrated in FIG. 31, the robotic arm may be part of the primary robotic surgical system (e.g., the main laparoscopic robotic surgical system). As illustrated in FIG. 32, the circular stapler may be attached to a separate robotic surgical system, for example, a single arm robotic surgical system 54334 or a docked support arm. This may enable the lead HCP sitting at the main console 54328 to indicate that care may be needed to prevent excessive pressure to the linear staple line while also confirming when the circular stapler distal head is within an acceptable range.

Systems, methods, and instrumentalities described herein may allow more than one independent surgical systems (e.g., an endoscopic robotic system and a laparoscopic robotic system) to act on same input data by processing two independent control requests (e.g., two independent and/or parallel direct user control requests). The two independent surgical systems may operate as if the input data was provided directly to each of the systems independently. Such an arrangement may allow the main console to send commands including control requests to each of the systems that may behave as independent surgical systems, yet perform coupled or linked operations.

In an example, more than one remote user requests may be synchronized. The synchronization of the remote user requests may originate from a common console that may have capability of generating the remote user requests using more than one protocols that are compatible with the surgical systems being controlled by the console. The commands used for user requests may be sent using wired, wireless, or other interfaces, for example, using a user input/output device (e.g., a touchscreen, keyboard, mouse, joystick, etc.).

FIG. 33 illustrates performing a pre-surgical procedure for identifying a cancerous tumor, which is followed by performing a colorectal surgical procedure to remove the identified cancerous tumor. As illustrated in FIG. 33, at 54370, a pre-surgical procedure (e.g., a CT scan 54337 as illustrated in FIG. 34) may be performed on a patient for identifying a tumor that may require removal. At 54372, vision system detection through tagging agents may be performed. At 54373 a combination of CT scan overlay and vision system detection through tagging agents may be utilized to identify a polyp or a cancerous colorectal tumor. Once the colorectal tumor is identified, at 54374, position of the colorectal tumor with respect to the laparoscopic robotic system may be determined. At 54375, position of the colorectal tumor with respect to the endoscopic robotic system may be determined. At 54376, proximity of each of the laparoscopic robotic system and the endoscopic robotic system may be computed (e.g., computer together) and sent independently to the two robotic systems. At 54377, the proximity of the colorectal tumor to the laparoscopic robotic system may be sent to the laparoscopic robotic system. At 54378, the proximity of the colorectal tumor to the endoscopic robotic system may be sent to the endoscopic robotic system.

Once the presence of the colorectal tumor is identified, and the proximity of the colorectal tumor to each of the robotic systems is computed and communicated (communicated separately) to each of the robotic surgical systems, each of the two independent robotic surgical systems may operate in a linked fashion during the surgical procedure, as illustrated in FIG. 34.

As illustrated in FIG. 34, laparoscopic robot arms 54327 that are part of a multi-arm robotic surgical system and an endocutter in combination with a scope 54336, which is a part of an endoscopic robotic surgical system may be utilized to perform a colorectal surgical procedure, as described herein. As described in FIG. 35A-35B, the endoscopic robotic system may be utilized to snare and fold the tissue wall pinching in closed and the endocutter can then be placed over the fold separating the endoscopic grasped tissue and sealing the hole that is created in the side. During the stapler firing step of the robotic surgical system controlling the endocutter, it may operate in tandem robotic surgical system controlling the endoscope such that the endoscopic retraction may be applied to pull inward as the stapler is pulled outward. Both the commands for executing the staple firing step and the endoscopic retraction step may be issued in parallel to the endocutter device (which may a part of the multi-arm laparoscopic robotic surgical system) and the endoscopic device (which may be a part of a separate independent robotic surgical system). In an example, the commands may be sent to the two robotic surgical systems (e.g., sent from a main console controlling the two independent robotic surgical systems) without the two robotic surgical systems exchanging information directly.

FIGS. 35A and 35B illustrate an exemplary colorectal tumor removal surgical procedure using techniques described herein. As illustrated in FIGS. 35A and 35B, once the presence of the colorectal tumor is identified using the CT scan device 54337 of FIG. 34, endoscopic robotic surgical system and laparoscopic robotic surgical system may be utilized to perform the surgical procedure.

During the actions illustrated in FIG. 35A and FIG. 35B, a lead HCP may control the laparoscopic robotic surgical system 54340 including the grasper 54344 and scope 54341. A second HCP may control the endoscopic surgical system 54346 including the stapler 54347 and the snare 54348. As illustrated in FIG. 35A, the lead HCP may send commands to position the grasper 54344 next to the tumor 54342 against the colon serosal layer 54343, and the second HCP, using endoscopic control 54346 may position the stapler 54347 and the snare 54348 in position next to the tumor 54342.

As illustrated in FIG. 35B, the lead HCP may send command to the laparoscopic grasper 54344 to push the tumor 54342 further into the colon (e.g., toward the endoscopic devices). The second HCP may send commands to endoscopic surgical system for applying the endoscopic snare 54348 to the inverted tumor 54342. The second HCP may then send commands to retract the snare to pull the tumor further into the colon. The movements of the stapler and the snare are shown by arrows next to the stapler 54347 and the snare 54348. The lead HCP may continue to push the tumor into the colon while the second HCP may fire an endoscopic stapler 54347 to separate the tumor from the colon tissue and staple the resulting incision. Commands may be sent independently to the laparoscopic robotic surgical system to push the tumor into the colon and to the endoscopic surgical system for stapler 54347 stapling and moving in the forward direction and for the snare 54348 to move in the opposite direction. The commands to the two surgical systems may be sent independently.

FIG. 36 illustrates an example tumor removal using techniques described herein. Colonoscopy-assisted wedge resection technique may be used for polyps located near the antimesenteric side of the colon. Laparoscopic wedge resection with intraoperative colonoscopy may be performed for removal of large tumors that may not be treated endoscopically.

As illustrated in FIG. 36, colonoscopy assisted laparoscopic edge resection (CAL-WR) surgical procedure may involve one HCP operating and controlling the laparoscopic robotic surgical system and the other HCP operating and controlling the colonoscopy robotic surgical system.

CAL-WR surgical procedure is initiated with the lead HCP performing diagnostic laparoscopy with the insertion of multiple trocars. The spot of the tumor 54353 in the colon 54355 may be identified and the corresponding part of the colon may be mobilized. Mobilization may be performed to enable the HCP the ability of placing the stapler 54352 (which may be part of the laparoscopic robotic surgical system) in the best possible position.

The second HCP may mobilize the endoscopic scope and place it next to the tumor site. The lead surgeon may laparoscopically place a suture near the tumor with intraluminal endoscopic visualization. Traction may be provided on the suture to enable positioning of the stapler 54352. The lead HCP may then send commands to fire the stapler 54352, which is part of the laparoscopic robotic surgical system and confirm total inclusion of the tumor 54353 using the endoscopic robotic surgical system 54354. The two commands may be sent in parallel without the laparoscopic robotic surgical system and the endoscopic robotic surgical system interacting with each other.

In an example, multiple surgical systems or robotic surgical systems may operate together in performing steps of a surgical procedure. In addition, the surgical systems may all be from different manufacturers and may have different control systems. Any of the surgical systems, therefore, may not be configured in a manner to surrender full control of its operation to another manufacturer's surgical system. Such an arrangement may be prohibited, for example, one or more of the following reasons: patient safety, one surgical system may not have a full understanding of operation of a second surgical system, nor the willingness to assume accountability for its safe operation, loss of the propitiatory data recorded or the operational program. However, in case of an integrated operating room, the surgical systems may operate independently as originally designed and certified but with an ability to accept requested commands on operation from an HCP via an intermediate smart system.

In an example, external imaging system (e.g., a cone-beam CT) may operate with another smart system and may need to be repositioned or the image focal location or orientation may need adjusting. In an example, for example in a thoracic surgical procedure, an imaging system may be used in cooperation with a flexible endoscopy surgical device (e.g., a hand-held endoscopy surgical device or a robotic endoscopy surgical device). A flexible scope may be extended further into the bronchus. Such extension of the flexible scope further into the bronchus may then need the imaging system is to adjust its orientation (for example, as illustrated in FIG. 39) to keep the distal tip of the flexible scope in the field-of-view or even centered in the field-of view. In such a case, the imaging system may receive a request from an HCP sitting at the main console of the flexible scope or an intermediate smart system to adjust the imaging system.

In another example, the imaging system may be automatically adjusted based on the relative position of the flexible scope in the bronchus of the patient. In such an arrangement, manner where the surgical system controlling the flexible scope or the flexible scope itself may provide updates regarding the scope position information to the imaging system and the imaging system may utilize the updates regarding the scope position information to adjust the its position accordingly. The updates regarding the scope position and operation information may include information about the operation, position, and adjustments of the position of the flexible scope.

In an example, the flexible scope, as part of preemptive alignment, may instruct the imaging device about the timing of movements and locations and/or directions associated with the movements (e.g., as illustrated in FIG. 39). The preemptive alignment may be applied proactively. For example, the imaging system may be instructed to move to a position tracking a specific part of the bronchus and then the flexible scope would be moved to that position in the bronchus.

In an example, a first smart surgical system may have discernment of its limitation relating to the actions of a second smart surgical system. This decision may result in the first smart surgical system requesting the second surgical smart system for operational instructions in remote controlled fashion.

In an example, an originating smart surgical system may determine that one or more actions to be performed as part of a surgical procedure are outside of its physical or processing capabilities. The originating smart system may discover and/or reach out to nearby neighboring smart surgical system for assistance. The originating smart surgical system may prepare to surrender control to the neighboring smart system that may have the capability of supporting one or more actions. In an example, neighboring smart system may yield itself and request alternate smart surgical system to perform action requested by the originating smart system.

Systems, methods, and instrumentalities are described herein for enabling full remote control of one smart surgical system by another smart surgical system. For example, in case of using robotic flexible endoscopy used with robotic laparoscopy, the laparoscopic robot console may be configured (e.g., may assume) as the primary robotic surgical system and the robotic surgical system controlling the flexible endoscopy unit may configured as another minion of the console just like the laparoscopic robotic arms of the laparoscopic robot system. In an example, a robotic surgical systems (e.g., a Hugo robotic system) may have multiple minion independent cart arms tethered to a control console. In this case the flexible endoscopy robot may be attached to either the main tower or directly to the robot control console allowing the controls to interactively control all of the attached arms interchangeably.

Features described herein may allow a primary surgical system or a primary robotic surgical system to have direct integrated control of a minion surgical system. In an example, operating mode of a minion system may be same (e.g., from the same manufacturer and operating on a compatible version of software) as that of a primary robotic surgical system. In such a case, the minion system may be integrated and/or attached to an arm of the primary robotic surgical system. The minion surgical system attached to one of the arms of the primary robotic surgical system may be inter selectable (e.g., like any of the other arms of the robotic surgical system). The minion surgical system may be controlled using the common interface or the main console of the primary robotic surgical system.

In an example, a dual visualization comprising the primary robotic system and the minion system may be presented on a common interface or the main console connected with the primary robotic system. For example, a dual visualization may be presented using one of the following: a picture-in-picture mechanism, it or an integrated mechanism, for example, using overlays or transparent views that may merge the imaging associated with the two surgical systems enabling an HCP to see through or behind tissues and/or organs. In examples, merging or overlaying may include using augmented reality to add or overlay imaging associated with one surgical system over the imaging associated with the other surgical system. In an example, the user interface or the main console display showing the HCP what they normally expect from a real-time visual light image of the surgical site while then being able to add or supplement portions of the view that the secondary imaging could add data about.

Features described herein may allow more than one surgical systems (e.g., a primary surgical system and a minion surgical system) to operate in tandem in a primary-minion mode (e.g., even if the two surgical systems may not be compatible to be integrated directly). In such an arrangement, an imaging stream (e.g., a video stream) may be ported from the minion surgical system to the user interface or main console that may be a part of the primary surgical system. In addition, the primary surgical system may be used as controller for controlling various aspects of the minion surgical system. In an example, the primary surgical system may send control signals and/or commands to the minion surgical system. The controls for effecting movement on the minion surgical system may be simulated or emulated by the primary surgical system allowing it to be an I/O system for the minion surgical system.

In example, multiple minion surgical systems may be controlled (e.g., simultaneously controlled) by a primary surgical system. A surgical system with integrated with a scope imaging system (e.g., Olympus EBUS scope) and a flexible endoscope may be configured as minion surgical systems that may be controlled by a primary surgical system (e.g., a Hugo laparoscopic robotic surgical system). In case of the primary-minion control model, the primary surgical system may autonomously establish partial control of the minion system(s).

In an example of removing gallbladder stones surgical procedure below, one surgical system (e.g., Hugo robot) may be configured and/or positioned as the primary robotic surgical system, for example, to perform cholecystectomy. The primary robotic surgical system may be used as the primary robot for imaging and as the main visualization source and main control console interface to be used by one of the HCPs (e.g., the surgeon) involved in the surgical procedure. Another surgical system (e.g., a Monarch flexible robotic system) may be used for controlling the endoscope portion of a scope imaging system (e.g., Olympus EBUS ultrasound secondary scope) for imaging of the stones and ducts. The ultrasound image from the scope may be overlaid on display of the primary surgical system (e.g., Hugo system) to visualize the underlining structures from the laparoscopic side. For example, the HCP controlling the primary surgical system may redirect the scope slightly to get a better image. The HCP may have direct control of the primary surgical system as well as requested, but independent, control of the scope imaging system.

In an example, and in addition, to obtain the desired imaging view using the scope imaging system, it may be reoriented (e.g., slightly reoriented) such that the primary surgical system may request the minion surgical system to adjust the control cables of the flexible scope such that the head location may allow the scope imaging system to have a better view. The request may be sent (e.g., autonomously sent) by the control system of the primary surgical system control system (e.g., without any intervention of a HCP). The request may be sent by the primary surgical system, for example, because the HCP was busy controlling the scope imaging system and a physical movement of the scope was need in addition to the control adjustments of the scope imaging. In such an example, the primary surgical system and the HCP may supply I/O interface data to a specific minion system, which in turn may operate as expected. In this case, the primary surgical system may direct the minion system(s) without taking control of the minion system(s).

Features described herein may provide reversible or bi-direction primary-minion control exchange. In this case, an HCP's interaction with various surgical systems may be used to identify the primary surgical system. For example, an HCP may move from one surgical system to another and the operational control of the first surgical system may be transferred with the HCP, as the HCP moves from one surgical system to another. In order to ensure that a primary control system is designated at all times without any interruption, each of the surgical systems may attempt to maintain its designation as a primary surgical system. The HCP presence in combination with authentication of the HCP may be utilized to designate a surgical system as the primary surgical system. In an example, authentication of the HCP used in designation of a primary may be performed by using one of more of the following authentication mechanisms: a digital key or token that may be required by a surgical system to establish primary control.

The surgical systems involved in the bi-direction primary-minion control exchange may be aware of each other and the control interface established for the HCP, for example, to track the HCP. In an example, the control interface established for the HCP may include a physical aspect, e.g., a switch, a digital identification, or a biometric monitoring system. In an example, a smart Hub (e.g., a separate Hub) from any of the other surgical systems may be uptilted to maintain control and access grants. The smart Hub may inform the surgical systems about the identification of the primary surgical system. The system based on the smart Hub may track the HCPs as they move between surgical systems to granting primary control to the surgical system with which the HCP may be directly interfacing and revoking the primary control when the HCP is no longer interacting with the surgical system.

Features described herein may be provided for dual generator control with both generators existing within the same Hub tower or rack. In an example, operations in combo energy devices such as monopolar-bipolar, bipolar-ultrasonic, or monopolar-ultrasonic energy may be combined. The operations may be combined at the same time or in a serial fashion. In such a case, two separate generators may work in tandem to provide the handpiece or a robotic surgical device the proper energy modality, power level, and/or communicate pressure needs in order to provide the advanced outcome desired. If the two generators are in the same module or in the same smart hub, one of the energy devices may receive commands from the other energy device or both the energy devices may take commands from a primary command source to coordinate their outputs.

Features described herein may be provided for independent smart system evaluation and determination of other system's controllability. In an example, one surgical system (e.g., surgical system A) may request control of the other surgical system (e.g., surgical system B).

A central arbitrator may be required to coordinate the transfer of control. The arbitrator may determine that the first surgical system has the required hardware and/or software to complete the full control transfer. If the arbitrator deems the appropriate level, it may allow for establishment of a direct high speed data/control transfer between the first surgical system and the second surgical system. If the arbitrator determines that full control is not within the capabilities of either the first surgical system or the second surgical system, it may generate an alert indicating the level of control that may be allowed and an indication whether this level of control will be sufficient for the upcoming surgical procedure steps.

A system may be configured with a default level of control which may be the highest degree of control allowed based on the setup of the two systems.

If the arbitrator determines that one surgical system has limitations that may compromise the direct control of the other system, the surgical systems involved and/or the arbitrator may determine if the risk of completing the actions is acceptable.

The final risk determination may cause the surgical system to lower the level of control one surgical system may have over the other surgical system. The determination may be based on the level of control the one of the surgical systems (e.g., the second surgical system) may be able to achieve and the properties and/or requirements of the upcoming surgical steps in a surgical procedure. In an example, higher levels of risk may cause the surgical systems to lower the level of control.

Each of the surgical systems involved in establishing the controllability may acknowledge the request for control to the arbitrator, and each of the three systems may agree on the transfer before proceeding.

Features described herein may be provided for arbitrator master control of multiple surgical systems. The arbitrator may act as the final decision maker as to the level or mode of cooperation between the more than one surgical systems. In addition, the arbitrator may make lower level decisions regarding whether the surgical systems are going to share a new temporary memory stack the systems share.

Features described herein may be provided for establishing shared memory and stack. Each of the surgical systems associated with this new network may utilize the shared memory and/or the stack to process the code and storage areas. Surgical systems may share the shared memory, which may allow up to date access and any modifications that may be needed to memory and/or task control.

The arbitrator may decide whether an additional high speed data line needs to be established between the cooperating systems. If the steps of the procedure require it, the arbitrator may set up this structure and then monitor (e.g., continuously monitors) the procedure, for example, as a safety mechanism.

Features described herein may be provided for a shared full control of one robotic surgical system with another. One of the robotic surgical systems may be designated as a primary surgical system and the second system may also be a primary surgical system. The second robotic surgical system may then allow the first robotic surgical system authority over at least some of the operational characteristics (e.g., not all the operational characteristics) of the second surgical system. The sub-primary robotic surgical system may retain control of all of the aspects of the coupled robotic surgical system, but may allow the primary robotic surgical system to request limited aspects of the sub-primary control.

The sub-primary robotic surgical system may monitor the remote controlled systems providing them additional, supplementary or override control of the remote controlled sections. In an example, a Monarch flexible robot may be designated as a primary robotic surgical system and an Otava robot may be designated as the sub-primary robotic surgical system. The sub-primary robotic surgical system may grant remote control of two of its four arms to the primary robotic surgical system for cooperative interaction, for example, while performing an Endo-Lap surgical procedure. The sub-primary robotic surgical system may also provide supplementary control of two remote controlled arms to provide interaction control of the portions of the arm outside the body relative to the patient, the table and the other two arms. An HCP using the primary robotic surgical system may move the two remote arms inside the patient, the sub-primary robotic surgical system may provide some direction to the joints outside the patient for both the robotic surgical systems to orchestrate their movement to prevent collisions outside the patient's body while the HCP is controlling the end-effector motions inside the patient's body.

In an example, supplementary surgical system modules may also establish primary and sub-primary relationship and operate in concert. Advanced energy generator, the advanced visualization modules, smoke evacuators or patient critical modules like insufflation or ventilation system maintaining their prime operational directives and the other systems allowed to interface with some control aspects unless those aspects interfere with their primary operational mode.

In an example, a smart ventilation system may be shared sub-primary controlled by a smart hub or a robot hub. For example, the ventilation system may allow the smart hub or the robot hub to vary the ventilation rate and the oxygen levels as long as they stay within a preconfigured and pre-set patient operation envelope. The smart hub or the robot hub may also operate other controls of ventilation system, including for example, air volume, air pressure, etc. to keep functioning as intended. If the remote control from the smart hub or the robot hub drives a controlled factor of the ventilation system to a point where the ventilation system is being pushed out of its normal operating mode, or one or more of the patient biomarker measurements indicate a critical situation then the sub-primary surgical system may regain full primary control of its system to re-balance the settings based on its primary control algorithms. In this case, the sub-primary surgical system may notify an HCP (e.g., an HCP on a remote system or the primary surgical system) the reason for the sub-primary surgical system taking back full control and/or rejecting a request the sub-primary surgical system may have received from the primary system. The sub-primary surgical system may allow for the HCP to control the sub-primary surgical system within this marginal range, but may prevent it from moving anything to critical or dangerous range.

FIG. 37 is a message sequence diagram illustrating control sharing between two surgical systems (first surgical system 54360 and second surgical system 54361) used in a surgical procedure. The surgical procedure may include a colorectal surgical procedure (as illustrated and described herein in 317 through 34, 35A, 35B, and 36) or a thoracic surgical procedure (as illustrated and described herein in FIG. 30, FIG. 38, and FIG. 39), the first surgical system may be a endoscopic robotic surgical system that may be autonomous or operated by one HCP and the second surgical system may be an laparoscopic robotic surgical system that may be operated by another HCP (e.g., the lead HCP).

As illustrated in FIG. 37, at 54362 discovery of surgical systems that support image porting and remote control may be performed (e.g., by smart surgical systems in an operating room, with or without a smart hub). Based on the discovery information, one or both of the first surgical system 54360 and the second surgical system 54361 supports image porting and remote control.

At 54363, the first surgical system 54360 may receive a request (e.g., second surgical system 54361 may send a request) associated with redirection of imaging and/or a control interface from the first surgical system 54360 to the second surgical system 54361 (e.g., remote control surgical system).

At 54364, the second surgical system 54361 (e.g., based on the request) may receive imaging and indication of controls (e.g., full control or partial control) associated with the first surgical system 54360. At 54365, the second surgical system 54361 may display imaging from the first surgical system 54360 and the control interface of the first surgical system (e.g., based on a received set of operational controls (OCs) that the second surgical system 54361 is permitted/enabled to change/modify). The imaging received from the from the first surgical system 54360 may be added to the display of the second surgical system 54361.

At 54366, the second surgical system 54361 may request a control setting change based on the set of OCs received from the first surgical system 54360. The first surgical system 54360 may determine whether to validate the control setting change. At 54367, the first surgical system 54360 may validate the control setting change. In case the validation of the control setting change is successful, at 54368 (i.e., remote operational control changes are allowed), at 54370, the first surgical system 54360 may change the control setting (e.g., a set of OCs) based on the received control settings from the second surgical system. At 54371, the first surgical system 54360 may send an acknowledgment to the second surgical system 54361 indicating the control setting change. The first surgical system 54360 may send an additional (e.g., updated) set of OCs that the second surgical system is enabled (e.g., permitted) to change.

At 54372, the second surgical system 54361 may display updated imaging from the first surgical system and an updated control interface of the first surgical system based on the received set of OCs that it is permitted to change.

In case the validation of the control setting change is not successful, at 54369 (i.e., remote operational control changes are not allowed), At 54373, the first surgical system 54360 may determine to reject the requested control setting change and change the OC based on local settings instead. At 54374, the first surgical system 54360 may send a negative acknowledgement (NACK) and/or a reason for NACK to the second surgical system 54361. At 54375, the second surgical system 54361 may update (e.g., remove) control settings based on the received NACK. The second surgical system 54361, based on the received NACK may determine to terminate remote control of the first surgical system by the second surgical system.

At 54376, the first surgical system 54360 may evaluate and/or monitor OCs that are set based on the control settings. At 54377, the first surgical system 54360 may monitor data (e.g., patient biomarker data), to determine control settings. Based on monitored data associated with a patient, the first surgical system 54360 may determine that the patient biomarker value has crossed a threshold value. The threshold value may be preconfigured or negotiated between the first surgical system 54360 and the second surgical system 54361. Based on the determination that the patient biomarker value has crossed a threshold value, the first surgical system 54360 may terminate remote control. At 54378, the first surgical system may send a notification indicating that termination of the remote control and/or the reason for termination of the remote control and that the first surgical system is assuming the control. At 54379, the second surgical system 54361 may update (e.g., remove) the control settings and/or imaging based on the received notification.

Features described herein may be provided for dual generator cooperative operation of combo devices that may have more than one generators (e.g., in separate hub towers or racks). For example, in case of two cooperative generators that may be configured for a single combo device in separate control or communication hubs, one of the cooperative generators may be designated as the primary system. The cooperative generator designated as the primary system may receive inputs (e.g., control inputs) from an HCP for controlling the main energy modality. The primary system may then request the second generator (e.g., the sub-primary system) to supply its energy when and how it may be needed to complement the primary system's energy. The non-primary generator may run its energy generator's main operation and safety aspects as normal, and may consider the shared control commands it may receive from the primary generator as instructions about where, when, and how to provide the supplemental combo energy to the primary generator for performing advanced operation of the combo device.

In an example, a uterine manipulator may be attached to a robotic control arm. Use of an uterine manipulator being introduced through an externally control robot arm control. Examples of the use of a robotically controlled uterine manipulator are described in U.S. Pub. No. 2023/0077141, entitled “Robotically controlled uterine manipulator,” filed Sep. 21, 2021, the disclosure of which is incorporated by reference herein. Primary motion of the dissection of the surgical procedure may be controlled from the main console controlling laparoscopic instruments control of a second system. The uterine manipulator can be at the console or at the bedside when at the console the commands to the uterine manipulator are limited to up down left right, providing for presentation of the dissection planes in the laparoscopic view of the balder and rectum respectively. The in/out motion of the uterine manipulator may be limited by the console commands (e.g., not able to be commanded at the console) to prevent perforation of the uterus. The in/out motion of the uterine manipulator may be limited to manual at the bedside via gravity compensated motion of the robotic arm manually moved, with optional geofencing of up down or left right movements.

Features described herein may be provided for multi-user control of a multi device smart system, for example, within a shared environment. In examples, surgical environment, for example, operating rooms may often be configured with more than one robots or robotic surgical systems and/or more than one smart systems along with multiple HCPs. Each of the HCPs and the surgical or smart systems may interface or interact with each other while performing a surgical procedure. Surgical instruments/surgical devices/surgical systems may allow access and/or control of a function by a unique or trusted HCP. However, in a multi-user environment and/or multi-device smart systems may create different challenges, for example, dealing with conflicting task/execution and or changing demands based on user preference. In this case, each smart system may deal with one or more of the following scenarios: when allowing access off of different systems the smart system may only display the usable command or options to a specific HCP based on defined level of controls (e.g., the surgeon may have full control in any situation, unless a senior surgeon overrides the surgeon's command, a nurse may be allowed to reposition a robot but only in safe conditions); negotiation between HCPs to resolve conflicting demands; override human errors; allow for collaboration with other HCPs (e.g., surgeons) either in or outside of the operating environment, which may allow for HCPs or surgeons from anywhere in the world to assist or guide a surgical procedure. The HCP or the surgeon may have credentials to control the commands for operation but not able to move robot location or instruments attached to the robots, which would require a different HCP to complete a task while the HCP or the surgeon may perform other tasks.

Manual or autonomous controls may be provided. For example, a surgical system capable of full control behavior may have the capability of potentially operating on any of the different levels. The surgical system may operate on different levels with different surgical system simultaneously with separate smart systems.

In an example, the most basic mode of operation may be independent by requested mode. In an example, various systems may be from the same manufacturer or may have been designed perform as such, the most comprehensive primary-minion control may be utilized. The shared control may be used as an optional addition to the primary-minion control while the control may be retained by the built-in control system. In this operational state a hierarchical order or control may be provided. The hierarchical order or control may be based on where the primary HCP is located. In an example, the hierarchical order or control may also be based on priority/safety/criticality, or the main controls (e.g., main controls may have primary priority over any remote controls). Verifying the authenticity of data communicated from a surgical instrument to the communication hub is described in U.S. Pub. No. 2019/0200844, entitled “Method of hub communication, processing, storage and display,” filed Dec. 4, 2018, the disclosure of which is incorporated by reference herein.

In examples, at least two HCP consoles from separate robot surgical systems may be utilized for controlling a separate single smart system simultaneously. Smart system may separate control of different actions of device to multiple controllers.

Features described herein may be provide for multi-user control of a single device smart system within a shared environment. Single device may be simultaneously controlled by multiple HCPs, for example, each HCP may utilize unique control methods.

In examples, device location, movement, and/or positioning may be controlled by smart vision system. Device energy activation/staple deployment may be controlled by an HCP (e.g., the lead HCP or a surgeon) or an alternate HCP who may be designated as controller.

FIG. 38 illustrates an example of an HCP 54389 in control of one robotic system being able to control or adjust other robotic surgical systems (e.g., other local surgical systems). As illustrated in FIG. 38, the HCP may be in control of a robotic endoscopic surgical system during a thoracic surgical procedure, for example. After positioning the flexible scope 54391 at the desired location 54390 inside the bronchus 54392, the HCP using the user interface 54387 may select the Cone beam CT C-arm selection 54386 to adjust or move the cone beam CT c-arm from its current position 54388 to a desirable position 54393 in order to correct the focal point of the cone beam from position 1 54394 to position 2 54395. The user interface 54387 may be provided on a fixed console or a mobile device (e.g., a tablet).

In an example, a handheld circular stapler may establish connectivity with the robotic console. The circular stapler may be configured and may be used and/or controlled as part of robotic and laparoscopic surgical procedures.

In an example, a circular stapler may be positioned and held by an HCP (e.g., an assistant to other HCP). The device firing and closure controls may switch back and forth between the HCPs (e.g., between an assistant and a lead surgeon). Operation of a circular stapler may require inserting and controlling by a non-sterile assistant, but stapler may require it is highly desirable for device feedback and control associated with the circular stapler to be provided to the lead surgeon, who is sterile.

A circular stapler with remote connectivity may provide feedback to an HCP or a surgeon operating the main console controlling a robotic surgical system and also control the circular stapler. However, when the circular stapler is to be inserted by one HCP and controlled by the other HCP, the balance and switching of controls may become complex.

A surgical procedure, for example a colorectal surgical procedure may involve a first HCP (e.g., a robotic surgeon) and a second HCP (e.g., an assistant to the robotic surgeon). The first HCP may at the console of the robotic surgical system and may take control of the closure and firing of various system including the circular stapler, for example, when the circular stapler is ready to attach the anvil, close on tissue and ready for firing. Prior to the first HCP being ready for filing the circular stapler, the second HCP may manually insert the circular stapler into the patient. The second HCP may need control of the trocar and anvil in order to safely insert and remove the stapler.

Prior to the insertion process into the body, the second HCP may need to open the anvil fully, remove the anvil, then retract the trocar. These steps may need to be controlled on the device itself, and may be done outside of the surgical field while the first HCP is busy completing other procedure steps. The handheld buttons/controls would need to be active and the console controls be deactivated.

During actual insertion into the body, the second HCP may retain control until the first HCP is ready to extend the trocar and puncture tissue. In an example, extending the trocar may be performed by or under supervision of the first HCP under the first HCP's control. In an example, the first HCP may delegate it to the second HCP who may be instructed to extend the trocar. The second HCP may physically hold the circular stapler and position the rectal stump relative to the end effector.

While the anvil is being installed onto the trocar, no circular device controls may be needed, unless the trocar extension position needs to be adjusted. The adjustment, if needed, would be covered by the first HCP.

When the anvil is fully installed by the first HCP, full device control may be shifted from the first HCP to the second HCP at the console for issuing control commands for closure and firing. After firing, and on removal of the device, the device control may shift back to the second HCP.

If excessive force is noted on the anvil during removal, alerts may be shown to the first HCP at the console, either allowing the first HCP control of opening the anvil further, or prompting the second HCP to open it further. In an example, the circular stapler may automatically adjust itself and user control to both the first HCP and the second HCP are deactivated.

In examples, for a combination harmonic device, one system may be used to control the device positioning and another system may be used to control the device activation. In another example, for a combination harmonic device, one system may be used to control the device positioning, and a second system may be used to control the device RF activation, and a third system may be used to control the device harmonic activation.

In examples, a first robotic surgical system (e.g., an Otava robotic system) with may have a first console that may be controlled by a lead HCP. A second robotic surgical system (e.g., Monarch robotic system) may have a second console that is controlled by another HCP (e.g., an assistant surgeon). Both the surgical systems may interact with each other, for example, to control a single of the second robotic system. A working channel may be autonomously operated from the bending of the flexible scope. One of the HCPs may perform a snaring task while another HCP may perform the positioning of the snare, as described herein.

FIG. 39 illustrates an example of exchangeability of imaging streams and control between various surgical systems and smart surgical systems (e.g., imaging console systems). As illustrated in FIG. 39, various HCPs (e.g., the lead HCP and a assistant HCP) and surgical systems (e.g., robotic surgical systems), smart imaging systems, and a surgical hub may be used to perform a surgical procedure (e.g., a thoracic surgical procedure or a colorectal surgical procedure).

The HCPs involved may include the lead HCP operating the robotic laparoscopic surgical system 54324 using the robotic laparoscopic surgical system console 54328 and/or a single arm robotic surgical system 54334. The lead HCP may also utilize the monitor 54385 that is a part of the robotic laparoscopic surgical system 54324. A second HCP (e.g., an assistant HCP) may operate and control the robotic endoscopic flexible scope 54322. The second HCP may utilize the monitor located above the tower controlling the endoscopic flexible scope 54322. A third HCP (e.g., a radiologist 54382) may operate and control C-arm cone-beam CT system 54380 via the C-arm console and monitor 54381.

In an examples, controls and imaging streams may be shared between the robotic laparoscopic surgical system 54324 and the robotic endoscopic flexible scope 54322. For example, the lead HCP operating and/or controlling the robotic laparoscopic surgical system console 54328 may control the robotic endoscopic flexible scope 54322, for example, to adjust the location of the endoscope to a desired location. In such a scenario, image streaming may be established between the robotic endoscopic flexible scope 54322 and the robotic laparoscopic surgical system 54324 allowing the lead HCP to observe on the robotic laparoscopic surgical system console 54324 what the second HCP may be observing on the monitor located above the tower controlling the robotic endoscopic flexible scope 54322.

In examples, controls and imaging streams may be shared between the robotic endoscopic flexible scope 54322 and the C-arm cone-beam CT system 54380. For example, the HCP operating and/or controlling the robotic endoscopic flexible scope 54322 may require to adjust the focal point of the C-arm cone-beam CT system 54380, as illustrated in FIG. 39. The HCP either using the user interface of the robotic endoscopic flexible scope 54322 or using a separate device, for example, a tablet (as illustrated in FIG. 38) may send commands to the C-arm cone-beam CT system 54380 in order to adjust its focal point such that it is aligned with the desired location of the HCP and the lead HCP. Imaging (e.g., imaging in real time) may be shared between the C-arm cone-beam CT system 54380 and the user interface the HCP is interacting with such the HCP can observe exactly what the HCP controlling the C-arm cone-beam CT system 54380 is observing. In an example, the HCP's user interface may overlay the image generated by the C-arm cone-beam CT system 54380 over that generated by the robotic endoscopic flexible scope 54322.

In an example, the images generated by both the robotic endoscopic flexible scope 54322 and the C-arm cone-beam CT system 54380 may also be streamed to the console of the lead HCP's console may then over lay the image generated by the C-arm cone-beam CT system 54380 over that generated by the robotic endoscopic flexible scope 54322.

In examples, one or more energy devices that include an air suction device, an energy delivery device, etc. one smart system (e.g., a vision system) may be used to monitor the visibility of the area where smoke may be generated and in response the smart system may control the air suction ON/OFF or rate, and a second system may be used to control drug delivery.

Device feedback and algorithms may switch between control systems. For example, when one of the HCPs is positioning a surgical device and/or manipulating a closure system, internal device feedback control of the surgical device may be used to control closure knob or other buttons to limit closure speeds. In an example, this may occur before the surgical device may start communicating with a robotic console.

In an example, when the lead HCP (or surgeon) at the console takes control of closure and firing system, more advanced console based algorithms adjust firing and closure accordingly.

The controls of the device may switch back and forth between HCPs depending on the surgical procedure step, the lead HCP's choice, device feedback, etc. Control switching may be performed manually or automatically. For example, control switching may be performed manually based on user input. The control switching may be performed automatically based on contextual surgical procedure data (surgical procedure step, etc.), or internal device feedback (e.g., load status, etc.).

Some combinations of controls may be active simultaneously for both users. For example, control may be provided to a lead HCP (or surgeon) for firing control, while another HCP (e.g., an assistant HCP) may retain the closure control.

In operating rooms, multiple surgical devices may operate in close proximity to one another. In addition, the devices may all be from different manufacturers and may have different control systems. The devices may not be aware of the presence of other devices. Even if the devices are aware of other devices, the devices may not be able to communicate to coordinate their actions. This lack of coordination may cause the surgical devices to become entangled with each other and, in the worst case scenario, injure a patient.

A system (e.g., a dual system) may have independent yet simultaneous control of more than one (e.g., two) smart instruments (e.g., by the same user). Feature(s) described herein may provide medical professionals the ability to operate an actuatable instrument from a first robot (e.g., endoscopic working channel tool) at the same time as a second actuatable instrument from a second robot (e.g., a lap powered device). For example, operating multiple actuatable devices at once (e.g., an endoscopic device and a laparoscopic device) may be used to hand off a piece of tissue or anatomy from one to the other.

FIG. 40 illustrates an example surgical operating room with robotic surgical instruments. As shown, a user (e.g., a surgeon) may move from one device to another during an operation. In the example of FIG. 40, the surgeon is shown moving between a laparoscopic device control panel, an endoscopic device control panel, and a manual device (e.g., legacy device located in proximity to the patient).

FIG. 41A illustrates example smart surgical devices. For example, smart devices may include a smart surgical stapler 54420, a smart robotic arm (e.g., Hugo robot) 54422, a multi-arm robot (e.g., da Vinci robot) 54424, and/or a smart flexible endoscope (e.g., Monarch flexible endoscope) 54426.

FIG. 41B illustrates example legacy surgical devices. For example, legacy devices may include a ventilator 54430, a magnetic resonance imaging (MRI) machine 54432, a manual endoscope 54434, and/or a computed tomography (CT) machine (e.g., cone-beam CT scanner) 54436.

In operating rooms, multiple surgical devices may operate in close proximity to one another. In addition, the devices may all be from different manufacturers and may have different control systems. The devices may not be aware of the presence of other devices. Even if the devices are aware of other devices, the devices may not be able to communicate to coordinate their actions. This lack of coordination may cause the surgical devices to become entangled with each other and, in the worst-case scenario, injure a patient.

A system (e.g., a dual system) may have independent yet simultaneous control of more than one (e.g., two) smart instruments (e.g., by the same user). Feature(s) described herein may provide medical professionals the ability to operate an actuatable instrument from a first robot (e.g., endoscopic working channel tool) at the same time as a second actuatable instrument from a second robot (e.g., a lap powered device). For example, operating multiple actuatable devices at once (e.g., an endoscopic device and a laparoscopic device) may be used to hand off a piece of tissue or anatomy from one to the other.

As illustrated in FIG. 42, a first surgical device 54440 may include a processor 54442, a sensor 54444, and at least one movable component 54446. The first surgical device 54440 may be (semi) autonomously controlled. The first surgical device 54440 may be in proximity to a second surgical device 54450, which may be controlled by medical personnel (e.g., a surgeon). The first surgical device 54440 may receive a motion control parameter 54448. For example, the motion control parameter 54448 may be preconfigured or determined based on a user input. The motion control parameter 54448 may be a minimum and/or maximum distance to be maintained between the first and second surgical devices, an indication for the first surgical device 54440 to keep the second surgical device 54450 in its field of view, and indication to maintain a constant tissue tension between the first and second surgical devices, and/or the like.

As illustrated in FIG. 42, the sensor 54444 may sense a motion event caused by the second surgical device (e.g., movement of the second surgical device, a shift in tissue tension, etc.). The sensor 54444 may inform the processor 54442 of the detected motion event. The processor 54442 may analyze the motion event in view of the motion control parameter 54448. The processor 54442 may autonomously adjust motion of the movable component 54446 based on the motion control parameter 54448 and the motion event. For example, the processor 54442 may autonomously adjust motion of the movable component by determining an effect of the motion event based on a quantity defined by the motion control parameter; and determining a way in which to adjust the motion of the moveable component to provide a corresponding canceling effect to cancel out the determine effect of the motion event.

For example, if the motion event indicates that the second surgical device 54450 is moving away from the first surgical device 54440 and the motion control parameter 54448 indicates a maximum distance between the surgical devices, the processor 54442 may move the movable component 54446 toward the second surgical device 54450 to keep the devices within the maximum distance from each other. Similarly, if the motion event indicates a decrease in tissue tension for a piece of tissue being held by the first and second surgical devices and the motion control parameter indicates a fixed tissue tension, the movable component 54446 may move away from the second surgical device 54450 to increase the tissue tension back to the original tissue tension. In examples, the first surgical instrument may be an endoscopic instrument and the second surgical instrument may be a laparoscopic instrument.

In an example, the movable component may be a grasping device, the motion event may be a change in tissue tension associated with tissue held by the grasping device, and the motion control parameter may be a tensile range. In this example, adjusting motion of the movable component based on the motion control parameter and the motion event may involve adjusting a load control of the movable component to keep the tissue tension within the tensile range.

In another example, the motion event may be the second surgical instrument moving away from the first surgical instrument, and the motion control parameter may be a maximum distance between the first surgical instrument and the second surgical instrument. In this example, adjusting motion of the movable component based on the motion control parameter and the motion event may involve moving the movable component toward the second surgical instrument to keep a distance between the first surgical instrument and the second surgical instrument below the maximum distance.

In yet another example, the motion event may be the second surgical instrument moving towards the first surgical instrument, and the motion control parameter may be a minimum distance between the first surgical instrument and the second surgical instrument. In this example, adjusting motion of the movable component based on the motion control parameter and the motion event may involve moving the movable component away from the second surgical instrument to keep a distance between the first surgical instrument and the second surgical instrument above the minimum distance.

FIG. 43A is a block diagram illustrating an example control loop in a surgical device (e.g., the first surgical device 54440 of FIG. 42). As illustrated in FIG. 43A, the device may receive an input (e.g., an external input) that may be sensed by a sensor 54444. The sensor 54444 may determine, based on the input, a sensed motion event. The sensor 54444 may indicate the sensed motion to the processor 54442. The processor 54442 may compare the sensed motion to the motion control parameter 54448 to determine an error (e.g., a differential error between the motion control parameter 54448 and an effect caused by the sensed motion). The processor may use the error as an input to a control transform 54454. The control transform 54454 may output a motion for the movable component 54446 to perform. The processor 54442 may indicate for the movable component 54446 to perform the motion. The motion of the second surgical instrument may continue or may stop while the movable component 54446 is moving. The sensor may continue to monitor the motion of the second surgical instrument (e.g., relative to the movable component 54446, which may be moving as well). The control loop may repeat until the error is within an acceptable range.

FIG. 43B illustrates an example of a surgical instrument autonomously moving to maintain a proximity boundary with another surgical instrument. A first grasper (e.g., grasper 1) may be in proximity to a second grasper (e.g., grasper 2). Grasper 1 may have a proximity boundary around it (e.g., the control parameter may indicate for grasper 1 to maintain at least a minimum distance away from other devices). At a time T1, grasper 1 may sense that grasper 2 is moving toward grasper 1. At a time T2, grasper 1 may determine that grasper 2 is about to enter the proximity boundary around grasper 1. In response, at a time T3, grasper 1 may autonomously move away from grasper 2 to maintain the minimum distance between the graspers.

In an example, the moveable component may be a scope, the motion event may be the second surgical instrument is moving out of a field of view of the scope, and the motion control parameter may be a maximum distance that the second surgical instrument can be away from the center of the field of view of the scope. In this example, adjusting motion of the movable component based on the motion control parameter and the motion event may involve moving the scope to keep the second surgical instrument within the maximum distance away from the center of the field of view of the scope.

FIG. 43C illustrates an example of a scope autonomously moving to maintain another surgical instrument in a field of view of the scope. As shown, the scope may have a grasper centered in the scope's field of view. The scope may be used to allow a surgeon to monitor where the grasper is relative to a target structure (e.g., a tumor). At a time T1, the scope may sense that the grasper is moving. At a time T2, after the grasper has moved, the scope may calculate the error between the center of the field of view and the location of the grasper in the field of view. The scope may adjust its motion, based on the error, to keep the grasper centered in the scope field of view. At a time T3, the scope may have moved to re-center the grasper in the scope field of view.

The robot console control may have a hybrid control with a first controller operating a first robot arm (e.g., coupled to a first robot) and a second controller operating a second robot arm (e.g., coupled to a second robot). The system may include a display. The display may be a side-by-side display (e.g., from each of the independent robot visualization means). The display may be a composite display (e.g., where one of the visualization streams is augmented with the other display to form one composite display). The composite display may have the ability to shift to a percent (e.g., any percent) of the two hybridized views (e.g., so the user may customize the angle and/or level of transparency to see both approaches and instruments simultaneously on a single imaging means).

For example, a flexible endoscopic scope (e.g., endoscope) may be used to resect (e.g., mucosally resect) a tumor within a patient's stomach from the serosal layer (e.g., with a monopolar blade). FIGS. 44A and 44B illustrate this procedure. At 54460-54464, the surgeon may be controlling endoscopic device(s) (e.g., smart endoscope and/or monopolar energy device) from a first control station. At 54460, the tumor may be completely connected to the stomach wall in the mucosal layer. At this time, the monopolar energy device may experience relatively low impedance as it begins applying the monopolar energy to the stomach lining.

At 54462, the monopolar energy device may continue to apply the monopolar energy to separate the mucosal and submucosal layers. As the tumor is separated from the submucosal layer, the monopolar energy device may experience increased impedance. At 54464, the monopolar energy device may continue to apply the monopolar energy to separate the mucosal and submucosal layers. At this point, the monopolar energy device may be experiencing a relatively high impedance.

Once tumor is separated about ¾ of the way, the monopolar device may be removed from the working channel. The tumor may then be removed laparoscopically, as illustrated in FIG. 44B. As shown, the surgeon may control the laparoscopic device(s) at a second control station (e.g., a control station completely separate from the first control station used for the endoscopic devices). To control the tumor and perform the flip to the laparoscopic side, one or more grasper(s) may be introduced. The grasper(s) may be used to grasp the stomach near the internal grasping point. The grasper(s) may be used to cut the opening. The grasper(s) may be used to grasp the tumor adjacent to the remaining connection to the stomach. The grasper(s) may then be placed in a station keeping mode.

Once the opening is created, the surgeon may hybridize the approach. For example, the surgeon may release the grasper holding the overall stomach into a station keeping mode. The surgeon may replace control of the grasper (e.g., laparoscopic grasper) with a console control associated with a flexible endoscopic grasper. The endoscopic grasper may therefore be moved at the same time as the remaining laparoscopic grasper. The remaining laparoscopic grasper may be inserted into the incision. The endoscopic grasper may hand off the tumor to the laparoscopic grasper (not shown in FIG. 44B). The laparoscopic grasper may be retracted through (e.g., back through) the incision. The endoscopic grasper may push to get the tumor through the incision (e.g., following the laparoscopic grasper). At 54466a-b, the grasper(s) may hold the tumor while a surgical stapler staples the incision closed. The tumor may then be completely separated from the stomach and removed using the laparoscopic grasper(s), as shown at 54468.

The displays of the two systems (e.g., the endoscopic and laparoscopic systems) may be shown as a side-by-side (e.g., with an integrated overlap and sizing). The integrated overlap and size may allow the portions (e.g., two portions) of the image to join as a synchronized hybrid image. The synchronized hybrid image may prevent a mismatch from confusing the size (e.g., rapid changes in scale due to differing magnifications or alignments) of an object (e.g., in this example, the tumor) as it is passed from one side to the other (e.g., internal to external, for example, through the incision).

FIGS. 45A-C illustrate another example hybrid surgical procedure (e.g., a colorectal tumor removal). As shown at 54470, both endoscopic and laparoscopic device(s) may be inserted into a patient. During the actions illustrated in FIG. 45A, a surgeon may control the laparoscopic device(s), while the endoscopic device(s) are held in an autonomous (e.g., station-keeping) mode. At 54472, the surgeon may use a laparoscopic grasper to push the tumor further into the colon (e.g., toward the endoscopic device(s).

As shown in FIG. 45B, the surgeon may move to a second control station (e.g., separate from the first control station) to control the endoscopic device(s). The laparoscopic device(s) may be placed in an autonomous (e.g., station-keeping) mode. At 54474, the surgeon may apply an endoscopic snare to the inverted tumor. At 54476, the surgeon may retract the snare to pull the tumor further into the colon. The snare may be clenched to reduce the amount of colon tissue that is removed. At 54476, the surgeon may fire an endoscopic stapler to separate the tumor from the colon tissue and staple the resulting incision. At 54478, the surgeon may retract the endoscopic device(s) and the tumor. As illustrated in FIG. 45C, the surgeon may move back to the first control station (controlling the laparoscopic devices). At 54480, the surgeon may retract the laparoscopic device(s).

FIGS. 46A-C illustrate another example hybrid surgical procedure (e.g., a gastric tumor removal). As shown in FIG. 46A, an endoscopic grasper may be used to grasp a tumor in the stomach lining. As shown, the tumor may still be partially attached to the stomach. A laparoscopic incision device may be located on the outside of the stomach. The coordinates of the endoscopic grasper and the laparoscopic incision device may be known by an independent (e.g., robotic) system (e.g., a surgical hub). The laparoscopic incision device may be used to make an incision through the stomach.

As shown in FIG. 46B, once the incision has been made, the endoscopic grasper may be used to invert the tumor. The endoscopic grasper may push the tumor through the incision opening. A laparoscopic grasper may be used to grasp the tumor. The laparoscopic grasper may retract to pull the tumor through the incision opening. The independent system may track the coordinates of the endoscopic and laparoscopic graspers as they pass the tumor through the incision.

As shown in FIG. 46C, a surgical stapler (e.g., a laparoscopic stapler) may be used to staple the stomach lining to close the incision. The surgical stapler may cut the tumor from the stomach. The laparoscopic grasper may be retracted to remove the tumor from the patient's body.

One or more controllers may be used in combination to control one or more independent tools simultaneously. Example controllers may include one or more of: left/right hand controllers, left/right foot pedals, voice commands, and/or the like. For example, a left-hand controller may operate an endoscopic device and a right hand controller may operate a laparoscopic device.

Dual simultaneous device motion may involve a device (e.g., one of the devices) being actively controlled (e.g., with assistance) by the system (e.g., semi- or fully-autonomously). For example, the controlled device may take its lead of movement from another device (e.g., instrument) that is being actively controlled by a user.

Example actuation control schemes are provided herein. Actuation control may involve a force control (e.g., relative to a shared tissue contact). For example, the motion control parameter may be a force relative to a tissue contact shared between surgical instruments (e.g., a first and second surgical instrument).

A load control between two or more smart tissue control jaws (e.g., graspers) may be used. The load control may ensure an ideal amount of tissue tension is applied. The ideal amount of tissue tension may be an amount of tension that assists in endoscopic tissue dissection (e.g., sufficient tension to enable tissue separation without causing tissue tearing). Loads on the grasper shafts and/or jaws may be utilized for this function. The loads may be measured based on motor control feedback, by load sensors (e.g., integrated into the devices), and/or the like. The loads from the devices (e.g., individual devices) may be combined with the orientation of the devices. This combination may be used to estimate the amount of tension on the tissue between the devices.

Tissue tension can be automatically adjusted according to the endoscopic energy device feedback. Tension can be adjusted to optimize sealing or cutting. The Harmonic energy devices train users to not have tension on the organ/tissue/vessels when applying the energy. Utilizing a grasper or other instrument to be able to detect if load is being placed on the organ/tissue/vessel when applying energy could be used to alter the grasper or instrument to minimize the tension during the energy activation.

For example, a uterine manipulator may sense a force within the uterus as the uterus is displaced downward (e.g., as a laparoscopic instrument creates a tissue separation dissection of the outer wall of the uterus to the bladder). The force may be measured endoluminally on tissue that is being affected laparoscopically.

An external mechanism may measure the force on a main function element (e.g., the I-beam of an endocutter). The force measurement may be a motor source measured force (e.g., torque sensing of the output shaft or a proxy, for example, the current through the motor). The force measurement may be a load control on a trocar (e.g., holding a robot arm). The user may change the orientation of arms (e.g., relative to each other) by using the load control on the trocars (e.g., compared to on the instrument robot arms). That is, the trocar may be used as an extra joint for leverage or resisting the force applied by the instrument.

The motion control parameter may be associated with a positional relationship between surgical instruments (e.g., a first and second surgical instrument). A pre-defined positional relationship may be used for actuation control. For example, proximity control may be used for actuation control. The automated system may use feedback (e.g., from a camera) or other relational measurement between the actively controlled device and the automated control device to maintain a proximity of devices. The automated system may be capable of receiving adjustment commands (e.g., from a user) to modify the proximity.

For example, a laparoscopic sleeve gastrectomy may use tools together (e.g., to minimize unintended tissue trauma). During the procedure, the surgeon may utilize other tools (e.g., to be used in conjunction) to hold the tissue in a defined space (e.g., while an endocutter is navigated into position). The other tool(s) may move in a synchronized motion to not contact the endocutter. A grasper may hold the tissue up and away from other objects (e.g., to ensure that, as the device is fired, it does not pinch or staple unintended tissue to the staple line). If an endoscopic instrument (e.g., in the stomach, rectum, or bladder) approaches a lesion of interest; a robotic arm with a laparoscopic instrument (e.g., to be used for grasping and outer organ wall stability for the endoluminal resection or biopsy) may move with a set position of the endoscopic instrument.

Antagonistic positional station keeping control may be used for actuation control. An automated control arm may be capable of maintaining a three-dimensional positional location (e.g., station keeping). For example, the arm may be capable of maintaining the positional location even if forces are applied to the end-effector or the grasped tissue from an outside source. The position control may be capable of resisting externally applied force both in the direction of motion as well as opposite the direction of motion (e.g., without fluctuating).

For example, a powered articulation shaft may be designed to apply force in the direction of motion and resist forces applied opposite to the direction of motion. In a single link system, externally applied forces in the direction of motion may accelerate motion in that direction. An antagonistic system may have actuators in both directions (e.g., with a pre-defined force couple between the actuators). The differential of forces may drive motion. A force in either direction may add to the resisted load in that system opposed to that direction (e.g., the force may be reduced or slowed down if assisted, or increased or sped up if resisted).

Positional control of the control arm (e.g., not just the end-effector location) may be used for actuation control. A flexible endoscope may be able to hold a grasper in a known location. The endoscope may be able to hold the endoscope itself in a known location. Articulations may be used to position an organ in a desired location or orientation. The shaft of the device may provide a (pre)defined retraction or organ manipulation control (e.g., which may be as desired as the end-effector location or motions).

Hybrid load-strike control may be used for actuation control. Switchable state control may be used for actuation control. For example, load control may be used if displacements are small. If displacements over a (pre)defined amount (e.g., over a (pre)defined time), the device may operate in a position control mode.

A control loop with secondary limits may be used for actuation control. For example, position control with a maximum or minimum applicable force limit may be used for actuation control. Load control with a maximum displacement over a time and/or velocity limit may be used.

Dual loop control may be used for actuation control. For example, two separate/independent feedback monitors may be used. One may monitor the motor. One may monitor the driven part (e.g., the actual driven part) of the device.

In an example, the movable component may be a grasping device, and the motion event may be the second surgical instrument changing position, and a change in tissue tension associated with tissue held by the grasping device. In this case, the motion control parameter may be a tensile range, a displacement threshold, a window of time, and a range of distances between the first surgical instrument and the second surgical instrument. Adjusting motion of the movable component based on the motion control parameter and the motion event may involve, on a condition that the first surgical instrument moves a distance smaller than the displacement threshold during the window of time, adjust a load control of the movable component to keep the tissue tension within the tensile range. On a condition that the first surgical instrument moves a distance greater than the displacement threshold during the window of time, the device may move the movable component to keep a distance between the first surgical instrument and the second surgical instrument within the range of distances.

Control loop parameters may be used for actuation control. For example, proportionate, integral, derivative (PID) control loop parameters may be used. The proportionate parameter may be associated with a magnitude of force or velocity. The integral parameter may increase action in relation to the error and the time for which the error has persisted (e.g., duration of error). The derivative parameter may be associated with a magnitude of the error.

Predictive control may be used for actuation control. Predictive control may be based on one or more elements (e.g., three key elements). For example, predictive control may use a predictive model, an optimization in range of a temporal window, and/or feedback correction. The predictive model may be used to predict a future output based on historical information (e.g., about the process,) and/or an anticipated future input. For example, a state equation, transfer function, and/or a step or impulse response may be used as the predictive model.

In an example, an endoscopic grasping device may have a grip on a mucosal tumor (e.g., that has been mostly resected from inside the stomach). The endoscopic articulation may be used to control the tumor position and/or the orientation of the stomach (e.g., to prevent loss of acid control when a trans-wall incision is made). The user may move the receiving grasping device from the laparoscopic side into close proximity to the abdominal cavity side of the stomach wall. The receiving grasping device may take hold of the outside wall to control the stomach for the incision step. The laparoscopic grasper and the endoscopic grasper may be released for autonomous control. The endoscopic grasper may be given a proximity distance to maintain to the laparoscopic grasper. The laparoscopic grasper may be placed in a station keeping mode. For example, the device may receive an indication to enter a station-keeping mode. The device may maintain a global position of the moveable component in response to the indication.

A third laparoscopic blade (e.g., monopolar blade, conventional blade, or scissors) may be brought in to make a cut along the base of the still-intact connection of the tumor and the inside wall of the stomach. The blade force may be resisted by the laparoscopic grasper station keeping retraction. Orientation to prevent acid spill may be controlled by the endoscope (e.g., based on shape and the grasper holding the tumor base). Once the incision is made, the cutting element may be removed. The laparoscopic grasper may be used to grasp the tumor base from the laparoscopic side. The proximity control may be used to keep the endoscopic hold of the tumor relative to laparoscopic grasper (e.g., for fixation). The proximity distance may be adjusted (e.g., may be reduced to bring the devices closer together, if needed). Once the lap grasper has the base of the tumor, the endoscopic grasper may release its hold on the tumor. The endoscopic grasper may maintain (e.g., still hold) its position (e.g., thereby allowing the user-controlled laparoscopic grasper to flip the tumor to the abdomen space. The laparoscopic grasper may be placed in station keeping mode. The user may take control of the endocutter. The user may position the endocutter across the incision and the base of the tumor. The user may fire the endocutter. The user may then simultaneously cut the tumor loose and seal the incision. The endocutter may be placed in station keeping mode (e.g., while still clamped on the tissue). The endoscopic tools may be retracted (e.g., first). The laparoscopic grasper may be (e.g., may then be) placed in a user control mode. The endocutter jaws may be opened to enable removal of the tumor from the surgical site.

A laparoscopic endoscopic cooperative surgery (LECS) may be used for stomach tumor dissection. A tumor may be located adjacent to the esophageal sphincter on the greater curvature posterior side. Tumor removal may involve mobilization and retraction of the stomach into an irregular shape to access, dissect, and remove the tumor laparoscopically. Endoscopic sub-mucosal dissection with trans organ wall flexible endoscopic access may be combined with laparoscopic manipulation and specimen removal. Laparoscopic and endoscopic cooperative surgery may be used to remove gastric tumors.

A gastroscope (e.g., 5-12 mm overtube) may have working channel sizes of 2-4 mm and/or a local visualization scope. One or more (e.g., several) laparoscopic trocars, a laparoscope, and/or tissue manipulation and dissection instruments may be introduced (e.g., in an operating room).

Identification of the tumor location may be performed on the endoscope side. The endoscopic side may communicate the tumor location to the laparoscope side (e.g., where the stomach needs to be mobilized to allow for stomach retraction and manipulation). The gastroepiploic artery surrounds the perimeter of the stomach and is fixed to surrounding structures. The gastroepiploic artery may be freed to enable mobilization and separation of connective tissues. During this, bleeding may occur. The laparoscopic side may intervene if bleeding occurs.

The stomach may be manipulated and held in a position where the stomach acids are not over the portion of the stomach where the tumor resection will be performed (e.g., where the intra-cavity cut will be made). The stomach acids may be managed (e.g., with respect to gravity) to prevent inadvertent escape of the acids into the abdomen cavity.

Electro cautery may be used to free the perimeter of the mucosal layer within the stomach. Mucosal and sub-mucosal dissection may be expected (e.g., based on the depth of the tumor in the stomach wall). Inadvertent perforation of the serosal tissue layer may create leaks from the stomach to the abdomen. Controlled energy usage may be used to get deep enough to peel the tumor off (e.g., but not too deep to burn through the entire wall thickness).

Energy assisted dissection may be stopped with a portion of the tumor still connected to the stomach lining. The portion that is still connected may be pivoted through the incision (e.g., to keep hold of the tumor during extraction).

An incision may be made outside of the tumor margins (e.g., to allow for removal of the tumor from the laparoscopic side, and ensure that the cancer is removed). The location of the incision may be initiated from the laparoscopic side. The location of the incision may be coordinated with the endoscopic side and the tumor location. The tumor may be controlled and/or manipulated (e.g., during the incision creation) to prevent inadvertent cutting of the tumor. A tumor margin may be used to make sure that the retained tissue is cancer free. The tumor may be too large for oral extraction.

The tumor may be pivoted from the endoscopic space to the laparoscopic space. The stomach orientation may be controlled to prevent stomach acid from escaping into the abdomen. Localized bleeding may be controlled with advanced energy (e.g., from the laparoscopic and/or the endoscopic spaces). The tumor may be pivoted from the control and interaction of the endoscopic (e.g., endoluminal) instruments to the control and interaction of the laparoscopic instruments. During the hand-off there may be at least one point in time during which both sets of instruments are interacting with the same tumor tissue.

An endocutter may be introduced (e.g., from the laparoscopic side) and positioned to transect the tumor from the remaining stomach wall and seal the opening through which the tumor was passed. Poor positioning of the endocutter may result in a hole being made through the organ (e.g., that will need to be closed before completion of the procedure). The stapler jaws may be overloaded by tissue thickness. In this case, the staples may be inadequately formed. The inadequately formed staples may not seal the organ (e.g., which may result in localized bleeding).

Limits may be added to create bounds for the assisting control. For example, in a transurethral resection of a bladder tumor (TURBT) (e.g., when the endorobotic instrument is near the tumor on the bladder wall), a laparoscopic device may be moved into position to affect a concave (e.g., from laparoscopic side) positioning of the tumor area (e.g., to assist in the transurethral resection). As another example, if a patient has been prepared for indocyanine green (ICG) fluoroscopy, the florescent may be used to monitor the proximity of the working robotic laparoscopic instrument to a surgical structure (e.g., a critical vessel, for example, during dissection of the liver lobe near the hepatic artery, dissection of the descending colon mesentery near the Inferior Mesenteric Artery (IMA), etc.)

A user may select a monitored parameter to operate the device movement within the closed loop control. A device (e.g., each device, for example, a grasper) may know which devices should move at a given time. For example, the device may be able to determine which device should move based on an operational window (e.g., which device has the space available, for example, based on tissue and/or organ proximity). The device in furthest proximity from a hazard or high risk area (e.g., carotid, nerves, critical structures, etc.) may be the device that should move. A predetermined hierarchy of control may be determined (e.g., prior to the procedure starting). The determination of which device should move may be based on a procedure plan and/or a user selection. The determination of which device should move may be based on a physical location of the device in proximity to a smart control system. For example, the grasper closest to controlling smart system may be the device that will move. In this case, the surgeon may know (e.g., always know) the grasper in closest proximity to the smart system. In some examples, a device on a dominant side/hand of the surgeon may be the device that is autonomously controlled.

When transecting a vessel using an energy device, graspers may work in unison with each other (e.g., and possibly a vision system) to control tissue tension. If too much tension is applied, hemostasis issues may occur. If too little tension is applied, undesired thermal damage may occur.

Force load control may be used if multiple (e.g., two) smart systems are holding the same piece of tissue. Position control may be used to help systems not bump into each other. A local command unit may regain command and/or delegate tasks to complete a procedural step.

To successfully hand off tissue from one device to another, the single control system may choreograph control (e.g., by calculating and displaying, to the user, an anticipated trajectory path for intercept, velocity, and distance to intercept). The control system may track (e.g., in real time) the movement of the devices compared to the anticipated/proposed device trajectory path. Defined paths may include the anticipation of next steps and/or obscured visibility (e.g., due to device, organ, or environmental interference).

If the trajectory path has an obstruction, the trajectory path may be updated. The updated trajectory path may be displayed to the user. The updated trajectory part may be a trajectory path (e.g., an ideal trajectory path) that bypasses the obstacle. If a visual condition is interrupted by an organ, which may be positioned with a smart retractor, the smart retractor may shift its position to maintain the desired visual condition.

If the user veers off the trajectory path, the system may reassess the trajectory path. The system may display the modified trajectory path (e.g., for hand off). For example, if the system cannot make a maneuver in the initial trajectory path, the system may recalculate the best direction(s) (e.g., updated trajectory path) and update trajectory instructions as needed. The control system may provide the user with a proposed or ideal trajectory path (e.g., if the currently anticipated path is an obstructed path or comes in close proximity to a critical structure). If the device(s) veer off the predetermined trajectory, the trajectory may be updated.

Predefined visual condition(s) may be identified. The visual conditions may be monitored (e.g., continuously monitored) and/or evaluated for interference. For example, during a gastric cancer procedure, the surgeon may want the stomach and/or the surrounding area visible and/or accessible (e.g., to dissect lymph nodes, remove the tumor, and/or reconstruct the stomach). In such a procedure, the liver may be lifted (e.g., with some form of organ retractor) to gain space and visibility. Smart organ retractors and/or smart graspers (e.g., utilized to retract organs) may be controlled based on a visual analysis of the surgical field. A visual condition may be interrupted by the organ itself shifting. The retractors may compensate for such an obstruction. The surgeon may shift the target tissue into a position that is no longer visible. If the liver shifts out of position, critical steps of the procedure may be interrupted. This may cause patient harm if the surgeon loses visibility.

The smart system displaying the trajectory path may display nearby structures (e.g., critical structures) to provide the user with awareness. If the trajectory path is in close proximity to a critical structure, the trajectory path may continue to be displayed along with a proposed trajectory path (e.g., to mitigate the potential hazard).

Cooperative alternating movements of a smart system may be used for anticipated events (e.g., next steps in a procedure). A common field of view may be maintained. The devices may rotate being stationary or changing position. A first system may hold its current position. A secondary system may move to the next anticipated area of visibility.

If a smart device approaches the outer extent of the vision system, the user may pause the device (e.g., momentarily). While the smart device is stationary, the vision system may reposition itself (e.g., to recenter the device within its frame). The system that will maintain its current position (e.g., and the system that will move) may be determined based on which vision system the current action is centered within. Using a combined registration, the vision system movement may be seamless. For example, the stationary system may be able to maintain fixed imaging on the current event, while the moving system may prepare for the next area of view (e.g., the direction of motion of the surgeon).

In an example, a surgeon may be separating an organ from surrounding tissue and may pause. The vision system may recognize the pause and anticipate that the surgeon will continue soon. The vision system may reduce its speed of movement (e.g., but continue to move) while the device is stationary (e.g., anticipating that the device will continue moving once the surgeon reengages). The vision system may anticipate the seam between the organ and tissue to follow (e.g., best fit follow) the surgeon's anticipated trajectory.

One or more camera systems (e.g., independent camera systems) may be affixed to the same laparoscope. The camera system may have independent rotation features to allow combined visualization within the same plane. Independent visualization systems or techniques may be used. For example, a visualization system may be a white light camera system, and/or an alternative imaging modality. For example, a procedure may use a C-arm and/or ultrasound that may relate back to a white light laparoscope. An endoscope may have multiple cameras (e.g., mounted within its tip). The cameras may track (e.g., independently track) devices that have been inserted and are moving within the body cavity.

A first and second vision system (e.g., vision system A and B) may be used. A critical event may occur that is centered in the vision system A. In this case, the vision system A may hold in one spot. The vision system B may move to include an area of visibility (e.g., a required area of visibility).

Those skilled in the art will appreciate that the features described herein may be implemented using any appropriate means for motion sensing/tracking. For example, motion sensing may be performed using sensing techniques based in ultrasonic, infrared, microwave, light detection and ranging (lidar), radio detection and ranging (radar), sound navigation and ranging (sonar), hybrid or dual-technology, and/or the like.

Some techniques for motion sensing may have benefits that are desirable in a surgical environment. For example, passive infrared sensors may be small, inexpensive, and use relatively low power. As another example, microwave sensors may be able to sense motion at farther distances than some other technologies.

FIG. 47 illustrates an example method that may be performed by a surgical instrument. As shown, the method may involve, at 54482, receiving a motion control parameter. The motion control parameter may include one or more of: a force relative to a tissue contact shared between the first surgical instrument and the second surgical instrument; or a positional relationship between the first surgical instrument and the second surgical instrument. The method may involve, at 54484, sensing a motion event caused by a user control input to a second surgical instrument. The method may involve, at 54486, adjusting motion of the movable component based on the motion control parameter and the motion event.

FIG. 48 illustrates an example surgical operating room in which several different devices are present. Some of the devices may be “smart” surgical devices (e.g., robotic arms, smart endoscope, etc.) while other devices may be legacy devices (e.g., a CT machine) that do not have the same level of awareness as the smart devices. The legacy devices may not be able to communicate with any other device, whereas the smart devices may have communication capabilities.

The operational areas of activities by a first system and a second system may be synchronized. The systems may have synchronized devices that are separately controlled by the systems. The individual actions of a first device to a second device may not be synchronized. For example, the shape or location of a operational smart device envelope may be altered (e.g., passively altered). For example, the operation envelope may be altered based on the active modification (e.g., by a user) of another related smart device movement. A predefined balance between two operations envelopes may be used to determine how the operational envelope(s) are altered. For example, an operational envelope may be altered when the other operational envelope is modified by the active control of the user. For example, the loci of actions or envelope of functional limits of a first system may be changed based on the movements of a second autonomous system.

The operational envelope of the motions of a first system may be adapted based on the motions, activities, and/or location of another device (e.g., a moving smart device). For example, the activities, movements, and/or operation of a first smart system may cause a second system to change its operational envelope (e.g., through the second system's own decisions and operations). The second system may not be told whether to operate in a predefined space.

FIG. 49 is a block diagram illustrating example processing modes in a surgical device (e.g., a robotic arm). The surgical device may have a processor. The processor may receive control inputs (e.g., user control input from a console controlled by a user) and/or data input from another device (e.g., via an API). For example, the user surgical control input may include at least one of: moving a joystick, squeezing a trigger, touching a display, pressing a button, flipping a switch, turning a dial, and/or the like.

If the surgical device is in a first mode (e.g., Mode 1), the device may only use the console inputs to determine the device's movements (e.g., motor controls). That is, the device may be completely controlled by the user. For example, the surgical device may perform an operation responsive to the user surgical control input. The operation may include at least one of: actuation of joints external to a patient's body, a position of the surgical device, displaying an image on a display, and/or the like.

If the surgical device is in a second mode (e.g., Mode 2), the device may consider the console inputs and the data input from the other device when determining the device's movements. For example, the user may direct the surgical device to move a robotic arm to a new area of the patient. The surgical device may know (e.g., from the data input from the other device) that the other device is in that area. In this case, the surgical device may indicate a warning to the user (e.g., indicating a potential collision between the devices). The surgical instrument may know that it would have to cross over where the other device is located to reach that area. In this case, the surgical device may constrain its operation to avoid collision between the surgical device and the other (e.g., negotiating) surgical device. For example, the surgical device may determine a different path to take to the area than the path originally indicated by the user. The different path may be calculated to avoid a collision between the two devices, while still allowing the surgical device to reach the indicated area of the patient.

The data provided between multiple (e.g., two) moving systems may be used to determine the operational space of the systems. One or more of the system(s) may use the data to adjust its functional space, orientation, position, etc. (e.g., based on the monitored changes in the other system). The adjustments may be, for example, follow-along, preemptive repositioning to avoid anticipated confrontational use of occupied space, avoidance of an electrical interference zone, and/or changes in orientation to improve the functional interactions between the systems.

In an example, multiple separate robot arms may be used to retract and dissect an attachment of an organ (e.g., the stomach or other organ with interrelationships to other organs or fixation to the substructure of the body). A first system (e.g., robot arm) may be used to retract a portion of the organ (e.g., stomach) in the laparoscopic space via a percutaneous insertion point. A second system (e.g., instrument) may be used to generate forces between the first system (e.g., through the tissue) to dissect the tissue. The second system may constantly provide the first system position, orientation, and/or force direction information. The first system may use this information to reposition its external arm position to provide the best stable counter force for the second system. The systems may not be at risk of colliding. The user may not have given a direct command for the first system to move. The second system may determine that the first system repositioned itself to provide better support to the second system.

As illustrated in FIG. 50, multiple Hugo robot arms may be used simultaneously during an operating procedure. Each robot arm may have acceptable operation zone(s) (e.g., both inside and outside of the patient's body). Other device(s) (e.g., a Monarch smart endoscope) may be introduced to the operating room. The system may inform the robot arms of the operational envelopes 54400 that overlap between the robot arms and other device(s). The overlaps may create shared air space 54402 between the operational zones, which are shown as shaded regions in FIG. 50.

One or more of the devices may attempt to reduce any shared air space 54402. The shared air space 54402 may be treated as a negotiated zone between the devices. One of the devices may send an indication of a preferred operational envelope to a negotiating surgical device. For example, if a first device intends to enter the shared air space (e.g., in response to a user surgical control input), the first device may send a request to a second device that is sharing the shared air space. The request may be used to determine a negotiation protocol between the devices. The first device may send an indication of the preferred operational envelope according to the negotiation protocol. The first device may determine to send the indication of the preferred operational envelope based on at least one of: a class of the surgical device, or a class of the second (e.g., negotiating) surgical device. The indication may further indicate one or more suggested reduced operational envelopes (e.g., in addition to the preferred operational envelope). The second device may send a response that indicates a selection of the reduced operational envelope.

The second device may respond to grant or deny the first device access to the shared air space. The second device may send a response that indicates a selection of the reduced operational envelope (e.g., from the options presented by the first surgical device). The second device may send a response that indicates a rejection of the preferred operational envelope and an instruction to use a different reduced operational envelope.

The first device may constrain its operation responsive to the user surgical control input based on a reduced operational envelope that is determined by the response (e.g., from the negotiating surgical device) to the indication of the preferred operational envelope. The reduced operational envelope may result in a greater constraint on the operation responsive to the user surgical control input than the preferred operational envelope. The greater constraint on the operation (e.g., operation responsive to the user surgical control input compared to the preferred operational envelope) may involve a movement restriction that limits a physical space that the surgical device may enter, a display restriction that restricts portions of the surgical device's field of view that may be displayed, a time restriction that limits times during which the surgical device has access to a physical space, and/or the like.

The response from the negotiating surgical device may be based on at least one of: an operational envelope of the negotiating surgical device, a present state of the negotiating surgical device relative to the preferred operational envelope, a procedure being performed using the surgical device, or a step in the procedure being performed using the surgical device.

In an example, the second (e.g., negotiating) surgical device may be a robotic surgical device and the operation responsive to the user surgical control input may be a robotic joint actuation. The preferred operational envelope may be a physical space that accommodates potential robotic joint positions. In this case, the first surgical device may constrain operation responsive to the user surgical control input (e.g., based on a reduced operational envelope) by restricting access of the first surgical device to a portion of the physical space.

Adaptive robot-to-robot no fly zones may be based on an aspect of a first robot arm (e.g., location of the second robot cart, its robot arm position, movements, required operational envelope, etc.). The no-fly zones may place limitations on a second robot arm. The second robot arm may receive the limitations from the first robot arm or a separate robot.

In an example, a first laparoscopic multi-cart robot may be used to for dissection and resection of a mid-parenchyma tumor (e.g., that is on the junction of two segments). A surgeon may want to avoid removing two full segments. The surgeon may attempt to separate the tumor from the artery and vein. The surgeon may determine (e.g., during surgery) that the tumor has invaded the bronchus. The surgeon may determine penetration depth and the extent of invasion (e.g., using a flexible endoscopy scope controlled with a separate robot). The introduction of the second robot may not involve repositioning an existing first robot cart. A cart positioned towards the head of the patient may have a working envelope outside of the body that encompasses some of the space occupied by the flexible endoscopy robot and its operating envelope.

Once operational, the second robot may establish communication with the first robot. The second robot may communicate its location and size dimensions. The second robot may define the space it intends to use (e.g., at a minimum) to operate and inform the first robot of the reduced operational envelope available in which the first robot can operate to avoid entanglement. This regulation of the first robot by the second robot may involve defining the space reduction and active monitoring of the first robot arm. The restriction may involve defining a portion of the full operating envelope in which the first robot can no longer operate. The restriction may be an actively adjusted regulation of the space (e.g., that changes as the two robot arm coordinate their operation with the flexible endoscopy robot).

The space may be reduced (e.g., only reduced) as needed for the second robot and the endoscopy robot to move. In this case, the first robot may be allowed to occupy shared space (e.g., as long as it does not intend to always be in that space). If the first and second robots intend to occupy the same shared space, the robots may negotiate (e.g., based on priority, user input, or computational ordering) to choreograph the robots' motions. This may allow the robots to move around each other (e.g., through a series of pre-calculated alternating motions to allow them to move around each other) without adverse interactions.

Smart system(s) may be able to identify the location of other smart systems relative to each other. In the previous example, as the flexible endoscopy robot is brought into the OR and set up, the user may input the location and operational window for operation or a smart system may define the location and operational envelopes of the robots (e.g., relative to each other).

The surgical hub and a room-based camera may be used to identify the exact location, shape, and operational window to be used by a device (e.g., based on the setup of the devices in the OR). For example, multiple perspective cameras with overlapping image coverage may be used. Fewer cameras may be used if, for example, light detection and ranging (Lidar) is used to detect distances and/or structured light is used to define shapes and volumes. The robot towers/carts may integrate laser alignment and Lidar positioning to define the location of the arms, carts, and control systems.

For example, a Hugo robot may use a laser alignment and positioning system to determine where its arms are relative to the patient. If an ion or Monarch flexible endoscopy robot is positioned in the OR, the Hugo robot may use the alignment system (e.g., and information from the flexible endoscopy system) to identify the location of the endoscopy system within the room and around the patient.

Physical docking locations or mechanical linkages may be used to place the movable robot carts and towers in known (pre)defined locations (e.g., relative to any stationary larger robot systems). For example, an Ottava table-based robot may have a docking location with a physical aligning and locking system. The aligning system may enable a monarch mobile flexible endoscopic robot to know the location of the tower, and be placed around the tower accordingly.

The working envelope of the arms, instruments, and end-effectors of each of the smart system may be determined. Overlapping spaces of the operational envelopes as shared space may be identified, as shown in FIG. 50. The utilization of the shared space by each of the systems may be organized. Subtractive operational envelope reduction may be used to reduce the space a smart system is allowed to use (e.g., based on the need or priority of the other system to use that shared envelope).

If tight cooperative use of the shared space is planned, the two systems may develop a plan for sequential choreographed motions. The systems may determine which system will determine the choreographed motion plan. The systems may choose between multiple movement options (e.g., who moves first, whether to coordination will be similar to a multi move chess match, etc.).

Individualized reactive isolated step operation may be used for the robots to move one step at a time. At each step, the robots may reassess the new situation (e.g., rather than fully planning out multiple moves to choreograph together).

As illustrated in FIG. 50, the operational envelopes of the robots overlap each other. Robot #1's envelope may be reduced by the overlap with robot #2 and robot #3. Similarly, robot #4's and robot #3's envelopes may be reduced. In this case, the reduced envelopes may be due to an adjacent robot having a higher priority use of the space (e.g., so the operational envelope of the lesser priority robot is reduced). This rule may be reversed or the “shared” space may be reallocated (e.g., dynamically on-the-fly). An overlap may be partially owned by a system, or the sharing may be choreographed over time (e.g., as one moves out of the space, the other may move through the space). These shared motions may be stepwise (e.g., with each robot making one move, and then the other, for example, to better utilize the shared spaces).

The systems may define and regulate shared operation envelopes (e.g., inside and/or outside of the body). For example, in a thoracic procedure, a plurality of (e.g., five) Hugo robots may be arrayed around the patient with at least one accessing the space over the patient's head. The operational envelope overlaps may be managed by one or more of the Hugo robot(s). A monarch device may be introduced to the OR next to the at least one Hugo robot by the head of the patient. This may create new (e.g., two new) overlap zones. A first overlap zone may be the space the Monarch deems necessary for operation (e.g., extension and retraction space). A second overlap zone may be a portion of the shared space that the monarch device or the Hugo robot is allowed to use (e.g., but not at the same time). The two robots on the left of the patient may be repositioned to allow for the flexible endoscopy robot (e.g., monarch) to be positioned by the head and the flexible endoscopy scope to be introduced in the mouth.

There may be a no-fly zone around the flexible endoscopy robot because it cannot move out of the way for the adjacent robot station to share the space. Accordingly, the robots must avoid that space. In the shared space, two arms of the same controlled robot overlap and therefore could collide, but the robot controller may limit the zone to only one robot arm at a time.

The no-fly zone may change over time (e.g., in a choreographed manner). In this case, the robot arms may consider alternate configurations to get a first arm out of the space through which another arm intends to move. Adaptive robot-to-robot no-fly zones may be determined based on the movement of one or more smart devices. The no-fly zones may be inside or outside the patient's body.

The activation state of a device may affect the viable operation envelope of another. For example, a first smart device's position, motions, articulation, energy potential, or activation state may cause the system to adjust adjacent smart end-effectors in near proximity.

For example, conductive end-effectors zone of occlusion and interaction may be modified (e.g., depending on whether one of the end-effector's monopolar energy is active, or is active and above a certain threshold. This may prevent inadvertent energizing of a non-monopolar device (e.g., based on either inadvertent contact or close proximity in contact with the same tissue where the second device could become part of the return path). The zone modification may minimize inadvertent burns to the patient away from the surgical site (e.g., based on the continuity path of the second device's other tissue contacts).

An aspect of an advanced energy device may be monitored. The monitored aspect may be used to derive an envelope within which are handling specification (e.g., associated with the surrounding tissue or the other instruments). The handling specifications may be used to define active envelopes of restricted operation. These envelopes may be static representations based on the monitored parameter. The envelopes may be adapted or morphed (e.g., based on second device composition implications). The adaptations may be performed if (e.g., under (pre)defined conditions) there are exceptions to a rule governing the envelope. The system may determine that certain devices (e.g., special insulations in the devices or the trocar) or certain circumstances (e.g., the user acknowledged a risk and proceeded anyway, certain tissue conditions, or device orientations) may cause the systems to interact more closely. In this case, the system may actively adjust the envelopes (e.g., on the fly).

The operational window that is adjusted due to the proximity of a second device may limit the approach of the second device if the first device is active. The system may wait to activate the first device if the second device is within a range from the first device. For example, the activation (e.g., energizing) of a first device may be limited if the first device is in close proximity to a second device that has sensing means that would be affected by the energy being used near the second device. For example, a sensing means may be affected by monopolar RF, microwave, or irreversible electroporation.

A system may adjust the usable input situation of another device to bound its operation. A (e.g., independent) system may define another system's operational envelope.

One or more functions of related systems that affect the physiologic parameter of the patient may be adjusted (e.g., if the physiological parameter is out of pre-established bounds). Multiple systems may be synchronized by defining preventative or allowable response to undesirable events.

The operational envelope(s) may be adapted based on one or more constraints (e.g., real-time patient status/condition, surgeon usage, and/or adverse or undesirable events). For example, FIG. 51 illustrates an example block diagram illustrating a technique for optimizing outputs based on inputs and a constraint. As shown, one or more inputs may be considered by an operational envelope control device. The inputs may be sent through a first kinematic equation and a second kinematic equation. The kinematic equations may (e.g., each) output a top result.

The top results may be analyzed based on the constraint. For example, if the constraint is a no-fly zone for a robot arm and the top results would result in the robot arm entering the no-fly zone, the constraint may feedback this information to the kinematic equations. The kinematic equations may be re-calculated, based on the inputs and the information related to the constraint, to output new top results. The process may be repeated until one or more of the top results do not conflict with the constraint. This optimized result may be output to the robot arm to indicate how the robot arm should move. Accordingly, the device may constrain its operation by selecting a first kinematic solution (e.g., for joint positions of a robotic arm) that is different than a second kinematic solution associated with the preferred operational envelope.

As illustrated in FIG. 52A, two or more surgical instruments may communicate with each other (e.g., via an API). In some examples, the operational envelope control device may be in one of the surgical devices, and that surgical device may output the determined results to the second surgical device to control the second device's movements.

The focus of a given imaging source may be adapted based on motions/actions of a first device and/or adverse reactions detected in the field of view. For example, as illustrated in FIGS. 52B and 52C, an imaging scope (e.g., an endoscope) may be configured to track the movement of a surgical instrument (e.g., a laparoscopic device). As shown, if the endoscope detects that the laparoscopic device has moved, the endoscope may automatically move to keep the laparoscopic device in the endoscope's field of view. The endoscope may be configured to track the laparoscopic device by another device (e.g., a surgical hub). The surgical hub may receive the configuration information from a user (e.g., a surgeon may indicate for the endoscope to track the laparoscopic device) and the surgical hub may forward that configuration information to the endoscope.

A smart visualization scope being able to track and move with the nexus of detected motion based on the instruments currently actively controlled by the user, could adjust the scope location, focal length, and field-of-view to follow the instrument end-effectors. The robotic laparoscopic hub may provide movement data to the scope to define the moving nexus of operation of the devices being controlled by the user. The scope may provide the data of its location and field of view.

The smart scope may (e.g., actively) act on the nexus location data it receives relative to the calculated center of field of view trying to keep the two measures synchronized. The robot console may use the field of view width and center to determine device locations and event outside of the field of view. The robot console may display those representations to the user to maintain peripheral awareness (e.g., while allowing the user to keep their field of view narrowed on the current action activities). Devices (e.g., each device) may control their own actions relative to the data it receives and the data it generates. The actions may not be synchronized with the motions of the other device in this case. The devices may provide data on their location and activities to the other so that the other may act accordingly to avoid issues (e.g., collisions or other interference).

Based on the movement of a surgical device (e.g., endocutter), the system may be able to track and predict where to position a camera so that the surgeon can maintain an uninterrupted field of view.

A scope with high resolution and/or digital zoom may be used to monitor the entire surgical field. The scope may only focus on a specific area at a given time. The scope may be positioned close to the body wall. The scope may have enough resolution to capture the entire internal abdominal cavity. An area of interest may be magnified (e.g., digitally), for example, instead of repositioning the scope, or optically adjusting the physical lens focus of the scope. The digital focus of the image, and what is visible to the user may be controlled manually by the user, or based on synchronized motions with other devices, as described herein.

By using digital and/or optical zoom and tracking, the entire surgical field may be monitored in the background while the user is able to focus on specific areas. With the entire surgical field being monitored by the system, alerts to the user and/or automatic refocusing of the image may help manage adverse reactions (e.g., bleeding).

For example, in a sleeve gastrectomy, delayed bleeding of the staple line may occur (e.g., several minutes after the stapling event when the surgeon is focused on other parts of the procedure). The imaging system may track and focus the camera based on the endocutter in use. As the surgeon moves between firings, the endocutter may be kept in focus so the surgeon can see the current transection. In an example, while the surgeon is working on the fourth firing of the sleeve, the first firing may start to bleed. The first firing may be outside the field of view of the current focus. Because the imaging system is tracking the entire surgical field, but focusing the visible image on the endocutter, the system may identify that bleeding is occurring. The system may send an alert to the user indicating that bleeding is occurring off screen. Reaction types may include a text-based alert warning of off-screen bleeding, a picture-in-picture showing the bleeding, an automatic refocusing of the image to zoom out and show the entire surgical field to include the bleeding, an alert based on a severity of bleeding or other adverse reaction (e.g., critical vs. nuisance bleeding may be picture-in-picture vs. text), and/or the like.

Visualization may be used as a bounding means for inhibiting actuations in obscured locations. Secondary data sources may be used to overcome obscured hazards to replace the obscured location.

The scope may be used to determine one or more portions of the visualization field inside of the patient that can be interacted with (e.g., manipulations, end-effector movement, energy device or endocutter repositioning, etc.) based on the ability to accurately visualize the spaces. Smoke from previous firings may occlude clear visualization of tissue. The scope may be used to constrain the actionable portions of the field based on what can be seen. This may be an absolute go/no go indication or a suggested no-fly zone with an override condition so that the user may enter the space if needed.

Data gathered from surrounding smart devices may be used to provide information (e.g., otherwise unavailable information) to another smart device. For example, that information may be otherwise unavailable due to lack of visibility in an obscured hazard.

For example, a pre-operative CT scan may indicate a series of metallic clips from a previous surgery. The pre-operative CT may utilize a surgical plan to indicate (e.g., in a real-time image) the projected location of the clips identified in the pre-op imaging. This may be done even if the body is in a different position during the surgery compared to during the full body CT. Data may provide fiducial location information associated with the locations of staples, bolts, plates, screws, and/or the like. If harmonic is used and a clip is accidentally clamped and energized, the blade may break. If a staple line is fired over a pre-existing clip and the blade of the staple contacts the clip, the blade may drag the staple along, damaging the staples its deploying. The exchange of data may be used to overcome the obscured hazard in these cases.

In another example, if a uterine manipulator is obscured in the laparoscopic view during a hysterectomy; the laparoscopic robotic controlled instruments may be limited from moving directly against the distal portion of the manipulator. For example, the laparoscopic instruments may (e.g., may only) move over or under the manipulator to create the separation planes to the bladder and rectum, respectively. After the dissection is complete (e.g., as manually entered by a user or based on pressure of uterine manipulator indicating free motion of uterus), the laparoscopic instruments may be unlocked from moving distally toward the cervix. The cooperation of laparoscopic energy and uterine manipulator colpotomy cup may be used to create a colpotomy.

The bounding means may be based on a configuration or state of the surgical site or organ (e.g., instead of the absences of direct visualization). For example, an endoluminal view (e.g., as in a colonoscope) may be checked by computer vision and compared to a bounding zone. The view within the bounding zone may indicate an ‘open’ lumen with proper balance of laparoscopic pressure and endoscopic pressure. If the lumen is occluded (e.g., smaller) than the bounding window, the endoscopic instrumentation may not deploy. The endoscopic instrumentation may be manually deployed (e.g., with Bluetooth) by a main system. The main system may be used for instrument identification and augmentation, which may be displayed on a user display (e.g., a main user interface control screen).

The operation envelope of a device may be adapted based on device performance, device error codes, alarms, and/or device capability. For example, if a user is using a harmonic device and the blade is running hot, the operational envelope may adjust to prevent undesired thermal spread. The operational envelope may be made smaller (e.g., immediately after the transection is made) until the blade cools to a temperature in which it won't damage the patient if inadvertent contact occurs. Once blade has cooled, the operational envelope of device may expand.

The operational envelope of a device may be adapted based on user performance. For example, if the user is using a harmonic device with continuous activation and a tiny tip, the shaft of the device may get warm. The operational envelope may adjust to protect critical structures from undesired thermal spread from the shaft.

In an example, a harmonic device may have a designated area of operation. Once staples have been deployed in a specific area, the designated area of operation for the energy device may be modified to exclude the area, the system may provide a warning to the user that metal is in that area (e.g., and that caution should be exercised), or an override condition may be necessary to activate the device in that area.

In an example, if unexpected, thin, diseased tissue is found on the intestines, the operational window of the areas available for graspers to grab may be updated to exclude this area.

Vision systems may be synchronized based on a specific tissue response and/or anatomy. If an endoscopic vision system and a laparoscopic vision system is used, a user may switch between cameras to view the moving system. The alternation of which camera is stationary or moving may be determined by a tissue response or selected anatomy.

In another example, after a low anterior resection (LAR), surgeons may use a colonoscope and a laparoscope to inspect the anastomosis for leaks. The colonoscope may be used to insufflate the colon and visualize the staple line intraluminally. The laparoscope may be used to inspect for air bubbles extraluminally from the abdominal cavity. If air bubbles are detected extraluminally, the colonoscope may orient the view to approximate the location of the air bubbles seen on the extraluminal side. Conversely, if bleeding is seen intraluminally, the laparoscope may orient its view to visualize the same extraluminal location.

At the end of an LAR, the surgeon may closely example the staple line internally to see the anastomosis through a colonoscope. At the same time, the laparoscope may be used to view the anastomosis from the abdominal cavity. The tracking may be used to look for bubbles due to fluid and/or air that entered into the system. In this case, it may be useful for the surgeon to be able to alternate between the laparoscope (e.g., identifying where the bubbles are coming out) and the colonoscope with a matching view from inside the colon.

Digital aspects (e.g., visualization, data exchange, processing, and/or monitoring) of devices may be synchronized. Devices may cooperatively exchange and process digital data to provide improved (e.g., optimal) operational envelope data.

Interference avoidance between visualization systems may be improved (e.g., by selectively adjusting imaging control for multiple systems to combine their outputs into a useable overlay). Discrete frequency spectral imaging may be used. For example, discrete frequency spectral imaging may be used to prevent inadvertent leakage of one frequency imaging into the other's domain. Structured light may use infrared laser spectrum to project a matrix array onto organs (e.g., to determine volumes and shapes. Because the structed light operates in this known spectral range, a multi-spectral camera may filter or adjust its normal imaging of infrared to filter out or avoid the added energy provided by the structured light. The multi-spectral camera may avoid the use of certain wavelengths, place the system temporally out of phase with the structured light projector, and/or filter out the additive aspect from the projector.

Multi-frequency sweeping scanning may be used to avoid interference between visualization systems. A first scan's sweeps may affect another scan's measurements or induce a local physiologic response, field sweeping scanning arrays, laser Doppler flowmetry (LDF), blood flow, etc. To avoid this interference, tissue impedance, ultrasound reflection, and/or sweeping scanning arrays may be staged out of phase with each other.

Multi-intensity imaging may be used to avoid interference between visualization systems. For example, CT resolution and/or reflection may be used. The multi-intensity imaging may be used depending on the material of an object of interest (e.g., metal, plastic, ceramic, bone, soft tissue, calcification, etc.). Reflective monochromatic light may be used to avoid interference between visualization systems. Surface conditions and/or shadows (e.g., reflectivity) may be used to avoid interference between visualization systems.

The intensity of a system may be adjusted to filter out interactions between systems. The intensity of a system may be adjusted to better visualize portions of the patient that another system is having trouble viewing (e.g., due to the energy used and/or the location to be imaged).

Aspects of separate visualization systems may be linked or coupled to provide complimentary data. For example, the ablation confirmation system “Set-up CT scans” may use previous scans and/or scans completed at the start of a procedure. During a “Target” phase, the system may use the scans to define the target ablation and select the desired margins and/or zone to set on the tumor (e.g., based on tumor location to critical structures that may alter the margin/zone). The user may place one or more probe(s) in location(s) determined based on the CT scans. The location(s) of the probe(s) may be selected based on a best path for access. Ultrasound may be used for guidance (e.g., into a tumor). Ultrasound may be used to control the depth based off information from the CT scans. After the probes are placed, another CT scan may be used to confirm the probe(s) are at the target location(s). This process may be repeated until the probe(s) are at the planned location(s) (e.g., within the tumor).

Patient movement, breathing, etc. may alter the targeted probe location and actual probe location. The ultrasound guidance may increase the likelihood of proper placement (e.g., with minimal probe replacements). The system may register the set-up scans and probe scans to overlay. The registration may indicate the initial tumor scan and probe placement scan to the user. The registration may allow the user to change the ablation zone prior to ablating. The user may ablate and perform a final CT scan. The final CT scan may be overlayed with the initial scan to compare the actual ablation to the intended ablation. The ablation may be continued if the initial ablation did not fully ablate the intended ablation zone.

If a full thickness resection is completed with assistance from laparoscopic and endoscopic devices, the light from one side may wash out the view from the other side. A common system may monitor and control the illumination and vision systems to avoid this problem. If a non-visible signal (e.g., such as NIR wavelength >800 nm) is detected on a first side, the system may turn off the light on a second side to improve the view from the first side.

FIG. 53 illustrates an example method that may be performed by a surgical instrument. As shown, the method may involve, at 54450, receiving a user surgical control input. The method may involve, at 54452, sending an indication of a preferred operational envelope to a negotiating surgical device. The method may involve, at 54454, constraining operation responsive to the user surgical control input based on a reduced operational envelope that is determined by a response, from the negotiating surgical device, to the indication of the preferred operational envelope. The reduced operational envelope may result in a greater constraint on the operation responsive to the user surgical control input than the preferred operational envelope.

In operating rooms, multiple surgical imaging devices may operate in close proximity to one another. For example, FIG. 54 illustrates an operating room in which two laparoscopes 54402, 54404 and an endoscope 54406 are being used simultaneously. The imaging devices may all be from different manufacturers and may have different control systems. When devices, such as these, do not coordinate with each other they may limit the field of view of a user (e.g., surgeon). Such limited visibility may cause the user to miss important events during surgery, such as an unintended bleed. The coordination between and/or among imaging systems as disclosed herein may enhance the field of view of the user resulting, for example, in fewer missed surgical events and improving patient outcomes.

In an example, a first imaging device, such as an endoscope 54406, may include an imaging sensor and a processor. A second imaging device, such as any of laparoscopes 54402, 54404 may each include a respective imaging sensor and processor. The first and second imaging devices may be manual surgical instruments (e.g., as illustrated in FIG. 54). The first and second imaging devices may be robotic surgical instruments. The processor of the first imaging device may be configured to receive a video data stream captured by a second imaging device and determine, based on the video data stream, a coupled field of view between the first imaging device and the second imaging device.

A field of view may include a visual area that can be seen via an imaging device such as an endoscope 54406, and/or laparoscopes 54402, 54404. The field of view provided by the imaging devices may be dynamically adjustable. For example, the field of view may be adjusted by affecting changes in the device such as changes to the lens and/or optics, by changing the focal length of the optics, by affecting the zoom of the imaging device via physical optics and/or digitally, by affecting the effective sensor size (e.g., larger effective sensors generally have wider field of views), by affecting the position of the imaging device (e.g., physically moving the device may change the portion of the observable space; movements may include pan, tilt, shift, dolly, and the like), image stitching (e.g., multiple images may be stitched together to create a panoramic view to increase field of view), adjusting aperture size (e.g., to influence depth of field and perceived field of view), and the like.

In an example, imaging systems, for example a first and second imaging system, may coordination their respective operation. For example, the first and second imaging system may coordinate their respective operation regarding their respective fields of view. For example, synchronized imaging of two system may be used to maintain a common field-of-view or perspective for a plurality of systems (e.g., to transition objects from one field of view to another). Cooperative multiple scope synchronized motion may be used to maintain the relational field-of-views. The motion of multiple (e.g., two) independent imaging scopes may be coupled. The coupled motion may involve the movement of one scope initiating movement of a second scope to maintain the couple field-of-view of the two scopes.

In an example, the coordinate of fields of view may be implemented via the adjustment of an imaging parameter, as disclosed herein. For example, the processor of the first imaging device may determine, based on the video data stream, that the second imaging device has moved and may adjust an imaging parameter of the first imaging device to maintain the coupled field of view. The imaging parameter may include any parameter that represents a quantity suitable for altering a field of view of an imaging device, such as an electronically controlled field of view (e.g., digitally controlled resolution and/or windowing), a position of the first imaging device, a focal length of the first imaging device, a portion of a field of view associated with the first imaging device that is displayed to a user, and the like.

In the surgical environment, coordinated operation of two or more imaging systems regarding fields of view may be employed when the imagining systems are viewing within a common anatomical space and/or when the imaging systems are viewing within separate anatomical spaces. An anatomical space may include a cavity and/or compartment within the body. An anatomical space may include a space that contains organs, tissues, or other structures. The anatomical space may include a space accessible by a particular instrument. For example, in laparoscopic surgery, a small incision is made in the abdomen to insert a laparoscope which provides a view of the abdominal anatomical space (e.g., a laparoscopic anatomical space). For example, in endoscopic surgery, an endoscope may be inserted through natural orifice, such as the mouth or anus, and guided to the surgical site. Such spaces accessible in this way may include endoscopic anatomical space. In examples, the endoscope may be inserted through a small incision. In addition to surgical procedures, anatomical spaces may also be visualized using non-invasive medical imaging techniques such as X-rays and CT scans, for example.

In an example, with the two scopes in a common anatomical space (e.g., laparoscopes 54402, 54404 in a common laparoscopic space), coordination regarding field of view may include adjustments to a common observable area. In an example, the two scopes may cooperatively maintain a filed-of-view that is larger than either scope is capable of capturing alone. The composite image may be the entire displayed field-of-view (e.g., captured by both scopes). The synchronized motion may be used to maintain the spacing between the scopes (e.g., to maintain the overall field-of-view).

In an example, with two scopes in different anatomical spaces (e.g., either of laparoscopes 54402, 54404 in a laparoscopic space and endoscope 54406 in an endoscopic space) coordination regarding field of view may include adjustments to each scope's respective side of a common anatomical barrier between the two anatomical spaces. For example, visualization of the common anatomical barrier may be performed from differing sides (e.g., the endoscopic and laparoscopic sides) of a tissue wall (e.g., an organ wall). Coordinate of field of view may include adjusting the field of view to maintain viewing of opposite sides of the common anatomical barrier (e.g., maintaining respective views on both sides of the barrier in sync). For example, adjustments that affect the monitored position and/or orientation may be used to keep the two cameras focused on the same intermediate portion of a tissue wall. In an example, synchronization may be maintained through external imaging (e.g., a cone-beam computerized tomography or electromagnetic sensing of another camera).

Cone-beam computerized tomography (CBCT) may include a medical imaging technique that uses a cone-shaped X-ray beam to produce 3D images of the patient's body. CBCT is similar to traditional computed tomography (CT) but may use a lower radiation dose and may provide higher resolution images of the area of interest. CBCT can also be used to guide surgical procedures.

For example, CBCT may be used to determine the position of a surgical instrument during a procedure, such as the position of one or more scopes disclosed herein. Here, the tracking system of the surgical instrument may be configured to determine the position/orientation of each scope head (e.g., each camera's location) based on the relative position of the respective scope head as observed by the CBCT. The position information may be provided to a processor, which can be programmed or configured to determine, with the camera intrinsics, a complete coordinate model for each scope within a common geometry. Such a model may be used for registration and/or iterative tracking of the scopes.

Electromagnetic (EM) sensing may be used to determine the position/orientation of the one or more scopes disclosed herein. Each scope may include an EM sensor system. The sensor may include one or more conductive coils that are subjected to an externally generated electromagnetic field. When subjected to the externally generated electromagnetic field, each coil of the EM sensor system may produce an induced electrical signal having characteristics that depend on the position and orientation of the coil relative to the externally generated electromagnetic field. In an example, the EM sensor system may be configured and positioned to measure six degrees of freedom. e.g., three position coordinates X, Y, Z and three orientation angles indicating pitch, yaw, and roll of a base point or five degrees of freedom. e.g., three position coordinates X, Y, Z and two orientation angles indicating pitch and yaw of a base point. For example, the EM sensor system may include that disclosed in U.S. Pat. No. 6,380,732 (filed Aug. 11, 1999 and incorporated by reference herein). The position information may be provided to a processor, which can be programmed or configured to determine, with the camera intrinsics, a complete coordinate model for each scope within a common geometry. Such a model may be used for registration and/or iterative tracking of the scopes.

Field of view coordination may include the incorporation of certain visualizations to one and/or both of the video streams. Visualizations may include any operation to the visual information of the video stream of one imaging device based on information from the other imaging device. Here, coordination of the field of view includes the ability to operationally augment the field of view from one device with information from the other. For example, with imaging devices in different anatomical space, a visualization may include an operation to visually subtract, or make transparent, parts of the anatomical barrier (e.g., tissue wall) separating them. This may allow a user to section the common view and see depth, underlining structures, or instruments on the other side of the tissue wall.

In other examples, a multi-scope system may use visualization cooperative movement and/or an extended viewable display. Synchronized visualization may involve electronic and/or algorithmic field-of-view limiting and overlapping imaging to enable each camera to capture images to produce a composite image (e.g., by adapting the synchronized imaging arrays). The synchronization may employ misaligned (e.g., slightly misaligned) CMOS arrays (e.g., that image differing wavelengths of energy). Such an overlap mode of operation may enable the overlay of multi-spectral imaging over visible light imaging on a common display (e.g., with the different imaging originating from side-by-side CMOS arrays).

FIG. 55 illustrates an example of a first and second imaging devices with cooperative operation. Here, a tracking imaging scope 54408 may adjust an imaging parameter to maintain a coupled field of view with another imaging scope (e.g., a navigating imaging scope 54410). As shown, the two scopes may be on opposite sides of an anatomical barrier 54412 (such as a tissue barrier). A navigating imaging scope 54410 may be controlled by a surgeon. The navigating imaging scope 54410 may observe an object of interest 54414 (e.g., a tumor) and generate a corresponding video data stream.

The navigating imaging scope 55410 and the tracking imaging scope 54408 may exchange an initial handshake protocol to establish the corporative operation. The handshake protocol may include any data protocol suitable for device and capability discovery. For example, the handshake protocol may include a Universal Plug and Play UPnP protocol. Via UPnP, the navigating imaging scope 55410 and the tracking imaging scope 54408 may advertise their presence in the network and discover each other. The protocol may include sending responsive to discovery a description of available cooperative services available. The description may be an XML-based (Extensible Markup Language-based) description. The description may include identifying information about each device, such as manufacturer, model name, model number, serial number, and the like. The description may include capability-specific information including service type, service Id, Service description URL, control URL, eventing URL, and the like. The service description may include information about the device's capability to act as in a tracking and/or navigating capacity. The service description may include information regarding a destination for communicating video data stream information for purposes of cooperative field of view operation.

The navigating imaging scope 54410 may communicate the video data stream information to the tracking imaging scope 54408. The tracking imaging scope 54408 may receive the video data stream from the navigating imaging scope 54410.

Having video information from the navigating imaging scope 54410, the tracking imaging scope 54408 may establish a common coordinate system. To establish a common coordinate system, the system may use any computer vision, photogrammetry, and/or robotics-control techniques suitable for synthesizing information from multiple cameras. In an example, the system may use a computer vision algorithm to develop a common coordinate system. The tracking imaging scope 54410 may receive intrinsic parameter information from the navigating imaging scope 54408, for example, parameters such as focal length, principal point, lens distortion, and the like. The tracking imaging scope 54410 may receive extrinsic parameter information from the navigating imaging scope 54408, for example, parameters such as relative positions and/or orientations. In an example, one or more features or keypoints may be identified in both images. For example, the object of interest 54414, when in the field of view of both scopes, may be selected as such as feature.

The video data stream information may indicate coordinate points associated with the object of interest 54414. For example, as shown in FIG. 55, the navigating imaging scope 54410 may determine that the object of interest 54414 is centered at a point in the navigating imaging scope's field of view that corresponds to the coordinate point (200, 225). The tracking imaging scope 54408 may capture its own video information about the object of interest. As illustrated, for example, the tracking imaging scope 54408 may determine that the object of interest 54414 is centered at a point in the tracking imaging scope's field of view that corresponds to the coordinate point (50, 355). The difference in coordinate points between the scopes may be due to difference in one or more factors between the scopes, for example, angle of the camera, size of the field of view, location of a reference coordinate system origin, and/or the like. And, the difference in coordinate points may be accommodated with an adjustment to any of such factors.

For example, as shown in FIG. 55, the tracking imaging scope 54408 may perform object registration for the object of interest 54414. The tracking imaging scope 54408 may synchronize the relational field of views of the primary and secondary scopes, for example. The tracking imaging scope 54408 may synchronize the field of views by calculating the delta between the two coordinate points (e.g., in this example, Δ=(150, −135). The tracking imaging scope 54408 may adjust one or more of its imaging parameters to establish and then maintain a coupled field of view with the navigating imaging scope. For example, the imaging parameter may include a physical location of the scope, an angle of view of the object of interest, a focal length of the scope, etc.

In an example, the tracking imaging scope 54408 may adjust an imaging parameter that includes an electronically controlled field of view, for example a digital window of its field of view, to establish a coupled field of view with the navigating imaging scope 54410. As illustrated in FIG. 55, the tracking imaging scope 54408 may determine the imaging parameter to be vector represented by the delta between the coordinate points identified in the object registration. The vector may be used to as a transform 54416 to be applied to the field of view of the tracking imaging scope 54408. The video image maybe translated and/or scaled in a manner proportional to the vector such that in the resulting field of view the objects of the object registration are aligned. The electronically controlled field of view may include digital windowing, CMOS segment activation/deactivation, optical and/or digital zoom, aperture control, focal length, and the like.

In an example, the tracking imaging scope 54408 may adjust an imaging parameter, for example the position of the camera of the tracking imaging scope 54408, to establish a coupled field of view with the navigating imaging scope 54410. Again, the tracking imaging scope 54408 may determine the imaging parameter to be vector represented by the delta between the coordinate points identified in the object registration. The vector may be used to as a control input to be applied to a surgical robot control of the tracking imaging scope 54408. The change in position of the camera may be proportional to the vector such that in the resulting field of view the objects of the object registration are aligned.

In an example, the tracking imaging scope 54408 may adjust more than one imaging parameter, for example the position of the camera of the tracking imaging scope 54408 and a digital window of its field of view, to establish a coupled field of view with the navigating imaging scope 54410. For example, the adjustment may apportion the vector represented by the delta between the coordinate points identified in the object registration to the more than one imaging parameters. The proportional adjustment of each may result in a field of view in which the objects of the object registration are aligned.

In an example, adjustment of the imaging parameter(s) may be done in unit steps, iteratively, gradient-based, or the like. In an example, the adjustment of the imaging parameter(s) may incorporate modeled aspects of the tracking imaging scope's performance, such a project camera model. Here, a project camera model with camera intrinsics and inverse pose may be solved to determine a world line corresponding to the image point corresponding to the tracking imaging scope's object identified in the object registration. Then, a new set of intrinsics and/or inverse pose may be solved for using the same world line and the target image point represented by the delta between the objects identified in the object registration.

FIG. 56 illustrates an example of a smart surgical scope updating imaging parameter(s) to maintain a coupled field of view with another imaging scope. As shown in FIG. 56, the navigating imaging scope 54410 may have moved (e.g., compared to its location in FIG. 55). The navigating imaging scope 54410 may continue to communicate its video data stream information to the tracking imaging scope 54408. The tracking imaging scope 54408 may detect the navigating imaging scope's movement based on the video data stream information. For example, the video feed information may indicate a different coordinate point associated with the object of interest than previously reported. For example, the tracking imaging scope 54408 may use video-based motion estimation of the video data stream information to detect the navigating imaging scope's movement. The video-based motion estimation may include approaches such as block matching algorithms, optical flow, phase correlation, gradient-based methods, Kalman filtering, and the like.

The tracking imaging scope 54408 may compare subsequent video images to determine a difference between the coordinate points of the two scopes (e.g., in this examples, Δ=(75, −100) after the movement of the navigating imaging scope 54410. The tracking imaging scope 54408 may adjust one or more of its imaging parameter(s), as described herein. Adjusting the imaging parameter(s) will change the coordinate point that the secondary imaging device associates with the object of interest. The tracking imaging scope may then iteratively compare the coordinate points of the two scopes and adjust imaging parameter(s) as needed until the fields of view are synchronized. The tracking imaging scope 54408 may determine the coupled field of view has been maintained on the condition field of the view of the tracking imaging scope 54408 and the video data stream are aligned.

In an example, the navigating imaging scope 54410 may communicate information indicative of its movement to the tracking imaging scope 54408. For example, a tracking imaging scope 54408 driven by a surgical robot control may provide the control inputs given by the surgeon to the surgical robot to the tracking imaging scope 54408 via a data channel (e.g., embedded in the video data stream and/or a channel separate from the video data stream). The tracking imaging scope 54408 may use the control inputs when determining the motion seen in the video data stream. In an example, other positioning data such as surgical x-ray, cone CT, EM sensing, the like, may be employed to determine motion.

In an example, other aspects of scope operation may be coordinated to enhance performance and/or usability. Operational aspects such as lighting intensity, wavelength, display features, and the like may be coordinated via communication between the scopes. For example, the motion of each scope and/or the direction of the motion from the light source from each individual scope may be used in cooperation (e.g., to enhance the view of each scope). Synchronizing the motion of the scopes on the surgical site and the light source of a scope may be used to enhance the view from the other scope. A laparoscopic scope may limit the luminescence or the amount of light the scope generates. The light source may be directed from behind the camera of the scope. During a procedure utilizing two scopes, the light sources of each camera may be synchronized to benefit the other. The light source of the scope may obscure the lighting back to the field of view (e.g., based on the surgical location and/or other instruments). In this case, the first scope may turn off its light source. The second scope may be used to direct its light source to optimize the view. Each scope may have unique wavelengths and/or color (e.g., to further optimize the feedback to the user).

Regarding display aspects, in an example, camera views may be altered (e.g., by establishing the same coordinate system for the two scopes, as described herein). The coordinate system synchronization may allow a surgeon to easily toggle back and forth between the two scopes, while still recognizing what is shown in both views. A user may be able to swap between primary and secondary live feeds. For example, there may be a solid part or organ within the surgeon's field of view. The system may allow the surgeon to switch to another field of view associated with a different scope.

In addition, scope coordination may enhance surgical operation through enhanced calibration. For example, calibration may be enabled via multi-system registration of common object. And a redundant imaging system may be used to view a common object between two systems (e.g., one of which is being affected by navigation distortion) to aid in the compensation for calibration. For example, the laparoscopic and endoscopic imaging systems may be coupled together for cooperative motion. And the cooperative motion may be used to enable one of the two visualization systems that is highly impact by distortion (e.g., an electromagnetic navigation system of the robotic flexible endoscopic system) to track common landmarks or easily identified structures (e.g., to help correct for the distortions in its navigation system without the need for frequent radio transmissive recalibration). The relational field-of-views position may indicate a global position of the position of a tumor. The system may use the global position of the tumor to augment the view of the tumor into the laparoscopic field of view. As the colon (or hollow organ, e.g., stomach, bladder, etc.) moves, the image recognition from the laparoscopic side (e.g., through TOF, structured light, or optical shape sensing) and proximity of the endoscope may be used to dynamically move the augmented tumor view.

FIG. 57 illustrates an example of emitting additional wavelengths from one scope through tissue to augment sensors in another scope. As illustrated, the laparoscopic scope 54420 may shine a light toward an object of interest 54422 on the opposite side of a tissue barrier 54424. The endoscopic scope 54418 may have a direct view of the object of interest 54422. The endoscopic scope 54418 may be able to detect the wavelengths of the light from the laparoscopic scope 54420. The wavelengths, as shown through the object of interest 54422, may inform the surgeon of one or more properties of the object of interest.

In an example, an illumination-providing scope or light source may be moved in advance of the imaging scope (e.g., to compliment the illumination of the imaging scope). In this predictive operating mode, the light source may be moved first (e.g., to remove shadows and improve visualization of recessed areas that the light from the primary scope has not yet illuminated). Using FIG. 57 as an example, the endoscopic scope 54418 may be placed first and may illuminate the location of the object of interest 54422. The laparoscopic scope 54420 may use the light from the endoscopic scope 54418 to locate the object of interest 54422 (e.g., even though it does not have a direct view of the object).

In an example, a visualization may be generated in the video output of one of the scopes based on information from the other scope. For example, the visualization may include information from a first imaging device that when incorporated into the video output of the second imaging device makes a portion of the viewed anatomical barrier appear transparent. To illustrate, video of the object of interest 54422 taken from the endoscopic scope 54418 may be overlaid the video from the laparoscopic scope 54420 to reveal an image of the object of interest 54422 in the laparoscopic view. As modified, a portion of the tissue barrier 54424 in the video may appear to be transparent. Such video operation may include a selective subtractive image modification operation.

Examples of procedures in which the techniques described with respect to FIG. 57 may be implemented are described in greater detail in U.S. Patent Application Publication No. US 2023-0095002 A1 (U.S. patent application Ser. No. 17/493,913), titled SURGICAL METHODS USING FIDUCIAL IDENTIFICATION AND TRACKING, filed Oct. 5, 2021, the disclosure of which is herein incorporated by reference in its entirety.

In an example, subtractive image modification may be used to fill in the shapes and colors of the part of the image that was occluded. Subtractive image modification may be used to enable user visibility of occluded areas. Subtractive image modification may be used to enable visualization of occluded areas.

Cooperative operation among imaging devices may enable the presentation of the image(s) modified with color. For example, an area made transparent (e.g., by subtractive image modification) may be shaded with a color. The area that is visible due to the transparency may be shaded with color. The image may be composed (or decomposed) into the relative areas and related colors. For example, a primary image may be blue and a secondary image may be red. The primary and secondary images may be composed into the blind spot/occluded image (e.g., which may be purple). The color may be distorted (e.g., using software) on each camera to be shaded with a different tint (e.g., red or blue to make it more obvious when a composite image is seen).

FIG. 58 illustrates an example of using color distortion to indicate that an image is a composite image. For example, a modified output video stream 54426 may have three portions. The first portion 54428 may represent corresponding video from video stream 54430 from a first imagine device. The second portion 54432 may represent corresponding video from video stream 54434 from a second imagine device. And the third portion 54436 may represent an overlay of a corresponding portion 54438 from both the video stream 54430 from the first imaging device and the video stream 54434 from the second imagine device. The composite image may use one or more such portions 54428, 54432, 54436 in any combination and/or orientation. The source identity of the portions 54428, 54432, 54436 may be indicated to the user by color. For example, the first portion 54428 may be a first color that corresponds to the video stream 54430 from the first imagine device. The second portion 54432 may be a second color that corresponds to the video stream 54434 from the second imagine device. And the third portion 54436, as an overlay, may be a third color, for example, a color that represents a proportional mix of the first and second colors.

In an example, a selective view of certain segments may be used. The ability to make the anatomy transparent may be enabled/disabled (e.g., by a user). The system may allow the surgeon to draw shapes around the areas they would like to be made transparent (e.g., using a tool such as a tablet).

Synchronization of multi-sourced imaging data (e.g., currently active vision systems or prior scans) may be used to generate or create a virtually-rendered field of view. This enables a field of view larger than that possible using a single vision system or overlapping. Vision systems may coordinate and alternate performing scans of the surrounding area (e.g., to determine whether to update the virtually rendered field of view). For example, a first vision system, vision system A, may be the currently active vision system. A second vision system, vision system B may run a scan of surrounding area. Vision system B may update the combined field of view (e.g., if necessary). If vision system B's area of view is currently active, vision system A may perform the sweeping scan to check for any field of view updates.

In an example, data input from multiple sources may be used to enable multi-directional modality projection. Multi-directional projection of energy modalities (e.g., light spectrum, gamma, x-ray, microwave) may be used. Each source may include a respective imagine sensor that senses light of a corresponding band. So a video data stream captured from one scope may then represent light from a first band, and a video stream captured by another scope may represent light of a second band that is different from the first band. The light from differing scopes and/or projectors may be received. Penetrating or backlit cooperative scope wavelength motion and detection system may introduce wavelengths, intensities, and/or combined wavelengths (e.g., for which the first scope may not have the bandwidth or capabilities). In the case of high intensity coherent light sources, the fiber optics of the shaft may conduct the light from a source outside of the body. As the size of the shafts gets smaller, the fiber bundles may get smaller. This may cause the fiber bundles to be overloaded with the magnitude of the energy being conducted (e.g., often melting or degrading the fiber optics). These externally-projected lights may be introduced with a larger system (e.g., from the lap side with a 12 mm shaft), for example, as opposed to the sending system (e.g., a 2 mm CMOS arrays on the flexible scope side). This may provide illumination to sub-surface structures (e.g., because the light is projected through the tissue rather than being projected onto the tissue). The larger system may allow the projection source to be stronger (e.g., orders of magnitude stronger) than the 2 mm fiber optics could provide. The projection source may be in addition to the local light imaging of the endoscopic scope (e.g., thereby minimizing the extra-spectral loss of the magnitude of the visible light spectrum). The projection source light may include wavelengths and/energy sources that are not compatible with the flexible scope system (e.g., wavelengths exciting a reagent, for example, ICG or tumor tagging fluorophores with wavelengths that cause excitation of the natural molecular nature of the tissue, radiation, microwave, etc.).

The movement of one or more visualization system(s) and/or smart device(s) may be synchronized so that the visualization system(s) are paired to the device. The movement of a device may initiate movement of one or more cameras (e.g., to maintain a common field of view). Vision systems may track a smart device to maintain field of view (e.g., using a sub-spectral or infrared tag). Vision systems may not (e.g., may not need to) keep a device centered in its field of view (e.g., if AE or critical area of tissue gains precedence, in which case the AE or critical area of tissue may be the center of the field of view).

FIG. 59 illustrates an example method that may be performed by a surgical instrument. At 54440, a video data stream captured by a second imaging device may be received by a first imaging device. For example, the first imaging device may be located on a first side of an anatomical barrier, and the second imaging device may be located on a second side of the anatomical barrier. The first side and the second side are opposing sides of the anatomical barrier. For example, the anatomical barrier may include tissue that defines a first anatomical space within which is the first imaging device and that defines a second anatomical space within which is the second imaging device. For example, an endoscopic view may be generated by a video stream captured by the first imaging device. The video stream captured by the second imaging device may represent a laparoscopic view. The first imaging device may include a first imaging sensor that senses light of a first band. The second imaging device may include a second imaging sensor that senses light of a second band. The first band may be different from the second band.

The reception of the video data stream may be responsive to a handshake protocol. For example, the first imaging device may communicate a handshake protocol with the second imaging device to establish cooperative operation.

At 54442, a coupled field of view may be determined, based on the video data stream. In an example, the coupled field of view may be determined by performing an object registration on an object and synchronizing a first field of view associated with the first imaging device with a second field of view associated with the second imaging device based on the object registration. Here, the object may be visible in the first field of view and in a second field of view. In an example, the coupled field of view may be determined by receiving relative field of view positioning information from cone-beam computerized tomography imaging that comprises a view of the first imaging device and the second imaging device. In an example, the coupled field of view may be determined by receive relative field of view positioning information from electromagnetic sensing of the second imaging device.

In an example, a visualization associated with the anatomical barrier in the video data stream may be generated based on information from an imaging sensor of the first imaging device. For example, the visualization may include information from the imaging sensor of the first imaging device that makes a portion of the anatomical barrier appear transparent.

At 54444, it may be determined, based on the video data stream, that the second imaging device has moved. And at 54446, an imaging parameter of the first imaging device may be adjusted to maintain the coupled field of view. In an example, the imaging parameter may include an electronically controlled field of view. In an example, the imaging parameter may include any of a position of the first imaging device, a focal length of the first imaging device, or a portion of a field of view associated with the first imaging device that is displayed to a user. The imaging parameter may be adjusted to maintain the coupled field of view by iteratively comparing a first field of view associated with the first imaging device to the video data stream and adjusting the imaging parameter based on the comparison. It may be determined that that the coupled field of view is maintained based on the condition that the first field of view and the video data stream are aligned with respect to a registered object.

In operating rooms, multiple surgical imaging devices may operate in close proximity to one another. FIG. 60 illustrates an example surgical operating room with several different devices. An imaging device may have a sensor that tracks the location of the imaging device. Other devices in the operating room may create electromagnetic fields that affect the accuracy of the sensor's ability to track the imaging device. FIG. 61 illustrates an example of electromagnetic distortion caused by metal staples/clips. As shown, the metal may distort the EM field. The distortion may impact the EM navigation (EMN) device. For example, the metal may cause the EMN sensed location to be skewed from the actual location of the EMN device.

For example, if a first surgical instrument with an EM sensor is near an ultrasound imaging probe and a metal object (e.g., a row of metal staples or clips), the EM sensor may experience distortion of a predicted location of the EM sensor. The EM field causing distortion of the predicted location may be created by proximity of the ultrasound imaging probe to the metal object. The first surgical device may determine an adjusted location of the EM sensor based on the received coordinate system by aligning expected anatomy associated with the predicted location with anatomy in the area around the EM sensor associated with the received coordinate system.

The devices may not be aware of the presence of other devices. Even if the devices are aware of other devices, the devices may not be able to communicate information such as information related to electromagnetic distortion. Without this knowledge, a user (e.g., surgeon) may not know the actual location of an imaging device, which may affect the user's ability to safely perform the operation.

To enable such devices to detect and compensate for distortion, a common reference plane may be created for multiple (e.g., two) imaging streams. For example, the common reference plane may be created using a reference plane from one imaging system as a means to compensate for a distortion of coordinates of another imaging system. Alignment of two oblique reference planes and distortion compensation for at least one of the sensed locations may be realized by utilizing another coordinate system originating from an independent system. For example, the other coordinate system may be used to form the basis for a common coordinate system for both reference planes). FIG. 61 further illustrates an example of the adjusted location of the EMN device (e.g., after distortion compensation).

The first coordinate system may be derived from the real-time measurement of a sensor (e.g., in three dimensional space). The first coordinate system may be used to determine the current sensed/measured location of the sensor. The first coordinate system may accumulate additive errors (e.g., due to distortion of the measurement).

An example device may sense, using the EM sensor, a predicted location of the EM sensor. The predicted location may be distorted by an EM field. The device may receive, from a second surgical instrument, a coordinate system associated with imaging of an area around the EM sensor. The device may determine an adjusted location of the EM sensor based on the received coordinate system.

The alignment of the two coordinate systems may rely on an association between the first system and the second system. The alignment of the two coordinate systems may rely on the distortion compensation of the second system's measurements (e.g., relative to the first imagine system's detector).

The reference planes may have a relative commonality. One plane may have a higher validity (e.g., due to a more closely controlled association to the patient-related reference plane(s)). A multi-level reference plane may be used to enable local and global registration. A sub or local reference plane may be formed within a linked global reference plane. A multi-level reference plane may be generated (e.g., to provide maximum available data to the user).

Multi-level data transfer may be established. The data transfer may enable registration of smart systems (e.g., including “simple” and/or “complex” registration). The simple registration may include minimal data points. The simple registration may include critical structures and enough data to render a general visual rendering. The complex registration may include the simple registration as a base. The complex registration may overlay the simple registration with the data available in that smart system (e.g., to include specific and detailed anatomy and/or blood flow). If the system shares the registration, the simple registration data (e.g., at least the simple registration data) may be transferred. Different levels of registration data may also be provided (e.g., on top of the simple registration data, for example, based on the capability of the receiving smart system).

An example surgical instrument may receive registration information of registered elements on a second surgical instrument. The surgical instrument may monitor positional changes of the second surgical instrument based on the registration information. The surgical instrument may adjust the adjusted location of the EM sensor based on the monitored positional changes.

For example, the surgical instrument may be a laparoscopic probe with an EM sensor. The EM sensor may be a magnetic tracker. The second surgical instrument may be a moveable CT machine. The EM field causing distortion of the predicted location may be created by proximity of the moveable CT machine to the first surgical instrument. The surgical device may determine the adjusted location of the EM sensor based on the received coordinate system by aligning expected anatomy associated with the predicted location with anatomy in the area around the EM sensor associated with the received coordinate system.

Feature(s) associated with creating a common global reference plane are provided herein. Multiple (e.g., two) systems may dynamically exchange their reference planes with each other (e.g., including the location of each other). A global reference plane may be established within each system. A device may determine an adjusted location of the EM sensor with respect to the global reference plane (e.g., that is associated with an operating room in which the first surgical instrument is located).

FIG. 62 illustrates example operations and communication by two surgical devices. As shown, a first device may capture image(s) of an area. The first device may determine a coordinate system associated with the image(s) of the area. The first device may send the coordinate system to a second device. The second device may receive the coordinate system. The second device may sense a predicted location of a sensor. The second device may determine an adjusted location of the sensor based on the received coordinate system.

FIG. 63 is a block diagram illustrating example components of a surgical device. As illustrated, an intra-operative imaging machine may capture image(s) of a surgical area (e.g., around a sensor on another device). The intra-operative imaging device may send, to a surgical device, a coordinate system associated with the image(s) of the surgical area. For example, the coordinate system may indicate a reference geometry used by the intra-operative imaging device.

The surgical device may receive the coordinate system (e.g., via a data input/output module). The surgical device may include a sensor (e.g., the sensor around which the intra-operative imaging device is capturing images). The surgical device may use the sensor to sense a predicted location of the surgical device. However, as described herein, the sensed location may be distorted from the actual position due to EM interference. The surgical device may therefore input the sensed location and the coordinate system into a coordinate system adjustment module. The coordinate system adjustment module may determine an adjusted location of the sensor based on the coordinate system from the intra-operative imaging device.

FIGS. 64A-D illustrate graphical representations of magnetic distortion and distortion compensation. FIGS. 64A and 64C illustrate the distortion of a square electromagnetic coordinate system. The distortion may be caused by the presence, movement, or introduction of metal objects in close proximity to the transmitter or sensor (e.g., within inches). If the system measures the distortion from another point of view or secondary sensing system, the system may compensate for the magnetic field distortion (e.g., with algorithms, for example, rather than merely changing calibration of the sensing system). FIGS. 64B and 64D illustrate examples of distortion compensation for the magnetic distortion shown in FIGS. 64A and 64C, respectively. The distortion compensation may be represented by a transform function. This transform function may be adapted with additional sensed input(s) (e.g., to more accurately compensate for the distortion).

A (e.g., single) physical lattice may be placed on and/or around the patient (e.g., as illustrated in FIG. 65). A (e.g., each) system may utilize a global reference plane from the physical lattice. Feature(s) associated with determining the orientation of the lattice are provided herein. An absolute position reference sensor may be used to determine the Z-orientation of the lattice. The lattice may include one or more (e.g., multiple) sensors (e.g., wireless beacons). The sensors may include a relatively predefined geometry. A sensor may be given a certain level of precedence/priority. Each sensor may include a unique identity. The unique identity may provide a known relative location. The other items referencing the relative location may be able to establish their own orientation (e.g., relative to that fixed point). The absolute orientation of the lattice may become irrelevant (e.g., once the other items establish their own orientation).

A (e.g., single) system may be used as the global coordinate system. The other system(s) may be established based on the global coordinate system. A sensor may be placed on a first system. A tracker (e.g., minion) may track the sensor (e.g., thereby allowing the tracker to adapt its system to the first system to form a unified reference plane).

For example, FIG. 65 illustrates an example of distortion compensation using sensors in an operating room. As shown, the operating table may have multiple (e.g., six in this example) sensors. The sensors may surround the patient. For example, as shown, there may be a sensor by the patient's head, several sensors on either side of the patient's torso, and/or a sensor near the base of the patient's torso. Although not shown, additional sensors (e.g., near the patient's feet) may be used as well.

Each sensor may detect a certain magnitude and direction of EM distortion. For example, the sensor at the top of the patient's head may detect a distortion magnitude of 2.5 (e.g., which may be a percentage of distortion relative to a base-level of distortion). The sensor near the patient's right shoulder may detect a distortion magnitude of 3.0 (e.g., slightly higher than that at the first sensor). The sensors may be used to determine a 3D distortion plane. For example, given n points with coordinates (x₁, y₁, z₁) . . . (xₙ, yₙ, zₙ), the best fit 3D plane for correcting the distorted location may be represented as zₙ=Axₙ+Byₙ+C where:

$$[ \begin{matrix}{\sum }_{i = 1}^{n} {x}_{i}^{2} & {\sum }_{i = 1}^{n} {x}_{i} {y}_{i} & {\sum }_{i = 1}^{n} {x}_{i} \\ {\sum }_{i = 1}^{n} {x}_{i} {y}_{i} & {\sum }_{i = 1}^{n} {y}_{i}^{2} & {\sum }_{i = 1}^{n} {y}_{i} \\ {\sum }_{i = 1}^{n} {x}_{i} & {\sum }_{i = 1}^{n} {y}_{i} & {\sum }_{i = 1}^{n} 1\end{matrix} ] [ \begin{matrix}A \\ B \\ C\end{matrix} ] = [ \begin{matrix}{\sum }_{i = 1}^{n} {x}_{i} {z}_{i} \\ {\sum }_{i = 1}^{n} {y}_{i} {z}_{i} \\ {\sum }_{i = 1}^{n} {z}_{i}\end{matrix} ]$$

Magnetic distortion may be caused by proximity of adjacent metallic objects within a typical OR environment. The magnetic distortion may result in 3 millimeters to 45 millimeters positional error in the EMN system of the robotic flexible endoscope. Endoscopic navigation location and orientation (e.g., of an endoscopic EMN system) may be sensed. For example, the endoscopic navigation location and orientation may be sensed using ultrasound. The endoscopic navigation location and orientation may be sensed using a force-strain measurement of the flexible scope drive.

Errors (e.g., distortion) may be sensitive to position and orientation of metal objects within the EM field and the relative location of the sensors to the impact-generating objects. The field distortion may change non-linearly as the metal objects move relative to the sensors and the emitter.

Predefined traceable calibration markers with room instrumentation may be used to monitor the 3D space of the room, tools, and movements within the room. A 3D assay of the OR may be monitored (e.g., by utilizing predictable calibrations and registration strategies).

A first coordinate system may be used to relate the current location of the flexible endoscope distal end (e.g., as it moves along a path, for example, a path defined by a pre-operative mapping of the hollow passages). For example, the distortion introduced by a fluoroscopy C-arm CT machine within the operating field near the table and patient may be a source of magnetic field distortion. A hybrid navigation framework (e.g., where EMN is used for continuous navigation and X-ray is delegated to on-demand use during delicate maneuvers or for confirmation) may be used. In this case, the X-ray machine may be a source of metallic distortion for the EMN system. The nature of the distortion may depend on the C-arm position (e.g., cannot be predetermined).

In an example, a robotic flexible scope may determine a position of its tip (e.g., based on the insertion of the scope and additive errors based on time and surrounding metal objects). A cone-beam CT arm movement may amplify the error as it moves into place to recalibrate. FIGS. 66A and 66B illustrate example distortion caused by a CT machine moving around a patient and example distortion compensation. The distorted and compensated coordinate locations of a sensor on an endoscopic probe are illustrated with coordinate systems. The scope may monitor distortion caused by proximity of the second surgical instrument (e.g., CT machine). The scope may identify a change in position of the CT machine. The distortion caused by proximity of the CT machine may be reduced due to the change in position. The scope may adjust the adjusted location of the EM sensor based on the reduced distortion.

As illustrated in FIG. 66A, while the CT machine is near the patient's head, the sensor may be impacted by relatively minor distortion. As shown, the flexible endoscopy local coordinate system may deviate from the global anatomy of the body. For example, as shown, the distorted coordinate system may be skewed counterclockwise from the original coordinate system (e.g., without distortion), but the origin of the coordinate systems may be close to the same. The cone-bean CT may be used to determine where the local coordinate system should be. The cone-beam CT may communicate the true alignment of the local coordinate system to the flexible scope (e.g., for the scope to correct its system). The realignment of the coordinates may realign the expected anatomy to that of the real anatomy.

As illustrated in FIG. 66B, as the CT arm is moved into position over the patient's chest, the distortion experienced by the sensor may increase (e.g., because the source of the distortion is closer). As illustrated by the dashed coordinate system, the measured location of the sensor may be skewed counterclockwise from the original coordinate system and shifted to the right. This may be problematic if the surgeon believes that the endoscope is in another bronchial tube. For example, the surgeon may incorrectly determine a location of a tumor and make an unnecessary incision.

The coordinate axis with white arrows in FIG. 66B illustrates a first part of a transform to compensate for the distortion. As shown, the coordinate axis with white arrows is a clockwise-shifted version of the measured coordinates. As a second part of the transform, the coordinate axis with white arrows may be shifted to the left until it is aligned with the actual coordinate axis.

If the CT is moved away, the movement may impact the signal (e.g., again). This may cause misalignment. The monitored misalignment as the cone-beam CT approached (e.g., the misalignment between FIGS. 66A and 66B) may be used as a template to computationally adjust for the return CT motion. Because the local flexible scope coordinates were monitored as the CT arm was brought into position, the system may use this as an estimate of the CT arm interference with the electromagnetic field. The estimate of the CT arm interference may be used to auto-compensate for the same adjustment as the CT arm is removed from the field (e.g., leaving the flexible scope with a true position of its coordinate system).

In an example, a pre-operative CT scan of the patient may be a reference plane (e.g., scanned in the supine position). The current (e.g., intraoperative) patient position (e.g., lateral position) may be the actual current global reference plane. The CT (e.g., cone beam CT) may be positioned by the surgeon. The CT may establish a local reference plane. The EMN of the flexible endoscope may create another local reference plane. A device (e.g., a Hugo robot) may have a (e.g., single) integrated reference plane. The integrated reference plane may be made of a plurality of (e.g., five) local reference planes (e.g., one for each arm of the device). FIG. 67 illustrates an overlayed comparison of a sensed location and an adjusted location of an endoscope.

An example device may receive EM field monitoring information from a monitoring device located at a first distance from an EM device causing the predicted location of the device to be distorted (e.g., by an EM field). The distance may be greater than a second distance between the device and the EM device. The device may adjust the predicted location of the EM sensor based on the EM field monitoring information. The distortion compensation/alignment may involve a local recalibration of the flexible endoscope electromagnetic navigation (EMN). The correction of the distortion of the EMN field sensing may be based on measurements at the sensor location and the current field distortion measured by at least one other separate (e.g., redundant) sensor. The second redundant sensor may be located at a distance from the first sensor. The distance may be greater than the size of the patient.

The C-arm for the cone beam CT may have registration elements on it. The registration elements may enable the cameras in the room to detect location, movement, and relationships to the EMN (e.g., and enable a better compensation factor for the EMN). The cameras may be enhanced with Lidar (e.g., to make accurate distance measurements in a 3D space with the spatial relationships handled by the imaging of the room visually). Predefined calibration stickers or markers may be used to reflect IR light or give the device a perspective measure (e.g., by the inclusion of several inter-related squares on each marker to allow the cameras to determine their angles with respect to the markers).

Externally-applied magnets (e.g., closer to the patient and in multiple 3D locations) may minimize the re-calibration interference caused by the C-arm. A more complete 3D assay of the room, measurement devices in the room, and the devices' relational location may be used to provide an improved in-situ calibration of the EMN (e.g., on-the-fly calibration).

Multiple imaging systems and coordinate systems may be used. A common reference plane for two imaging streams may be created (e.g., by utilizing a reference plane from a first imaging system as a means to compensate for a distortion of coordinates by a second imaging system). For example, a first imaging system may produce a laparoscopic view towards the uterus during a hysterectomy. A second imaging system (e.g., external to the patient) may track a fiducial system (e.g., a three-ball fiducial system) on the end of a uterine manipulator. The external image system may be registered to a global reference frame (e.g., by another three-ball system). The registration may be provided to the robot with a laparoscopic view. Kinetic chain errors may be reduced by using a three-ball fiducial system on laparoscopic or robotic instruments.

Reference planes from two separate visualization sources (e.g., including orientation) may be harmonized. Magnetic field navigation/tracking recalibration or distortion correction (e.g., on the fly magnetic field navigation/tracking recalibration or distortion correction) may be used. A smart system may adapt AR visualization to provide recalibration during data distortions.

Stereotactic surgery is a minimally invasive form of surgical intervention that makes use of a three-dimensional coordinate system to locate small targets inside the body. The system may then perform a procedure (e.g., ablation, biopsy, lesion, injection, stimulation, implantation, radiosurgery, etc.) on the targets. Predictable calibration and registration techniques may be used in stereotactic image-guided interventions. The predictable calibration and registration techniques may be used to minimize the burden of actively sensing distortion (e.g., distortion created by the interaction between the metal of the calibration system and the EMN).

In an example, an ultrasound (3D ultrasound) system may be used to achieve augmented reality (AR) visualization during laparoscopic surgery (e.g., for the liver). To acquire 3D ultrasound data of the liver, the tip of a laparoscopic ultrasound probe may be tracked inside the abdominal cavity (e.g., using a magnetic tracker). The accuracy of magnetic trackers may be affected by magnetic field distortion that results from the close proximity of metal objects and electronic equipment (e.g., which may be unavoidable in the operating room).

A temporal calibration may be used to estimate a time delay (e.g., between a first system moving and a second system receiving coordinate information for the first system). The temporal calibration may be integrated into the motion control program of the motorized scope control (e.g., to enable artifact magnitude identification that may be used to limit the magnitude's effect on the physical measurement of position).

Redundant electromagnetic field monitoring may be used. The EMF monitoring may be performed by a second magnetic sensor that is positioned at a distance from the primary source. The redundant measurements may be affected differently than the primary source by the metallic in the vicinity. The field distortions may be identified by the comparison of the two measurements. The identified field distortions may be minimized (e.g., removed) from the primary measurement.

Feature(s) associated with an endoscopic to laparoscopic clocking orientation multi light system are provided herein. The clocking of a flexible endoscope working channel may be aligned. The system may determine the orientation of the working channel and the camera of the flexible endoscopy device (e.g., relative to the laparoscopic view). This may provide proper coordination of the two systems. In an example, an endoscope within the colon may not know the rotational orientation of the position of the endoscope. The endoscope may not know the position of an identified lesion from the laparoscopic view. A surgeon may wiggle the tip of the scope against the colon and watch for the wiggle on the laparoscopic side. In this case, the orientation may be achieved through guesswork.

Lights may be configured on the sides of an endoscope. The lights may allow the laparoscopic view to orient to location of lesion including clocking (e.g., roll) of endoscope. The lights may provide laparoscopic-to-endoscopic support of resection of endoscope instruments. The lights may give the laparoscopic view the roll and clocking information associated with the endoscope. The working channel of the endoscope may be positioned at the desired position (e.g., 6:00 position) of the working channel to the lesion in the endoscope view. The laparoscopic view may have information of the endoscopic clocking. Laparoscopic support may be used through tissue positioning or full thickness lesion resection. Multiple color systems of LEDs may be projected circumferentially within the colon.

Laparoscopic to endoscopic light-based positional automation may be used. Based on the laparoscope view of the rotational position of the endoscope (e.g., as determined based on the pattern of projected lights), a tracking algorithm may create a positional zone in space. The zone may be laparoscopic-driven (e.g., automatically laparoscopic driven) by the robot. If the surgeon hits a ‘go to’ button on a controller interface (e.g., viewed on a console), the laparoscopic instrument may be moved into a zone near the lesion.

A time of flight distance sensor may confirm the distance to the colon and move the laparoscopic instrument a preset amount. If the preset amount is not achievable, the surgeon may receive operative communication (e.g., light, sound, haptic, etc.) of the failure. Manual motion may be allowed. For example, if the laparoscopic instrument is within the zone, an indication may be sent to the surgeon. Teleoperation of the instrument(s) may be resumed. At any time during the motion, the surgeon may move the teleoperation controls and resume surgeon-controlled motion.

One or more triggering events may initiate a calibration or indicate the increasing probability of error without recalibration. A device may redetermine an adjusted location of an EM sensor on the device if the device detects one or more triggering events. The triggering events may be temporal (e.g., recalibrate after a threshold time since the previous calibration). For example, recalibration may be triggered if the device detects that a maximum time since redetermining the adjusted location has passed. The triggering events may be positional. For example, the triggering events may be based on a change in positional relationship between the first surgical instrument and the second surgical instrument. The triggering event may be based on a distance that a surgical instrument has moved, a change of orientation or angle of a surgical instrument, and/or a change in velocity (e.g., position/time) of a surgical instrument.

The triggering events may be based on error terms. For example, the device may recalibrate if the device detects that an error associated with the adjusted location of the EM sensor is above an error threshold. The triggering events may be based on movements of a surgical device (e.g., quantity of movements, granularity of motion, and/or the like). The triggering events may be based on instrument configuration changes (e.g., articulation angle). The triggering events may be based on a risk or criticality associated with a procedure/operation step. For example, the device may recalibrate if the device detects that a criticality of an operation step is above a criticality threshold. The triggering events may be based on local monitoring of anatomy. For example, the device may recalibrate if the device detects that an anatomical structure in proximity to the first surgical instrument satisfies an anatomy condition (e.g., size of local passageways, variation from pre-operative imaging or plan, proximity to critical structures, etc.).

One or more types of recalibrations may be used. For example, the recalibration may be situational recalibration. External anatomic landmarks and/or proximity to known secondary sensing systems may be used to trigger recalibration. The recalibration may be continuous (e.g., on-the-fly continuous recalibration). Controlled/fixtured recalibration may be used. For example, robot articulation may be constrained within the trocar.

There may be a low bandwidth reference plane embedded within a high bandwidth reference plane, as illustrated in FIG. 68. If data becomes unavailable due to distortion, visual blockages such as hemostasis issues, camera fogging, device interference, reference plane may be reduced to the low bandwidth common reference plane (e.g., until coordinating system data becomes fully available). Low bandwidth imaging may use minimal data. Low bandwidth imaging may provide more general imaging until the high resolution data becomes available.

A vision system (e.g., the vision system that was not interfered with) may request specific data from another vision system (e.g., a vision system with an obscured view) in attempts to receive some information that has been labeled as critical or primary (e.g., low bandwidth information).

FIG. 69 illustrates an example method that may be performed by a surgical instrument. As shown at 54450, the method may involve sensing, using the EM sensor, a predicted location of the EM sensor. The predicted location may be distorted by an EM field. The method may involve, at 54452, receiving, from a second surgical instrument, a coordinate system associated with imaging of an area around the EM sensor. The method may involve, at 54454, determining an adjusted location of the EM sensor based on the received coordinate system. The method may involve, at 54456, outputting the adjusted location of the EM sensor.

If multiple systems capable of visualization are connected, the visualization data from the systems may be combined (e.g., within the primary system's processor), as illustrated in FIG. 70. A display may be used to merge the visualization sources from the systems. The merged visualization data may be displayed to the user. Common registration points may be used as the foundation of the merged the visualization data.

The first surgical imaging device may be an endoscopic device, and the second surgical imaging device may be a laparoscopic device (e.g., the first surgical imaging device and the second surgical imaging device may be separated by a tissue barrier). For example, as illustrated in FIG. 70, an endoscopic camera may identify a target object. The endoscopic camera may send the location information associated with the target object to an analysis engine. The analysis engine may receive another input 54402 (e.g., a global reference geometry, a transform equation, and/or the like). The analysis engine may adjust the location information based on the other input 54402 and output the adjusted location information 54404 to a laparoscopic camera (e.g., the analysis engine and laparoscopic camera may communicate via an API). The laparoscopic camera view may be from the opposite side of a tissue barrier as the endoscopic camera (e.g., inside and outside the stomach, as shown).

The laparoscopic camera may share location information associated with the orientation of the laparoscopic camera. For example, the laparoscopic camera may send its reference plane geometry to the analysis engine. The analysis engine may use the location information from the endoscopic camera and the laparoscopic camera to generate a transform between the reference geometries of the cameras. The analysis engine may reanalyze the location of the target object based on the generated transform to more accurately indicate where the target object is in the laparoscopic camera's view. The laparoscopic camera may send a live feed of its view to a display.

One of the smart surgical devices (e.g., cameras) may generate an augmented reality (AR) overlay of the first imaging data stream, the second imaging data stream, based on the transformation between the first reference geometry and the second reference geometry. The AR overlay may depict the first imaging data stream and the second imaging data stream overlapped onto one another based on the transformation. The AR overlay may include an indication of the location of the target object (e.g., determined based on the transform information from the analysis engine). The smart surgical device may output the AR overlay to a display device for displaying. The display may include the AR overlay.

Imaging systems (e.g., two separate imaging systems) may monitor a common surgical site from different points-of-view (e.g., possibly on differing side of a common organ wall). The views from the imaging systems may be switched and/or overlayed. Multiple imaging sources may be integrated. For example, the views may be overlaid on one another. The imaging systems may provide a switchable point of view (POV). Occluded viewing angles may be reduced. Organ(s) and/or tissue may be in the way (e.g., blocking the user's view). For example, a surgeon may not be able to see an instrument from a desired angle. For example, the surgeon may not be able to monitor an instrument in real time (e.g., using externally visible motion). The surgeon may want to monitor tissue positioning within jaws of an instrument. For example, the surgeon may need to be aware of an incompatible bite or tissue that is not fully captured within the active portion of the jaws. A user may desire to switch between imaging systems (e.g., two separate imaging systems having different approach angles). For example, the different approach angles may be from an endoscopic device and a laparoscopic device.

A plurality of systems may be integrated into a central system (e.g., a hub). Example communication layers are provided herein. A communication layer of a video signal may be used (e.g., by itself). Other information may be included in the video signal (e.g., an ML model to arrive at orientation, and/or the like). The communication layer of the video signal may be stamped or coupled with additional information (e.g., digitally). For example, the information may be timestamp information, camera orientation information, and/or the like. The information may aid in the synchronization of the systems.

A multi-camera (e.g., two-camera) procedure may be performed. For example, a first vision system may be located within an organ, and a second vision system may be located exterior to the organ. In a colorectal procedure, for example, a smart visualization system may be utilized for the patient. The visualization system may include multiple (e.g., two) coordinating camera systems. A first camera system may be inserted into the organ (e.g., to provide internal visualization), and a second camera system may be used to showcase a targeted area for a surgical instrument (e.g., a stapler).

In an example, a laparoscopic instrument may approach a tumor from the outside wall of the colon. A colonoscope within the colon may have robotic control of the local insertion, and/or movement of the camera (e.g., up, down, left, right, and rotational control). The surgeon may know where the endoscopic instrumentation (e.g., and associated view) is with respect to the laparoscopic instrument(s) outside the colon. The laparoscopic instruments may assist the access and resection procedures (e.g., that are taking place within the colon). Other hollow organs, such as the bladder, may use similar techniques as that described herein with respect to the colon.

An endoscopic device may calculate the transformation between the first reference geometry and the second reference geometry based on one or more lights projected, by the laparoscopic device, through the tissue barrier, and detected by the endoscopic device.

For example, a laparoscopic device with a light source (e.g., a sub-millimeter LED on laparoscopic tool) may be in contact with the colon. The light source may allow the endoscopic view to determine the position of the laparoscopic device. The endoscopic device may adjust its movements to ensure the endoscopic device and related instrumentation is driven to the location of the laparoscopic device (e.g., the cooperative site). In an example, a laparoscopic device may pinch and stretch an area around a tumor. The location of the laparoscopic device may be pinpointed using the light source. The endoscopic device may be driven to the light source (e.g., LED pinpoint) if the endoscopic device sees the light source on the endoscopic side. Once the endoscopic device reaches the cooperative site, working instruments such as graspers, injectors, cutters, and/or the like may be introduced to that site. Light (e.g., from an LED) may be projected as a laser dot from a laparoscopic shaft. The LED may be in a tethered or untethered capsule. The LED may be dropped into the laparoscopic field of view. The laparoscopic device (e.g., a laparoscopic grasper) may pick up the LED and (e.g., manually) position it as needed. In this case, the endoscopic device may use the LED pinpoint orientation as a guiding point (e.g., North Star).

A fluorescing system may be used to identify the inferior mesenteric artery (IMA) and/or adjunct vascular structures. The IMA (as seen from a first imaging system) may be used to generate an augmented view of another system (e.g., a laparoscopic view and/or endoscopic view). The IMA may be detected with a first imaging system through NIR with ICG. The imaging system may combine that position of IMA with an endoscopic and/or laparoscopic view.

The flexible endoscopy robot and the laparoscopic robot may be controlled by separate arms of the same Hugo robot. The Hugo visualization system may be coupled to the laparoscopic robot site. The flexible endoscopic scope may be an autonomous visualization unit or a second coupled unit within the same Hugo system. In this case, the use of IMA fluorescing may differ. If multiple independent systems are sharing registrations, critical structures may be used as the initial pathway to communicate imaging data bi-directionally.

A smart laparoscopic robot and visualization may couple to a hand-held flexible endoscope with an autonomous endoscope visualization source. If a smart system (e.g., that is capable of visualization) is connected to a second smart system (e.g., that is also capable of visualization), the systems may pre-identify themselves as primary or secondary. If a system requests capacity for an action, the visualization systems may alternate as needed to perform the action (e.g., based on the data capacity of the systems). If the primary systems visibility becomes obscured or limited, the visualization may switch to the secondary system. The secondary system may yield visualization to the primary system (e.g., as long as the primary systems visibility is within an operational envelope).

If the visualization system of a first device (e.g., a smart laparoscopic robot) is superior to that of a second device connected to the first device, the smarter system may prioritize its own visualization source to be displayed (e.g., and ignore the secondary, inferior source). The superior smart visualization source may communicate to the secondary source to stop transmitting visualization data (e.g., because it won't be used). The superior smart visualization source may mirror back the visualization data from the connected secondary source (e.g., so that the secondary source believes it is operating as normal and will not cause error codes). If the superior smart visualization source is not displaying data from the secondary connected source, the superior source may receive the secondary data as backup (e.g., if needed). The secondary data (e.g., second feed) may be used to confirm registration and/or the accuracy of the display. The secondary data may be used (e.g., if needed) to extend the visualization display area.

If multiple visualization systems are connected, a visual indicator (e.g., LED on device, vision system source display on screen, etc.) may be displayed to let the user know the source of the visual feed (e.g., if the vision feed is coming from an autonomous endoscope source or from a smart laparoscopic robot). The system that is being used to provide the visual feed may be based on the procedure or user preference.

FIG. 71 illustrates example components of a landmark identification and matching (LIM) engine (e.g., such as the analysis engine in FIG. 70). As shown, the LIM engine may include a processor with one or more modules. The LIM engine may be in communication with a first and second imaging device. One or more of the imaging devices and/or the LIM engine may be connected to a display.

As illustrated, at 54410, the LIM engine may receive (e.g., via a data input module) a first indication of a first location in the field of view of the first imaging device. The first location may be indicative of the location of an anatomical landmark (e.g., a tumor, major organ structure, etc.). At 54412, the LIM engine may receive a second indication of a second location in the field of view of the second imaging device. The second location may be indicative of the location of the same anatomical landmark (e.g., from a different angle and/or side of a tissue barrier).

At 54414, the LIM engine may receive a third indication of a third location in the field of view of the first imaging device. The third location may be indicative of the location of a target object (e.g., tumor, incision, etc.). The LIM engine may, at 54416, use a coordinate information module to generate coordinate information based on the first location and the second location. At 54418, the LIM engine may use a landmark identification and matching module to determine a fourth location in the field of view of the second imaging device that corresponds to the location of the target object. The LIM engine may determine the fourth location based on the coordinate information and the third location.

The second imaging device may display a live feed of its field of view on a display for a user. The LIM engine may, at 54420, output the coordinate information and/or the fourth location so that the fourth location can be displayed on the display. For example, the fourth location or the coordinate information may be added as an AR overlay on the imaging captured by the second imaging device. The overlay may allow a user (e.g., surgeon) to locate the target object without having a direct visual of the target object.

A secondary device may be integrated into a primary device. A handheld device may be integrated into a robotic device (e.g., where the robotic device is the central device). A robotic device may be integrated into a handheld device (e.g., where the handheld device is the central device).

A patient may be positioned differently for pre-operative imaging and intra-operative imaging. For example, one position may provide better visibility of internal structures being imaged pre-operatively, and another position may provide the surgeon better access to those structures during the operation, as illustrated in FIGS. 74A and 74B. Some example patient positions are described herein.

Supine position refers to a patient laying on their back horizontally. Supine position (e.g., horizontal supine position) may be used for head, face, chest, abdomen, and/or limb (e.g., lower limb) surgery. The supine position may be used in abdominal surgery, gynecological surgery, and/or orthopedic surgery.

Oblique supine position (e.g., patient tilted to one side by 45 degrees) may be used for anterior lateral approach, lateral chest wall, axillary surgery, etc. Side head supine position may be used for ear, maxillofacial, side neck, head surgery, and/or the like. Upper limb abduction supine position may be used for upper limb and breast surgery.

Lateral position (e.g., patient laying on their side) may be used for chest incision surgery, hip surgery, and/or the like. The lateral position may be used for neurosurgery, thoracolumbar surgery, hip surgery, and/or the like. The lateral position may provide sufficient exposure of the surgical field for convenient operation by the surgeon. The lateral position may cause changes in the patient's physiology (e.g., which may lead to complications such as circulation, breathing disorders, nerve damage, and/or skin bedsores).

General surgery chest lateral position may be used for lung, esophagus, side chest wall, and/or side waist (e.g., kidney and ureter middle and upper part) surgery, etc. Lateral position may be used for kidney and ureter middle and upper section surgery. The distance between the lower rib to the lumbar bridge may be three centimeters (e.g., which may be suitable for kidney surgery, nephrectomy, ureteral stone removal). If a lumbar bridge is not present, the “folding bed” may be in a flex position. Lateral position may be used for hip surgery (e.g., for acetabular fracture combined with posterior dislocation of the hip, artificial hip replacement, quadratus femoris bone flap transposition for the treatment of aseptic necrosis of the femoral head, open reduction and internal fixation of femoral shaft fractures, femoral tumors, femoral neck fractures or intertrochanteric fractures, internal fixation and upper femoral osteosynthesis, etc.).

The Trendelenburg position (e.g., supine position with head down) is a variation of the supine position. Trendelenburg position may be used in head, neck, thyroid, anterior cervical surgery, cleft palate repair, general anesthesia, tonsillectomy, tracheal foreign body, esophageal foreign body surgery, and/or the like. The Trendelenburg position may be accompanied by a small downward fold of the leg plate. The Trendelenburg position may be used for laparoscopic surgery, or pelvic or lower abdomen surgery. The patient's posture may be able to move to the supine position (e.g., under normal conditions or in emergency situations, for example, if the power supply is interrupted). FIGS. 74A and 74B illustrate how the Trendelenburg position may provide a surgeon with better access to some organs compared to the supine position.

Reverse-Trendelenburg (e.g., supine position with head up) is a variation of the supine position with a forward tilt. Reverse-Trendelenburg position may be used for head and neck surgery, and/or abdominal procedures (e.g., bariatrics). For example, the Reverse-Trendelenburg position may be used in head and neck surgery to reduce venous congestion and prevent gastric reflux (e.g., during the induction of anesthesia). In abdominal procedures, Reverse-Trendelenburg position may be used to allow gravity to pull the intestines lower (e.g., providing easier access to the stomach and adjacent organs).

In the Jack-knife position (e.g., sometimes referred to as the Kraske position) a patient's head and feet are lower than the patient's hips. Jack-knife position may be used for gluteal muscle and anal (e.g., rectal) surgery. In gallbladder and kidney surgery (e.g., in the absence of a lumbar bridge), the back and buttocks may be folded to form an arch (e.g., to replace the lumbar bridge). The arched starting point may be fixed. The height may be limited by the folding angle.

The patient surgical position may impact the insufflation pressure, the peripheral blood flow, the anesthesia magnitude, breathing, heart rate, and/or the like. While the patient surgical position may provide better access to the surgical site, the position may affect related system performance and thresholds (e.g., smart digital system closed loop performance and thresholds). For example, compared with the mean inflated volume for the supine position (e.g., 3.22±0.78), the mean inflated volume may increase by 900 ml for the Trendelenburg position or if the legs are flexed at the hips, and may decrease by 230 ml for the reverse-Trendelenburg position.

FIG. 72 illustrates an example method for object registration. At 54430, a system may establish a global coordinate system (e.g., using pre-operative imaging). The system may, at 54432, establish one or more local coordinate system(s) for intra-operative imaging. As discussed herein, the patient's position may have changed between the pre-operative imaging and the intra-operative imaging. Accordingly, anatomical structures may have shifted/changed locations.

At 54434, the system may use fiducial markers to define a reference configuration relative to the global coordinate system. The system may, at 54436, perform object registration of anatomical landmarks relative to the fiducial markers. At 54438, the system may interpret the intra-operative imaging based on the registration of the anatomical landmarks. For example, the system may identify the anatomical landmarks even though they have shifted since the pre-operative imaging was performed.

In examples (e.g., if multiple imaging platforms/technologies are in play), communication between the systems may help with coordination techniques described herein. The endoscopic view (e.g., flexible endoscopic view) may have visualization. The endoscopic view may have ultrasound imaging onboard. The laparoscopic camera may have (e.g., only have) the visual image. Connecting the information from the two systems and the pre-operative imaging (e.g., which may provide more or better information than either system alone) may enable the real-time identification of structures within either visualization platform (e.g., laparoscopic or endoscopic).

An example of fiducial alignment is provided herein. The global coordinate system may be established based on a pre-operative CT image. A laparoscopic system and endoscopic system may have their own local coordinate systems. Through the use of multiple fiducial markers (e.g., seen by each system), a reference coordinate system (e.g., a reference configuration) may be defined relative to the global coordinate system (e.g., with deformations roughly accounted for based on the current image). Coordinating the fiducials in real time may provide greater clarity and/or accuracy in the local coordinate systems for each system. The object registration may enable interpretation of structures in the current image that the local technology (e.g., visual laparoscopic camera) cannot definitively identify.

In an example, gallbladder stones may be occluded in the common bile duct. The common bile duct may be opened up and the gallbladder (e.g., which is the source of the stones) may be removed. This may involve multiple (e.g., two or three) cooperative smart systems (e.g., a robotic flexible endoscope, a robotic laparoscope, and optionally a high intensity ultra sound therapeutic system). At least two of the systems may interchange data, registrations, and/or the like. The systems may work from both sides of a tissue barrier (e.g., inside and outside an organ wall), as shown in FIG. 73. A third system may receive targeting information from one or more of the other systems. The third system may not provide data back to the other systems.

FIG. 73 illustrates an example of two scopes registering objects in their field of view. As shown, the scopes may be on opposite sides of a tissue barrier (e.g., a laparoscopic side and an endoscopic side). A first scope (labeled Cam1) may identify two objects (e.g., object 1 and object 2) in its field of view. The first scope may register the two objects. The first scope may determine an angle between the two objects.

As illustrated, a second scope (labeled Cam2) may also register the two objects. The second scope may further register a target object. The second scope may send location information (e.g., related to its field of view) to the first scope or to an independent system. The first scope or other system may determine, based on the first scope's registration of the two objects and the second scope's registration of the two objects and location information of the target object, where the target object is located in the first scope's field of view. For example, the first scope or other system may compare the first scope's registration of the two objects to the second scope's registration of the two objects to determine a coordinate transform between the views of the scopes. For example, in FIG. 73, the transform may indicate that the view of the first scope is a reflection of the second scope's view over the horizontal axis. The first scope may use the transform to determine where the target object is located in the first scope's field of view.

Gallstones may cause problems such as pain (e.g., biliary colic) and gallbladder infections (e.g., acute cholecystitis). Gallstones may migrate out of the gallbladder. The gallstones may become trapped in the tube between the gallbladder and the small bowel (e.g., common bile duct). In the common bile duct, the gallstones may obstruct the flow of bile from the liver and gallbladder into the small bowel. This may cause pain, jaundice (e.g., yellowish discoloration of the eyes, dark urine, and pale stools), and sometimes severe infections of the bile (e.g., cholangitis). Between 10% and 18% of people undergoing cholecystectomy for gallstones may have common bile duct stones.

The pancreas is a long, flat gland that sits tucked behind the stomach in the upper abdomen. The pancreas produces enzymes that help digestion and hormones that help regulate the way the body processes sugar (e.g., glucose). Pancreatitis refers to inflammation of the pancreas. Pancreatitis may occur if the bile duct is clogged and the pancreas enzymes cannot be transferred into the small intestines. In this case, the enzymes begin to break down the pancreas itself (e.g., from the inside out). Pancreatitis may occur as acute pancreatitis (e.g., pancreatitis that appears suddenly and lasts for days). Some people develop chronic pancreatitis (e.g., pancreatitis that occurs over many years). Mild cases of pancreatitis improve with treatment. Severe cases may cause life-threatening complications.

Treatment of gallstones may involve removal of the gallbladder and the gallstones from the common bile duct. There are several ways to remove the gallbladder and gallstones. Surgery may be performed to remove the gallbladder. The surgery may be performed through a large incision through the abdomen (e.g., open cholecystectomy). The surgery may be performed using keyhole techniques (e.g., laparoscopic surgery). Removal of the trapped gallstones in the common bile duct may be performed at the same time as the open or keyhole surgery. An endoscope (e.g., a narrow flexible tube equipped with a camera) may be inserted (e.g., through the mouth) into the small bowel to allow removal of the trapped gallstones from the common bile duct. This procedure may be performed before, during, and after the surgery to remove the gallbladder. A surgeon may have to determine whether to remove the common bile duct stones during surgery to remove the gallbladder (e.g., as a single-stage treatment), or as a separate treatment before or after surgery (e.g., a two-stage treatment).

The pre-operative CT or MRI may have a larger perspective view of the surgical area (e.g., compared to intraoperative imaging devices). The CT or MRI may come with limitations. For example, the CT or MRI may be taken at an earlier point of time and the anatomy and disease state progression may be different from that at the time of surgery. The pre-operative imaging (e.g., CT or MRI) may be taken in the supine portion, while surgery may be performed in the Trendelenburg position. This change in position may cause the organs and surgical site to distort (e.g., shift compared to the pre-operative imaging). The distortion may not be uniform with the retroperitoneal and peritoneal (e.g., structures behind the peritoneum or in front of the peritoneum, respectively) organs adjusting differently (e.g., due to different levels of fixation to more rigid portions of the body). For example, retroperitoneal structures may move less with changes in anatomic position (e.g., because such structures are more rigidly fixated to the back wall of the cavity).

In this example, the first surgical imaging device may be an intraoperative imaging device and the second surgical imaging device may be a pre-operative imaging device. The transformation between the first reference geometry and the second reference geometry may be calculated based on a difference between a first patient position during capture of the first imaging data stream and a second patient position during capture of the second imaging data stream, as described herein.

The change in position and its effect on the location of some anatomical structures is illustrated in FIGS. 75A and 75B. As shown, the gallbladder may be moved away from the small intestine and the pancreas may be elongated in Trendelenburg position. Similarly, gallstones may migrate downstream during the time between pre- and intra-operative imaging. GI motility or other movement of objects through the organs may distort the synchronization of imaging registration.

Patient anatomic positioning may be compensated for during surgery. The time dependent variable may be addressed (e.g., after compensating for the patient anatomic positioning). The pre-operative imaging may be used for guidance, registration, or identification of differing real-time surgery imaging. For example, a first surgical imaging device may be an intraoperative imaging device and a second surgical imaging device may be a pre-operative imaging device. The first imaging data stream may be captured with a patient in a first patient position and the second imaging data stream may be captured with the patient in a second patient position.

In this case, the anatomy may be adjusted to that of the surgical position. The adjustment may be done by identification of common landmarks that are less- or non-moving landmarks, 3D shape comparison, and/or local fiducial marker synchronization. For example, the reference landmark may be an anatomical structure that has relatively little movement between capture of the first and second imaging data streams. In the example illustrated in FIGS. 75A and 75B, the aorta and/or splenic artery may be used as the common landmark(s) since those structures moved very little, if at all, between the two images.

As illustrated, anatomy that is more controlled (e.g., with less movement and more constant 3D shape) may be used to differentiate structures. The system may determine local characteristics of the other structures to identify those structures. The system may display the adjusted images to the user. The adjusted images may be used for annotation and/or later navigation & intervention.

Local ultrasound imaging (e.g., using an EBUS ultrasound sensor) may identify sub-surface anatomical structures and/or foreign objects (e.g., within adjacent hollow structures like the common bile duct). A surgeon may use an EBUS ultrasound and/or visual light imaging flexible endoscope. The sensor may be enclosed in a balloon filled with saline (e.g., which makes contacting the duodenum wall easier for ultrasound imaging). The system may differentiate the objects from one another. The system may mark the objects' registration (e.g., for later interaction). Ultrasound may provide a good size and shape interpretation of the objects. Ultrasound may not provide information regarding other features (e.g., object density or composition).

The system may differentiate between detected objects (e.g., two detected objects in close proximity with similar size and shape). For example, the system may utilize the adjusted pre-operative imaging map (e.g., with focus on the local area of concern). This may account for retroperitoneal and peritoneal positioning to forecast where structures in pre-operative images are after the patient is moved. FIG. 76 illustrates an example display in which pre-operative and intra-operative imaging are overlaid on one another. The overlaid images may enable the surgeon to identify structures in intra-operative images based on their relative location/shape compared to the pre-operative images.

The system may utilize mapping of adjacent or enclosed secondary structures. For example, a gallstone may be in the hollow adjacent bile duct and the lymph nodes may not be in that bile duct. If the common bile duct location can be imaged or predicted (e.g., like a street mapping program), the system may be able to differentiate objects that are moving within the common bile duct. FIGS. 77A-C illustrate the change in location of anatomical structures (e.g., gallstones and lymph nodes) between pre- and intra-operative imaging.

If the system were unable to adequately identify and annotate the structures, a third (e.g., gray) visualization may be used to denote a structure having no identifier. The surgeon may move the scope closer to the unidentified object. The surgeon may identify the object (e.g., visually using the visual light imaging scope on the flexible endoscope).

Lymph nodes may not be part of the cholecystectomy (gallbladder removal) or Endoscopic Retrograde Cholangiopancreatography (ERCP) stone removal from the bile duct. The gallstones may be addressed/removed during the procedure. As the structures are identified, the annotation of the structures may allow the user to approach the structure (e.g., for removal). The structures may be noted for other vision system to use for common registration. The laparoscope may be able to differentiate gallstones in the duct from lymph nodes. The location of the gallstones may help the system determine where to transect (e.g., so as to not leave any remaining stone in the duct that may cause later complications).

Situational awareness may be applied to help determine differences between structures (e.g., that may not be definitively identified otherwise). For example, a gallstone and a lymph node may appear similar (e.g., similar size, density, etc.). With knowledge of the anatomy (e.g., such as the biliopancreatic duct structures), the surgeon may know that objects residing within the duct are gallstones and not a lymph node. Similarly, if the object resides outside the duct, the object cannot be a gallstone.

Pre-operative images may be adjusted to fit real time imaging. Once the local coordinate systems are defined (e.g., the local coordinate systems may be updated real time), an analysis of the available images (e.g., from multiple sources) may be performed to identify structures (e.g., key structures of interest). CT may be able to show structural elements and landmarks (e.g., the common bile duct, biliopancreatic duct, ampulla of Vater, gallbladder, liver, pancreas, duodenum, and/or gallstones, although results may be inconclusive). With a local coordinate system known relative to an adjusted global coordinate system, information from EBUS may identify gallstones, ampulla of Vater, common bile duct, biliopancreatic duct, etc. This information may be used to confirm the identity of structures from other views. For example, with coordination, the laparoscopic view may identify structures for the surgeon (e.g., based on the information gathered and interpreted from the CT image and/or the ultrasound image). This approach may be used to identify lymph nodes and/or other structures of interest.

Differentiating gallstones from other structures may be difficult on the full body CT. Differentiating gallstones from other structures may be easier under local ultrasound. The system may re-establish registration (e.g., compensate for patient positioning changes). The system may make a more decisive determination as to whether a structure is a gallstone. The system may tag the gallstones for communication to other smart system(s). The other system(s) may use the in-surgery registration to obtain location and positioning information. The presence of kidney stones and gallstones via CT may be detected 95-98% of the time.

The accuracy of a low dose CT to discriminate a gallstone from adjacent normal anatomic structures may be lower than that of other imaging devices. Radiologists may only be able to discriminate all the stones and their locations 72-100% of the time. Low dose CT scans may correctly identify kidney stones 90%-98% of the time. Low dose CT scans may correctly confirm no kidney stones were present 88%-100% of the time. Ultra-low dose CT scan may correctly identify kidney stones 72%-99% of the time. Ultra-low dose CT scans may correctly confirm no kidney stones were present 86%-100% of the time.

External ultrasonic imaging may be as accurate as CT (e.g., and possibly less accurate) in the identification of stones. Local ultrasound (e.g., on a flexible endoscope) may be more (e.g., significantly more) accurate in identifying stones and determining the location of stones. The local system may be used to determine the in-surgery registrations, determine whether a structure is a stone or anatomy, and/or adjusting other imaging systems to match the imaging seen in real time.

The EBUS scope may be removed (e.g., after the imaging, registration and annotation are complete). The EBUS may be replaced with a standard visualization scope. The EBUS system may have (e.g., may only have) a 2.2 millimeter working channel. A basket snare retrieval tool may use a 2.8 mm working channel to operate and retrieve stones in the common bile duct. A guide wire may be introduced down the working channel of the EBUS device. The EBUS scope may be removed while leaving the guide wire in place. A larger working channel visual scope may be introduced over the wire to get it back in the same position. The registration and imaging may be used to guide the new scope into the proper location for bile duct introduction and stone retrieval.

Some of the stones to be retrieved may be too far up the duct (e.g., and therefore out of reach of the physical retrieval basket. In this case, the doctor may use an extracorporeal (e.g., from outside the body) Shock Wave Lithotripsy. The Shock Wave Lithotripsy may generate sound waves that are focused on the stone from an outside high intensity ultrasound generator. The sound waves may crush the stone. The endoscopy visualization and pre-registrations may be used to target the stone. The sound waves may be used to crush the stone into small pieces.

Once the stones are handled (e.g., retrieved or crushed) the laparoscopic-side surgery may begin. The surgeon may select where in the duct from the gallbladder to transect. The duct, the vein, and the artery feeding the gallbladder may be transected with an endocutter. The gallbladder may then be removed. A stone may be located near the gallbladder (e.g., within the duct). To remove the stone (and the stones in the gallbladder), the transection may be made below the stone location. In this case, the stone may remain in the extracted specimen side of the transection (e.g., not the remnant side). To do this, the imaging of the EBUS system and the registration of the stone in the duct may be used to determine the exact transection site of the duct.

FIG. 78 illustrates an example method that may be performed by a surgical instrument. As shown at 54450, the method may involve capturing a first imaging data stream of a visualized surgical structure. The method may involve, at 54452, communicating registration information with a second surgical imaging device capturing a second imaging data stream of the visualized surgical structure. The method may involve, at 54454, identifying, based on the registration information, a first location, within the first imaging data stream, associated with the visualized surgical structure. The first location may be determinable based on: a first reference geometry associated with a field of view of the first surgical imaging device, a second reference geometry associated with a field of view of the second surgical imaging device, a transformation between the first reference geometry and the second reference geometry, and a reference landmark common to the first imaging data stream and the second imaging data stream.

3Di GmbH may use CT scans to create precisely adapted patient-specific implants. The CT scans may be taken of the patient. The data may be transferred to 3Di, which will process the data and create a virtual patient model. Once the patient model is confirmed, 3Di will create a 3D implant model that may be reviewed/confirmed by a doctor. The implant model may then be manufactured and sent to site for implant.

In this example, multiple scan sources may be combined to further enhance the model created (e.g., instead of only utilizing a CT scan). For example, in addition to CT imaging, ultrasound and/or MRI may be used to develop the 3D model.

The 3Di processor may interact with the CT scanning system (e.g., to identify inputs that do not generate correctly and/or anomalies identified during the virtual model). This coordination may minimize potential errors or differences within a patient to create a more accurate representation of the implant.

Photogrammetry may be used to enable one or more feature(s) described herein. Photogrammetry may use photographic cameras to obtain information about the 3D world. Photogrammetric measurements may be performed by recording a light ray in a photographic image. The light ray may correspond to observing a direction from the camera to the 3D scene point where the light was reflected or emitted. From this relation, the cameras may be oriented relative to each other, or relative to a 3D object coordinate frame, to reconstruct unknown 3D objects through triangulation.

For single-view geometry, a collinearity equation may be used. The mapping with an ideal perspective camera may be decomposed into two steps: a transformation from object coordinates to camera coordinates (e.g., sometimes referred to as exterior orientation), and a projection from camera coordinates to image coordinates using the cameras' interior orientation. The exterior orientation may be achieved by a translation from the object coordinate origin to the origin of the camera coordinate system (e.g., the projection center) and a rotation that aligns the axes of the two coordinate systems.

With the Euclidean object coordinates X₀^{e}=[X₀, Y₀, Z₀]^{T} of the projection center and the 3×3 rotation matrix R, the relationship may be expressed as:

$$\tilde{x} = MX = [ \begin{matrix}R & 0 \\ {0}^{⊤} & 1\end{matrix} ] [ \begin{matrix}1 & - {X}_{0}^{e} \\ {0}^{⊤} & 1\end{matrix} ] X$$

where upper case letters (e.g., X) refer to object coordinates, lower case letters with a tilde (e.g., \tilde{x}) to camera coordinates, and plain lowercase letters (e.g., x) to image coordinates.

With respect to the camera coordinate system, the image plane may be perpendicular to the z-axis. The z-axis may be referred to as the principal ray. The z-axis may intersect the image plane in the principal point, which has the camera coordinates \tilde{x}_{H}=\tilde{t}·[0, 0, c, 1]^{T} and the image coordinates x=t·[x_{H}, y_{H}, 1]^{T}. The distance c between the projection center and the image plane is the focal length (or camera constant). The perspective mapping from camera coordinates to image coordinates may be written as:

$$x = [ K \vert 0 ] \tilde{x} = [ \begin{matrix}c & 0 & {x}_{H} & 0 \\ 0 & c & {y}_{H} & 0 \\ 0 & 0 & 1 & 0\end{matrix} ] \tilde{x}$$

This relation holds if the image coordinate system has no shear (e.g., orthogonal axes, respectively pixel raster) and isotropic scale (e.g., same unit along both axes, respectively square pixels). If a shear s and a scale difference m are present, they amount to an affine distortion of the image coordinate system, and the camera matrix becomes:

$$K = [ \begin{matrix}c & cs & {x}_{H} \\ 0 & c ( 1 + m ) & {y}_{H} \\ 0 & 0 & 1\end{matrix} ]$$

with five parameters for the interior orientation.

By concatenating the two steps from object to image coordinates, the final projection (e.g., the algebraic formulation of the collinearity constraint) may be expressed as:

$$x = λ PX ∝ PX = KR [ I \vert - {X}_{0}^{e} ] X$$

If an object point X and its image x are both given at an arbitrary projective scale, they will only satisfy the relation up to a constant factor. To verify the constraint, (e.g., check whether x is the projection of X, one can use the relation:

$$x \times PX = {[ x ]}_{x} PX = 0$$

Note that due to the projective formulation only two of the three rows of this equation are linearly independent. Given a projection matrix P, the interior and exterior orientation parameters may be extracted. For example, the following equation may be used to determine the parameters:

$$P = [ M \vert m ] = [ KR \vert - {KRX}_{0}^{e} ]$$

The translation part of the exterior orientation immediately follows from X₀^{e}=−M⁻¹m. The rotation may (e.g., by definition) be an orthonormal matrix. The calibration may (e.g., by definition) be an upper triangular matrix. Both properties may be preserved by matrix inversion. The two matrices may be found by QR decomposition of M⁻¹=R^{T}K⁻¹ (or by RQ decomposition of M).

A person of ordinary skill in the art will appreciate that other geometries (e.g., two-view geometry) may be used to enable the features described herein.

The location and/or operational parameter(s) of the therapy modality may be controlled to define a three-dimensional therapeutic envelope shape of primary effect and/or secondary collateral interaction. A surgical system may adjust one or more parameter(s) of the therapeutic treatment in response to the qualitative feedback information (e.g., information related to the effectiveness of the treatment). Example parameters of the therapeutic treatment may include one of more of: a location of the therapeutic treatment; a drug concentration; an injection pressure; a power level of a surgical instrument associated with the therapeutic treatment; a temperature associated with the surgical instrument; or a resolution of one or more imaging scans. FIG. 79 illustrates an example surgical operating room with therapy monitoring.

Systematic coordinated control of two related but independent operations may be used to control the boundaries of treatments. The interaction between systems may be controlled around an intended zone of effect. For example, one system may be providing therapy, and the other system may be providing feedback control monitoring of the therapy or outcome.

The outcome of the treatment may be monitored (e.g., by a system that is independent of the system inducing the outcome). The monitoring may be used to control the magnitude of the effect applied. The monitored feed from a first smart system may be used to determine the magnitude of the effect of a second smart system. The treatment may be adjusted based on monitoring the outcome (e.g., by an independent system).

The primary therapy effect may be different from an intended collateral effect. The positional control may be balanced with the therapy applied effect to control the primary and collateral envelope zones. The intended primary effect may be a control percentage of cell death. The intended collateral effect may be an intended amount of cellular damage but not cellular death. The relational envelopes of the primary and collateral effects may be defined in terms of size and shape.

FIG. 80 illustrates an example therapy monitoring tool. The therapy monitoring tool may be an application (e.g., on a laptop or tablet) and/or incorporated in a surgical system (e.g., a surgical hub). The therapy monitoring tool may include a graphical user interface (GUI). An example GUI is illustrated at the top of FIG. 80. A user (e.g., medical professional such as a surgeon) may select (e.g., from drop down menus) a therapeutic treatment (e.g., a therapy type) and/or a corresponding medical data stream relevant to the therapeutic treatment (e.g., from a monitoring instrument). The monitoring instrument may be the surgical instrument that is capable of monitoring the level of therapy and/or the patient's response to the therapy (e.g., increased air inhalation, reduced heart rate, etc.). The user may select an output type. The user may select a threshold (e.g., 70%, 80%, or 90% in the example of FIG. 80). The threshold may indicate a threshold level of therapy to be applied, a threshold level of effect (e.g., biological response) of the treatment, and/or the like.

The user inputs may be used to generate a treatment profile. For example, the inputs may be sent to a database, and a profile matching the therapy ID and Monitor ID input by the user may be output. The therapy monitoring tool may display a graphical representation of the treatment profile. The graphical representation may include an indication (e.g., demarcation) of the threshold that was selected (e.g., as a dotted line showing the threshold). The graphical representation may include a graph showing the progress (and/or projected progress) of the treatment and/or the effect on the patient over time. For example, the graph may show a chemotherapy saturation level of a tumor over time. In this case, the user may be able to monitor the saturation level and cease treatment once the saturation level reaches 90%.

FIG. 81 illustrates example components of a therapy monitoring device. The therapy monitoring device may include a processor with one or more databases, a data input/output module, and/or a display. The data input/output module may receive user input indicating a selected therapeutic treatment. The user input may indicate a selected medical data stream from selected monitoring device(s) (e.g., or the therapy monitoring tool may select a suitable monitoring device based on the selected therapy).

The user input(s) may be funneled to respective database(s) (e.g., the selected therapy may be sent to a therapy database and the selected monitoring device may be sent to a monitoring device database). If a monitoring device is not selected, the therapy database may send an indication to the monitoring device database of an appropriate monitoring device to select. Based on the therapy and monitoring device(s) used, a profile database may determine available profile(s) that may be used to monitor the therapy. If the user selected an output type and/or threshold, the profile database may determine the available profile(s) based on that input as well. The profile(s) may be displayed (e.g., on a display of the therapy monitoring tool, and/or on a separate display).

The system may determine, based on the medical data stream and the selected therapeutic treatment, qualitative feedback information about the therapeutic treatment. The qualitative feedback may be indicative of an effectiveness of the therapeutic treatment. Determining the qualitative feedback information about the therapeutic treatment may involve determining an expected effect on a patient receiving the therapeutic treatment. The expected effect may be compared to the medical data stream. The qualitative feedback information may be determined based on the comparison.

Determining the qualitative feedback information about the therapeutic treatment may involve determining a threshold value associated with the medical data stream and the therapeutic treatment. The threshold value may be compared to the medical data stream. On a condition that the medical data exceeds the threshold value, the therapeutic treatment may be adjusted.

In an example, airflow inhalation and exhalation (tidal) volumes may be used as treatment indicators. Respiratory mechanics may refer to the interaction between the elastic/spongiform lungs, the elastic/rigid chest wall (thorax), and the bellows-like action of the diaphragm. During normal breathing, the diaphragm's excursion causes the lung to expand. The expansion creates a negative pressure (e.g., relative to normal air pressure) within the bronchial tree (e.g., 10-15 cm H₂O) with a resulting in-flow of room air at normal pressure. As the elastic recoil of the thorax and diaphragm occurs, the recently-inhaled gas escapes under slightly greater-than-room air pressure. This gas exchange allows the organism to ventilate (e.g., remove CO₂) and oxygenate. Two forms of mechanical ventilation are pressure ventilation (e.g., peak airway pressure constant, tidal volume variable) and volume ventilation (e.g., tidal volume constant, peak airway pressure variable.

In an example, the therapeutic treatment may be a tumor debulking therapy. The medical data stream may be a tidal volume of airflow at a given pressure. The qualitative feedback may be an indication of whether the tidal volume of airflow has reached a desired amount at the given pressure.

FIG. 82 illustrates example selection options provided by the therapy monitoring device. The user may select a therapy, in this example, cryoablation debulking. The user may then select a metric to monitor and/or a device to use for monitoring the metric. In this example, the user may select tidal volume as the metric. The therapy monitoring system may present two available profiles based on the user selections. For example, the profiles may have different thresholds and/or ranges (e.g., which may correspond to levels of treatment aggression). The therapy monitoring tool may display a graphical representation of the selected profile (e.g., Profile 2). The graphical representation may include a line illustrating when to stop ablation (e.g., a range of tidal volume that indicates that the tumor has been adequately debulked).

In this example, a smart ventilator may monitor the tidal volume of airflow at a given pressure. The smart ventilator may monitor the pressure at a given volume to monitor debulking therapy of a tumor. The debulking treatment may be used to reinstate airflow to a restricted portion of the patient's airways. The resumption of the amount of desired airflow at the desired pressure may be used as feedback to control the ablation system (e.g., to indicate that the tumor size has been sufficiently reduced). Tumor debulking may increase the chance that chemotherapy or radiation therapy will kill all the tumor cells. Tumor debulking may relieve symptoms or help the patient live longer.

Approximately one-third of patients with lung cancer will develop an airway obstruction. Many cancers lead to airway obstruction through metastases. Malignant Airway Obstruction (MAO) is when a tumor impinges on the bronchus connecting the main airway to the sub-sections of the lung. The treatment of MAO may involve a multi-modality approach. The treatment may be performed for palliation of symptoms in advanced lung cancer. Removal of an airway obstruction may be used to improve symptoms, quality of life, and lung function. Patients with short life expectancy, limited symptoms, and/or an inability to visualize beyond the obstruction may not be selected as candidates for this treatment.

FIG. 83A illustrates an example therapy device inserted in bronchial tubes. In this example, a bronchial segment (e.g., the bronchus to segment three of the right lung) may have obstructed airflow (e.g., because of a tumor). This may reduce the tidal volume of breathing by about 10% from full capacity. As shown in FIG. 83A, a flexible endoscope may be used to enter the bronchial segment. The flexible endoscope may have a treatment delivery system (e.g., a needle with which to inject a drug, an ablation tool, and/or the like). An endoscopic robotic bronchoscopy procedure may be performed with a cryoablation, radio frequency monopolar ablation, or irreversible electroporation to reduce the size of a tumor and reestablish air exchange and/or reduce the tumor size for other tumor treatments (e.g., radiation therapy, chemo-therapy, immuno-therapy).

As shown in FIG. 83B, a portion of the tumor may be fully killed (e.g., necrotized). The remaining portion of the tissue may be damaged (but not killed) so that the body's immune response may engage the remaining cancerous material. The killed portion of tissue may be exposed to sufficient levels of heat, cold, or electrical field to cause the destruction of the cells. Collateral interactions with adjacent cancerous cells may be limited in temperature, cold, or potential to leave the cells alive (e.g., but damage the cells to prevent undesired destruction of proteins that help the immune system target the remaining cells). The damaged cells may direct the immune system to the area to destroy the remaining cells (e.g., some proteins that initiate an immune response may not be destroyed). This treatment may cause the tumor to lose its hiding ability to the immune system so the immune system can remove and remodel the tissue.

The location of the fully-killed cells and the damaged cells may be controlled (e.g., based on the temperature used and the duration of the ablation and/or the magnitude of the patient's immune response). Heat or cold ablation may be used. Examples of the temperature ranges for heat ablation include temperatures greater than 60° C. to kill cells, and 35 to 45° C. to damage cells. Examples of the temperature ranges for cold ablation (e.g., cryoablation) include temperatures less than −30° C. to kill cells, and 0 to −10° C. to damage cells (e.g., a full therapeutic range for cryoablation is −30° C. to −75° C.).

The duration and temperature of the ablation may be used as control parameters for treatment. For example, a time-at-temperature transform may be used to determine cell death. For example, the therapeutic treatment may be an ablation therapy configured to produce a threshold amount of cell death. The medical data stream may be an ablation temperature and a time duration for which the ablation therapy has been applied. The qualitative feedback may include an indication of whether a time-at-temperature transform indicates that the threshold amount of cell death has been attained.

If a greater effect is desired, the time and/or temperature may be increased. The temperature ranges may be monitored by multi-spectral imaging to provide the data to the ablation system. The data from the multi-spectral imaging may be used as a predictive model of time and/or probe temperature (e.g., with no real-time real-tissue feedback on their effects on the temperature profile).

A portion of the tumor may be debulked (e.g., reduced in size to restore the air path allowing the segment three right to be functionally used). Debulking may be done using cryoablation. FIG. 83B illustrates the example therapy device of 9A providing cryoablation treatment to the tumor. In cryoablation, an extremely cold liquid (or an instrument called a cryoprobe) is used to freeze and destroy abnormal tissue. A cryoprobe may be cooled with substances such as liquid nitrogen, liquid nitrous oxide, or compressed argon gas. Cryoablation may refer to destroying tissue by freezing. Cryoablation may be used, for example, in the treatment of cancer. Cryoablation may shrink cancer tumor(s). Cryoablation may result in a significant decrease in pain (e.g., compared to pre-debulking and to other treatment methods). Cryoablation may be used to treat malignancy in a wide variety of organs (e.g., the eye, brain, head/neck, and esophagus). Cryoablation may be used in the treatment of liver, kidney, lung, prostate, and breast malignancy. Cryoablation may be performed via surgical (e.g., open or laparoscopic) or percutaneous approaches. Improvements in imaging (e.g., which allow earlier detection of smaller cancers) and a trend toward minimally invasive techniques in oncology have made image-guided oncologic intervention like cryoablation an attractive alternative to surgical treatments.

Cryoablation may be used to reduce the portion that is collapsing the bronchus (e.g., establishing two necrosis areas). A robotic flexible endoscope may use cryoablation to reduce the obstruction by 75%. A portion (e.g., 45%) of the tumor may be killed (e.g., by fully killing some cells). The endoscope may be used to control the position of the cryoablation. A closed control loop may use multi-spectral imaging (e.g., from the laparoscopic or endoscopic side) to monitor local temperatures within the kill, damage, and surrounding tissue zones. The control loop may balance the magnitude, pressure, and/or direction of the liquid (e.g., argon, nitrogen, or carbon dioxide) to keep the three zones within the desired ranges (e.g., and minimize bleed over from one zone to the next).

One or more parameters may be monitored by a smart system. For example, the system may monitor the location of the ablation device, the ablation temperature, power level, tissue impendence, tissue temperature, adjacent tissue temperature or thermal gradient, airway flow volume, volume (e.g., visualized 3D volume) of the tumor, and/or the extent of the acute ablative tumor debulking. FIG. 83C illustrates therapy monitoring associated with the example therapy device of FIGS. 83A and 83B.

A surgeon may determine that the debulking is sufficient if the surgeon sees a visualized size reduction of the impingement. The surgeon may monitor the tidal volume of the inhalation air flow as an indicator of the debulking sufficiency. For example, as illustrated in FIG. 83C, the surgeon may debulk the tumor and monitor the tidal volume until there is at least a 10% increase in volume at a set pressure. This technique may allow the debulking to be based on a size of the tumor reduction that was blocking the airway (e.g., rather than a fixed amount of tumor).

Examples of tumor obstructions of airways are provided herein. The airway obstructing tumor may affect an entire lung or lobe. Transbronchial tumors may be used to diagnose diffuse parenchymal lung disease (DPLD). The reported diagnostic yield may be between 70% to 80%. Tracheobronchial tumors may form in the inside lining of the trachea or bronchi (e.g., large airways of the lung). These may be a small portion of the overall Transbronchial tumors.

The patient's breathing may be used to control the de-bulking magnitude of the tumor located in the airway. Waveform capnography may indicate the amount of carbon dioxide (CO₂) in exhaled air (e.g., which may be used to assess ventilation). Waveform capnography may be depicted as a number and a graph. The number may represent the capnometry (e.g., the partial pressure of CO₂ detected at the end of exhalation). Transcutaneous oximetry (tcpO₂) may be used to measure the local oxygen released from the capillaries through the skin (e.g., reflecting the metabolic state of the lower limbs). tcpO₂ may be useful for wound healing prediction and qualification for hyperbaric oxygen therapy. tcpO₂ may be used to determine wound healing potential for patients undergoing amputation e.g., when used in conjunction with other clinical assessments).

The operational closed loop control features of device may be aggregated by a smart system. Some of the features may originate from the system itself. Some of the features may originate from another system external to the main control system. Data from the internal closed loop control and external critical operation features may be aggregated.

Local drug delivery may control cell growth or cell death. For example, the therapeutic treatment may involve injecting a chemotherapy (e.g., locally at a tumor). The medical data stream may be an imaging data stream of an area intended to receive the chemotherapy. The qualitative feedback may include a first indication of whether the chemotherapy has leaked outside of the area intended to receive the chemotherapy. The qualitative feedback may include a second indication of whether the area intended to receive the chemotherapy has reached a threshold level of chemotherapy saturation.

FIG. 84A illustrates an example therapy device providing chemotherapy treatment to a tumor. In this example, the lung cancer tumor occurs mid-parenchyma. The tumor may not be spherical and/or have irregular nodules. The robotic flexible endoscope may approach the tumor from the bronchus side. The endoscope may insert the treatment needle to a position close to the center of the tumor (e.g., by using the cone beam CT or ultrasound guidance).

In an example, a flexible endoscopic robot (e.g., used for a bronchoscopy) may have a biopsy needle. The endoscopic robot may deliver drugs locally to a tumor (e.g., for treatment at the time and site of biopsy). The endoscopic robot may be guided to the tumor through the bronchi. The needle may extend through the working channel to biopsy the tumor. A surgeon may determine a method and magnitude of local drug delivery. The system may determine the needle position, needle angle, needle depth, and drug fluid injection pressure. As illustrated in FIG. 84A, the endoscopic robot may begin administering a drug (in this example, chemotherapy). The endoscopic robot may begin injecting the drug at a first pressure P1.

A smart monitoring system may control the needle insertion depth, needle angle relative to the tumor, positioning of the needle within the tumor, drug fluid injection pressure, drug dosage, drug volume, and/or other parameters. The parameters may be monitored through advanced visualization systems (e.g., multi-spectral, cone beam CT, Ultrasound, etc.). These parameters may be monitored to control the overall saturation of the desired area of the tumor with the desired dosage of the local drug delivery. FIG. 84B illustrates the therapy device of FIG. 84A identifying a chemotherapy leakage.

An independent system may monitor control of the treatment dispersal system. Radio opaque drug monitoring may be used to identify leaks (e.g., magnitude and direction of leaks) and/or saturation/completeness of tumor treatment. CT scanning may be used to monitor the drug saturation and/or concentration of the treatment area(s). Multi-spectral imaging may provide the margin sizes and effects to a cone beam CT (e.g., based on vascularity, reflectivity, and fluorescence). Local margin boundary detection may be used. An endoscope may output navigation information (e.g., triangulation coordinates) for the cone beam CT machine to use in focusing and positioning.

The injection pressure of the drug may be monitored to ensure full saturation of the planned tumor area. Irregular mass ejections outside of the tumor (e.g., in undesired locations, which may result in tissue death in unintended areas) may be monitored. The drug concentration may therefore be controlled in the intended areas and accidentally exposed areas.

For example, the endoscope may inject a contrast-doped medicant to fully saturate the tumor (e.g., with up to a concentration of 20%). The medicant may bloom out from the needle location in a spherical manner. As the medicant approaches the first edge of the tumor, it may squirt out of the tumor through a crevasse into the adjacent non-tumor tissue. The cone-beam CT may identify the leak and change the injection pressure of the fluid.

The initial injection pressure level (e.g., P1 in FIG. 84A) may be high. As illustrated in FIG. 84B, a leak may form, causing the chemotherapy drug to escape into unintended/undesirable location(s). FIG. 84C illustrates the therapy device of FIGS. 84A and 84B adjusting the chemotherapy treatment in response to the identified leakage. As shown, the system may reduce the injection pressure to a second pressure P2 (e.g., to reduce additional leakage). The system may change the position and/or orientation of the needle to one or more other location(s) to inject towards the untreated portion of the tumor (e.g., to fill the rest of the tumor to the minimum desired concentration of the chemotherapy drug) and minimize additional leakage. The adjustments may continue until the tumor (e.g., all of the tumor) is treated (e.g., with between 20-30% concentration of the mendicant).

A first limiting threshold may indicate the maximum amount of leakage or exposure of tissue outside of the tumor. The first limiting threshold may include a margin that is acceptable before the application is stopped and/or adjusted. A second limiting threshold may indicate the minimum concentration of the drug within the tumor and within the margin. The minimum concentration may apply to the surface area of the desired extent of the tumor treatment (e.g., shape, size, concentration, and/or the like).

FIG. 84D illustrates therapy monitoring associated with the example therapy device of FIGS. 84A-C. In this example, the system may notify the surgeon when the leakage is 3 mm or more away from the edge of the tumor. The surgeon may use this indication as a cue to reduce the injection pressure and/or adjust the needle position. As another example, a drug concentration threshold may be used to indicate the end of the procedure. For example, if the system detects that 90% or more of the tumor has a threshold concentration of the drug, the surgeon may conclude the treatment. In these examples, if the position and/or pressure adjustments were not made, a significant amount of the medicant may have spread beyond the tumor. Similarly, a significant amount of the tumor may be under-treated if the adjustments were not made.

Situational control of smart system boundaries may be based on independent (e.g., unrelated) events. For example, if a chemotherapy drug has just been applied to the patient in a specific area, cooling of the patient in that area should be avoided (e.g., so that maximum effectiveness of drug delivery is obtained). If a drug is delivered to the patient while the patient temperature is in a cooled or cooling state, drug absorption may be impacted (e.g., reduced or delayed). In this case, upon preparing for drug delivery or post drug delivery, a boundary of where cooling therapies are applied may be reduced.

Multiple (e.g., two) systems that have cooperated to achieve a balance between them may be adjusted (e.g., for a discrete portion of time) to induce an imbalance that effects the tissue or surgical space (e.g., to improve a specific step of a surgical procedure).

For example, in an endoscopic mucosal resection (EMR) (e.g., as the energy on a hot snare from an endoscopic-colon robot increases), endoscopic insufflation pressure may decrease, and laparoscopic pressure may increase. This may reduce resistance while snaring the lesion to be resected (e.g., as the snare cuts). Robotic control of snare deployment (e.g., out of the working instrument distal tube) may trigger the synchronized change of the laparoscopic pressure and endoscopic pressure.

In an example, the therapeutic treatment may involve designing an implant for a patient. The medical data stream may include a first imaging scan of an anatomical area of a patient associated with the implant and a second imaging scan of the anatomical area of the patient associated with the implant. The qualitative feedback may include an indication of whether inconsistencies between the first and second imaging scans exist or whether anomalies exist in either the first or second imaging scans.

For example, CT scans may be used to create precisely adapted patient-specific implants. The CT scans of the patient may be taken. The data from the scans may be transferred to a system or outside company (e.g., 3Di). The system or outside company may process the data and create a virtual patient model. Once the virtual patient model has been confirmed, a 3D implant model may be created. The 3D implant model may be reviewed/confirmed by the doctor. The implant may be manufactured and sent to site for implant.

Multiple scan sources may be combined to further enhance the model created (e.g., rather than only utilizing a CT scan). For example, ultrasound and/or magnetic resonance imaging (MRI) may be used. The processing side may interact with the CT scanning system to identify inputs that do not generate correctly and/or anomalies identified during the virtual model. This interaction may minimize potential errors or differences within a patient to create a more accurate representation of the implant.

FIG. 85 illustrates an example method that may be performed by a therapy monitoring tool. As shown at 54450, the method may involve selecting a therapeutic treatment from a database of a plurality of therapeutic treatments. The method may involve, at 54452, selecting a corresponding medical data stream relevant to the therapeutic treatment, wherein the medical data stream is produced by a surgical monitoring device. The method may involve, at 54454, determining, based on the medical data stream, qualitative feedback information about the therapeutic treatment.

During an operation, multiple devices may have an effect on a patient. The devices may impact the functionality of other devices as a result. For example, two separate devices may contribute to a negative feedback loop that lowers the patient's core temperature indefinitely, which will harm the patient if left unchecked. The devices may have no knowledge of each other or the effects each has on the other or the patient. The devices may therefore be incapable of correcting the negative feedback loop. FIG. 86 illustrates an example surgical operating room in which two or more of the devices may create a negative feedback loop in the patient.

A first system may apply conditional restrictions on the function or operation of itself and/or a second system. For example, the first system may apply conditional bounding its functions based on monitoring from the second system. There may not be a (pre)defined upper or lower bounds. Instead, the systems may track the outcome of their actions, and use the outcome to determine a limit. For example, the systems may compare their directional behavior and use the comparison to determine how to control one or both of the system. For example, if adjustments to both systems result in better performance, the systems may allow the adjustments. If one or both of the adjustments results in undesired behavior, the adjustments may be limited or reversed.

The first system may include a control system for monitoring and controlling the activities of the first system. The second system may include a control system for monitoring and controlling the operation of the second systems. At least one of the parameters monitored by the second system may be relevant to the operation of the first system. The parameter may not be monitored by the first system. Communication between the two systems may provide the first system with access to the data collected by the second system. The first system may use the data to effect the operational bounding of the first system's operation.

FIG. 87 illustrates an example of a first surgical device (e.g., Surgical Device 1) limiting its functionality based on patient data received from a second surgical device (e.g., Surgical Device 2). The second surgical device may include a processor with a data input/output module. The second surgical device may include a display, which may optionally display medical information. The first surgical device may determine that it is capable of obtaining medical information from a second surgical device. The first surgical device may obtain (e.g., receive) the medical information from the second surgical device. For example, the first surgical device may read the medical information from the display of the second surgical device. In another example, the second surgical device may output the medical information directly to a data input/output module of the first surgical device.

The first surgical device may include a processor with a data monitoring module and/or operational bounding module, as shown. The data monitoring module may receive the medical information and determine that the medical information satisfies a condition (e.g., patient temperature too low and/or the like). For example, determining that the medical information satisfies a condition may involve determining that operation of the first surgical device and operation of the second surgical device have created a negative feedback loop that negatively impacts patient health. The processor may indicate for the operational bounding module to limit the functional capabilities of the first surgical device (e.g., based on the medical information satisfying the condition). The operational bounding module may control physical components of the first surgical device to limit the functionality of the first surgical device (e.g., until the medical information is within an acceptable range again).

As illustrated in FIG. 88, first system may curtail full operational capabilities based on information monitored and provided by a second system. For example, the system may limit functional capabilities of the first surgical device such that the first surgical device operates in a manner, different than the first surgical device would operate without access to the medical information, so as to curtail negative impacts on a patient due to a negative feedback loop caused by normal operation of the first and second surgical devices. The second system may operate within the same area as the first system. The second system may provide the first system with information relating to the first system's behavior. The first system may change its operations based on a counter response from the second system.

Two separate smart systems may have a normal operational bounding (e.g., upper and lower limits). The systems may have an understanding that their control loops are interrelated in impacting the patient. In some circumstances (e.g., patient biomarkers are outside of the desired operational zone for one or both devices), the systems may make exemptions for one of their bounded control sets so that the systems may operate in concert to bring the patient parameters back within acceptable limits. The exemptions may be based on a directional improvement of the patient parameters. For example, reinforced control loop adjustment (e.g., where the closed loop monitor on one systems affects the monitored closed loop parameter of the other system) may be used. While the undesired self-reinforcing loop may be undesirable at times, the loop may be tolerated as long as the patient's biomarkers are moving in the correct direction and have not exceeded a maximum steady state magnitude. Once the discrepancy has been corrected, the normal operation functionalities and interactions may be resumed.

In an example, the first surgical device may be a ventilator. The device may determine that the medical information satisfies a condition by determining that carbon dioxide levels in a patient are above a threshold. The device may limit its functional capabilities by maintaining or increasing a tidal volume of the ventilator.

For example, oxygen (O₂) and carbon dioxide (CO₂) levels may be balanced. The O₂ and CO₂ levels may be monitored in the patient skin or blood flow. The ventilation rate may be controlled through the balance of volumetric airflow, O₂ and CO₂ concentrations within the airflow. Using a minimum volumetric tidal flow and minimum added oxygen may cause a buildup of CO₂ in the body (e.g., in procedures with long durations). This CO₂ buildup may inhibit healing short-term, change the pH of the blood & body, and/or limit recovery when the patient off the respirator based on standard volume breathing of the patient.

In an example, a surgeon may set the ventilator to a minimum tidal volume (e.g., to minimize the possibility of lung damage during inhalation). The O₂ may be controlled (e.g., open looped) by the surgeon at the beginning of the surgery. If the O₂ were controlled by oxygen partial pressure (PaO₂) patient monitoring, the ventilator may compensate to a lower tidal volume when the O₂ concentration is higher. However, this would increase the CO₂ build up (e.g., which is entirely tidal volume and rate based).

Smart PaO₂, CO₂, and heart rate monitoring may allow the ventilator respiration rate/volume and the O₂ concentration to be used. In this case, the ventilator rate may be the leading control and the O₂ may be a trailing control relative to the respiration rate (e.g., to reduce CO₂ and sustain O₂ blood gas levels).

The drugs delivered to the patient may impact the patient's core body temperature and/or metabolism. Based on the temperature or metabolic changes, the O₂ and/or CO₂ absorption rates may be impacted. Drug absorption may be impacted based on the patient's metabolic rate. At lower temperatures, the body may use less O₂. In this case, the negative effects of CO₂ may be less impactful. At lower temperatures, the patient's metabolic rate may decrease (e.g., thereby delaying drug absorption).

Tidal volumes may be 80 to 25 ml/kg between inhalation and exhalation, respectively. As a patient is put on a ventilator, the surgeon may set the ventilator to the lowest level (e.g., to avoid inadvertent injury). The O₂ volume may be set to the minimum to maintain the PaO₂ level. The minimum level tidal volume may not pull as much CO₂ out as O₂ going in. In this case, the CO₂ may cause acid to build up, lowering the pH of the patient (e.g., acidosis occurs at 7.35 and lower). A CO₂ limit may be used to increase tidal volume and lower O₂ to balance the CO₂ outlet with the O₂ inlet. The limit may continue to make tidal changes (e.g., based on time or CO₂ equilibrium levels) until the CO₂ and the O₂ are at target levels.

Too much CO₂ in the blood may be a sign of many conditions (e.g., lung diseases, Cushing's syndrome, kidney failure, metabolic alkalosis, in which the blood is not acidic enough, etc.). Acidosis may be caused by a buildup of CO₂ within the body. Acute kidney failure may be caused by a patient having acidosis for a relatively short amount of time (e.g., as little as ½ hour).

Normal acceptable levels of PaO₂ may be 75 to 100 millimeters of mercury (mm Hg), or 10.5 to 13.5 kilopascal (kPa). Normal acceptable levels of partial pressure of carbon dioxide (PaCO₂) may be 38 to 42 mm Hg (5.1 to 5.6 kPa). Normal acceptable levels of arterial blood pH may be 7.38 to 7.42. Normal acceptable levels of oxygen saturation (SaO₂) may be 94% to 100%. Normal acceptable levels of bicarbonate (HCO₃) may be 22 to 28 milliequivalents per liter (mEq/L).

The leading control variable relationship may be reversed in some examples.

With O₂ introduction and long-term supplementation, the body may (e.g., normally) decrease tidal volume (e.g., naturally due to the body being keyed to O₂, not CO₂). This may amplify the desirability for the externally-measured systems to monitor CO₂ (e.g., on which the body does not have a closed loop control).

Feature(s) associated with controlled ventilation and respiratory drive are provided herein. O₂ and CO₂ may be balanced in mechanical ventilation.

Mechanical hyperventilation may deplete (e.g., rapidly and abnormally deplete) CO₂ tissue reserves and blood bicarbonate. This may undermine respiratory drive for hours (e.g., until metabolic activity can replenish CO₂ levels). Hyperventilated patients may breathe and oxygenate effectively. The respiratory drive of hyperventilated patients may depend on their conscious awareness of surgical pain and psychological stimulation that provides an artificial stimulus to breathe. During this vulnerable period, even very small doses of opioids may unexpectedly obliterate the sole remaining source of respiratory drive. In this case, seemingly awake, alert, and fully recovered patients may unpredictably stop breathing.

In an example, the first surgical device may be a ventilator. The device may determine that the medical information satisfies a condition by determining that carbon dioxide levels in a patient are below a threshold (e.g., that the patient is hyperventilated). The device may limit its functional capabilities by decreasing a tidal volume of the ventilator.

Ventilation rate and volume may be controlled. For example, a medical professional may perform a controlled lung collapse. The lung collapse may be controlled using thoracic cavity internal pressure.

Insufflation pressure, suction magnitude, and smoke evacuation magnitude may be controlled to maintain a functional surgical working space. In this example, one of the systems may cause the pressure imbalance that another system is trying to overcome. The first surgical device may be a smoke evacuation device. The device may determine that the medical information satisfies a condition by determining that a patient core body temperature is below a threshold. The device may limit functional capabilities of the first surgical device by reducing the rate of smoke evacuation.

For example, as illustrated in FIG. 89A, as the use of suction or smoke evacuation is increased, the rate of cold CO₂ insufflation may increase (e.g., thereby causing a hypothermic response). The abdominal pressure to main the working space may use more CO₂ as gas is vented and suctioned away. The patient monitoring may be used to regulate the system of the thermal instability. For example, the patient monitoring system may request that the smoke evacuator be used less or slower. In this case, the smoke evacuator may ask the advanced energy generator to use a lower power (e.g., thereby causing less smoke rather than causing the hypothermic response).

The smoke evacuation may remove the smoke to improve visibility and reduce abdominal pressure. The pressure drop may increase the input volume of cold gas, which in turn causes the patient's core temperature to drop. The core temperature loss may cause hypothermia. The hypothermic response may cause the surgeon to lower the smoke evacuation rate and/or the energy power level of the energy generator. FIG. 89B is a graph that illustrates the effect of these devices on the patient's core temperature.

Enforcing a boundary limit of a following device may cause a reverse in the closed loop control (e.g., back to the cause of the adjustment).

Patient core body temperature may be controlled through interactive environmental, localized patient heating and cooling, and incidental heat loss (e.g., using insufflation and/or suction magnitudes). In an example, the first surgical device may be a patient heating system. The device may determine that the medical information satisfies a condition by determining that a patient core body temperature or a patient extremity body temperature is below a threshold. The device may limit functional capabilities of the first surgical device by increasing patient heating.

As illustrated in FIGS. 90A and 90B, thermo-regulation may be used in response to the body's vasodilatation and/or vasoconstriction based on the patient core temperature deviation.

Sedation may cause the patient to lose core body temperature (1-2° C.), which may cause hypothermia (e.g., at around 35° C.). To compensate for this loss, a surgeon may use a patient heating device, which may be (pre) set to compensate for the cold room temperature and the impacts of sedation (e.g., a bear-hugger heating vest may be used to counteract the heat loss). The surgeon may set the thermo load input (e.g., at the beginning of the surgery) to compensate for the sedation loss. In some cases (e.g., colorectal surgery), local cooling may be used to prevent ischemia (e.g., of the colon) due to interruption of blood flow.

The combination of sedation cooling and local cooling may (e.g., initially) result in vasoconstriction (e.g., as the body tried to maintain core temperature with the aid of the systemic patient warming). Specifically, the body may vasoconstrict the blood flow to extremities.

As the procedure continues, the local cooling may have a more global effect on the body. If the patient's core temperature drops more than 2° C., the body may reverse the vasoconstriction to a vasodilation state. The vasodilation opens the flow of cold blood to the extremities, which may rapidly increase the core temperature loss. The patient heating system may operate in an open loop manner (e.g., set by the surgeon) or a closed loop manner in which the heating may change by request or after the body temperature falls below a threshold or the change in temperature exceeds a limit. If the patient heating system lags too much, the rapid re-heating may cause additional cold blood flow to the heart. This may cause arrhythmias and potentially heart failure.

It may be beneficial, therefore, for the heating system to monitor the extremity temperature or blood flow as a means to preemptively determine an appropriate heating rate (e.g., so that the heating system does not fall too far behind, causing the rapid heating issue described above). ΔT_{c} may refer to the drop in core temperature. ΔTₑ may refer to the drop in the temperature of the extremities. Either may be used as an open-closed loop control of the heating

Metabolic uptake of medicine may be based on thermal levels of the patient core temperature. As the core temperature is reduced, the medication dosage may be (e.g., automatically) adjusted based on the lower metabolic uptake. If the body then reheats itself, the dosing and the existing levels of medicine have to be reduced before the metabolic uptake increases too much.

For closed loop control of the local cooling and the patient heating, the patient core temperature and extremity temperatures may be monitored. If the system detects the vasodilation trigger, the system may stop increasing local cooling and/or increase the patient systemic heating to prevent the excessive rapid loss of heat (e.g., which would trigger a secondary rapid heating response).

Physiological compensation may cause the system to reverse its closed loop control adjustments. For example, in the case of heart surgery, the surgeon may (e.g., intentionally) put the patient into a therapeutic hypothermic state (e.g., to prevent ischemic damage during the intervention in the blood supply to the heart). malignant airway obstruction (MAO) deterministic monitoring may be implemented. The most reliable measure of endotracheal intubation may be with direct visualization (e.g., during laryngoscopy) and the presence of persistently elevated end-tidal carbon dioxide (ETCO₂).

Positive return of end-tidal carbon dioxide alone may or may not confirm endotracheal placement of the endotracheal tube (ETT). Greater than 30 mm Hg ETCO₂ being sustained for three breaths minimum may be used to confirm tracheal placement. If a patient drank a bicarbonate solution or carbonated beverage before intubation, this also alter the measured ETCO₂. Capnography may use cardiac output and the gas exhaust, which may, for example, be used to quantify CO₂ outlet.

In an example, the viable operational envelope of a system or device may be limited based on the possibility of two moving smart systems occupying the common space at the same time to avoid collisions. For example, the first surgical device may be a first robotic arm in control of a first surgical instrument and the second surgical device may be a second robotic arm in control of a second surgical instrument. The first surgical device may determine that the medical information satisfies a condition by determining that a current movement trajectory of the first surgical device will cause a collision between the first surgical instrument and the second surgical instrument. In response, the first surgical device may limit functional capabilities of the first surgical device by stopping or changing the trajectory of the first surgical device.

An adaptive robot-to-robot no-fly zone based on an aspect of a first robot arm (e.g., location of the second robot cart, its robot arm position, movements, required operational envelope, etc.) may be used to limit the space that a second robot arm (e.g., from either the same robot or a separate robot) is allowed to use. The no-fly zone may be inside or outside of the patient.

For example, a first laparoscopic multi-cart robot may be used to for dissection and resection of a mid-parenchyma tumor that is on the junction of two segments. The surgeon may attempt to separate out the tumor from the artery and vein (e.g., to avoid removing two full segments). The surgeon may realize during surgery that the tumor has invaded the bronchus. To determine penetration depth and the extent of invasion, the surgeon may bring in a flexible endoscope controlled with a separate robot. The introduction of the second robot may not involve repositioning of the existing first robot cart. One of the carts may be positioned towards the head of the patient and have a working envelope outside of the body that encompasses some of the space now occupied by the flexible endoscopy robot and its operating envelope.

The second robot may establish communication with the first robot. The second robot may identify a its location and size dimensions. The second robot may define a minimum space in which it intends to operate. The second robot may inform the first robot of the reduced operational envelope in which the first robot will be able to operate without entangling the robots. This regulation of the first robot by the second robot may involve defining a space reduction and actively monitoring the first robot arm. The restriction may involve defining a portion of the full operating envelope in which the first robot may no longer operate. The restriction may involve actively adjusting regulation of the space (e.g., that changes as the first robot coordinates its operation with the flexible endoscopy robot). In this case, the space may be reduced only as needed to perform certain actions, while allowing the first robot to occupy the shared space (e.g., if the second robot does not need that space). If both robots intend to occupy the same shared space, the robots may negotiate based on priority, user input, or computational ordering that would allow the motions to be choreographed. The choreographs motion may allow the robots to move around each other through a series of pre-calculated alternating motions (e.g., to allow them to move around each other without adverse interactions).

As the flexible endoscopy robot is brought into the OR and setup, the user may input the location and operational window for the endoscopy robot, or one of the smart systems may define the location and operational envelopes of both robots (e.g., relative to each other).

A hub and a room-based camera may be used to identify the exact location, shape, and/or operational window of the devices (e.g., based on the setup of the devices). For example, multiple perspective cameras with overlapping image coverage may be used. As another example, fewer cameras and laser alignment and/or light direction and ranging (Lidar) may be used as a means to detect distances and even structured light as a means to define shapes and volumes.

Subtractive operational envelope reduction may be used to reduce the space used by a smart system. For example, one of the smart systems may be capable of using a space, but may no longer be allowed to use that space based on a use or priority of another system with regard to the shared envelope.

The first smart device's position, motions, articulation, energy potential, and/or activation state may cause an adjustment of an adjacent device in near proximity. For example, a conductive end-effector's zone of occlusion and interaction may be modified depending on whether or not one of the end-effectors monopolar energy is active or active and above a certain threshold. This may prevent inadvertent energizing of the non-monopolar device based on either inadvertent contact or close proximity in contact with the same tissue (e.g., where the second device could become part of the return path). The coordination may minimize inadvertent burns to the patient away from the surgical site based on the continuity path of the second device's other tissue contacts.

The operational window of a first device may be adjusted due to the proximity of another device to limit the proximity of the second device (e.g., if the first is active). The first device may be prevented from activating based on proximity of the second device (e.g., in order to reduce the risk of interference between devices). For example: the activation of energizing a first device may be limited if the first device is in close proximity to a second device that has sensing means that would be affected by using the energy nearby.

FIG. 91 illustrates an example method that may be performed by a surgical device. As shown at 54450, the method may involve determining that the first surgical device is capable of obtaining medical information from a second surgical device. The method may involve, at 54452, receiving the medical information from the second surgical device. The method may involve, at 54454, determining that the medical information satisfies a condition. Determining that the medical information satisfies a condition may involve determining that operation of the first surgical device and operation of the second surgical device have created a negative feedback loop that negatively impacts patient health. The method may involve, at 54456, limiting functional capabilities of the first surgical device based on the medical information satisfying the condition.

Systems, methods, and instrumentalities associated with inter-connectivity of data flows between various surgical devices and/or systems are disclosed. The surgical devices and/or surgical systems may be interrelated or independent smart surgical devices and/or surgical systems. Data sourced by a first surgical device/system may be communicated to and/or accessible by a second surgical device/system for interactive use and storage. The data exchange may be bi-directional or unidirectional, which, for example, may enable the surgical devices/systems to interface and use each other's data. The data exchange may affect one or multiple surgical device/system's operation.

For example, a first surgical system may operate using a first operation configuration (e.g., first operation configuration parameters). The first surgical system may determine capability information associated with a surgical environment (e.g., operating room (OR)). The surgical environment may include surgical systems (e.g., surgical hub, surgical devices, etc.). The capability information may include information associated with what a surgical system may be capable of generating and/or providing. The first surgical system may receive first data and associated metadata (e.g., first metadata) from a second surgical system. The first metadata may indicate whether the first data is control data or response data (e.g., which portion of the first data is control data or response data). The first surgical system may select an operation configuration (e.g., operation configuration parameter) based on the first data and/or first metadata. For example, the first surgical system may determine the first operation configuration if the first data is response data. The first surgical system may determine the second operation configuration if the first data is control data. The first surgical system may generate second data based on the determined operation configuration. The first surgical system may determine data packages (e.g., including at least a portion of the second data) to send to target systems. The data packages may indicate whether the data in the data package is control data or response data.

For example, the first surgical system may determine capability information associated with the surgical environment based on a discovery procedure. The discovery procedure may include determining the surgical systems present or used in a surgical environment. The discovery procedure may include determining capabilities associated with each surgical system present or used in the surgical environment. The discovery procedure may be performed before determining an operating configuration to use. The first surgical system may determine capability information based on a pre-configuration (e.g., checklist, boot-up sequence). The pre-configuration may include information indicating surgical systems and their respective capabilities associated with the surgical environment.

The first surgical system may determine that received data is inaccurate and/or incomplete based on the received first data and the determined capability information. For example, the first surgical system may determine that a data or data type is missing. The first surgical system may send an indication indicating that the data is missing or the surgical system that generated and sent the data (e.g., second surgical system) is not operating properly.

Interrelated surgical systems may communicate and send data between each other. Systems that communicate with each other to share data may use the data to impact a receiving system's operation (e.g., function, processing, and/or the like). For example, the data may impact the operating configuration of a system. Data generated at a first surgical system may be sent to a second surgical system. The data received at the second surgical system may be used to modify the second surgical system's operating configuration (e.g., from a first operating configuration to a second operating configuration). This may lead to a detrimental cascade of feedback affecting functionality. For example, the second surgical system may generate data based on the updated operating configuration and send the data back to the first surgical system, which may then operate its operating configuration based on the return data. The detrimental feedback loop would continue to have the surgical systems altering their operating configuration based on continually updated parameters for generating data.

Interrelated systems that communicate and share data that may impact an operating configuration may use a data exchange framework to address the cascading feedback. Avoiding the cascading feedback may enable the systems to operate accurately.

A data exchange framework to address the cascading feedback may include data and/or metadata indicating whether the data is control data or response data (e.g., control indication or response indication). Metadata may be generated for data produced by a surgical system. The data and/or metadata may indicate whether the associated data is response or control data. Response data may indicate that a receiving surgical system should not modify its operating configuration based on the data Control data may indicate that the receiving surgical system may adapt its operating configuration (e.g., operating parameters) based on the received data.

FIG. 92 illustrates an example flow of interrelated surgical systems exchanging data within a surgical environment. The surgical environment 54600 (e.g., operating room or theater, or medical facility) may include a surgical hub 54601 and a plurality of surgical systems (e.g., surgical system 1 54602, surgical system 2 54603, surgical system N 54604, etc.) The surgical system(s) may be interrelated and may generate data and/or communicate with each other (e.g., send data, such as, for example, control data or response data). The surgical system(s) may determine capability information associated with other systems in the surgical environment. The surgical system(s) may operate using configured operating configurations. The operating configurations may be changed and/or modified. The operating configurations may be changed based on data (e.g., generated data and/or data received from other systems). Surgical systems may generate data and/or perform surgical operations and/or actions according to an updated operating configuration.

As shown in FIG. 92, at 54605, discovery associated with the surgical environment may be performed. Discovery may be performed by surgical systems (e.g., surgical hub) in the surgical environment (e.g., before determining an operating configuration to use). The discovery may include determining the surgical systems present and/or being used in the surgical environment. The discovery may include determining the number of surgical devices and/or surgical systems in the surgical environment. The discovery may be performed by communicating with the surgical systems in the surgical environment. The discovery may be performed based on data received by the surgical hub in the surgical environment.

For example, discovery may be performed in association with a surgical environment start up (e.g., boot up process). A surgical environment may be set up to perform a surgical procedure. A surgical environment may be set up based on the surgical procedure. For example, a colorectal surgical procedure may be associated with using a first surgical system (e.g., a laparoscopic surgical system) and a second surgical system (e.g., an endoscopy surgical system), and a cardiovascular surgical procedure may be associated with the second surgical system and a third surgical system (e.g., a surgical system with energy device). The surgical systems used in the surgical environment may depend on the surgical procedure, patient, and/or healthcare professionals performing the surgical procedure (e.g., surgeon may have preferences for using specific surgical systems and/or tools). In examples, a checklist for surgical systems used in a surgical procedure may be used. The checklist may be used to perform discovery of the surgical systems in the surgical environment.

Discovery may include scanning a surgical environment based on a surgical procedure checklist and/or performing a dynamic discovery process associated with determining which surgical systems and/or tools are present in the surgical environment. The dynamic discovery process may validate the surgical procedure checklist, for example, to confirm whether a specific surgical system is present. The dynamic discovery process may validate whether a surgical system is properly configured (e.g., generating the proper data and/or able to communicate the data). The discovery process may validate that surgical systems within the surgical environment are operating correctly.

As shown in FIG. 92 at 54606, capability information associated with the surgical environment may be determined. The capability information may be determined for each surgical system and/or surgical hub in the surgical environment. Each surgical system and/or surgical hub may be associated with different capabilities (e.g., types of data produced, types of processing performed, and/or the like). For example, a first surgical system may be associated with a first capability and a second surgical system may be associated with a second capability. The first capability of the first surgical system may be associated with generating a first type of data, a second type of data, and/or a third type of data. The second capability of the second surgical system may be associated with generating the third type of data and/or a fourth type of data. The capabilities of the surgical systems may be used to determine whether or not appropriate data is being generated and/or communicated between systems.

For example, a first surgical system may be determined to be capable of generating and communicating a first, second, and third type of data (e.g., indicated in capability information). A surgical hub or second surgical system may receive the first and second type of data from the surgical system. The surgical hub or second surgical system may determine that the third type of data is missing from the first surgical system, for example, based on the determined capability information.

As shown in FIGS. 92 at 54607a, 54607b, and 54607c, the surgical systems and/or surgical hub may generate data (e.g., according to their respective operating configurations). The generated data may be sent between the interrelated systems and/or the hub. For example, as shown at 54608a, 54608b, and 54608c, the first surgical system may receive data and/or respective metadata sent from the other surgical systems and/or surgical hub. The first surgical system may use the received data and metadata to perform according to its current and/or default operating configuration. The first surgical system may use the received data and metadata in determining which operating configuration to apply (e.g., whether to use the currently used operating configuration or switch operating configurations).

Metadata may provide context to bi-directional exchanged data. The metadata may provide context, for example, to enable (e.g., allow) bracketing usage implications for one or multiple systems. A bi-directional data feed may include an originating system utilization of the feed and a priority of the feed to a system's primary function.

Determination and/or communication of which data streams are being used as control streams and which ones are response streams may be performed. Systems may share data streams, where some of the data streams may be interrelated originating from different systems. Each system may use the data it is receiving to either control an aspect of its own function (e.g., operation) monitor for leading indicators of upcoming changes, or verify changes by monitoring the responses of the related systems. For example, if two systems both used feeds from each other that are interrelated, both as control feeds, there may be a detrimental cascade where each device may keep changing based on the received data and the change is monitored by the other system to in turn make another change that then may initiate a third change back at the first system. The detrimental cascade of feedback may be overcome if the bi-directional data exchanged indicated in metadata whether the feed is a driving control data feed or if it is a monitored response feed.

Systems may indicate which data streams are needed and the intended use of the data streams. The intended use may be included in metadata. For example, intended use may be indicated (e.g., via a notification of intended use). Systems may determine what data types are used by a different system to control an active element of operation. The notification of intended use may prevent both systems treating interrelated data-feeds as controlling. Relational determination of open and close loop control may be performed between two systems that are interrelated or interdependent on convoluted data streams. For example, one system of two systems may deactivate or operate in an open loop operation. The other system may be a controlling system.

Smart surgical systems may exchange information bi-directionally that each system may use or react to. Datasets may be non-interactive, interactive, or convoluted.

Non-interactive data sets may include a hub-to-smoke evacuator module bi-directional data flow, which may be independent uni-directional from each system to the other. A smoke evacuator may indicate a power consumption rate to another system for power management of the interrelated systems sharing the same power source. Smart systems (e.g., a hub or mantle backplane) may communicate power levels, hand piece energy density/duration (e.g., likelihood of causing smoke), energized status of generator, etc.

A convoluted data set may include data associated with smart controlled irrigation for advanced monopolar and/or bipolar cooling. The convoluted data may be exchanged between smart controlled irrigation devices and a generator. A smart irrigation device may receive power level or electrode temperature and activation status from a generator to control saline flow-rate, saline pressure, and/or saline temperature. The controlled rates may be communicated to the generator for adaptation of the control operations (e.g., algorithms). For example, the generator may receive a saline flow-rate, pressure, fluid temperature, and/or the like, to control power level, constant current versus power, energy modality, etc. The generator may control the system under normal parameters and allow the smart irrigation system to adjust, or the smart irrigation system may be predefined and the generator may adjust to minimize charring and/or tissue sticking. Both systems may be enabled to adjust some (e.g., not all) of the control parameters each using (e.g., relying) on the other system to adjust the other impacting parameters.

In examples a generator (e.g., a first system) and an energy device (e.g., a second system) may interact. The energy device may be determined to have a resistance over time below a nominal threshold (e.g., cutting or sealing too fast). The generator may react by changing the power level for the next activation. A third system (e.g., graspers/robotic arm), which covertly reviewed the data and sees the data transfer and changes applied between the two systems, may determine that the tension and/or load that is on the tissue when fired is too much based on the data transferred. The third system may alter the position at which it is holding the tissue, for example, to minimize the tension when cutting and/or sealing.

Translation ability and/or the ability to understand data in output form (e.g., raw form) may be enabled. Metadata head or cipher may be included in data, for example, to define format, language, relational organization of the data, and/or the like (e.g., to enable interpretation of the data). For example, a patient monitor device may collect biomeasures associated with a patient (e.g., PO2, CO2, EKG, blood pressure, core temperature, etc.) but may not have dedicated real-time output ports for the measures. The patient monitor device may use a single output port with a combined data stream. The monitors may not be collected using a sampling rate. The data stream may vary point by point of what data is being exported. Metadata cipher may be used to determine what measures are within a data point or may provide a format or identifier as to which data points allow an external system to decompose one stream into independent steams.

As shown in FIG. 92 at 54609, the first surgical system may determine an operating configuration based on the received surgical data and/or associated metadata. The first surgical system may be operating using a first operating configuration (e.g., first operating configuration parameter). The first surgical system may change the applied operating configuration (e.g., from the first operating configuration to a second operating configuration). The change in applied operating configuration may be determined based on data, for example generated by the first surgical system and/or received data associated with other surgical systems and/or hubs.

The first surgical system may determine which operating configuration to apply based on a classification associated with received data (e.g., data generated by another system or hub). For example, received data may be control data (e.g., data indicating to control the system receiving the data) or response data (e.g., data indicating that the data is generated in response to an aspect associated with the first surgical system).

For example, data may be control data and sent to the first surgical system to affect the operating configuration. For example, first data received by the first surgical system may be control data and the first surgical system may determine operating parameters and/or a suitable operating configuration based on the received control data. The control data indicates that the first surgical system may modify its operating configuration as needed.

For example, data may be response data and sent to the first surgical system. The response data may be generated by a second surgical system. The second surgical system may have been operating using a third operating configuration and may have switched to a fourth operating configuration based on data the second surgical system received (e.g., control data sent by the first surgical system to the second surgical system). The second surgical system may generate data based on the fourth operating configuration and send the data as response data to the first surgical system. The first surgical system may determine that the data received is response data and may refrain from modifying its operating configuration based on the data being response data.

The classification of control data and response data may prevent a cascading feedback problem between communicating surgical systems. For example, if there is no indication of control or response data, systems may continually change their operating configurations and generate data accordingly based on incoming received data. For example, a first surgical system may send data to a second surgical system. The second surgical system may update its operating configuration from a first operating configuration to a second operating configuration based on the data received from the first surgical system. Then the second surgical system may generate data based on the second operating configuration and send the data to the first surgical system. The data received that was generated by the second surgical system using the second operating configuration may impact the operating configuration of the first surgical system. For example, the first surgical system may then alter its operating configuration from a third operating configuration to a fourth operating configuration and generate data using the fourth operating configuration. The data generated by the first surgical system using the fourth operating configuration may be sent to the second surgical system. The second surgical system may continue the cascading feedback loop and continually update the operating configuration. The cascading feedback loop would negatively impact the operation of the surgical systems.

The control data and response data classification may prevent the cascading feedback loop. For example, a surgical system receiving control data may be indicated to update its configuration based on the control data. A surgical system receiving response data may be indicated to refrain from updating its configuration based on the response data. The response data may act as a termination of the cascading feedback loop.

As shown in FIG. 92 at 54610, the first surgical system may generate surgical data (e.g., surgical procedure data, surgical equipment data, patient information data, OR information, facility information, surgical system data, surgical event data, and/or the like) using the determined operating configuration. The generated data may be in response to control data received by the first surgical system. The first surgical system may send its generated data, for example, to the interrelated systems in the surgical environment. As shown at 54611, the first surgical system may determine data packages to send to respective interrelated systems. As shown at 54612a, 54612b, and 54612c, the data packages may be sent to the respective surgical systems. The data packages may indicate whether the data in the respective data packages are control data or response data.

FIG. 93 illustrates an example flow of determining an operating configuration based on received data and/or associated metadata. A first surgical system may determine an operating configuration (e.g., determine whether to maintain a current operating configuration or apply a different operating configuration) based on received data (e.g., based on whether received data is control data or response data). The first surgical system may determine to modify its operating configuration if the received data is control data. The first surgical system may determine to maintain its current operating configuration if the received data is response data. The first surgical system may generate data based on the applied operating configuration after receiving the initial data.

As shown at 54615, the first surgical system may determine capability information associated with a surgical environment. The capability information may indicate the surgical systems and/or surgical devices that may be communicated with and/or interrelated in the surgical environment. The capability information may indicate the types of data the surgical systems in the surgical environment are capable of generating and communicating. Surgical systems may use the capability information to determine whether another surgical system sending data is a closed loop or open loop system. The capability information may indicate whether another surgical system sends control data or response data.

As shown at 54616, the first surgical system may receive first data and associated metadata from a second surgical system. The first data and associated metadata may indicate whether the first data is control data or response data. As shown at 54617, the first surgical system may determine whether the first data and associated metadata is control data or response data.

As shown at 54618, the first surgical system may determine an operating configuration based on the received first data. For example, if the first data is control data, the first surgical system may determine whether to modify its operating configuration based on the first data. If the first data is response data, the first surgical system may refrain from considering the data to determine its operating configuration.

As shown at 54619, the first data may be response data. The first surgical system may be operating using a first operating configuration (e.g., when it received the first data). Because the first data is determined to be response data, the first surgical system may determine to continue using the first operating configuration (e.g., refrain from switching operating configurations and/or parameters).

As shown at 54620, the first data may be control data. The first surgical system may be operating using the first operating configuration (e.g., when it received the first data). Because the first data is determined to be control data, the first surgical system may determine to consider the first data in determining its operating configuration (e.g., whether to maintain the first operating configuration or switch to a second operating configuration). For example, the first surgical system may determine to apply a second operating configuration based on the first data.

As shown at 54621, the first surgical system may generate data based on the applied operating configuration. The generated data may be sent to surgical systems (e.g., as described herein with respect to FIG. 92). For example, a third surgical system may be determined. The first surgical system may determine (e.g., generate) second data and associated metadata to send the second surgical system (e.g., as shown at 54622). The second data may be generated based on the applied operating configuration. Metadata may be generated and/or determined that is associated with the second data. For example, the metadata associated with the second data may indicate whether the second data is control data or response data. Accordingly, the first surgical system may send the determined second data and associated metadata to the second surgical system (e.g., as shown at 54623).

In examples, a first surgical system may operate using a first operating configuration. The first surgical system may determine capability information associated with a surgical environment. The first surgical system may receive surgical environment information that indicates surgical system information associated with a default surgical system environment (e.g., for a specified surgical procedure). The first surgical system may perform a discovery operation associated with the surgical environment. The first surgical system may determine surgical environment information based on the performed discovery. The capability information may include the default surgical system environment and the discovered surgical environment information. The capability information may indicate surgical system information (e.g., including a list of surgical systems associated with a surgical environment). The capability information may indicate the data (e.g., type of data) that a surgical system is capable of generating, measuring, communicating, and/or providing. The first surgical system may receive first data from a second surgical system (e.g., indicated in the surgical system information. The first data may include first metadata that may indicate that the first data is control data or response data. The first surgical system may select an operating configuration based on the first metadata (e.g., a first operating configuration or a second operating configuration). The first surgical system may select the first operating configuration (e.g., maintain the first operating configuration) if the first metadata indicates that the first data is response data. The first surgical system may select the second operating configuration if the first metadata indicates that the first data is control data. The first operating configuration may be associated with the first surgical system operating using a first operating parameter and the second operating configuration may be associated with the first surgical system operating using a second operating parameter. The second operating parameter and/or second operating configuration may be determined based on the first data. The first surgical system may generate second data based on the selected operation configuration. The first surgical system may determine a third surgical system in the list of surgical systems associated with the surgical environment to send at least a portion of the second data (e.g., where the third surgical system is the same system as the second surgical system). For example, the first surgical system may determine that the third surgical system is to receive a portion of the second data. The first surgical system may send (e.g., via a data stream) the portion of the second data, for example, to the third surgical system. The data stream may include second metadata that may indicate that at least a portion of the second data is control data or response data.

FIG. 94 illustrates an example flow of determining an operating configuration based on received data. As illustrated in FIG. 94, a first surgical system (Surgical System 1, 54625) may be interrelated with a second surgical system (Surgical System 2, 54626). The interrelated surgical systems may share data between each other. The interrelated surgical systems may modify their operating configuration based on received data from each other.

As shown at 54627, the second surgical system may generate first data and associated metadata (e.g., as described herein). The first data may be surgical data. That first data may be determined to be control data or response data. For example, surgical control data may be used for controlling a surgical system. The first data may be control data if first data is intended to be considered by the receiving surgical system (e.g., first surgical system) in determining an operating configuration that may be used by the receiving surgical system. The first data may be response data if the first data is intended to be refrained from being considered (e.g., not be considered) by the first surgical system in determining an operating configuration. As shown at 54628, the second surgical system may send the first data and associated metadata to the first surgical system.

As shown at 54629, the first surgical system may determine that the first data is control data. Based on the first data being control data, the first surgical system may consider the control data in determining an operating configuration to use. For example, the first surgical system may be using a first operating configuration before receiving the first data and associated metadata. The first surgical system may determine whether to change the operating configuration based on the first data. For example, the control data may not warrant changing the first surgical system's operating configuration. In this case, the first surgical system may determine to refrain from changing the operating configuration.

In examples, the first surgical system may determine to change the operating configuration to a second operating configuration (e.g., as shown at 54630). The determination to change the operating configuration may be based on the first data being control data and/or the contents of the first data.

As shown at 54631, the first surgical system may generate second data and associated metadata based on the changed operating configuration. The first surgical system may send the second data and associated metadata, for example, to the second surgical system (e.g., as shown at 54632). The second data and associated metadata may indicate that it is response data (e.g., in response to the control data received from the second surgical system), for example, to prevent a cascading feedback loop.

The second surgical system may determine that the second data is response data (e.g., as shown at 54633), for example, based on the second data and/or associated metadata. The second surgical system may determine to refrain from considering the second data in determining an operating configuration. For example, the second surgical system may determine to maintain its currently applied operating configuration (e.g., based on the second data being response data), as shown at 54634. Accordingly, the second surgical system may generate third data and associated metadata based on the maintained operating configuration (e.g., as shown at 54635). The second surgical system may send the third data and associated metadata, for example, to the first surgical system (e.g., as shown at 54636). The third data and/or associated metadata may indicate whether the control data (e.g., if the second surgical system wants to update the operating configuration of the first surgical system) or response data (e.g., if the second surgical system wants the first surgical system to refrain from considering the third data in determining an operating configuration to use.

Interrelated systems may use and/or rely on other systems to provide complete data, for example, to properly function. For example, a first system that is capable of providing a first type of data and a second type of data may send the two types of data to a second system. The second system may use both types of data to operate correctly. If the first system only sends one of the two types of data (e.g., only the first type of data or only the second type of data), the second system may not be able to operate properly. In other examples, a system using incomplete or inaccurate data in its operation may operate incorrectly.

For example, a first system may expect first data, second data, and third data from a second system. The second system may have only sent the first data and the third data. Without the second data, the first system may not operate properly.

Capability information associated with the surgical environment may be leveraged to determine whether correct data is being communicated between interrelated systems. The capability information associated with the surgical environment may be determined (e.g., as described herein, specifically in reference to FIG. 92). The capability information of the surgical environment may be used (e.g., by a surgical system) to check that received data from a sending system is accurate and/or complete (e.g., includes all the data that the sending surgical system is capable of generating and/or sending). For example, the capability information may be used to determine that a surgical system is capable of providing first, second, and third data. A complete data set would include the first, second, and third data.

Based on the capability information and received data, whether received data is incorrect or incomplete may be determined. Based on a determination that the data is incorrect or incomplete, a receiving system may send an indication to the sending system (e.g., system that generated the incomplete and/or incorrect data). The indication may indicate that the data was incorrect and/or incomplete. The indication may indicate to correct the issue (e.g., send the missing data or correct the improper operation).

FIG. 95 illustrates an example flow of determining capability associated with a surgical environment and determining whether a dataset is complete and/or accurate. As shown in FIG. 95, a surgical environment 54638 may include surgical hub(s) (e.g., Surgical Hub 54639) and/or surgical system(s) (e.g., Surgical system 1 54640, surgical system 2 54641, and surgical system N 54642). The surgical systems and/or hubs may be interrelated (e.g., may send data between each other).

As shown at 54643, discovery associated with the surgical environment may be performed (e.g., by the first surgical system). The discovery may be performed to determine the surgical systems and/or hubs within the surgical environment, and/or determine the capability associated with the surgical systems and/or hubs in the surgical environment.

As shown at 54644, capability information associated with the surgical environment (e.g., capability information associated with respective surgical systems) may be determined (e.g., by the first surgical system). The capability information may indicate the capabilities associated with each surgical system within the surgical environment. For example, the capability information may indicate that a second surgical system is capable of providing first data (e.g., first type of data), second data (e.g., second type of data), and N data (e.g., Nth type of data). The capability information may indicate respective data and/or data types that surgical systems may be able to generate. For example, the surgical hub 54639 may generate data at 54645 according to the capability information associated with the surgical hub. Similarly, surgical system 2 54641 and surgical system N 54642 may generate data 54645b and 54645c respectively, for example, according to their respective capabilities indicated in the capability information. The generated surgical data may be sent, for example, as shown at 54646a, 54646b, and 54646c.

As shown at 54647, it may be determined that there is missing data in the received surgical data, for example, based on the capability information. In examples, the received data may not be complete or the data may be inaccurate. The completeness and/or accuracy of the data may be determined, for example, based on the capability information and received data. For example, the capability information may indicate that a first data type and a second data type may be received from a surgical system. It may be determined that received data is incomplete if it includes (e.g., only includes) the first type of data. The received data may be validated (e.g., cross-checked) with the capability information associated with a sending system.

A first surgical system may be configured to determine that first data received from a second surgical system is incomplete and/or inaccurate. The first surgical system may obtain capability information associated with the second surgical system (e.g., as described herein). The first surgical system may receive data from the second surgical system. The first surgical system may determine that the data received from the second surgical system is incomplete and/or inaccurate based on capability information associated with the second surgical system and the received data (e.g., contents of the received data). For example, the capability information may indicate that the second surgical system is capable of measuring and/or sending a first type of data and a second type of data, and the received data may include the first type of data (e.g., only the first type of data). The first surgical system may send an indication, for example, to the second surgical system. The indication may indicate to the second surgical system that the data is incomplete and/or inaccurate.

For example, the second surgical system may send processed data to the first surgical system. The first surgical system may desire to use raw, unprocessed data. Raw and/or unprocessed data may include surgical data that is sensed by a surgical system. The raw and/or unprocessed data may include surgical data that is not transformed. The first surgical system may indicate to the second surgical system to provide the unprocessed, raw data.

As shown at 54648, an indication may be sent. An indication may be sent to a surgical system that indicates that the previously sent data is incomplete and/or accurate. Based on a determination that received data is incomplete or inaccurate, the indication sent may indicate to the sending surgical system (e.g., an indication may be sent from surgical system 1 to surgical system 2 as shown in FIG. 95) that the data is incomplete or incorrect. The indication may further indicate to send the correct/appropriate data. The indication may further indicate to check the operating configuration to determine that the surgical system is operating as expected. The indication may further indicate to rectify issues associated with the sent data.

Real-time data exchange and processing may be performed. The real-time data exchange and processing may be associated with partitioned smart systems (e.g., surgical systems). Real-time exchange data may be partitioned. The real-time exchange data may be temporally partitioned. The temporally partitioned real-time exchange data may be used.

For example, smart systems out of sequence with a surgical procedure may determine that devices are capable of performing in the surgery operation. In examples, smart chargers for battery powered devices may be used. Smart chargers for battery powered staplers may communicate a status of the battery. The smart chargers may receive anticipated demand associated with the smart charger, a smart battery, the smart stapler, and a smart hub (e.g., surgical hub).

The smart charger may detect battery properties. For example, the smart charger may detect battery recharge capacity, status of current charge level, completion of charge timing, and/or the like. The battery properties may be communicated, for example, to other smart systems and/or the smart hub. The battery properties may be considered and/or used (e.g., by the hub) to define surgical procedure parameters (e.g., start/stop times, operating room throughput, etc.).

In examples, the data from the smart hub on utilization rate and/or timing may be used by the charger. For example, the smart charger may determine to switch between trickle charging (e.g., better for battery capacity). The smart charger may determine when to finish charging (e.g., maintaining the battery at a specified percentage (e.g., 80%) charge in maintenance mode. The smart charger may determine to complete the charging (e.g., just in time) for the procedure (e.g., which may be better for battery health longevity).

The smart charger may receive information associated with scheduling of surgical procedures and/or the next series of surgical procedures that may use a battery operated surgical device (e.g., power stapler and/or ultrasonic hand-held devices). The smart charger may determine charging parameters (e.g., charging speed, order of batteries being charged) based on the received information. The smart charger may determine charging parameters based on the usage information (e.g., usage for the surgical procedure). The smart charger may determine charging parameters (e.g., order of charging) to ensure that a fully charged battery is ready to be used at the time of each procedure. The smart charger may review the usage cycle of the batteries. The smart charger may charge a battery most appropriate for use based on balancing the usage between a set of batteries. The smart charger may determine to use all the charge of a first battery (e.g., intentionally), for example, to allow time for a replacement to be acquired without other batteries using a replacement simultaneously.

FIG. 96 illustrates an example of a smart battery charger sending usage information. As shown in FIG. 96, the smart battery charger for a powered stapler may have two batteries on the charge (e.g., at 25% charger and 55% charge respectively). The hospital surgical hub may inform the charger about the number of upcoming surgical procedures, the expected number of firings during each of those surgical procedures, and/or the timing of the firings, for example. The charger may inform the surgical hub when the first powered surgical stapler may be ready, and/or whether the charger will use fast charge mode for a second pack to be ready for a subsequent surgical procedure. The charger may send information indicating that the first battery pack will have sufficient charge for a first surgical procedure and a third surgical procedure (e.g., with a second procedure performed in between) that do not overlap in timing.

FIG. 97 illustrates an example of smart chargers for battery power stapling. As shown in FIG. 97, the smart charger may communicate with a smart hub and a surgical device (e.g., smart hand-held device). The smart charger may communicate with the smart hub regarding procedure plans, timing, cadence, and/or the like. The smart charger may communicate with the smart hand-held device regarding real-time updates on the battery levels and the consuming of the power. In the case of a procedure where there is less power used than expected (e.g., predicted), the smart charger may adjust charging rates and/or timing of the other batteries, for example, to balance the use of the sufficiently charged batteries with the desire to charge slowly (e.g., which may prolong the lifespan of the battery). As shown in FIG. 97, a power stapler may use (e.g., require) 50% of a charge for six 60 mm thick tissue firings in a gastric sleeve. A second device may be ready for a first procedure at 75% charge, and because three firings were used in the first surgical procedure, fast charging may be refrained from being performed (e.g., not be needed), and the second device may be ready for a second use for a third surgical procedure. A different battery may not use (e.g., require) a special charging for the three procedures to be handled by the two battery packs.

Real-time updates from the smart stapling device may be used by other surgical devices, for example, such as non-wired battery powered smart devices. The real-time updates may indicate a higher use of the battery or less use, which may impact the smart charger's charging parameters (e.g., cleaning and charging parameters for the data to support the usage needs). The real-time updates may enable a charger to monitor the rate at which the battery was discharged and/or the magnitude of the discharge, for example, to enable the smart charger to adjust its conditioning during recharging (e.g., to minimize effects on the battery chemistry for heavy use procedures). The conditioning may include a slow charge, a charge of a portion and holding for a period of time, a small incremental pining on/off charging (e.g., to induce the chemistry within the battery to re-balance after being heavily used or fully discharged), and/or the like. The conditioning may reduce the effects on the electrolyte mix (e.g., of the battery) and/or reduce event reverse corrosion or damage to the electrodes.

An event may be detected based on the communication between smart devices. For example, a dependent event may be detected and the detection may be confirmed. Confirmation of an action by a first system may be reflected in an output of an unconcerned system.

For example, biopsy needle penetration depth may be monitored (e.g., by ultrasound imaging of the tumor and needle). A robotic surgical system may change the depth of the needle and may use (e.g., require) confirmation that the current ultrasound image includes the updated motion before another motion is permitted. The robotic surgical system may monitor the ultrasound output to detect a related action to confirm the ultrasound stream is up to date before performing subsequent actions. The ultrasound may provide a metadata tag relating to time which the robot may use as a measure to ensure the action it took within a related timing is up to date.

Surgical procedure data (e.g., full surgical procedure data) from a smart system may be downloaded and/or processed. The download and/or processing of the full surgical procedure data may be accessible outside the surgical event (e.g., after the surgical procedure and/or event). The downloaded surgical data from a first surgical procedure and associated post completion and a second surgical procedure and associated post completion may be compiled and analyzed for updating control parameters (e.g., control program) of a smart device (e.g., before performing a third surgical procedure). For example, a hand-held ultrasonic device may not include a communication array for real-time transfer of data. The hand-held ultrasonic device may transfer surgical procedure information (e.g., usage information) after the surgical procedure or use. The device may transfer the information after a first surgical procedure, or after the first surgical procedure and a second surgical procedure. A hub or smart device may use the information (e.g., usage information) to determine an improvement for the hand-held ultrasonic device (e.g., improve the algorithm or operation). The hand-held device may receive feedback (e.g., during its charge cycle) for its operation in subsequent procedures.

Real-time data collection may enable real-time data usage. The real-time data may be collected. The real-time data may be transformed and/or used. The exchange of the real-time data (e.g., transformed and/or processed real-time data) may be exchanged between surgical systems (e.g., interrelated surgical systems).

The collected data may be transformed in real-time (e.g., a field programmable gate array (FPGA) may be used to collect the data). The transformed real-time data may be used (e.g., acted on) in a real-time flow form (e.g., during a surgical procedure. The organizational structure of the data may be used for a reaction to the data (e.g., using a predefined algorithm to use the data stream to control another portion of the system without understanding the meaning of the data or the trends).

Data may be exchanged and/or moved from electronic health records (HER) to a surgical system, for example, using a database field structure to determine what data streams belong where. Real-time data may be exchanged between control systems and smart systems (e.g., continuous data stream with no context). The real-time data may be used with predefined limits, rules, and/or based on specific conditions being satisfied (e.g., notifications and/or actions).

For example, a lack of signal may imply an issue with a sensor lead and/or a monitored parameter. Either issue may warrant a notification to the HCP. The system may determine that the measurement and/or data is outside specific bounds (e.g., thresholds). For example, an electrocardiogram (ECG) lead may produce data outside expected bounds, e.g., patient crashing or loss of adhesion of the ECG lead. Regardless of the issue, both issues may be brough to the HCP's attention to rectify. In examples of finger pulse oximetry data, data outside of expected bounds may not be urgent to rectify.

Raw data in real-time may be transformed, for example, to be used or acted on by other smart systems in real time. The meaning of the content of data may be understood, for example, to enable action and/or use of the data. For example, the information contained within transferred data may be represented using common language to understand the meaning of the content. Smart systems may be enabled to access and/or process data from multiple sources without losing meaning. The smart systems may integrate that data for mapping, visualization, and/or other forms of representation and analysis. Real-time data may be exchanged between advanced imaging or penetration imaging devices. For example, sharing the location of detected relevant items from a detecting system to a system that may be impacted by the location (e.g., because the detection of the metal may be transferred and highlighted) may imply that a system knows the meaning of the data and highlighting its implication to the system that may depend on the determined location.

For example, overlapping staples in a surgical procedure may be problematic. For an endocutter, staple locations may be problematic in one or more of the following: knife part through the metal; notch knife and reduce performance of knife, dragging staples along a length, grabbing buttress and dragging along. For a circular cutter or circular stapler, staple locations may be problematic in one or more of the following: multiple layers of tissue (e.g., compression selected based on 2 or 4 layers of tissue); circular firing through a linear line; blade damage via contact with metal.

Hidden metal via previous surgeries may be problematic, and may need to be determined for a surgical procedure. Adjacency of metal may be problematic for imaging and/or surgical devices (e.g., cutter, stapler, etc.). Instrument outcomes may be affected based on unknown metal being within an action portion of the jaws of a device. Still resistance during closing (e.g., of a stapler or cutter) hall effect sensor abnormalities, unusual rapid impedance drops, and/or the like may result from an unknown metal. Location of unknown metal may be used to prevent breaking a blade or a short out in the direct contact of an unanticipated piece of metal.

Unidirectional communication may be used to broadcast data known by one system to other interrelated systems. The data may be broadcasted to independent systems via encrypted unidirectional communication, for example.

For example, a surgical procedure step may be identified by a first smart system. The first smart system may broadcast the surgical procedure step to other smart systems. The other smart systems may use the data (e.g., if needed) or discard the data (e.g., if determined to not be useful). Multiple broadcast channels (e.g., with various levels of encryption) may be used and/or available to communicate with other smart systems. Unidirectional communication may be used to transfer data to smarter systems (e.g., from a system with limited capabilities to a system with comparably more capabilities). For example, a system may not be aware of what it is connected to based on a unidirectional nature (e.g., a first surgical system may not be aware that it is connected to a second surgical system). Broadcasting data may allow for a higher chance of sending data to a smart system that may use it (e.g., it may be useful for). A smart system may turn off (e.g., need to turn off) a system with unidirectional communication. Accordingly, the smart system may notify an HCP about a malfunctioning system. The smart system may remove power of to a system (e.g., malfunctioning system).

Systems, methods, and instrumentalities are disclosed associated with adaptation of operation associated with monitoring of surgical devices. A first surgical device may monitor operation of a second surgical device. The monitoring of the second surgical device may affect the operation of the first surgical device (e.g., impact data generation). The first surgical device or the second surgical device may change its operating configuration, for example, based on the monitoring.

For example, a first surgical system may operate using a first operating configuration (e.g., first parameter). The first surgical system may determine first data, for example, using the first operating configuration. The first surgical system may determine that it is being monitored (e.g., by a second surgical system), for example, based on the first data. The first data may be impacted based on the monitoring. The first surgical system may determine an impact associated with the surgical system being monitored (e.g., impact on the first data). The first surgical system may determine an operating configuration to use based on the impact. The first surgical system may compare the impact with a threshold. For example, the first surgical system may determine to use (e.g., maintain) the first operating configuration if the impact is less than the threshold. For example, the first surgical system may determine to use (e.g., change to) a second operating configuration (e.g., second parameter) if the impact is greater than or equal to the threshold. The first surgical system may operate (e.g., generate second data) using the determined operating configuration.

For example, a first surgical system may monitor a second surgical system. The first surgical system may try to remain undetected by the second surgical system. The first surgical system may use a first operating configuration, for example, to monitor the second surgical system. The first surgical system may obtain first data (e.g., via the monitoring) associated with the second surgical system. The first surgical system may determine an impact associated with the second surgical system being monitored. The impact may be determined based on the first data. The first surgical system may determine an operating configuration to use based on the impact associated with the second surgical system being monitored. For example, the first surgical system may compare the impact with a threshold. For example, the first surgical system may determine to use (e.g., maintain) the first operating configuration if the impact is less than the threshold. For example, the first surgical system may determine to use (e.g., change to) a second operating configuration if the impact is greater than or equal to the threshold. The first surgical system may operate (e.g., monitor the first surgical system and/or obtain second data associated with the second surgical system) using the determined operating configuration.

Interrelated systems may exchange data. The interrelated systems may be located within a surgical environment, surgical facility, hospital, and/or the like. Interrelated systems may use the exchanged data to operate, for example, in a surgical procedure, post-procedure analysis, maintenance determination, and/or the like.

Data degradation may occur in exchange of data and/or monitoring of surgical systems providing data. For example, a first surgical system may obtain data associated with a second surgical system (e.g., via monitoring the second surgical system). Monitoring the second surgical system may affect the data, for example, being generated, measured, etc. The data quality may be affected. For example, monitoring a device may affect the processing, latency, and/or sampling rate associated with data collection, generation, and/or measurement. The affected data may lead to inaccurate data. Inaccurate data may affect other systems that use and/or rely on the data being generated and/or collected.

Monitoring of data of a surgical system may be unwanted. A first surgical system may monitor a second surgical system for data. The first surgical system's monitoring may be performed covertly (e.g., without the second surgical system's knowledge) and/or transparently (e.g., with the second surgical system's knowledge). A surgical device (e.g., second surgical system) may not want to be monitored, for example, to preserve the integrity of the data collection and/or operation. The surgical device may not want to be monitored, for example, based on privacy concerns associated with the data collection and/or surgical system's operation. The surgical device may not want to be monitored, for example, by unauthorized access (e.g., only wants to be monitored from trusted sources).

Based on detection of being monitored, a surgical system may want to adapt to preserve data integrity and/or operation integrity (e.g., making sure the data is accurate and unaffected and/or operation/processing is accurate and unaffected). A first surgical system may determine that it is being monitored by a second surgical system. The first surgical system (e.g., surgical system being monitored) may determine (e.g., think) that the monitoring is affecting the data collection and/or first surgical system's operation. The first surgical system may want to adapt operating configurations (e.g., parameters) to avoid data issues (e.g., data inaccuracy) or operation issues (e.g., improper processing/operation). The first surgical system may employ techniques to prevent the monitoring from occurring and/or techniques to prevent the monitoring from affecting the first surgical system's operation and/or data collection.

The second surgical system (e.g., performing the monitoring of the first surgical system) may want to stay covert (e.g., outside of the first surgical system's awareness), for example, to monitor data based on an original operating configuration used by the first surgical system. The second surgical system may not want the first surgical system to change its operating configuration (e.g., parameters). The second surgical system may employ techniques to stay hidden from the first surgical system's detection of monitoring.

A first surgical system may adapt its operating configuration (e.g., parameters) to prevent monitoring (e.g., unwanted monitoring) of its operation and/or data. FIG. 98 illustrates an example flow of adapting operating configurations associated with data being monitored or received. The first surgical system may operate using a first operating configuration. As shown at 54650, first data associated with a first surgical system may be determined (e.g., using a first operating configuration).

A second surgical system may monitor the first surgical system. The monitoring of the first surgical system by the second surgical system may affect the data (e.g., data accuracy) generated by the first surgical system and/or the operation/processing of the first surgical system. The second system may be intercepting, copying, and/or viewing the data generated by the first surgical system.

As shown at 54651, it may be determined that the first surgical system is being monitored. A first surgical system may determine that it is being monitored based on the data it is generating and/or an impact on its operation. For example, the first surgical system may determine a data degradation and/or signal loss. The degradation and/or signal loss may be attributable to being monitored. The first surgical system may determine that data quality is below a threshold (e.g., as described herein, with respect to FIG. 99). Based on the determination that a portion of the first surgical system's operation is being impacted, the first surgical system may determine that it is being monitored.

For example, an impact associated with the monitoring of the first surgical system may be determined (e.g., as shown at 54652 in FIG. 98). The impact may be associated with how the monitoring of the first surgical system is impacting the operation and/or data collection by the first surgical system. The impact may include an impact on the data collection. For example, the impact may be a difference in expected measurements in data as compared with actual recorded and/or sensed measurements. The impact may include a latency and/or delay in sensor measurements. The impact may include an impact on the operation and/or processing of the first surgical system. For example, the impact may include a delay or effect on operation speed.

The first surgical system may adapt its operating configuration (e.g., parameters) based on the determined impact. As shown at 54653, an operating configuration to use may be determined based on the determined impact. The operating configuration may be the same operation configuration (e.g., first operating configuration) being currently used, for example, if the impact is insignificant (e.g., below a threshold). For example, if the monitoring impact on the first surgical system is insignificant and/or negligible, the first surgical system may determine to maintain the current operating configuration. The first surgical system may determine to use a second operating configuration based on the impact (e.g., the impact being greater than a threshold). The second operating configuration may include a modified operating parameter. The second operating configuration (e.g., operating parameter) may be determined based on the determined impact.

The determined operating configuration may include an operating configuration that prevents the monitoring from occurring. The determined operating configuration may include an operating configuration that maintains the integrity of the first surgical system's operation while also allowing the monitoring to continue. For example, the monitoring may be allowed as long as the first surgical system can maintain data and/or operation integrity.

As shown in FIG. 98 at 54654, second data may be determined (e.g., measured, generated, sensed, etc.) based on the determined operating configuration. The second operating configuration may be used to perform processing and/or operation associated with the first surgical system.

FIG. 99 illustrates an example flow of adapting operating configurations associated with data being monitored or received with respect to an impact on the data. As described herein, an impact on operation and/or data collection may be determined for a surgical system being monitored. For example, a first surgical system may determine that a second surgical system monitoring the first surgical system impacts the operation and/or data collection of the first surgical system. The first surgical system may determine whether the impact is severe enough to warrant adapting an operating configuration to maintain the integrity of operation and/or data integrity.

As shown at 54660 in FIG. 99 first data may be determined (e.g., collected, received, sensed, measured, processed, etc.). The first data may be associated with the first surgical system. The first data may be associated with the first surgical system using a first operating configuration. It may be determined that the first surgical system is being monitored (e.g., as shown at 54661 and as described herein (e.g., with respect to FIG. A)). For example, it may be determined that the first surgical system is being monitored based on the first data (e.g., data is inaccurate and/or impacted).

An impact associated with the monitoring may be determined (e.g., as shown at 54662 in FIG. 99). The impact may be associated with a difference in expected data as compared with the actually collected and/or measured first data. In examples, first data may indicate that an EKG lead had a loss of signal at a point in time. The expected data associated with the first data may indicate that there is no EKG lead signal loss. Therefore, an impact may be determined based on comparing the expected data indicating that there is no signal loss at the point in time versus the actual first data indicating signal loss at the point in time. The impact may affect the first surgical system's operation and/or operation associated with other surgical systems that use the data generated from the first surgical system.

An impact associated with a magnitude of a signal may be determined. For example, the system may use a known signal or digital response to determine whether a magnitude of the signal within the line is being monitored (e.g., taped for another reading). For example, the magnitude may differ from the expected known magnitude if the surgical system is being monitored. The surgical system may introduce a known temporal based signal to a main signal and examine the return speed or transmission speed of a phase to determine whether the cables or connectors have a different speed than anticipated.

An impact associated with electronic properties of interconnections may be determined. The electrical properties of interconnections may be monitored and compared with a previous and/or known value, for example, to determine the transmissibility of the interconnections and any intercepts (e.g., monitoring by other devices).

For example, an echo cardiogram (ECG) sensor may use multiple leads (e.g., 3 to 7 leads). The ECG sensor may sense measurements. Interception of a small electrical signal from the ECG signal may lead to signal loss due to being monitored by one or more sources. The primary source may then interpret the inaccurate data (e.g., due to signal loss). The interpretation may be inaccurate (e.g., interpretation may indicate a physical flaw or abnormality that only exists because of the monitoring. The processing of the incorrect data may lead to an incorrect interpretation of a heart issue, which may cause a system to incorrectly operate or send incorrect notifications.

Transformed data may be impacted. For example, transformed data may be intercepted (e.g., based on a monitoring). The interception of the transformed data may introduce a latency in the signal. The latency of the signal, for example, may affect data (e.g., heartbeat shape), which may lead to inaccurate interpretation of the data.

A sensor or smart probe may be used to determine an impact, for example, to determine whether a signal is outside of an expected range. If the signal is determined to be outside the expected range, it may be determined there is an impact and that the system is being monitored.

Signal leads and/or power leads may be used to determine monitoring behavior. For example, a power lead may be used to monitor any additional draw associated with a monitoring system. If the system has the capacity to handle the additional load and the dip in voltage is within specifications, monitoring may be determined to be acceptable (e.g., from a power standpoint). Signal leads may be used to monitor signal lines for signal loss, phase shift, and/or impedance loss.

Impact may be determined based on mapping data streams. For example, mapping of data streams may indicate emitted pulses associated with the surgical systems. Based on the emitted pulses, changes in the emitted pulses may be determined. An impact may be determined based on the determined changes in emitted pulses.

An operating configuration to use may be determined based on the determined impact. A threshold associated with an impact based on monitored may be determined. The threshold may be preconfigured. The determined impact may be compared with the threshold to determine an operating configuration to use (e.g., as shown in FIG. 99).

A surgical system may use identification of monitoring to determine the impact of the monitoring. For example, the surgical system may determine an anticipated impact rating that is associated with the data being monitored. For example, the impact may be associated with a determination of an associated risk of the data being monitored. For example, patient health information, treatment information, business critical information, business intellectual property information, and/or general data may have respective risks of being monitored. At least based on the privacy requirements, risk associated with patient health information may be higher than other types of information, if it is monitored. Business critical information may be associated with data that may include network access keys to critical infrastructure. Business intellectual property information may include information associated with algorithms for operations of devices. General data may include information associated with power consumption levels of a device.

An inherent and/or detected risk associated with the monitoring may be determined. For example, interference with therapeutic functions and/or interference with business functions may be determined. Interference with therapeutic functions may be associated with a monitoring of an EKG lead which may lead to reduced accuracy of the lead (e.g., based on an impact of the electric signal associated with the monitoring). Interference of business functions may be associated with a monitoring of an intellectual property stream which may impact the quality of service of the network, which may reduce the ability of the network to function as intended.

An indirect determination of the impact of monitoring and/or sensing may be determined. A system being monitored may have partial or incomplete information to detect a total impact of the monitoring. The system may indirectly assess the monitoring and/or the impact on the operating system. For example, the system may indirectly assess the monitoring and/or impact based on coordination with other systems and/or known prior correlations. For example, a smart system may detect an anomaly in part of its operation, but may have limited insights into the anomaly. The smart system may alert a secondary system (e.g., surgical hub) which may confirm and/or provide insight on the anomaly. In examples, a system may flag an anomaly if a lead (e.g., new, unexpected lead) is connected to it. A system may flag an anomaly if the leads are incorrect and/or mis calibrated. After a number of occasions of altered leads being detected, the system may flag the error and indicate that the error is likely not because of the leads, but rather an unauthorized use of the system and/or leads.

A first surgical system may determine whether a second surgical system monitoring a data stream is attempting to observe, obtain, or edit data. For example, the second surgical system may be observing or obtaining data associated with device ID, manufacturing ID, device component and/or sub-assembly, calibration data, use and/or count cycle, electrical data (e.g., to check device functionality), patient data, and/or the like. The monitoring performed to observe or obtain data may be performed by surgical hubs and/or maintenance systems to determine maintenance information associated with surgical systems. For example, the second surgical system may be attempting to edit and/or adulterate data, such as, for example, updating calibration information, updating use/count cycle (e.g., device reprocessability), editing algorithm and/or operating specifications (e.g., optimizing device functionality), modifying patient data, and/or the like.

The surgical system may determine the importance associated with the data being monitored. For example, the surgical system may determine whether important data or unimportant data is being monitored. The surgical system may determine whether the system is in a closed loop (e.g., responds to isolated system). The surgical system may determine an amount of data being measured.

The surgical system being monitored may identify a (e.g., each) signal line that is being monitored. For example, the monitored system may monitor the monitored lines to ensure the output signal specifications stay within design requirements of the original signal. If the monitored lines fall outside specified levels, the surgical system may perform one or more of the following: halt signals to minimize hazards that a lower level signal may create, increase output signal to compensate for loss to outside of specified levels, alert for the potential of corrupted signals, and/or the like. The monitored lines information may be collected into a data set for future analysis (e.g., to prevent unforeseen issues in the future).

The surgical system may react to being monitored. For example, the surgical system may select an operating configuration based on the fact that it is being monitored.

In examples a first operating configuration may be selected (e.g., determined) based on the impact. As shown in FIG. 99 at 54663, the first operating configuration may be selected. The first operating configuration may be selected based on the determined impact being less than the threshold. For example, it may be determined that the monitoring has an insignificant impact, and therefore it may be determined to maintain the current operating configuration (e.g., first operating configuration) being used.

In examples, a second operating configuration may be selected (e.g., determined) based on the impact. As shown in FIG. 99 at 54664, the second operating configuration may be selected. The second operating configuration may be selected based on the determined impact being greater than the threshold. For example, it may be determined that the monitoring has an impact (e.g., greater than a threshold) that is negatively affecting operation and/or data collection. Based on determining that an impact is negatively affecting operation and/or data collection, a second operating configuration (e.g., operating configuration parameter) may be determined. The second operating configuration may be predefined and/or determined based on the impact.

In examples, the surgical system may alter functional operations (e.g., reduce functionality) to minimize the impact of the monitoring/surveillance on its operations. For example, a smart system may minimize a number of operating system files, modules, sub-programs being used. This may prevent the monitoring system from impacting the inherent operating of the smart system. For example, the surgical system may alter functional operations by performing one or more of the following: change access or levels of activity; increased protections and/or mitigation; cease of further transmission; denial of audible transmission; video denial; system shutdown; system lockout′ multiplexed data signals; etc.

The surgical system may change access or levels of activity. For example, the change in access or levels of activity may depend on a surgical procedure. For example, it may depend on a surgical procedure step risk level and/or a stage of the surgical procedure. The surgical system may reduce available services, such as, for example, refraining from performing functions, if/when the surgical determines that it is being monitored. For example, a surgical procedure may be planned. Before the surgical procedure begins, the surgical system may determine and/or detect that it is being monitored (e.g., by an unknown entity). The system may disable its communication and alert a user (e.g., an HCP) that it is refraining from functioning, for example, based on the determination and/or detection that it is being monitored. For example, a surgeon may be preparing to fire an endocutter across a critical structure. At the same moment, a surgical system may detect that it is being monitored by an unknown entity. The surgical system may determine (e.g., based on the detected monitoring) a risk of ceasing operation associated with the firing of the endocutter. The surgical system may determine the risk of ceasing the firing of the endocutter is too impactful (e.g., would lead to patient risk). The surgical system may determine to continue operation despite the monitoring, for example, because the determined risk is high (e.g., higher than a threshold).

The surgical system may increase protections or mitigate monitoring in response to a detected monitoring. For example, key strength adjustments may be used. FIG. 101A illustrates an example of a system adapting operating parameters based on a detection that the system is being monitored. A system may determine that a potential adversary is monitoring the transmission of messages. The system may determine to adapt one its operating parameters or features (e.g., encryption keys that are used for coding the communication channels between various devices of the system). For example, the system may change one of the operating parameters or features (e.g., the encryption keys from 128-bits to 256-bits). In an example, the system may make the change for increased protection. The increase may result in increased computational costs, but it may also increase the complexity thereby resulting in reduction of the unwanted monitoring. As shown in FIG. 101A, communication may be exchanged between cluster heads, cluster heads and the ground controller, and/or between cluster heads and the other devices of a cluster. Any of these communication may be altered, for example, based on a detected monitoring. FIG. 101B illustrates an example of communication exchanged between cluster heads, the main controller, and/or devices of a cluster. As shown in FIG. 101B, a surgical system 1 may communicate with one or more of surgical system 1 (e.g., device within the same cluster, Cluster Head 1), the cluster head 1, the main controller, a different cluster head (e.g., cluster head 2 as shown in FIG. X), surgical system 4 (e.g., a device of cluster head 2), and/or the like. For example, surgical system 1 may determine to change an operating configuration. Surgical system 1 may communicate the change of operating configuration to the other devices in the same cluster head, devices of other cluster heads, the main controller, and/or the like. The changes in the operating parameters associated with any of those communications may prevent unwanted monitoring. Other adaptable operating parameters may include dynamic key adjustments, dynamic cyclic redundancy check (CRC) adjustments, changing the encryption protocol (e.g., from a static to a dynamic encryption type) and/or the authentication mechanism between the devices to provide robust communication channels that are less susceptible to monitoring.

The surgical system may cease further transmission, for example, in response to a detected monitoring. The system may halt transmitting further data along a determined section (e.g., compromised section).

The surgical system may deny audible transmission in response to a detected monitoring. For example, an audible jamming tone may be created and/or used. The audible jamming tone may be outside the audible region for staff but within a frequency response range of audible equipment. The audio output may be adjusted. White noise may be inserted. The white noise may target specific frequency ranges.

The surgical system may deny video. For example, a non-visible light source that may be captured within a screen may be used. The non-visible light source may blind a video source.

The surgical system may perform a system shutdown in response to a detected monitoring. The system may delete data from itself. The system may corrupt internal memory, for example, to deny access to its data. For example, repeated attempts to access portions of the system (e.g., that do not feature a clinical aspect, such as, removing a panel that provides access to a circuit card assembly) may result in the circuit card assembly wiping out readable memory so it may not be accessed.

The surgical system may perform a system lockout, for example, in response to a detected monitoring. The system may lock out access to the system for a period of time. The lockout may be in response to repeated unsuccessful access attempted. The system may lock out access to the system based on how the system is being accessed. For example, a system may detect that panels related to the system for accessing a microcontroller assembly (e.g., which is usually only performed during maintenance servicing) or may detect that a service port is actively in use. In response, the system may shut down operation of unrelated portions of the system, for example, as a safety precaution and/or to limit the ability to monitor the system.

The surgical system may multiplex data signals, for example, in response to a detected monitoring. A carrier wave (e.g., larger carrier wave) may be used for transmission of data. The data may be embedded onto frequencies on the larger carrier wave. In examples, an AC power line may include digital information and may serve a purpose of providing data to a system (e.g., in addition to powering transmission of the data).

The surgical system may disable external access points that may not be part of the data collection segment(s). For example, the surgical system may remove accessibility of universal serial buses (USBs), output ports, video outputs and/or audio outputs to external monitors, turning off wireless receivers, and/or the like. The smart system may shift network connections to an alternative, separately secured network (e.g., virtual private network (VPN), etc.).

The surgical system may use offensive counter responses to a determination that it is being actively monitored. For example, the system's counter response may attack the system that is performing the monitoring. For example, an offensive counter response may include one or more of the following: denial of wireless transmission; intentional transmission of incorrect information; noise generation; cybersecurity data attack (e.g., embed attack vectors within data being transmitted); methods of prevention; denial of power; inclusion of a parameter into a control loop to introduce monitoring disturbance, and/or the like.

The surgical system may perform denial of wireless transmission as an offensive counter response. The denial of wireless transmission may include frequency jamming. For example, the system may perform wideband frequency jamming. For wideband frequency jamming, the system may attempt to wirelessly spam available frequencies, for example, to halt data transmission along those frequency channels. For example, the system may perform selective frequency jamming. The system may attempt to wirelessly spam selective frequencies to halt data transmission along the frequency channels.

The surgical system may perform intentional transmission of incorrect information as an offensive counter response. The system may intentionally embed false and/or misleading information within surgical data that may be sent or copied by the monitoring system.

The surgical system may perform noise generation as an offensive counter response. For example, the surgical system may generate an acoustic pulse. The acoustic pulse may be generated within the audible range or outside the audible range. The system may actively generate noise within or external to the audible range, for example, to block signals or drown out relevant information from the other system attempting to monitor or collect it. In examples, the system may become aware that a secondary system is monitoring it. As a result, the system may emit a high frequency signal at random intervals. The high frequency signal may fall outside the audible range, for example, to avoid bothering staff or patients. The high frequency signal may be within the frequency response range of microphones. Therefore, the random intervals of noise may block out signals that may be difficult to filter out.

The surgical system may generate light noise. For example, light source noise may be interfered with using an infra-red light. The surgical system may detect that someone is filming the system. The system may enable an infra-red light source that may not be human detectable. The infra-red light source may impact the quality of recordings.

The surgical system may adopt methods of prevention, such as, for example, changes in communication method, time based actions (e.g., intentionally swapping data across lines at predefined random intervals, for example, where a data pathway may indicate to other lines which data pathways may switch), using multiple data lines, movement of the system, changes in signal voltage, differential signal utilization (e.g., using differential signals to require a competing signal to actively monitor multiple lines to monitor a single data stream), timestamping of data (e.g., rejecting data as falsified if data is not received during a period of specified time), and/or the like. For example, communication methods may include overloading lines to allow multiple communication methods. The surgical system may switch from a first communication (e.g., UART to ModBus) to another, for example, to help prevent the ability of the monitoring system from reading data. Multiple data lines may be used, for example, such as intentionally redundant data lines which may allow switchover in response to data monitoring. Cryptographic splitting may be used. Cryptographic splitting may include intentional transmitting of half of an encrypted message on a first channel and a second half of the encrypted message on a second channel. The receiving system may recompose the message from both data streams. Movement of the system may be used to alert the system that it is being monitored (e.g., detection of physical movements that do not intrinsically make sense. For example, a piece of capital equipment may detect (e.g., using an accelerometer) that it is being moved during a surgical operation. The movement may be analyzed to determine whether monitoring of the equipment is occurring. For example, physical movement of the system may include removing of installed panels. Changes in signal voltage may be used to prevent monitoring. For example, voltage on a signal may be reduced, for example, to lower a signal to noise ratio to reduce the ability of competing equipment to effectively monitor the system without using dedicated equipment. Bandwidth detection and/or monitoring of data transmission physical parameters (e.g., voltage and current of transmitted data) may be monitored.

The surgical system may use denial of power as an offensive counterattack on being monitored. The system may actively deny power to competing systems, for example, so the competing systems do not function. The system may notify OR staff and may request to remove power from a monitoring device. The system may turn off power not a bank of circuits, for example, via a smart power strip.

The surgical system may comprise a process control to monitor certain conditions or measurements that may need to be corrected. The surgical system may include a known monitoring disturbance in such process control and observe reaction of the surgical system's (e.g., the controller of the surgical system) response to the included or injected monitoring disturbance. FIG. 102 illustrates an example of a system adapting operating parameters to disturb a detected monitoring. As illustrated in FIG. 102, a set point or a target value may be defined for a process variable associated with a process. When all conditions of the process are satisfied, the process variable is equal to the setpoint. A disturbance, D may be injected or included in the process that is being monitored. The disturbance may alter the system operation such that some corrective action is required. Once the measurement sensor measures the disturbance, the controller of the control loop may receive a non-zero error signal e(t)=(Set point value, SP-Measured process variable signal, PV). The controller may generate an output to activate the final control element (FCE), which may be an electronic or a mechanical system used to control the manipulated variable. The manipulated variable may control the process variable, which may be the controlled variable part of the process that is being controlled. The process may be controlled until the value of the setpoint is again the same as the process variable.

The surgical system may determine minimum operating parameters for device operation (e.g., for operation in safe mode). For example, minimum operating parameters may include one or more of the following: reverting to manufacturing default; excluding non-original equipment manufacturer (OEM) sensor inputs; airplane mode (e.g., disabling features but preserving laparoscopic video, and/or system switching off everything streaming); eliminating everything except video (e.g., all data displays turn gray); blank out EKG and show heart rate (e.g., only heart rate); and/or the like.

As shown at 54654, second data may be generated based on the selected (e.g., determined operating configuration). The second data may be exchanged with other surgical systems within the surgical environment.

In examples, a surgical system may determine that it is being monitored and determine an operating configuration to use based on the monitoring. For example, first data associated with a first surgical system using a first operating configuration may be determined. The first surgical system may determine that it is being monitored based on the first data. The first data may be impacted based on the first surgical system being monitored. The first surgical system may determine an impact (e.g., impact value) associated with the first surgical system being monitored. The first surgical system may determine (e.g., select) an operating configuration to use based on the impact associated with the first surgical system being monitored. The operating configuration determined may be the first operating configuration or a second operating configuration. The operating configuration may be determined based on the impact being compared with a threshold (e.g., select the first operating configuration if the impact is less than the threshold and select the second operating configuration if the impact is greater than the threshold). The operating configuration may be determined based on the impact being outside a threshold range (e.g., range associated with proper operation of the surgical system). The operating configuration may be determined based on the impact being greater than a threshold associated with the first data being inaccurate and/or incomplete data. The impact of the surgical system being monitored may be determined based on one or more of the following: a type of data associated with the first data; a risk associated with operating using the impacted data; a type of monitoring that is being used to monitor the surgical system; an importance associated with the first data; etc. The monitoring may be associated with observing data and/or editing the data. Based on the selected operating configuration, second data may be generated and/or determined.

In examples, the operating configuration selected based on the determined impact may be associated with preventing the monitoring. For example, the operating configuration may be associated with a safe mode parameter for operating. The safe mode parameter for operating may be associated with a reduction in functionality. The operating configuration selected based on the determined impact may be associated with a selective intervention operating configuration. For example, based on a determination that the impact is greater than the threshold, the determined operating configuration parameter may be associated with reducing collection of protected data and/or ensuring an integrity associated with generated data. The operating configuration selected based on the determined impact may be associated with a transform parameter, wherein second data is generated based on the transform parameter.

A first surgical system may desire to monitor a second surgical system covertly (e.g., without the second surgical system knowing that it is being monitored). For example, the first surgical system may want to covertly monitor a second surgical system so the second surgical system does not change its operating configuration. For example, the first surgical system may want to intercept data associated with the second surgical system without the second surgical system preventing access to the monitoring and/or operating with reduced functionality.

A system being monitored may request ceasing and desisting of intercepting, monitoring, and/or recording of a signal. The system may request cease and desist for parts of data stream that it recognizes as being impacted by monitoring. For example, the system may identify portions of the observation that may be inhibiting, affecting, interfering, or adulterating, and notify the observing system of the needs for the system to stop what it is doing (e.g., because it is negatively affecting the system being monitored). The monitoring may be diminishing the signal received by a main system, or noise or error may be introduced in the signal which may impact an ability to maintain proper resolution and/or speed. Impacts that may be relevant to a monitored data stream may include one or more of the following: changing the balance of the noise/signal ratio; introduction of latency; alteration of calibrated expected magnitude or ratio of signals; adulteration of headers or packet identifiers; impact on phase of signal oscillation; signal interference from recording system; creation of electro-magnetic fields that introduce error into a shielded system; drifting the relative ground to which the signal is measured from; bleed-over of the monitor signal back into the primary line through capacitive coupling or other electrical coupling means which could cancel out, reinforce, or additively or substantively impact a primary signal.

A system being monitored may communicate with surrounding systems (e.g., smart systems in the surgical environment or other devices or people in the surgical environment). The communication may include a warning and/or heads up to enable other smart systems to proactively prepare for monitoring. Proactive preparation may include enabling defensive protocols, such as, for example, using encryption codes and/or keys. For example, a visualization system may detect an intrusive system monitoring it, which may be physically attached to the system. The visualization system may notify a separate smart system that it has been compromised.

To covertly monitor a second surgical system, the first surgical system may adapt its operating configuration to avoid detection. For example, the first surgical system may use an operating configuration associated with minimizing an impact on the second surgical system's operating and/or data collection.

A surgical system may transform data based on a determination that it is being monitored. The transformation of the data may be performed to confuse the monitoring system. For example, the data streams may be transformed. The coordinate system (e.g., coordinate transformations) may be transformed. For example, the coordinate system information may include a number line, cartesian coordinate system, polar coordinate system, cylindrical and/or spherical coordinate system, homogenous coordinate system, curvilinear coordinates, log-polar coordinate system; Plucker coordinate system; generalized coordinates; canonical coordinates; barycentric coordinate system; trilinear coordinate system; and/or the like. The surgical system may transform data between the various coordinate systems to confuse a monitoring system.

Adaptive behavior of a surgical system may be used based on monitoring by another system that may not be stopped. For example, the system may determine portions of the control system that may be affected by monitoring and may remove that portion from a closed loop control aspect of the affect system control. Identification of sensor feeds that are adulterated, impacts, or no longer reliable due to external monitoring and/or indicating that feed as unreliable or requiring supplementary conformation may be performed. A closed loop control signal may be replaced with an open loop with a predefined parameter. Exchanging an effective closed loop control signal with an alternative signal (e.g., that is not monitored or not affected by monitoring) may be used. Additional oversight monitoring or constraining adjustable parameters of a closed loop system may be applied, for example, to ensure affected control loop adulterated sensor feeds do not have a large impact on the operation of a closed loop operating system.

FIG. 100 illustrates an example flow of a surgical system changing its operating configuration to avoid being detected that it is monitoring a different surgical system. A first surgical system may monitor a second surgical system. As shown at 54670, first data associated with a second surgical system using a first operating configuration may be obtained (e.g., by a first surgical system).

As shown at 54671, an impact associated with the second surgical system being monitored may be determined. For example, the first surgical system may determine whether its monitoring of the second surgical system is impacting the operation and/or data collection of the second surgical system. The impact may be determined as described herein with respect to FIG. 99. The first surgical system may want to minimize the impact on the operation of the second surgical system, for example, so it remains undetected. If the impact on the operation of the second surgical system is noticeable, then the second surgical system may adapt its operating configuration to prevent the first surgical system from monitoring the second surgical system.

As shown at 54672, an operating configuration to use for the first surgical system may be determined, for example, based on the determined impact. The first surgical system may select a second operating configuration (e.g., current operating configuration) for example, if the determined impact is below a threshold (e.g., the second surgical system would not notice the impact and/or the impact is negligible). The first surgical system may select a third operating configuration, for example, if the determined impact is above the threshold. The third operating configuration may include a parameter that is adapted to avoid detection from the second monitoring system.

As shown at 54673, second data may be obtained from the second surgical system using the determined operating configuration associated with the first surgical system. The first surgical system may continue to monitor and/or intercept data from the second surgical system using the determined operating configuration.

A first surgical system may adapt its operating configurations to covertly monitor a second surgical system and avoid detection of the monitoring. The first surgical system may obtain (e.g., using a first operating configuration) first data associated with a second surgical system. The first surgical system may determine an impact associated with the second surgical system being monitored, for example, based on the first data. The impact may be associated with inaccurate data and/or degraded data. The first surgical system may determine an operating configuration to use (e.g., maintain the first operating configuration or determine a second operating configuration) based on the determined impact. The operating configuration may be determined based on comparing the determined impact to a threshold (e.g., range of values). The determined operating configuration may include using an operating parameter. The operating parameter may be associated with an impedance. For example, a first operating configuration may be associated with using a first impedance and the second operating configuration may be associated with using a second impedance. The operating configuration may be determined, for example, based on a type of data or a level (e.g., privacy level) associated with the data. The first surgical system may continue to monitor the second surgical system and/or intercept data associated with the second surgical system. Second data associated with the second surgical system may be obtained, for example, using the determined operating configuration.

In examples, a surgical system may monitor another smart system using a predefined means to export the data from one system to another. The data export may be a unidirectional stream. The data export may not be monitored by the first system to determine whether systems are watching the output.

For example, data export streaming may be used for external use or monitoring. A first smart system may have a data output port that streams data out for other systems to use. The first smart system may not monitor or care if the data is monitored or not. In examples, an EKG output of a heart rate monitor may be connected to other systems, for example, allowing them to monitor the feed provided by the system (e.g., without interfering with the system's monitoring and display of patient heart rate). The output may be ported into the imaging system of the catheter ablation system and/or displayed on the screen adjacent to a live image (e.g., allowing the HCP to see the heartbeat irregularities visually as well as a neuro component).

For example, data export streaming may be used for oversight. A smart system may be designed to be a first portion of a cooperative biomarker monitor, a leading indicator to a second monitored system, and/or a conformational means of the second control system. A raw or transformed data stream may be provided from the system with no feedback to a receiving system. The receiving system may use the provided secondary stream to confirm or ensure that its measure or monitored aspect is correct, the signal did not drift, and/or the stream is not out of sync with real-time aspects of the procedure.

In examples, a heartbeat may be measured by a blood pressure cuff. The blood pressure cuff may be capable of measuring the pressure within the extremity to which it is attached. The blood pressure cuff may include a built in beat monitor used to determine if pressure is exceeded and when it re-establishes. Automated cuffs may have an orientation issue if placed on the extremity (e.g., the internal sensor may need to be aligned to the artery of the extremity, such as, for example, the tube of the automated cuffs may need to be in the hinge of the elbow and the cuff may need to be an inch above the elbow). In surgery, a patient may be draped off to HCPs may no longer see the cuff or extremity as they are on the other side of the drape. The patient may be instrumented with EKG leads and a pulse oximeter (e.g., other smart systems that monitor heartbeat rate and timing). If the EKG leads and pulse oximeter supply the beat cadence to the blood pressure cuff it may differentiate between erroneous measurements of its monitor of blood flow from irregularities of the heartbeat itself.

Surgical systems may synchronize smart system outputs. An output stream from a first smart system may be provided to input into a second smart system, for example, to ensure that the two signals are synchronized and the use of the data together is ensured to be in sync. A third smart system may export a stream that two cooperative monitors use, or the third smart system may use a stream from one cooperative system to synchronize a second stream. In examples, an EKG monitoring system and a flexible scope catheter view may be used in cooperation during a surgical procedure (e.g., AFIB focal ablation). In between heartbeats, the catheter RF electrode may be advanced and single point ablation may be performed, and retracted before a subsequent heartbeat. The catheter control and imaging may be performed within the same smart system, but there may be a lag between detection and display of the video feed. An external heartbeat measure or EKG may be used to synchronize the systems. The EKG (e.g., used to determine whether ablation sufficiently stopped the irregular beat) may determine whether follow on point ablations are necessary. Both systems may benefit from ensuring synchronization of the signals to real-time patient heartbeats.

Surgical systems may listen to each other, for example, other systems within an audible range. An audible pulse of a first system may be used to alert a second system. The audible pulse may indicate an action to be taken. The audible pulse may indicate an error has occurred. For example, an audible emission may be generated and emitted, for example, to indicate that an endocutter is about to be fired.

Surgical systems may perform visual observation of other systems. The visual observations may include a light source. A light emitted diode (LED) alignment system may be used to enable visual observation of systems.

Signal surveillance of data streams via interception of extractions of external systems may be performed. For example, intercepted monitoring may include where a data stream is intercepted, copied, and parallel transmitted. The signal may be extracted from physical properties of a primary system's electrical or mechanical system. A second signal beside the first system primary use may be generated, for example, without the knowledge and/or cooperation of the first system. The second signal may be monitored and/or used to control a closed loop aspect of a second smart system.

For example, multi biomarker smart monitoring systems (e.g., CareOne, which may monitor Po2, CO2, ECG, temperature, blood pressure, etc., and a ventilator), may intercept return feeds or display outputs to obtain data. A system monitoring a data stream may show a graph, for example, based on the data streams. For example, a ventilator may intercept data from a CareOne device and show a graph representing a tidal volume changing to balance bloodgases CO2 from CareOne with exhalation gas CO2 from the ventilator in a closed loop manner.

A portion of a monitoring feed or related aspect of a smart system may be tapped into, for example, to enable simultaneous monitoring of a data feed by multiple systems or simultaneous monitoring of systems monitoring each other. For example, non-smart smoke evacuators may use a current sensor buckle that may be placed of a wire of an RF monopolar system wire that may detect the presence or absence of current in the cable (e.g., to know whether the generator is activated). The non-smart smoke evacuator may turn on when a current is detected. The turning on when a current is detected may be a form of covert monitoring. A smart smoke system may react to different intensities of activation with different velocities of motor speed. A smart smoke system may consider combined frequency and intensity of activation, for example, to adjust magnitude of smoke evacuation.

Data streams and/or a series of data points that have been intercepted, copied, and resent may be synchronized to other signals. Timestamping and/or packet ordering of data may be used to synchronize data. Intentional delay may be introduced, for example, to synchronize data. Dynamic delays in hardware or software (e.g., which may allow time to copy data and transmit) may be used. Dynamic delays may include speeding up slower pathways and slowing down faster pathways for better synchronization. Estimation theory may be used to create an illusion that a system is monitoring faster than it actually is. Mathematical synchronization may be used. Prior knowledge of system latency may be considered. Accommodation of the latency may be used to determine an optimized injection point. For example, a wirelessly enabled monitor may receive (e.g., need to received) messages within 50 mS of transmission). A system may artificially timestamp data to show it has been there in the correct order. A system may bypass other parts of the system where actions may be performed faster so the monitor can receive the data within 50 mS.

Signal loss and/or degradation minimization due to monitoring (e.g., surreptitious surveillance) may be mitigated and/or minimized. For example, fingerprints of surveillance may be removed to avoid detection of monitoring. In examples, temporal sampling, throughput, or speed of signal transmit may impact minimization. Phase matching and/or periodic spatial modulation of a coefficient may be used to minimize signal loss and/or degradation.

Dynamic impedance matching may be used to minimize an effect on a data signal from surveillance. For example, signal loss, impedance mismatch, or resistive change to the data stream may be monitored by intercepting or measuring the signal over time. A correction factor may be applied to the line impedance, resistance, or a signal boost, for example, to adjust for losses and/or changes induced by taking the measure itself or attaching the sensing system in-line with the signal. Dynamic impedance changes may be used. The system may use the impedance of the system as a flag to indicate if something has been attached to the system and may be monitoring it. A block box interception system may be used in-series with a signal and measure an expected impedance of the line before its introduction. The system may use parallel resistors to simulate the expected impedance in the parallel circuit to trick the independent system into thinking the black box does not exist in the system (e.g., to avoid detection).

Coupling of signals may be used to minimize an effect on a data signal from surveillance.

A microstrip may be used to electromagnetically couple a signal without physically interacting with it. A high speed data line may be monitored similar to an AC line. Overlaying of digital communication stream over an AC signal may be performed.

A man-in-the-middle technique may be used to minimize an effect on a data signal from surveillance. A monitoring circuit may be inserted to mimic an input and/output impedance of a larger circuit and may mimic associated amplitudes or other properties/characteristics. A power booster may be used to maintain signal level integrity so the signal comes out as it went in.

High impedance monitoring may be used to minimize an effect on a data signal from surveillance. A high impedance circuit may be used to monitor a signal with a minimal possibility of being monitored.

Type of data and/or level of data may be considered in determining how to monitor a data stream. For example, monitoring of therapeutic signals may use different monitoring techniques as compared with monitoring data signals. Risk based assessments may be performed for monitoring therapeutic data streams. Monitoring may change and/or adapt based on the type of data coming through. For example, if low energy sensing pulse from a generator is received, a different monitoring technique may be used from a data signal coming from a main therapeutic signal. A system may be interrogated to determine a monitoring technique. A change in monitoring technique may be used based on priority system information (e.g., system specification information, system data information).

Primary signal compensation may be used to minimize an effect on a data signal from surveillance. A primary signal may be compensated to accommodate that a monitoring signal may impact it. The monitoring network may acknowledge that it may impact the data, but may compensate the original signal so original characteristics are maintained. For example, a high frequency signal may have a known voltage peak, slew rate, and current draw (e.g., functions of its matched circuit). A monitoring circuit may be highly inducive, for example, due to additional wiring that is used to monitor it. A compensating circuit may be additionally installed to have more capacity and boost the signal to mimic the signal in addition to the monitoring circuit.

A data signal from a sensor to an amplifier and/or circuit board may be measured without impacting the signal strength or measure. Remote monitoring by an independent external source may be used. A data signal may be measured in line and a technique to resupply the data signal using transformers may be used.

An external supplemental monitoring system may be used to monitor capital box operation. For example, a smart power distribution system strip and/or surge protector may be used to determine which device is which (e.g., manually determine). Power may be used from the wall to check magnitude and plugs and tones may be used to determine which magnitude goes to which device. External monitoring systems may use secondary sensors to determine what it wants to measure (e.g., smart pad under a generator).

A smart device may be operated outside a larger communication network (e.g., may not be coupled to the larger communication network). The smart device external to the larger communication network may be monitored. For example, a smart network may have fuse level control over a power supply of the external device. Smart safety control of a Cone Beam CT may be enabled based on sensing, equipment, and/or location of HCPs. Localized communication networks may be interceded. For example, a system may use a denial of service style mitigation, for example, such as, for example, occupying existing local Bluetooth advertising channels, which may prevent a device from successfully pairing with another device.

An external system (e.g., uncontrolled system) may have impact on a smart system within the smart network. For example, the smart network may be adjusted to compensated for an uncontrolled system's action. The smart network may be adjusted, for example, to avoid unintended interaction. For example, an advanced monopolar energy generator that may not include an output monitoring port but whose activation may interfere with a patient biomarker monitoring system (e.g., EKG, pulse rate, PO2) may be used. The smart network may be adjusted to compensate for the uncontrolled system's action. For example, a smart system may control a smart AC distribution panel. The AC system may not be able to control what is plugged into it, but it may coordinate with controlled elements to mitigate risks, for example, such as dynamically enabling or disabling AC power ports to prevent tripping an entire breaker.

Comprehensive data may be collected. Expansion of the collected comprehensive data may occur from monitoring systems to determine the comprehensive data. Aggregation of monitored and communicated data may be performed to determine efficiencies.

Covert monitoring and/or surveillance may be non-intrusive. For example, a system may monitor actions associated with a surgery robot. The system may determine wear, maintenance intervals and/or the like based on the action, correction, or recalibrating that is used. The monitoring for the usage may affect the performance of the actions during surgery. The monitoring of the usage data may be performed at the end of the surgery, for example, to avoid impact on the actions.

Device operation may be adapted, for example, based on monitored patient biomarker data. Biomarker patient monitoring may enable identification of physiologic reactions that may affect smart device operation. For example, peripheral blood flow dilation may be linked to hypothermia, which may affect whether a patient heating system is used (e.g., based on monitoring the hypothermic indicating data). Controlled rate of change of the closed loop to adapt the magnitude of heat transfer may be used.

For example, blood sugar, heart rate variability, blood pressure, and/or the like may be monitored to determine implications associated with metabolic load or blood flow rate. The monitored information may be used to influence devices associated with drug uptake, core temperature generation, blood or tissue oxygenation, sedation dosage and/or depth, and/or the like.

For example, externally applied physiologic adaptation may drive a physiologic reaction that may drive a desired outcome. Use of therapeutic hypothermia to reduce the body's need for oxygen and changing the ratio of oxygenated blood to “critical” organs to re-balance CO2 based on O2-CO2 patient biomarker monitoring and mechanical ventilation constraints may be performed. Localized organ cooling may be used to induce localized therapeutic hypothermia to minimize ischemic tissue damage and reduce local bleeding relative to surgical intervention.

A first system may monitor a second system by monitoring the display of the second system. The first system may use a camera to monitor the room or display screen of the second system. The first system, based on the monitoring, may determine power level, activation, times, and/or the like. The monitoring via the display screen may not impact or be detected by the second system. For example, an electrosurgical generator may be monitored to identify which type of energy (e.g., bipolar, monopolar, etc.) is in use, power level settings, cut modes, coagulation modes, number of firings, etc., for example, which may be displayed on the display screen.

Encryption key passing may be used, for example, to covertly monitor a system. FIG. 103 illustrates an example of encryption key passing to enable discreet monitoring of devices. A host or central device (e.g., in Bluetooth connection to the device) may pass a copy of its encryption key to a surgical hub or surveilling device, for example, to enable the device to additionally monitor the communication between the device and host. The device may not have knowledge that the communication is being monitored, for example, because of the use of encryption key passing.

Identification systems may be used, for example, to covertly monitor a system. A system may be aware of adversaries within the operating room. The awareness may include awareness of devices that try to infiltrate surgical devices. A system may determine that a connected smart system used to acquire system information may be connected. A notification may be sent to notify to remove the competing device or the indicate to be on high alert for intrusion.

A system may be aware of friendly devices within an operating room. A visual identification marking may be used to detect friendly devices. Notification may be generated to HCPs to indicate compatible devices. A confirmation or rejection of compatible devices may enable the smart system to learn. Instructions may be automatically displayed for friendly devices.

A system may use enabling technology. For example, a transponder may be used (e.g., transmitter and responder). The transponder may change responses based on received transmissions. A system may use automatic gain control, for example, to spoof real data by drowning it out with clutter and/or noise.

1. A method for operating a surgical smart system in an operating room, the method comprising:
accessing a database, wherein the database comprises a surgical manifest and a lookup table of surgical equipment;
identifying, using a camera of the surgical smart system, a piece of equipment present in the operating room by comparing the piece of equipment with information in the database;
determining data from the identified piece of equipment using optical character recognition (OCR); and
displaying the data, including information from the piece of equipment, on a display of the surgical smart system.

2. The method of claim 1, wherein the method further comprises:
quantifying a degree of interactivity between the surgical smart system and the piece of equipment; and
modifying the operation of the surgical smart system based on the quantified degree of interactivity.

3. The method of claim 1, wherein determining data from the identified piece of equipment using OCR includes:
identifying an identification number from the identified piece of equipment; and
recording the identification number in association with the piece of equipment in the surgical manifest.

4. The method of claim 1, wherein the method further comprises:
establishing an operating parameter based on an operation between the surgical smart system and the piece of equipment;
determining whether the established operating parameter is outside a threshold; and
adjusting the operating parameter on a condition that the parameter is outside the threshold.

5. The method of claim 1, wherein determining data from the identified piece of equipment using OCR comprises:
updating the surgical manifest within the lookup table using the data from the identified piece of equipment; and
saving the updated surgical manifest in the database.

6. The method of claim 1, the method further comprising:
cross-referencing the identified piece of equipment with the surgical manifest; and
generating an alert on a condition that the piece of equipment is not found in the surgical manifest.

7. The method of claim 1, the method further comprising:
determining an operational status of the piece of equipment using the OCR-determined data;
displaying the operational status on the display of the surgical smart system; and
generating an alert on a condition that the operational status indicates that the identified piece of equipment is not suitable for use.

8. The method of claim 1, the method further comprising:
determining whether wired communication with the piece of equipment is possible;
providing instructions, via a user interface of the surgical smart system, for connecting the surgical smart system to the piece of equipment based on the determination that wired communication with the piece of equipment is possible; and
adjusting an operation of any of the surgical smart system or the piece of equipment based on the user connecting the surgical smart system to the piece of equipment.

9. A surgical smart system in an operating room, the surgical smart system comprising a processor:
the processor configured to:
access a database, wherein the database comprises a surgical manifest and a lookup table of surgical equipment;
identify, using a camera of the surgical smart system, a piece of equipment present in the operating room by comparing the piece of equipment with information in the database;
determine data from the identified piece of equipment using optical character recognition (OCR); and
display the data, including information from the piece of equipment, on a display of the surgical smart system.

10. The surgical smart system of claim 9, wherein the processor is further configured to:
quantify a degree of interactivity between the surgical smart system and the piece of equipment; and
modify the operation of the surgical smart system based on the quantified degree of interactivity.

11. The surgical smart system of claim 9, wherein the processor configured to determine data from the identified piece of equipment using OCR comprises the processor being configured to:
identify an identification number from the identified piece of equipment; and
record the identification number in association with the piece of equipment in the surgical manifest.

12. The surgical smart system of claim 9, wherein the processor being configured to determine data from the identified piece of equipment using OCR comprises the processor being configured to:
update the surgical manifest within the lookup table using the data from the identified piece of equipment; and
save the updated surgical manifest in the database.

13. The surgical smart system of claim 9, wherein the processor is further configured to:
cross-reference the identified piece of equipment with the surgical manifest; and
generate an alert on a condition that the piece of equipment is not found in the surgical manifest.

14. The surgical smart system of claim 9, wherein the processor is further configured to:
determine an operational status of the piece of equipment using the OCR-determined data;
display the operational status on the display of the surgical smart system; and
generate an alert on a condition that the operational status indicates that the identified piece of equipment is not suitable for use.

15. The surgical smart system of claim 9, wherein the processor is further configured to:
determine whether wired communication with the piece of equipment is possible;
provide instructions, via a user interface of the surgical smart system, for connecting the surgical smart system to the piece of equipment based on the determination that wired communication with the piece of equipment is possible; and
adjust an operation of any of the surgical smart system or the piece of equipment based on the user connecting the surgical smart system to the piece of equipment.

16. A surgical smart system in an operating room, the surgical smart system comprising a processor:
the processor configured to:
access a database, wherein the database comprises a surgical manifest and a lookup table of surgical equipment;
identify a piece of equipment present in the operating room by comparing the piece of equipment with information in the database;
determine data from the identified piece of equipment; and
display the data, including information from the piece of equipment, on a display of the surgical smart system.

17. The surgical smart system of claim 16, wherein the processor is further configured to:
quantify a degree of interactivity between the surgical smart system and the piece of equipment; and
modify the operation of the surgical smart system based on the quantified degree of interactivity.

18. The surgical smart system of claim 16, wherein the processor configured to determine data from the identified piece of equipment comprises the processor being configured to:
identify an identification number from the identified piece of equipment using optical character recognition (OCR); and
record the identification number in association with the piece of equipment in the surgical manifest.

19. The surgical smart system of claim 16, wherein the processor is further configured to:
cross-reference the identified piece of equipment with the surgical manifest; and
generate an alert on a condition that the piece of equipment is not found in the surgical manifest.

20. The surgical smart system of claim 16, wherein the processor is further configured to:
determine whether wired communication with the piece of equipment is possible;
provide instructions, via a user interface of the surgical smart system, for connecting the surgical smart system to the piece of equipment based on the determination that wired communication with the piece of equipment is possible; and
adjust an operation of any of the surgical smart system or the piece of equipment based on the user connecting the surgical smart system to the piece of equipment.
